(19) United States
(12) Patent Application Publication
(10) Pub. No.: US 2026/0256807 A1
(43) Pub. Date: Sep. 3, 2026

(54) SUBSTITUTED PYRAZOLES AND IMIDAZOLES AS OREXIN RECEPTOR ANTAGONISTS COMPOSITION, METHODS FOR TREATING NEUROLOGICAL AND PSYCHOLOGICAL DISORDERS

(71) Applicant: HAGER BIOSCIENCES, LLC, Bethlehem, PA (US)

(72) Inventors: Belew MEKONNEN, Bethlehem, PA (US); Hemantbhai PATEL, Bethlehem, PA (US)

(73) Assignee: HAGER BIOSCIENCES, LLC, Bethlehem, PA (US)

(21) Appl. No.: 19/104,451

(22) PCT Filed: Aug. 25, 2023
(86) PCT No.: PCT/US2023/031132

§ 371 Date: Feb. 18, 2025

Related U.S. Application Data
(60) Provisional application No. 63/400,894, filed on Aug. 25, 2022.

(51) Int. Cl. A61K 31/551; A61K 31/444; A61K 31/506; A61K 31/5383; C07D 471/04; C07D 487/04; C07D 498/04; C07D 519/00

(52) U.S. Cl. CPC A61K 31/551; A61K 31/444; A61K 31/506; A61K 31/5383; C07D 471/04; C07D 487/04; C07D 498/04; C07D 519/00

(57) ABSTRACT

The disclosure relates to substituted Imidazole and Pyrazole ring system derivatives of compounds that are antagonists of orexin receptors, and which are useful in the treatment or prevention of neurological and psychiatric disorders and diseases in which orexin receptors are involved or implicated. This disclosure is also directed to pharmaceutical compositions comprising these compounds and the use of these compounds and compositions in the prevention or treatment of such diseases in which orexin receptors are involved.

### RELATED APPLICATIONS

This application claims priority to U.S. Provisional Ser. No. 63/400,894 filed on Aug. 25, 2022, which is incorporated into this application in its entirety.

### FEDERALLY SPONSORED RESEARCH

This invention was made with government support under Cooperative Agreement UF1DA054817 awarded by the National Institute of Drug Abuse. The government has certain rights in the invention.

### FIELD OF THE DISCLOSURE

This disclosure relates to and provides compounds, compositions, and methods for making and using orexin antagonists to treat or ameliorate human and animal diseases as therapeutic agents. In particular, any pathological disorder in which both types of orexin receptors are pharmacologically involved or implicated. These important therapeutic applications include but are not limited to treating central nervous system (CNS) disorders and neurological diseases that involve or are modulated by orexin receptors including but not limited to disorders that are responsive to orexin receptor antagonists, e.g., substance addiction and dependence, cognitive impairment, Alzheimer's disease (AD), posttraumatic stress disorder (PTSD), schizophrenia, panic, anxiety, autism, pain, and depression.

### BACKGROUND OF THE DISCLOSURE

The orexins (also known as hypocretins) are comprised of two excitatory hypothalamic neuropeptides: orexin A (OX-A: a 33 amino acid peptide) and orexin B (OX-B: a 28 amino acid peptide). They were simultaneously discovered in 1998 by two research groups searching for new signaling molecules, (1) Sakurai and co-workers (who named them orexin-A and -B) (Sakurai, T. et al, Cell 1998, 92, 573) and (2) de Lecea and co-workers (who named them hypocretin 1 and 2, respectively) (de Lecea, L. et al, Proc. Natl. Acad. Sci. U.S.A. 1998, 95, 322.). These neuropeptides are endogenous ligands for two G protein-coupled receptors (GPCR) named OX₁R and OX₂R (also referred to as Hcrt1 and Hcrt2, respectively) and are derived proteolytically from the same precursor peptide called pre-pro-orexin polypeptide (Sakurai T., et al. The Journal of biological chemistry. 1999; 274, 17771-17776). Though structurally related, the binding affinities of these endogenous ligands for the two GPCRs differ. Orexin A binds to OX₁R with about 100-fold higher affinity than Orexin B, whilst both Orexin A and Orexin B bind to OX₂R with the same affinity (Kodadek, T.; Cai, D. Mol. BioSyst., 2010, 6, 1366-1375). Soon after the discovery of orexins, modulation of the orexin signaling was originally considered for potential novel treatments of narcoleptic or insomniac patients since the role of orexins in regulation of sleep and wakefulness was well-studied and understood, and the discovery of small-molecule modulators of orexin signaling facilitated the development of this class of compounds. Narcoleptic patients show a diminished activity in hypothalamic orexin neurons thereby lowering the amounts of circulating orexins in the cerebrospinal fluid. In contrast, activation of orexin neurons maintains wakefulness and arousal. The effects of orexin signaling on feeding and energy homeostasis were also established earlier and found to be coordinated to the sleep-wake cycle (Kodadek, T.; Cai, D. Mol. BioSyst., 2010, 6, 1366-1375). More recent studies have established the role of orexin signaling in other key physiological pathways such as neuroendocrine functions (Inutsuka, A.; Yamanaka, A. Front. Endocrinol. 2013, 4:18. doi: 10.3389/fendo.2013.00018), glucose metabolism (Tsuneki, H., et al., Endocrinology, 2016, 157, 4146-4157), stress-adaptive responses (Xiao, F., et al. Neuropharmacology, 2013, 67, 18-24), and addiction/reward-seeking (Aston-Jones, G., et al. Brain Res., 2010, 1314, 74-90). Small molecule orexin antagonists have been broadly categorized into three classes based on their overall receptor selectivity profiles: (1) DORA (dual-acting, or non-selective OX₁R/OX₂R antagonists), (2) SORA-1 (selective OX₁R antagonists), and (3) SORA-2 (selective OX₂R antagonists). It has been shown that while OX₂R knockout mice and OX₁R/OX₂R double knockout mice both show a narcoleptic phenotype, the effect is very muted in OX₁R knockouts (Wang C., et al. Neurosci., 2018, 11, 220. doi: 10.3389/fnmol.2018.00220). Additionally, both DORA and SORA-2 compounds inhibit wakefulness, but SORA-1 compounds do not—thus suggesting that narcoleptic effects are mediated through OX₂R or a combination of OX₁R and OX₂R, but not through OX₁R alone. Thus, it is clear that the discovery and development of selective orexin antagonists is crucial to the advancement of this field but most importantly to the development of therapeutic agents for dysregulated biological processes that involve the orexin receptor; especially for non-sleep related indications such as substance addiction.

### SUMMARY OF THE DISCLOSURE

This disclosure addresses the aforementioned therapeutic and/or other needs by providing compounds of Formula I and II:

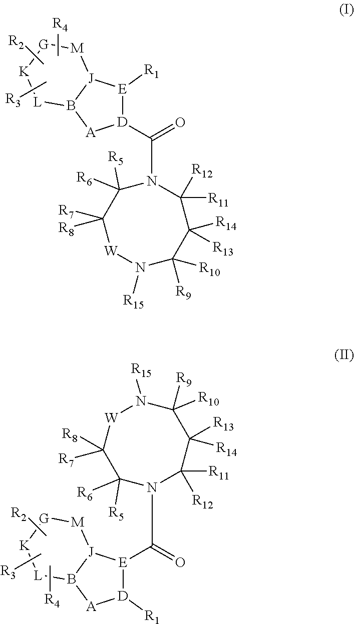

wherein the variables are as defined herein, including any pharmaceutically acceptable salts, solvates, adducts, polymorphs, and isomers thereof, as well as compositions comprising the same. Compounds of Formula I and/or II (and/or preferred embodiments thereof) and compositions comprising the same can be used to treat the conditions such as those described herein, such as through activity as orexin receptor antagonists. The compounds and/or compositions disclosed herein can be antagonists for one or more orexin receptor antagonists, such as either one of OX₁R or OX₂R, or both OX₁R or OX₂R. The compounds and/or compositions disclosed herein can be selectively more antagonistic for one or more orexin receptor as compared to another orexin receptor, such as in being more or less antagonistic of OX₁R or OX₂R as compared to the other orexin receptor. Thus, the compounds and/or compositions of the same can be referred to herein as “Orexin Receptor Antagonists”.

In some embodiments, the present invention also provides compositions that comprise the above compounds or a pharmaceutically acceptable salt thereof. In another aspect of the invention, there is provided a method for treating CNS disorders such as, among others, substance addiction and dependence, posttraumatic stress disorder (PTSD), schizophrenia, panic, anxiety and depression, pain, cognitive impairment and Alzheimer's disease (AD) in a subject in need or at risk thereof, the method comprising the step of administering to said subject a therapeutically effective amount of orexin receptor antagonists or a pharmaceutically acceptable salt thereof. In certain embodiments of the invention, the orexin receptor antagonists or a pharmaceutically acceptable salt thereof could be formulated to be administered periodically, for example every 3, 6 to 24 hours as deemed clinically beneficial. Other aspects and embodiments are contemplated herein as would be understood by those of ordinary skill in the art.

### DETAILED DESCRIPTION OF THE DISCLOSURE

This disclosure provides fused six (6) and five (5) membered ring system derivatives of Formulas (I) & (II) wherein the fused six (6) and five (5) membered rings are as described structurally, pharmaceutically acceptable salts thereof, compositions thereof, and methods for preparing and using the same. In some embodiments, this disclosure describes pharmaceutical compositions containing one or more compounds of Formulas (I) & (II), the preparation of the same, and to their use as pharmaceuticals and therapeutic agents, particularly (i.e., in preferred embodiments) to their use as orexin receptor antagonists. These novel agents as described by Formulas (I) & (II) which are non-peptide antagonists of human orexin receptors and are potentially useful in the treatment of disorders relating to orexinergic dysfunctions; including but not limited for such disorders like substance addiction (e.g., cocaine, opioids, alcohol and the like), anxiety, panic, cognitive dysfunctions, mood, or appetite, sleep, Alzheimer's Disease (AD), metabolic syndrome, pain, and/or hypertension. In preferred embodiments, the compounds disclosed herein are of great therapeutic value in the treatment of anxiety disorders, addiction disorders, and/or sleep disorders.

In some embodiments, this disclose provides compounds of Formulas (I) and (II):

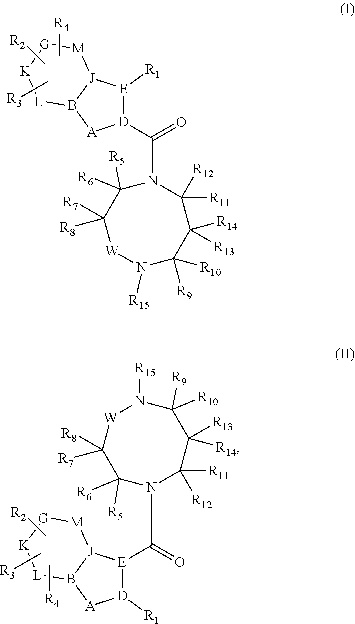

wherein:
R₁, includes E in which is Carbon (C) but not Nitrogen (N), and E is linked to J or D with a double bond, and R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₄, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ and R₆ are independently selected from the group consisting of H, CH₃, alkyl, substituted alkyl, (C₁₋₃)fluoroalkyl, cycloalkyl, R₆ connected to either R₁₀ or R₁₁ or R₁₃ as alkyl to form a (C₁₋₃)alkyl bridge cyclic structure, and R₅ and R₆ linked together to form a spiro moiety such as but not limited to cyclopropyl, or cyclobutyl;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₁₀ or R₁₁ as alkyl to form a (C₁₋₃)alkyl bridge cyclic structure:
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, and R₁₁ or R₁₂ are connected to either R₇ or R₈ as alkyl to form a (C₁₋₃)alkyl bridge cyclic structure, wherein in some embodiments R₁₁ and R₁₂ linked together can form a spiro moiety such as but not limited to cyclopropyl or cyclobutyl;
R₁₃ and R₁₄ are independently selected from the group consisting of H, CH₃, alkyl, substituted alkyl (e.g., Fluoroalkyl), cycloalkyl, and R₁₃ or R₁₄ are connected to either R₇ or R₈ as alkyl to form a (C₁₋₃)alkyl bridge cyclic structure;
R₁₅ is aromatic or aryl, such as but not limited to heteroaryl (e.g., preferably a 5 or 6 membered ring), substituted aromatic or aryl, substituted heteroaryl (e.g., preferably a 5 or 6 membered ring), and fused two heteroaryl ring system; wherein said aromatic, aryl or heteroaryl is unsubstituted, mono-, or di-substituted or tri-substituted, and wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

In some preferred embodiments, this disclosure provides compounds of Formulas I-a and II-a, wherein the ring system (illustrated by A-B-J-E-D variables in Formulas (I) and (II) fused to the 6-membered ring is preferred as an imidazolo ring system as represented by the embodiments shown in Formulas I-a and II-a herein:

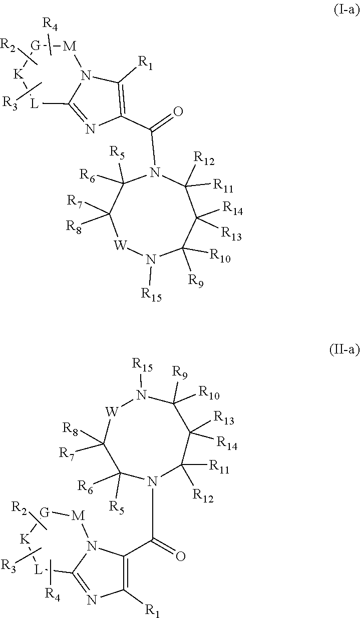

wherein:
R₁, includes E in which is Carbon (C) but not Nitrogen (N), and E is linked to J or D with a double bond, and R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ and R₆ are independently selected from the group consisting of H, CH, alkyl, substituted alkyl, (C₁₋₃)fluoroalkyl, cycloalkyl, R₆ connected to either R₁₀ or R₁₁ or R₁₃ as alkyl to form a (C₁₋₃)alkyl bridge cyclic structure, and R₅ and R₆ linked together to form a spiro moiety such as but not limited to cyclopropyl, or cyclobutyl;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₆ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, and R₁₁ or R₁₂ are connected to either R₇ or R₈ as alkyl to form a (C₁₋₃)alkyl bridge cyclic structure, wherein in some embodiments R₁₁ and R₁₂ linked together can form a spiro moiety such as but not limited to cyclopropyl or cyclobutyl;
R₁₃ and R₁₄ are independently selected from the group consisting of H, CH₃, alkyl, substituted alkyl (e.g., Fluoroalkyl), cycloalkyl, and R₁₃ or R₁₄ are connected to either R₇ or R₈ as alkyl to form a (C₁₋₃)alkyl bridge cyclic structure;
R₁₅ is aromatic or aryl, such as but not limited to heteroaryl (e.g., preferably a 5 or 6 membered ring), substituted aromatic or aryl, substituted heteroaryl (e.g., preferably a 5 or 6 membered ring), and fused two heteroaryl ring system; wherein said aromatic, aryl or heteroaryl is unsubstituted, mono-, or di-substituted or tri-substituted, and wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

In some preferred embodiments of Formula I (e.g., preferably Formula Ia) and Formula II (e.g., preferably Formula IIa), W is CH₂; and R₇, R₈, R₁₃, and R₁₄ are H; the carbons bearing substituents R₇, R₈, R₁₃, and R₁₄ are joined directly to provide a 5-5 bridged bicyclic ring system shown below:

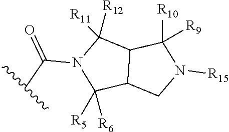

In some preferred embodiments of Formula I (e.g., preferably Formula Ia) and Formula II (e.g., preferably Formula IIa), W is absent (i.e., not present) to provide seven-membered ring system shown below with all other groups as defined herein:

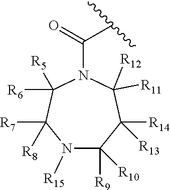

In some preferred embodiments, this disclosure also provides compounds of Formulas I (e.g., preferably Formula Ia) and II (e.g., preferably Formula IIa) comprising:
a fused ring system A-B-J-D-E which is a five-membered heteroaryl, for example imidazole (wherein A, J=Nitrogen while B, E, D=Carbon) or pyrazole (wherein A, B=Nitrogen, while D, E, J=Carbon), fused or unfused with an additional ring systems;
a fused ring system B-J-M-G-K-L which is six (6)-membered aromatic or aryl, substituted aromatic or aryl, substituted or unsubstituted heteroaryl, cycloalkyl, or heterocycloalkyl;
wherein A, B, J, D, E, M, G, K and L are preferably as shown below:
   A is Nitrogen or Carbon;
   B is Carbon or Nitrogen;
   J is Carbon or Nitrogen;
   D is Carbon;
   E is Carbon, wherein R₄ is as defined above;
   M is Carbon, CH, CHR₂, CHR₃, CR₂R₃, CR₂, CR₃, CR₄, O, or N;
   G is Carbon, CH, CHR₂, CHR₃, CR₂R₃, CR₂, CR₃, CR₄, or O;
   K is Carbon, CH, CHR₂, CHR₃, CR₂R₃, CR₂, CR₃, CR₄, or O; and,
   L is Carbon, CH, CHR₂, CHR₃, CR₂R₃, CR₂, CR₃, CR₄, O, or N.

In some preferred embodiments, the disclosure provides a compound of Formula I-b or II-b, wherein the ring system (illustrated by A-B-J-D-E variables in Formulas (I) and (11) fused to the 6-membered ring, preferably a pyrazolo ring system as represented by the embodiments shown in Formula I-b or II-b herein:

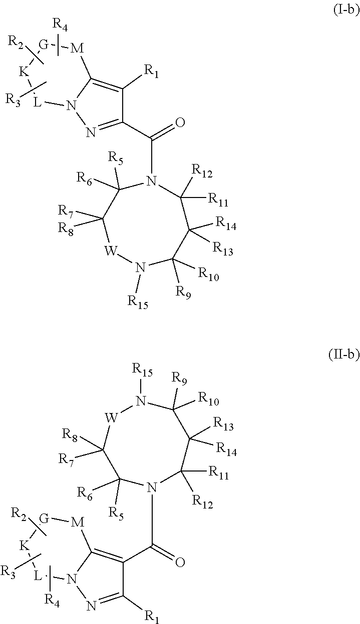

wherein:
R₁, in which E is Carbon (C) but not Nitrogen (N), and E is linked to J or D with a double bond) is selected from the group consisting of H, alkyl, alkoxy, cycloalkyl, aromatic or aryl (e.g., phenyl), heteroaryl (5-6 membered ring), substituted aromatic or aryl, substituted heteroaryl (5-6 membered ring); when R₁ is heteroaryl, it is preferably a 5 or 6-membered heteroaryl selected from the group consisting of pyrazolyl, thiazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as but not limited to F, Cl, Br), alkyl, substituted alkyl, (C₁₋₄)alkyl, (C₁₋₄)alkoxy, (C₁₋₄)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one or two or all R₂, R₃ and R₄;
R₅ and R₆ are independently selected from the group consisting of H, CH, alkyl, substituted alkyl, (C₁₋₃)fluoroalkyl, cycloalkyl, R₆ connected to either R₁₀ or R₁₁ or R₁₃ as alkyl to form a (C₁₋₃)alkyl bridge cyclic structure;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₁₀ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, R₁₁, and R₁₂ connected to either R₇ or R₈ as alkyl to form a (C₁₋₃)alkyl bridge cyclic structure;
R₁₃ and R₁₄ are independently selected from the group consisting of H, CH₃, alkyl, substituted alkyl (e.g. Fluoroalkyl), cycloalkyl, R₁₃, and R₁₄ connected to either R₇ or R₈ as alkyl to form a (C₁₋₃)alkyl bridge cyclic structure; and,
R₁₅ is selected from the group consisting of aromatic or aryl, heteroaryl (5-6 membered ring), substituted aromatic or aryl, substituted heteroaryl (5-6 membered ring) or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy. (C₃₋₇)cycloalkyl, and (C₃₋₇)heterocycloalkyl.

In some preferred embodiments, this disclosure provides compounds of Formula I-b or II-b, wherein W is CH₂; and R₇, R₈, R₁₃, R₁₄ are H, the carbons bearing substituents R₇, R₈, R₁₃, and R₁₄ (as described above) can be joined directly to provide a 5-5 bridged bicyclic ring system shown below:

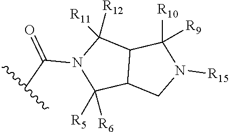

In some preferred embodiments, this disclosure provides compounds of Formula I-b or II-b wherein W is not present (i.e., is absent) to provide seven membered ring system shown below with all other groups as defined herein;

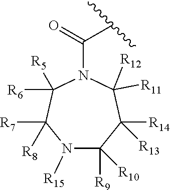

In other preferred embodiments, this disclosure provides compounds wherein the imidazole fused six-membered ring is as shown herein according to the embodiments shown in Formulas (I-a1) and (II-a1):

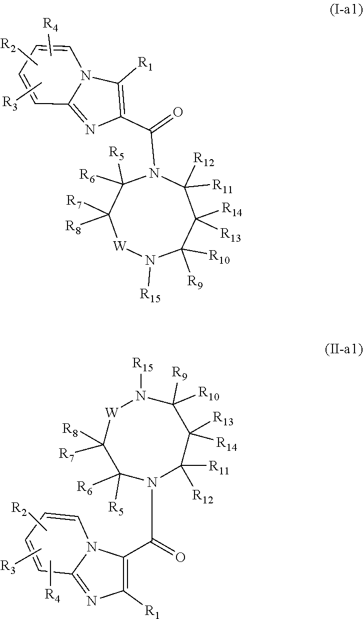

wherein:
R₁, in which E is Carbon (C) but not Nitrogen (N), and E is linked to J or D with a double bond) is selected from the group consisting of H, alkyl, alkoxy, cycloalkyl, aromatic or aryl (e.g., phenyl), heteroaryl (5-6 membered ring), substituted aromatic or aryl, substituted heteroaryl (5-6 membered ring); when R₁ is heteroaryl, it is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as but not limited to F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one or two or all R₂, R₃ and R₄;
R₅ and R₆ are independently selected from the group consisting of H, CH₃, alkyl, substituted alkyl, (C₁₋₃)fluoroalkyl, cycloalkyl, and R₆ connected to either R₁₀ or R₁₁ or R₁₃ as alkyl to form a (C₁₋₃)alkyl bridge cyclic structure, and R₅ and R₆ linked together can form a spiro moiety such as cyclopropyl, or cyclobutyl;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₁₀ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, R₁₁, and R₁₂ connected to either R₇ or R₈ as alkyl to form a (C₁₋₃)alkyl bridge cyclic structure;
R₁₃ and R₁₄ are independently selected from the group consisting of H, CH₃, alkyl, substituted alkyl (e.g. Fluoroalkyl), cycloalkyl, R₁₃, and R₁₄ connected to either R₇ or R₈ as alkyl to form a (C₁₋₃)alkyl bridge cyclic structure;
R₁₅ is selected from the group consisting of aromatic or aryl, heteroaryl (5-6 membered ring), substituted aromatic or aryl, substituted heteroaryl (5-6 membered ring) or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;

In some preferred embodiments, this disclosure provides compounds of Formula I-a or II-a1, wherein W is CH₂, and R₇, R₈, R₁₃, R₁₄ are H, the carbons bearing substituents R₇, R₈, R₁₃, and R₁₄ as defined herein can be joined directly to provide a 5-5 bridged bicyclic ring system shown below:

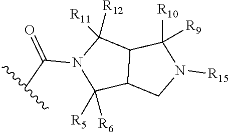

In some preferred embodiments, this disclosure provides compounds of Formula I-b or II-b wherein W is absent (i.e., not present) (to provide seven membered ring system shown below) with all other groups as defined herein;

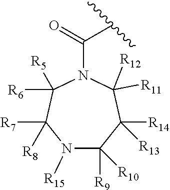

In some preferred embodiments, this disclosure provides compounds wherein the imidazole fused six-membered ring is preferred as shown herein according to the embodiments shown in Formulas (I-a2) and (II-a2):

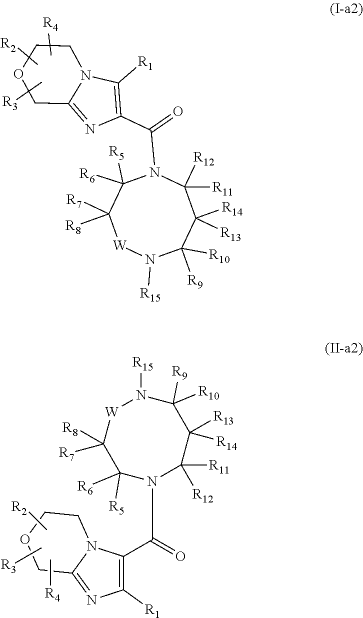

wherein:
R₁, in which E is Carbon (C) but not Nitrogen (N), and E is linked to J or D with a double bond) is selected from the group consisting of H, alkyl, alkoxy, cycloalkyl, aromatic or aryl (e.g., phenyl), heteroaryl (5-6 membered ring), substituted aromatic or aryl, substituted heteroaryl (5-6 membered ring); when R₁ is heteroaryl, it is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as but not limited to F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one or two or all R₂, R₃ and R₄;
R₅ and R₆ are independently selected from the group consisting of H, CH₃, alkyl, substituted alkyl, (C₁₋₃)fluoroalkyl, cycloalkyl, and R₆ connected to either R₁₀ or R₁₁ or R₁₃ as alkyl to form a (C₁₋₃)alkyl bridge cyclic structure;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₁₀ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, R₁₁, and R₁₂ connected to either R₇ or R₈ as alkyl to form a (C₁₋₃)alkyl bridge cyclic structure;
R₁₃ and R₁₄ are independently selected from the group consisting of H, CH₃, alkyl, substituted alkyl (e.g., Fluoroalkyl), cycloalkyl, R₁₃, and R₁₄ connected to either R₇ or R₈ as alkyl to form a (C₁₋₃)alkyl bridge cyclic structure; and,
R₁₅ is selected from the group consisting of aromatic or aryl, heteroaryl (5-6 membered ring), substituted aromatic or aryl, substituted heteroaryl (5-6 membered ring) or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

In some preferred embodiments, this disclosure provides compounds of Formula I-a2 or II-a2 wherein W is CH₂, and R₇, R₈, R₁₃, R₁₄ are each H, the carbons bearing substituents R₇, R₈, R₁₃, and R₁₄ are joined directly to provide a 5-5 bridged bicyclic ring system shown below:

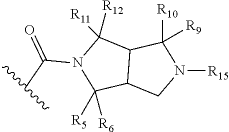

In some preferred embodiments, this disclosure provides compounds of Formula I-a2 or II-a2 wherein W is absent (to provide seven membered ring system shown below) with all other groups as defined herein:

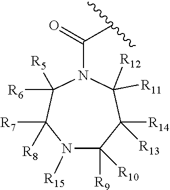

In some preferred embodiments, this disclosure provides compounds wherein the imidazole fused six-membered ring is preferred as shown herein according to the embodiments shown in Formulas (I-a3) and (II-a3):

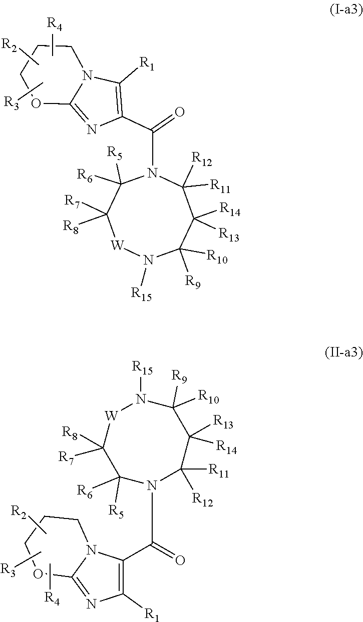

wherein:
R₁, in which E is Carbon (C) but not Nitrogen (N), and E is linked to J or D with a double bond) is selected from the group consisting of H, alkyl, alkoxy, cycloalkyl, aromatic or aryl (e.g., phenyl), heteroaryl (5-6 membered ring), substituted aromatic or aryl, substituted heteroaryl (5-6 membered ring); when R₁ is heteroaryl, it is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as but not limited to F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one or two or all R₂, R₃ and R₄;
R₅ and R₆ are independently selected from the group consisting of H, CH₃, alkyl, substituted alkyl, (C₁₋₃)fluoroalkyl, cycloalkyl, and R₆ connected to either R₁₀ or R₁₁ or R₁₃ as alkyl to form a (C₁₋₃)alkyl bridge cyclic structure;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₆ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, R₁₁, and R₁₂ connected to either R₇ or R₈ as alkyl to form a (C₁₋₃)alkyl bridge cyclic structure;
R₁₃ and R₁₄ are independently selected from the group consisting of H, CH₃, alkyl, substituted alkyl (e.g. Fluoroalkyl), cycloalkyl, R₁₃, and R₁₄ connected to either R₇ or R₈ as alkyl to form a (C₁₋₃)alkyl bridge cyclic structure; and,
R₁₅ is selected from the group consisting of aromatic or aryl, heteroaryl (5-6 membered ring), substituted aromatic or aryl, substituted heteroaryl (5-6 membered ring) or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

In some preferred embodiments, this disclosure provides compounds of Formulas (I-a3) or (l-a3) wherein W is CH₂, and R₇, R₈, R₁₃, R₁₄ are H the carbons bearing substituents R₇, R₈, R₁₃, and R₁₄ can be joined directly to provide a 5-5 bridged bicyclic ring system as shown below:

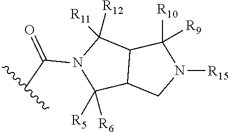

In some preferred embodiments, this disclosure provides compounds of Formula I-a3 or II-a3 wherein W is absent (i.e., not present)(to provide seven membered ring system shown below) with all other groups as defined herein;

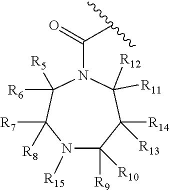

In another preferred embodiment, this disclosure provides compounds wherein the pyrazole fused six-membered ring is preferred as shown herein according to the embodiments shown in Formulas (I-b1) and (II-b1):

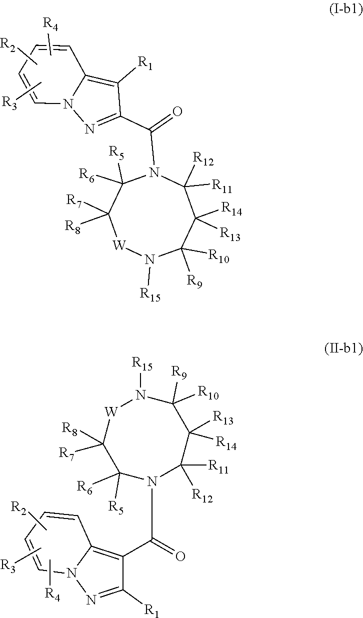

wherein
R₁, in which E is Carbon (C) but not Nitrogen (N), and E is linked to J or D with a double bond is selected from the group consisting of H, alkyl, alkoxy, cycloalkyl, aromatic or aryl (e.g., phenyl), heteroaryl (5-6 membered ring), substituted aromatic or aryl, substituted heteroaryl (5-6 membered ring); when R₁ is heteroaryl, it is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as but not limited to F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one or two or all R₂, R₃ and R₄; R₅ and R₆ are independently selected from the group consisting of H, CH₃, alkyl, substituted alkyl, (C1-3)fluoroalkyl, cycloalkyl, R₆ connected to either R₁₀ or R₁₁ or R₁₃ as alkyl to form a (C1-3)alkyl bridge cyclic structure, and R₅ and R₆ linked together can form a spiro moiety such as cyclopropyl, or cyclobutyl;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₁₀ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, R₁₁, and R₁₂ connected to either R₇ or R₈ as alkyl to form a (C₁₋₃)alkyl bridge cyclic structure;
R₁₃ and R₁₄ are independently selected from the group consisting of H, CH₃, alkyl, substituted alkyl (e.g. Fluoroalkyl), cycloalkyl, R₁₃, and R₁₄ connected to either R₇ or R₈ as alkyl to form a (C₁₋₃)alkyl bridge cyclic structure; and,
R₁₅ is selected from the group consisting of aromatic or aryl, heteroaryl (5-6 membered ring), substituted aromatic or aryl, substituted heteroaryl (5-6 membered ring) or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

In some preferred embodiments, this disclosure provides compounds of Formulas (I-b1) or (II-b1) wherein W is CH₂, and R₇, R₈, R₁₃, R₁₄ are each H, the carbons bearing substituents R₇, R₈, R₁₃, and R₁₄ can be joined directly to provide a 5-5 bridged bicyclic ring system shown below:

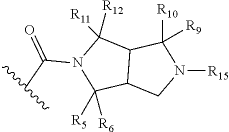

In some preferred embodiments, this disclosure provides compounds of Formulas (I-b1) or (II-b1) wherein W is absent (i.e., not present) (to provide seven membered ring system shown below) with all other groups as defined herein:

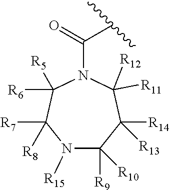

In some preferred embodiments, this disclosure provides compounds wherein the pyrazole fused six-membered ring is as shown herein according to the embodiments shown in Formulas (I-b2) and (II-b2):

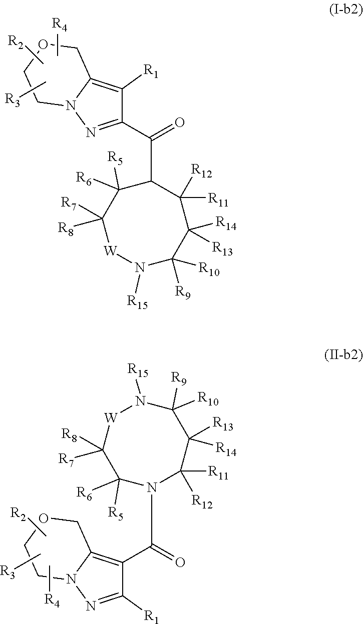

wherein:
R₁, in which E is Carbon (C) but not Nitrogen (N), and E is linked to J or D with a double bond is selected from the group consisting of H, alkyl, alkoxy, cycloalkyl, aromatic or aryl (e.g., phenyl), heteroaryl (5-6 membered ring), substituted aromatic or aryl, substituted heteroaryl (5-6 membered ring); when R₁ is heteroaryl, it is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as but not limited to F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one or two or all R₂, R₃ and R₄;
R₅ and R₆ are independently selected from the group consisting of H, CH₃, alkyl, substituted alkyl, (C₁₋₃)fluoroalkyl, cycloalkyl, R₆ connected to either R₁₀ or R₁₁ or R₁₃ as alkyl to form a (C1-3)alkyl bridge cyclic structure, and R₅ and R₆ linked together can form a spiro moiety such as cyclopropyl, or cyclobutyl;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl. (C₁₋₃)fluoroalkyl, cycloalkyl, and R₉ connected to either R₁₀ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, R₁₁, and R₁₂ connected to either R₇ or R₈ as alkyl to form a (C₁₋₃)alkyl bridge cyclic structure;
R₁₃ and R₁₄ are independently selected from the group consisting of H, CH₃, alkyl, substituted alkyl (e.g. Fluoroalkyl), cycloalkyl, R₁₃, and R₁₄ connected to either R₇ or R₈ as alkyl to form a (C₁₋₃)alkyl bridge cyclic structure; and,
R₁₅ is selected from the group consisting of aromatic or aryl, heteroaryl (5-6 membered ring), substituted aromatic or aryl, substituted heteroaryl (5-6 membered ring) or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

In some preferred embodiments, this disclosure provides compounds of Formulas Ib2 or IIb2 wherein W is CH₂, and R₇, R₈, R₁₃, R₁₄ are each H, the carbons bearing substituents R₇, R₈, R₁₃, and R₁₄ can be joined directly to provide a 5-5 bridged bicyclic ring system shown below:

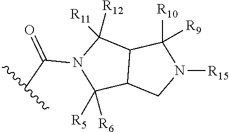

In some preferred embodiments, this disclosure provides compounds of Formulas Ib2 or IIb2 wherein W is absent (i.e., not present) (to provide seven membered ring system shown below) with all other groups defined herein;

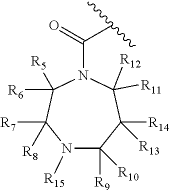

In some preferred embodiments, this disclosure provides compounds wherein the pyrazole fused six-membered ring is as shown herein according to the embodiments shown in Formulas (I-b3) and (II-b3):

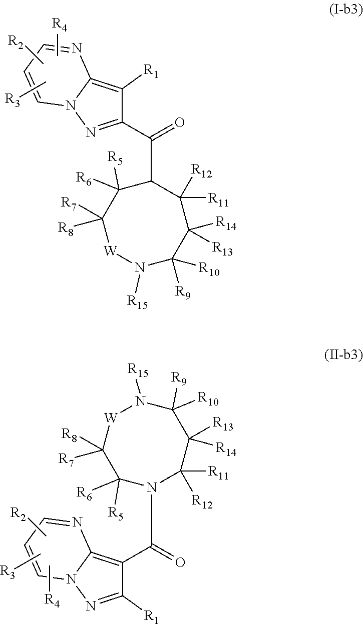

wherein:
R₁, in which E is Carbon (C) but not Nitrogen (N), and E is linked to J or D with a double bond is selected from the group consisting of H, alkyl, alkoxy, cycloalkyl, aromatic or aryl (e.g., phenyl), heteroaryl (5-6 membered ring), substituted aromatic or aryl, substituted heteroaryl (5-6 membered ring); when R₁ is heteroaryl, it is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as but not limited to F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one or two or all R₂, R₃ and R₄;
R₅ and R₆ are independently selected from the group consisting of H, CH₃, alkyl, substituted alkyl, (C₁₋₃)fluoroalkyl, cycloalkyl, R₆ connected to either R₁₀ or R₁₁ or R₁₃ as alkyl to form a (C1-3)alkyl bridge cyclic structure, and R₅ and R₆ linked together can form a spiro moiety such as cyclopropyl, or cyclobutyl;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₁₀ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, R₁₁, and R₁₂ connected to either R₇ or R₈ as alkyl to form a (C₁₋₃)alkyl bridge cyclic structure;
R₁₃ and R₁₄ are independently selected from the group consisting of H, CH₃, alkyl, substituted alkyl (e.g. Fluoroalkyl), cycloalkyl, R₁₃, and R₁₄ connected to either R₇ or R₈ as alkyl to form a (C₁₋₃)alkyl bridge cyclic structure; and,
R₁₅ is selected from the group consisting of aromatic or aryl, heteroaryl (5-6 membered ring), substituted aromatic or aryl, substituted heteroaryl (5-6 membered ring) or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

In some preferred embodiments, this disclosure provides compounds of Formulas (I-b3) or (II-b3) wherein W is CH₂, and R₇, R₈, R₁₃, R₁₄ are each H, the carbons bearing substituents R₇, R₈, R₁₃, and R₁₄ would be joined directly to provide a 5-5 bridged bicyclic ring system shown below:

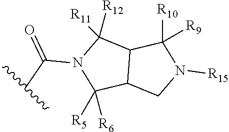

In some preferred embodiments, this disclosure provides compounds of Formulas (I-b3) or (II-b3) wherein W is absent (i.e., is not present) (to provide seven membered ring system shown below) with all other groups defined herein;

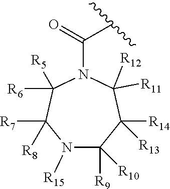

In some preferred embodiments, this disclosure provides compounds wherein the pyrazole fused six-membered ring is as shown herein according to the embodiments shown in Formulas (I-b4) and (II-b4):

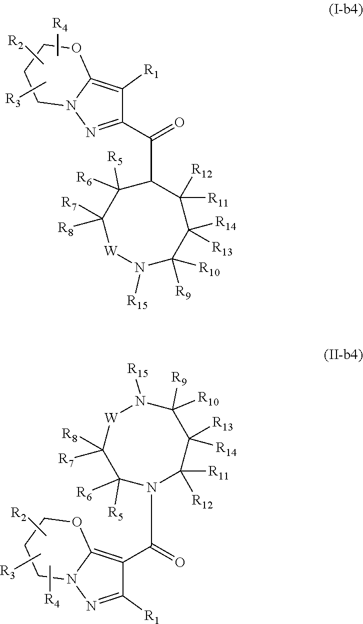

wherein:
R₁, in which E is Carbon (C) not Nitrogen (N), and E is linked to J or D with a double bond, ═H, alkyl, alkoxy, cycloalkyl, Phenyl, aromatic or aryl, heteroaryl (5-6 membered ring), substituted aromatic or aryl, substituted heteroaryl (5-6 membered ring); when R₁ is heteroaryl, it is preferred as a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C1-4)alkyl, (C1-4)alkoxy, halogen, (C1-3)fluoroalkyl, (C1-3)fluoroalkoxy and (C3-7)cycloalkyl;
R₂, R₃, and R₄ are independently selected from the group consisting of H, halogen (such as F, Cl, Br), alky group, substituted alkyl, (C₁₋₄)alkyl, (C₁₋₄)alkoxy, (C₁₋₃)fluoroalkyl, (C₁₋₃)fluoroalkoxy, and (C₃₋₇)cycloalkyl; wherein each of R₂, R₃ and R₄ is independently and optionally substituted at each substitutable position with up to 3 substituents independently selected from one or two or all R₂, R₃ and R₄;
R₅ and R₆ is selected from the group consisting of H, CH₃, alkyl, substituted alkyl, (C₁₋₃)fluoroalkyl, cycloalkyl, R₆ connected to either R₁₀ or R₁₁ or R₁₃ as alkyl to form a (C₁₋₃)alkyl bridge cyclic structure, and R₅ and R₆ linked together can form a spiro moiety such as cyclopropyl, or cyclobutyl;
R₇ is selected from the group consisting of H, CH₃, alkyl, substituted alkyl, (C₁₋₃)fluoroalkyl, cycloalkyl, R₇ connected to either R₁₀ or R₁₁ or R₁₃ as alkyl to form a (C1-3)alkyl bridge cyclic structure;
R₈ is selected from the group consisting of H, CH₃, alkyl, substituted alkyl, (C₁₋₄)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₄)fluoroalkyl, cycloalkyl, and R₉ connected to either R₆ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, or R₇, R₁₂ connected to either R₇ or R₈ as alkyl to form a (C₁₋₃)alkyl bridge cyclic structure;
R₁₃ and R₁₄ is selected from the group consisting of H, CH₃, alkyl, substituted alkyl (e.g. Fluoroalkyl), cycloalkyl, and R₁₃ or R₁₄ connected to either R₇ or R₈ as alkyl to form a (C₁₋₃)alkyl bridge cyclic structure; and,
R₁₅ is selected from the group consisting of aromatic or aryl, heteroaryl (5-6 membered ring), substituted aromatic or aryl, substituted heteroaryl (5-6 membered ring) or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl:

In some preferred embodiments, this disclosure provides compounds of Formula (I-b4) or (II-b4) wherein W is CH₂, and R₇, R₈, R₁₃, R₁₄ are each H, the carbons bearing substituents R₇, R₈, R₁₃, and R₁₄ would be joined directly to provide a 5-5 bridged bicyclic ring system shown below:

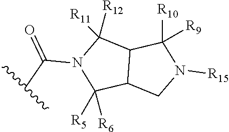

In some preferred embodiments, this disclosure provides compounds of Formula (I-b4) or (II-b4) wherein W is absent (e.g., is nothing) (to provide seven membered ring system shown below) with all other groups as defined herein;

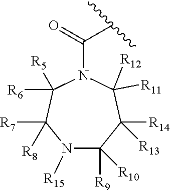

In some especially preferred embodiments, this disclosure provides compounds wherein the stereogenic centers and main scaffold rings are according to the embodiments shown in Formulas (I-a5), (I-b5), (II-a5), and (II-b5):

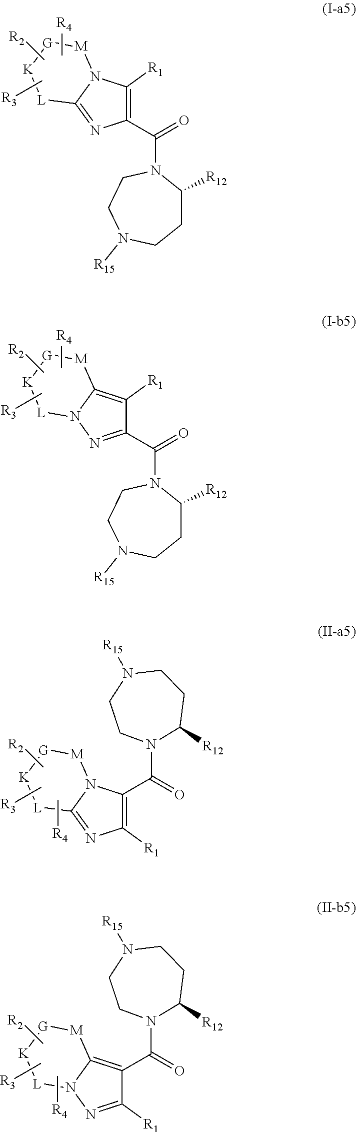

wherein:
all of variables or substituents (i.e., R1-R15) depicted are as defined earlier and above.

In some preferred embodiments, this disclosure provides compounds wherein the stereogenic centers and main scaffold rings are preferred as shown in the exemplification formula as shown herein according to the embodiments shown in formulas (I-a6), (I-b6), (II-a6), and (II-b6):

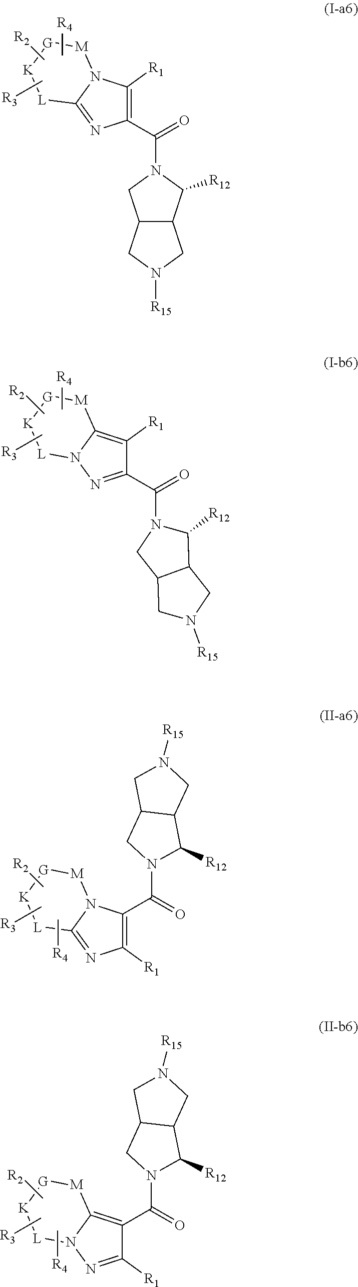

wherein:
all of the variables or substituents (i.e., R1-R15) depicted are as defined earlier and above.

In some preferred embodiments, this disclosure provides compounds having any of Formula I-a7, Formula I-a8, Formula I-a9, Formula II-a7, Formula II-a8, or Formula II-a9:

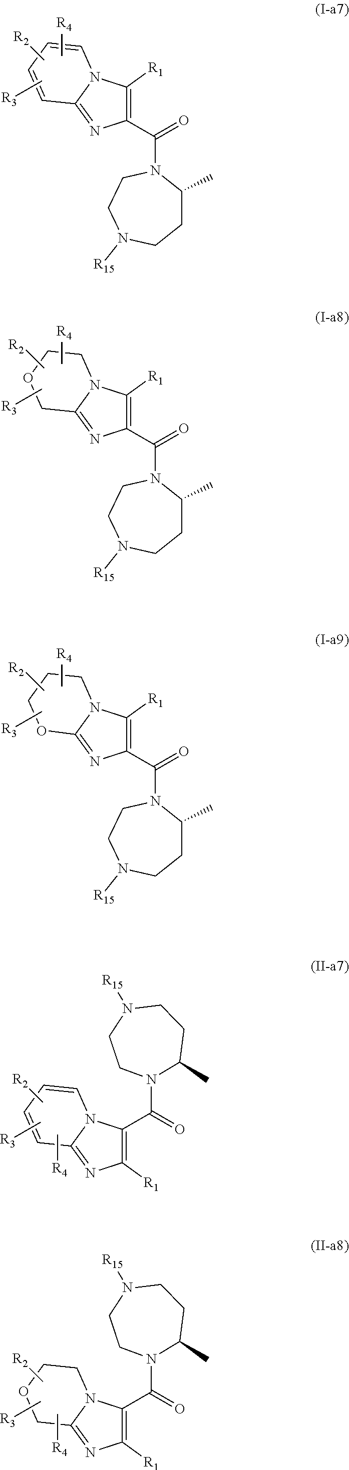

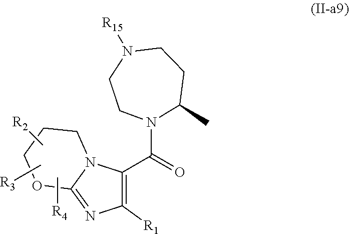

or a pharmaceutically acceptable salt, hydrate, solvate, polymorph, isomer, or combination thereof, wherein:
R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl (5-6 membered ring), substituted aromatic or aryl, substituted heteroaryl (5-6 membered ring); when R₁ is heteroaryl, it is preferred as a 5 or 8-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, and (C₃₋₇)cycloalkyl;
R₂, R₃, and R₄ are independently selected from the group consisting of H, halogen (such as F, Cl, Br), alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to 3 substituents independently selected from one or two or all R₂, R₃ & R₄; and,
R₁₅ is selected from the group consisting of aromatic or aryl, heteroaryl, a 5-6 membered heteroaryl, substituted aromatic or aryl, a substituted 5-6 membered heteroaryl or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₄)fluoroalkoxy, (C₃₋₇)cycloalkyl, and (C₃₋₇)heterocycloalkyl;

In some preferred embodiments, this disclosure provides compounds having any of Formula I-b7, Formula I-b8, Formula I-b9, Formula I-b10, Formula II-b7, Formula II-b8, Formula II-b9 and Formula II-b10:

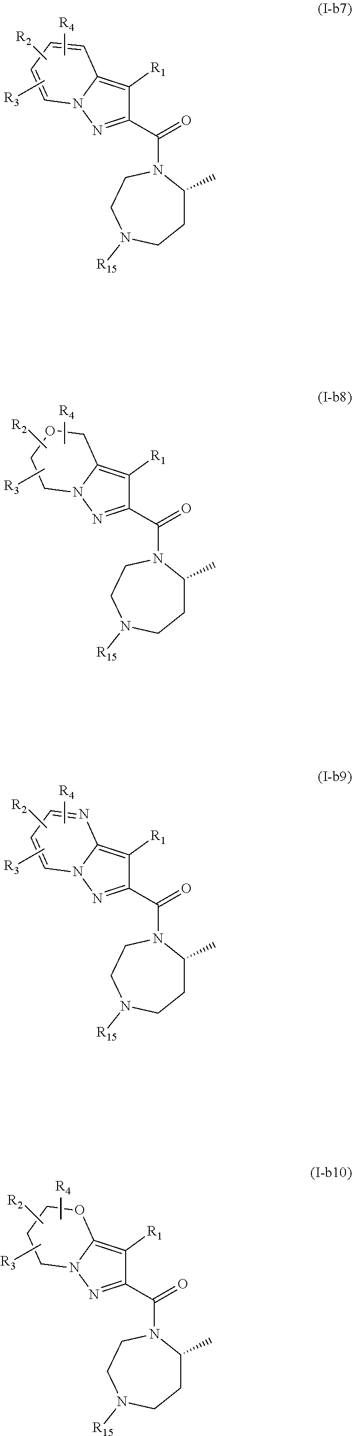

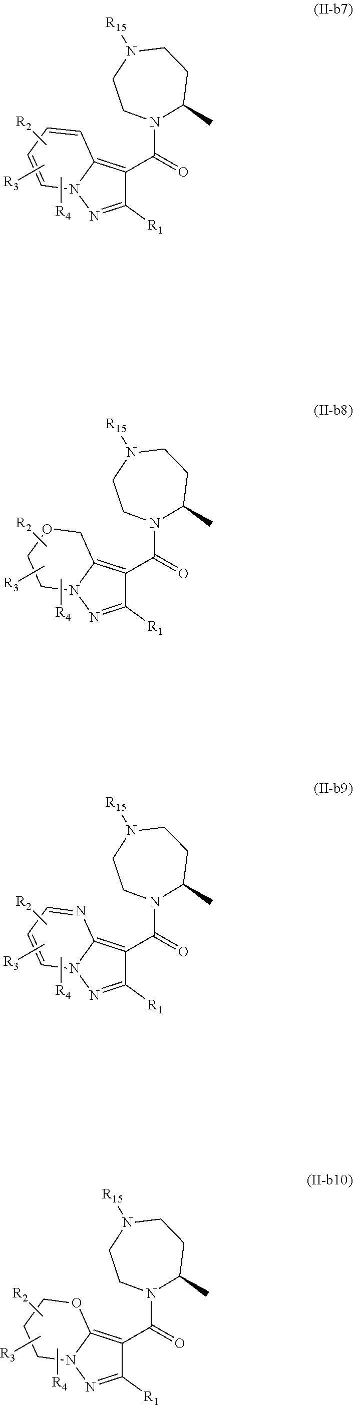

or a pharmaceutically acceptable salt, hydrate, solvate, polymorph, isomer, or combination thereof, wherein:
R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl, a 5-6 membered heteroaryl, substituted aromatic or aryl, substituted heteroaryl, substituted 5-6 membered heteroaryl; when R₁ is heteroaryl, it is optionally a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₄)fluoroalkoxy, and (C₁₋₄)cycloalkyl;
R₂, R₃, R₄ are each independently selected from the group consisting of H, halogen that is optionally F, Cl, or Br, alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to 3 substituents independently selected from one or two or all R₂, R₃ and R₄; and,
R₁₅ is selected from the group consisting of aromatic or aryl, heteroaryl, 5-6 membered heteroaryl, substituted aromatic or aryl, substituted heteroaryl, substituted 5-6 membered heteroaryl and a fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen. (C₁₋₃)fluoroalkyl, (C₁₋₃)fluoroalkoxy, (C₃₋₇)cycloalkyl, (C₃₋₇)heterocycloalkyl.

In some preferred embodiments, this disclosure provides compounds having any of Formula I-b11, Formula I-b12, Formula I-b13, Formula I-b14, Formula II-b11, Formula II-b12, Formula II-b13 and Formula II-b14:

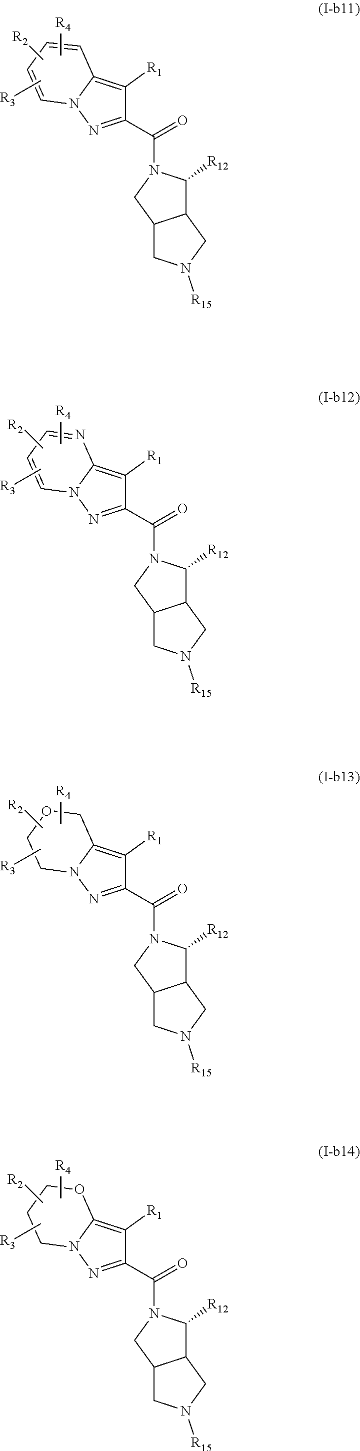

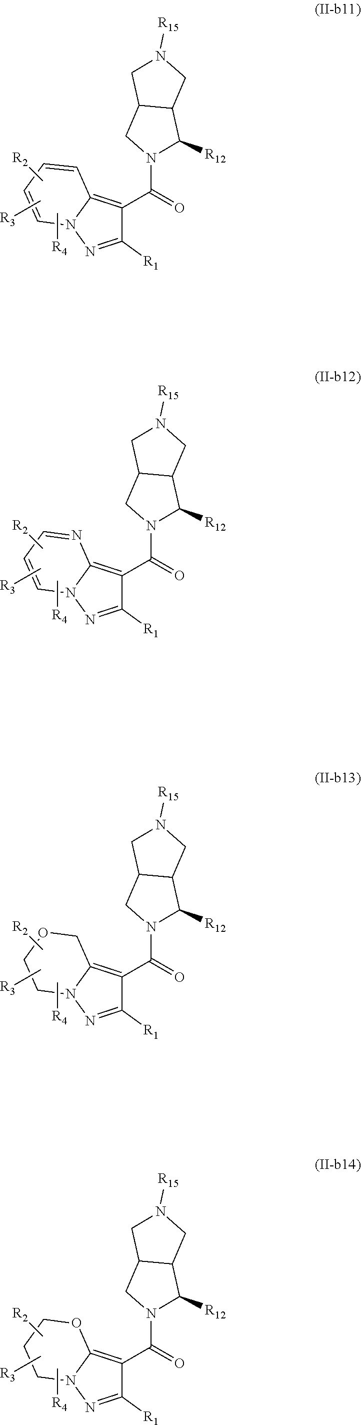

or a pharmaceutically acceptable salt, hydrate, solvate, polymorph, isomer, or combination thereof, wherein:
R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl, 5-6 membered heteroaryl, substituted aromatic or aryl, substituted heteroaryl, substituted 5-6 membered heteroaryl; when R₁ is heteroaryl, optionally a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₄)fluoroalkoxy and (C₃₋₇)cycloalkyl;
R₂, R₃, R₄ are each independently selected from the group consisting of H, halogen, F, Cl, or Br, alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to 3 substituents independently selected from one or two or all R₂, R₃ and R₄;
R₁₂ is selected from the group consisting of H, CH₃, alkyl, substituted alkyl, (C₁₋₃)fluoroalkyl, and cycloalkyl; and,
R₁₅ is selected from the group consisting of aromatic, aryl, heteroaryl, 5-6 membered heteroaryl, substituted aromatic, substituted aryl, substituted heteroaryl, substituted 5-6 membered heteroaryl or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

In most preferred embodiments, this disclosure provides examples of compounds of Formula I and/or Formula II in Table 1.

Any embodiment presented herein is also intended to represent unlabeled forms as well as isotopically labeled forms of the compounds, unless otherwise indicated. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, sulfur, fluorine, chlorine, and iodine such as ²H, ³H, ¹¹C, ¹³C, ¹⁴C, ¹⁵N, ¹⁸F, ³¹P, ³²P, ³⁵S, ³⁶Cl, ¹²⁵I, respectively. This disclosure also includes various isotopically labeled compounds as defined herein, for example those into which radioactive isotopes, such as ³H, ¹³C, and ¹⁴C, are present. Such isotopically labelled compounds are useful in metabolic studies (preferably with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an 18F or labeled compound may be particularly preferred for PET or SPECT studies. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Many of the compounds disclosed herein, and useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The compounds of this disclosure therefore also include any and all stereoisomeric forms such as enantiomeric and diastereoisomeric forms of the compounds or mixtures thereof (including all possible mixtures of stereoisomers). See, e.g., WO 01/062726. Furthermore, in some embodiments, multiple substituents on a piperidinyl or pyrrolidinyl ring can also be in either cis or trans relationship to each other with respect to the plane of the piperidinyl or the pyrrolidinyl ring. Such forms or geometric isomers, although not explicitly indicated in the formulae described herein, are intended to be included within the scope of the present disclosure. With respect to the methods and compositions of the present disclosure, reference to a compound or compounds is intended to encompass that compound in each of its possible isomeric forms and mixtures thereof unless the particular isomeric form is referred to specifically.

Pharmaceutically acceptable salts, as used herein, refer to an agent or a compound according to this disclosure that is a therapeutically active, non-toxic base or acid salt form of the compounds. The acid addition salt form of a compound that occurs in its free form as a base can be obtained by treating said free base form with an appropriate acid such as an inorganic acid, for example, a hydrohalic such as hydrochloric or hydrobromic, sulfuric, nitric, phosphoric and the like; or an organic acid, such as, for example, acetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclic, salicylic, p-aminosalicylic, pamoic and the like (see, e.g., WO 01/062726, U.S. Pat. No. 8,492,416 B2, US 2017/0022208 A1, and US 2017/0253603 A2).

Compounds of this disclosure containing acidic protons may be converted into their therapeutically active, non-toxic base addition salt form, e.g., metal or amine salts, by treatment with appropriate organic and inorganic bases. Appropriate base salt forms include, for example, ammonium salts, alkali and alkaline earth metal salts (e. g., lithium, sodium, potassium, magnesium, calcium salts and the like), salts with organic bases, and salts with amino acids (e.g., arginine, lysine and the like). Conversely, said salt forms can be converted into the free forms by treatment with an appropriate base or acid. Compounds of this disclosure and their salts can be in the form of a solvate, which is included within the scope herein (e.g., hydrates, alcoholates and the like).

The term “aryl” as used herein means a monocyclic or bicyclic carbocyclic aromatic or aryl ring system. Phenyl is a non-limiting (unless otherwise indicated) example of a monocyclic aromatic or aryl ring system.

The term “heteroaryl” as used herein means a monocyclic or bicyclic aromatic or aryl ring system having 1 to 3 heteroatom or heteroatom groups in each ring selected from O, N, NH or S in a chemically stable arrangement. In such a bicyclic aromatic or aryl ring system embodiment of “heteroaryl”: both rings may be aromatic or aryl; and one or both rings may contain said heteroatom or heteroatom groups. Non-limiting examples (unless otherwise indicated) of heteroaryl rings include 2-furanyl, 3-furanyl, N-imidazolyl, 2-imidazolyl, 4-imidazolyl, 5-imidazolyl, benzimidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, benzofuryl, benzothiophenyl, indolyl (e.g., 2-indolyl), pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, purinyl, pyrazinyl, 1,3,5-triazinyl, quinolinyl (e.g., 2-quinolinyl, 3-quinolinyl, 4-quinolinyl), and isoquinolinyl (e.g., 1-isoquinolinyl, 3-isoquinolinyl, or 4-isoquinolinyl).

The term “cycloalkyl or cycloalkenyl” as used herein refers to a monocyclic or fused or (C₁₋₃) alkyl bridged bicyclic carbocyclic ring system that is not aromatic or aryl. Cycloalkenyl rings have one or more units of unsaturation. Preferred cycloalkyl or cycloalkenyl groups include cyclopropyl, cyclobutyl, cyclopentyl, cyclohexyl, cyclohexenyl, cycloheptyl, cycloheptenyl, norbornyl, adamantly and decalinyl.

In some embodiments, the compounds of this disclosure can be in the form of prodrugs, analogs, and/or derivatives. The term “prodrug” is a term recognized by those of ordinary skill in the art in the field and is intended to encompass compounds or agents which, under physiological conditions, are converted into orexin antagonists. A common method for making a prodrug is to select moieties which are hydrolyzed or metabolized under physiological conditions to provide the desired compound or agent. In other embodiments, the prodrug is converted by an enzymatic or chemical activity of the host animal to an orexin antagonist.

The compounds of this disclosure also includes isotopically labelled, especially ²H (deuterium) labelled compounds of all formulas, which compounds are identical to the compounds of all formulas described herein except that one or more atoms have each been replaced by an atom having the same atomic number but an atomic mass different from the atomic mass usually found in nature. Isotopically labelled, especially ²H (deuterium) labelled compounds of all formulas and salts thereof are within the scope of the present invention. Substitution of hydrogen with the heavier isotope 2H (deuterium) may lead to greater metabolic stability resulting, e.g., in increased in-vivo half-life or reduced dosage requirements, and/or may lead to reduced inhibition of cytochrome P450 enzymes, resulting in, e.g., an improved safety profile. In another aspects of embodiment of the invention, the compounds of all formulas are not isotopically labelled. However, isotopically labelled compounds of all formulas disclosed herein could be prepared by anyone skilled in the art in analogy to the methods described hereinafter but using the appropriate isotopic variation of suitable reagents or starting materials.

In some embodiments, this disclosure provides a composition comprising any one or more of such a compound, and/or pharmaceutically acceptable salt, hydrate, solvate, polymorph, isomer, or combination thereof. In some embodiments, this disclosure provides a pharmaceutical composition comprising a compound, pharmaceutically acceptable salt, hydrate, solvate, polymorph, isomer, or combination thereof; and at least one pharmaceutically acceptable excipient, carrier, adjuvant, or vehicle. In some embodiments, this disclosure provides a therapeutically effective amount of such a compound, or a pharmaceutically acceptable salt, hydrate, solvate, polymorph, isomer, or combination thereof. In some embodiments, this disclosure provides such a pharmaceutical composition further comprises at least one second therapeutic agent. The disclosure also provides pharmaceutical compositions comprising one or more compounds of this disclosure (or the like, such as a pharmaceutically acceptable salt thereof) (i.e., as an active agent, as a therapeutic agent), and one or more pharmaceutically acceptable carriers or excipients. A pharmaceutical composition contains a therapeutically effective amount of one or more of such compounds or the like (i.e., active agent(s)), or an appropriate fraction thereof. A composition can optionally contain an additional active agent. In some embodiments, a peptide product is at least about 90%, 95% or 98% pure. Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)}, and organic solvents (e.g., dimethyl sulfoxide and alcohols [e.g., ethanol, glycerol and propylene glycol]). Except insofar as any conventional excipient or carrier is incompatible with a peptide product, the disclosure encompasses the use of conventional excipients and carriers in formulations containing a peptide product. See, e.g., Remington: The Science and Practice of Pharmacy, 21st Ed., Lippincott Williams & Wilkins (Philadelphia, Pennsylvania) (2005); Handbook of Pharmaceutical Excipients, 5th Ed., Rowe et ah, Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Florida) (2004). The appropriateness of a particular formulation can depend on various factors, such as the route of administration chosen. Potential routes of administration of a pharmaceutical composition comprising the compounds or the like disclosed herein can include, without limitation oral, parenteral (including intradermal, subcutaneous, intramuscular, intravascular, intravenous, intra-arterial, intraperitoneal, intracavitary and topical), topical (including transdermal, transmucosal, intranasal (e.g., by nasal spray or drop), ocular (e.g., by eye drop), pulmonary (e.g., by oral or nasal inhalation), buccal, sublingual, rectal (e.g., by suppository), vaginal (e.g., by suppository), and/or other suitable route as would be known to those of ordinary skill in the art.

In some embodiments, the compounds and compositions of this disclosure can be used as orexin receptor antagonists. In preferred embodiments, the compounds and/or compositions disclosed herein can be antagonists for one or more orexin receptor antagonists, such as either one of OX₁R or OX₂R₇ or both OX₁R or OX₂R. The compounds and/or compositions disclosed herein can be selectively more antagonistic for one or more orexin receptor as compared to another orexin receptor, such as in being more or less antagonistic of OX₁R or OX₂R as compared to the other orexin receptor. Thus, the compounds and/or compositions of the same can be referred to herein as “Orexin Receptor Antagonists”.

In some embodiments, the compounds may be antagonists of the K-opioid receptor (or kappa opioid receptor, abbreviated KOR or KOP for its ligand ketazocine, which is a G protein-coupled receptor that in humans is encoded by the OPRK1 gene). In some embodiments, the compounds and/or compositions of this disclosure may be antagonists of one or more types orexin receptors (i.e., Orexin Receptor Antagonist) but not a KOR. In some embodiments, for instance, one or more compounds of this disclosure, and/or a combination thereof, designed and preferred to provide the following characteristics as measured using standard in vitro cellular assays (e.g., those described in the Examples herein), for effective therapeutic efficacy: 1) OX, R Kb<30 nM, OX₂R Kb>1000 nM, KOR Ki<500 nM; 2) OX₁R Kb<30 nM, OX₂R Kb>30 nM, KOR Ki<500 nM; or, 3) OX₁R Kb<30 nM, OX₂R Kb>1000 nM, KOR Ki>1000 nM. In some embodiments, this disclosure provides methods of preventing or treating a condition associated with an orexin receptor (i.e., as an Orexin Receptor Antagonist) and/or one or more K-opioid receptors. For in vivo assays, the compounds and/or compositions of this disclosure may be tested using an animal model such as the rat by measuring primary dependent measures (e.g., such as but not limited to the time to obtain first drug injection, total drug injections taken, rate of drug intake, and total inactive lever presses), the progressive ratio (e.g., such as but not limited to time to obtain first drug injection, breakpoints, final ratio completed, and total active and inactive lever presses), and reinstatement (e.g., such as but not limited to time to first lever press as well as total active and inactive lever presses) These tests and in-vivo assessments are designed, planned and expected to demonstrate disclosures of compounds therapeutic utility and in vivo efficacy—towards attenuation of intake and motivation towards illicit drugs of abuse using techniques known to those of ordinary skill in the art (e.g., Brain Research 1731 (2020), edited by James, et al. (see, e.g., Brodnik, et al. Article 145894); Gentile, et al. Addict Biol 2018, 23(1):247-255: Brodnik, et al. Behav Brain Res. 2015 Sep. 15; 291: 377-384, doi:10.1016/j.bbr.2015.05.051). Other testing methods may also be suitable as would be understood by those of ordinary skill in the art.

In some embodiments, this disclosure provides methods of preventing or treating a condition selected from the group consisting of a central nervous system (CNS) disorder, substance addiction, dependence, panic, anxiety, depression, posttraumatic stress disorder (PTSD), neurodegeneration, autism, schizophrenia, and Alzheimer disease (AD) in a subject in need thereof, by administering to the subject any of such one or more compounds and/or composition comprising one or more of such compounds, or pharmaceutically acceptable salt, hydrate, solvate, polymorph, isomer, or combination thereof. In some embodiments, the methods can include administering a composition comprising a therapeutically effective amount of the compound, pharmaceutically acceptable salt, hydrate, solvate, polymorph, isomer, or combination thereof. In some embodiments, the composition comprises a pharmaceutically acceptable salt or isotope of such a compound. In some embodiments, the composition can comprise an unlabeled form of the compound or an isotopically labeled form of the compound in which the compound has a structure depicted by the formula wherein one or more atoms are replaced by an atom having a selected atomic mass or mass number. In some embodiments, this disclosure provides for the use of a compound, pharmaceutically acceptable salt, hydrate, solvate, polymorph, isomer, or combination thereof, disclosed herein in the preparation of a medicament for preventing and/or treating a condition selected from the group consisting of a central nervous system (CNS) disorder, substance addiction, dependence, panic, anxiety, depression, posttraumatic stress disorder (PTSD), neurodegeneration, autism, schizophrenia, and Alzheimer disease (AD) in a subject in need thereof. Substance addiction, for instance, can include addiction of a person to one or more of an opioids (e.g., such as but not limited to heroin, morphine, oxycodone (e.g., OxyContin, Percocet), fentanyl, and/or hydrocodone (e.g., Vicodin)), one or more stimulants (e.g., amphetamine (e.g., Adderall, Ritalin), cocaine, crack cocaine, methamphetamine, one or more sedatives and/or tranquilizers (e.g., such as but not limited to benzodiazepines (e.g., Valium, Xanax, Klonopin) or barbituates (e.g., Nembutal, Luminal, phenobarbital), or other addictive substance(s) as may be known to those of ordinary skill in the art. In some embodiments, the use can include a composition comprises a therapeutically effective amount of the compound, pharmaceutically acceptable salt, hydrate, solvate, polymorph, isomer, or combination thereof. In some embodiments, such use can comprise a composition a pharmaceutically acceptable salt or isotope of the compound. In some embodiments, such use can comprise a composition comprises an unlabeled form of the compound or an isotopically labeled form of the compound in which the compound has a structure depicted by the formula wherein one or more atoms are replaced by an atom having a selected atomic mass or mass number. This disclosure also provides intermediates of the compounds disclosed herein as well as methods for preparing the same. In some embodiments, such methods for preparing can include using any of the intermediates disclosed herein. Other embodiments are also contemplated here as would be understood by those of ordinary skill in the art.

This disclosure provides various aspects and embodiments of the compounds, compositions, as well as methods for preparing and using the same. In preferred embodiments, this disclosure provides the following aspects:
Aspect 1. A compound of Formulas (I) or (II),

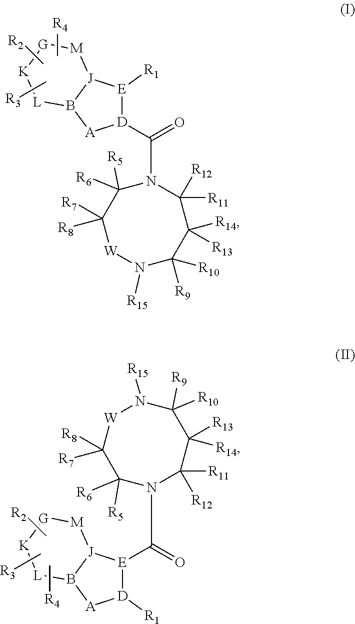

or a pharmaceutically acceptable salt, hydrate, solvate, polymorph, isomer, or combination thereof; wherein:
wherein:
R₁, includes E in which is Carbon (C) but not Nitrogen (N), and E is linked to J or D with a double bond, and R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₄, R₃, and R₄ are independently selected from the group consisting of H, halogen (such as F, Cl, Br), alkyl, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ and R₆ are independently selected from the group consisting of H, CH₃, alkyl, substituted alkyl, (C₁₋₃)fluoroalkyl, cycloalkyl, R₆ connected to either R₁₀ or R₁₁ or R₁₃ as alkyl to form a (C₁₋₃)alkyl bridge cyclic structure, and R₅ and R₆ linked together to form a spiro moiety such as but not limited to cyclopropyl, or cyclobutyl;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure; R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₁₀ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃) fluoroalkyl, cycloalkyl, and R₁₁ or R₁₂ are connected to either R₇ or R₉ as alkyl to form a (C₁₋₃)alkyl bridge cyclic structure, wherein in some embodiments R₁₁ and R₁₂ linked together can form a spiro moiety such as but not limited to cyclopropyl or cyclobutyl;
R₁₃ and R₁₄ are independently selected from the group consisting of H, CH₃, alkyl, substituted alkyl (e.g., Fluoroalkyl), cycloalkyl, and R₁₃ or R₁₄ are connected to either R₇ or R₈ as alkyl to form a (C₁₋₃)alkyl bridge cyclic structure;
R₁₅ is aromatic or aryl, such as but not limited to heteroaryl (e.g., preferably a 5 or 6 membered ring), substituted aromatic or aryl, substituted heteroaryl (e.g., preferably a 5 or 6 membered ring), and fused two heteroaryl ring system; wherein said aromatic, aryl or heteroaryl is unsubstituted, mono-, or di-substituted or tri-substituted, and wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;
W is:
   a) selected from the group consisting of CH₂, and R₇, R₈, R₁₃, R₁₄=H, the carbons bearing substituents R₇, R₈, R₁₃, and R₁₄ would be joined directly to provide a 5-5 bridged bicyclic ring system shown below:

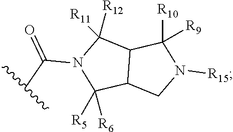

b) absent to provide seven membered ring system shown below:

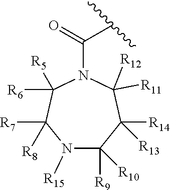

wherein:
   fused ring system A-B-J-D-E is five (5) membered heteroaryl, optionally imidazole wherein A and J are Nitrogen while B, E, and D are Carbon, or pyrazole wherein A and B are Nitrogen and D, E, and J are Carbon: said fused or unfused with an additional ring systems;
   fused ring system B-J-M-G-K-L is a six (6) membered aromatic or aryl, substituted aromatic or aryl, substituted or unsubstituted heteroaryl, cycloalkyl, heterocycloalkyl;
   A is Nitrogen or Carbon;
   B is Carbon or Nitrogen;
   J is Carbon or Nitrogen;
   D is Carbon;
   E is Carbon;
   M is selected from the group consisting of Carbon, CH, CHR₂, CHR₃, CR₂R₃, CR₂, CR₃, CR₄, O, and N;
   G is selected from the group consisting of Carbon, CH, CHR₂, CHR₃, CR₂R₃, CR₂, CR₃, CR₄, and O;
   K is selected from the group consisting of Carbon, CH, CHR₂, CHR₃, CR₂R₃, CR₂, CR₃, CR₄, and O;
   L selected from the group consisting of Carbon, CH, CHR₂, CHR₃, CR₂R₃, CR₂, CR₃, CR₄, O, and N.
Aspect 2. A compound of aspect 1 of Formula I-a7, Formula I-a8, Formula I-a9, Formula II-a7, Formula II-a8, or Formula II-a9

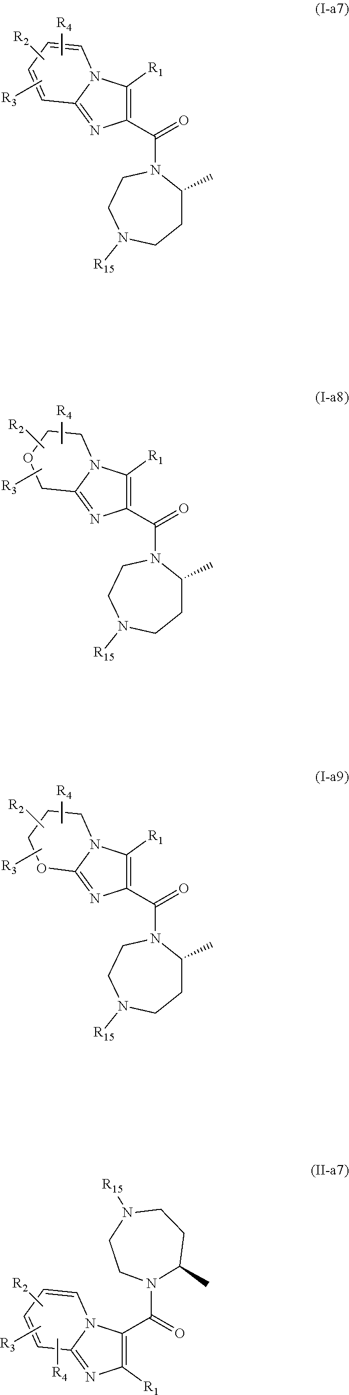

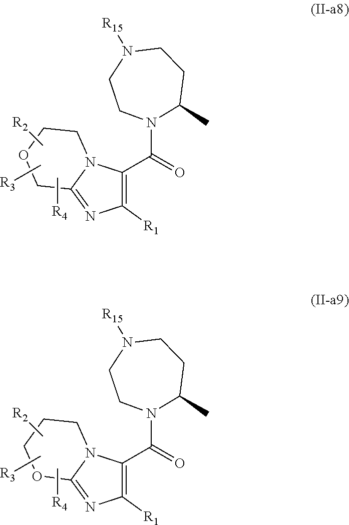

or a pharmaceutically acceptable salt, hydrate, solvate, polymorph, isomer, or combination thereof,
wherein:
   R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl (5-6 membered ring), substituted aromatic or aryl, substituted heteroaryl (5-6 membered ring); when R₁ is heteroaryl, it is preferred as a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, and (C₃₋₇)cycloalkyl;
   R₂, R₃, and R₄ are independently selected from the group consisting of H, halogen (such as F, Cl, Br), alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to 3 substituents independently selected from one or two or all R₂, R₃ & R₄; and,
   R₁₅ is selected from the group consisting of aromatic or aryl, heteroaryl, a 5-6 membered heteroaryl, substituted aromatic or aryl, a substituted 5-6 membered heteroaryl or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.
Aspect 3. A compound of aspect 1 having Formula I-b7, Formula I-b8, Formula I-b9, Formula I-b10, Formula II-b7, Formula II-b8, Formula II-b9 and Formula II-b10:

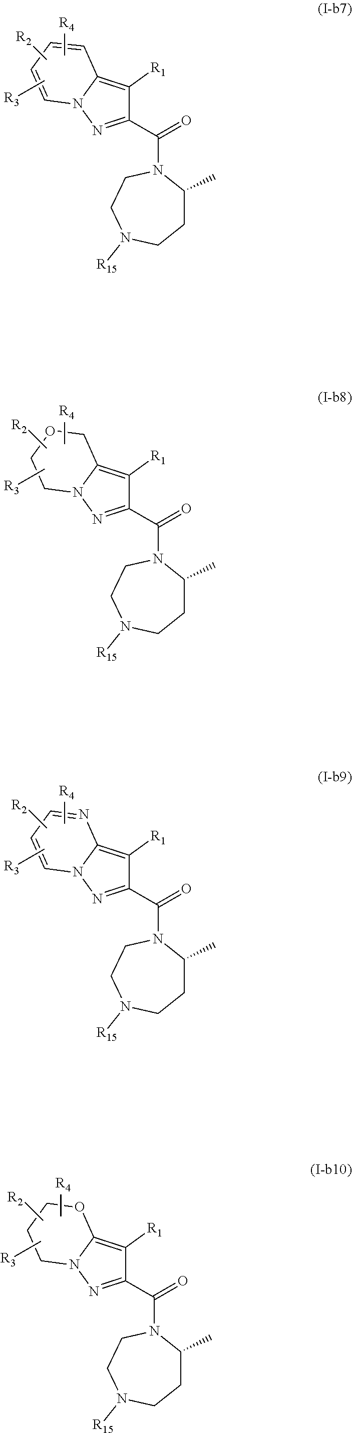

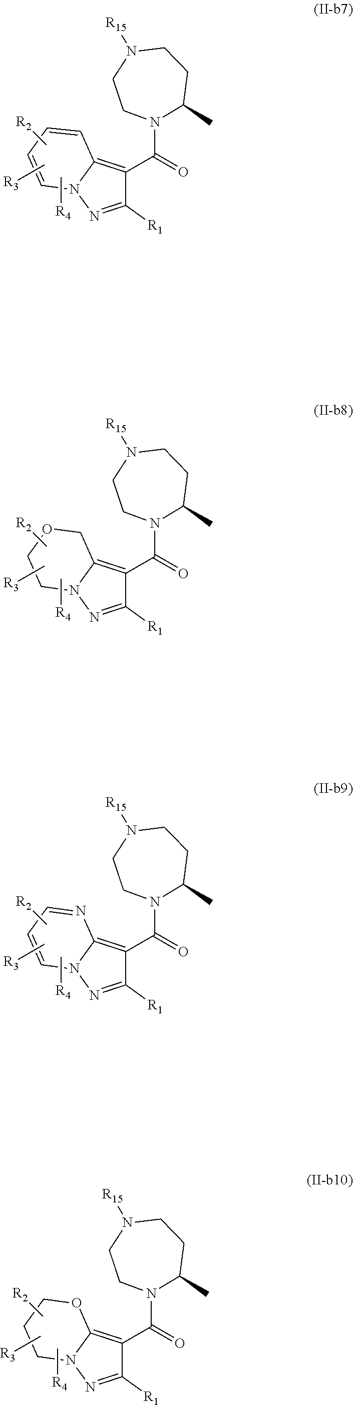

or a pharmaceutically acceptable salt, hydrate, solvate, polymorph, isomer, or combination thereof,
wherein:
   R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl, a 5-6 membered heteroaryl, substituted aromatic or aryl, substituted heteroaryl, substituted 5-6 membered heteroaryl; when R₁ is heteroaryl, it is optionally a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, and (C₃₋₇)cycloalkyl;
   R₂, R₃, R₄ are each independently selected from the group consisting of H, halogen that is optionally F, Cl, or Br, alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to 3 substituents independently selected from one or two or all R₂, R₃ and R₄; and,
   R₁₅ is selected from the group consisting of aromatic or aryl, heteroaryl, 5-6 membered heteroaryl, substituted aromatic or aryl, substituted heteroaryl, substituted 5-6 membered heteroaryl and a fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocyloalkyl.
Aspect 4. A compound of aspect 1 having Formula I-b11, Formula I-b12, Formula I-b13, Formula I-b14, Formula I-b11, Formula II-b12, Formula II-b13 and Formula II-b14:

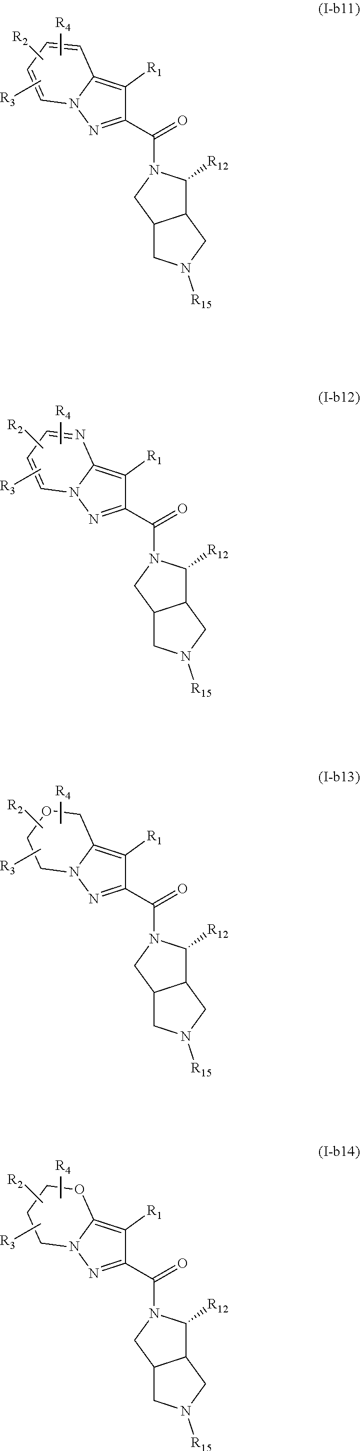

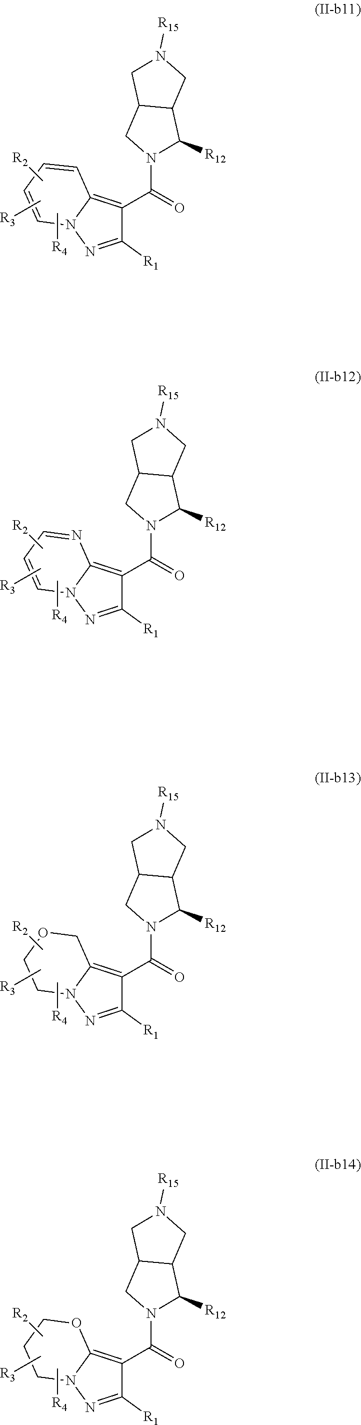

or a pharmaceutically acceptable salt, hydrate, solvate, polymorph, isomer, or combination thereof,
wherein:
   R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl, 5-6 membered heteroaryl, substituted aromatic or aryl, substituted heteroaryl, substituted 5-6 membered heteroaryl; when R₁ is heteroaryl, optionally a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
   R₂, R₃, R₄ are each independently selected from the group consisting of H, halogen, F, Cl, or Br, alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to 3 substituents independently selected from one or two or all R₂, R₃ and R₄;
   R₁₂ is selected from the group consisting of H, CH₃, alkyl, substituted alkyl, (C₁₋₃)fluoroalkyl, and cycloalkyl; and,
   R₁₅ is selected from the group consisting of aromatic, aryl, heteroaryl, 5-6 membered heteroaryl, substituted aromatic, substituted aryl, substituted heteroaryl, substituted 5-6 membered heteroaryl or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.
Aspect 5. A compound of aspect 1 selected from the group consisting of:

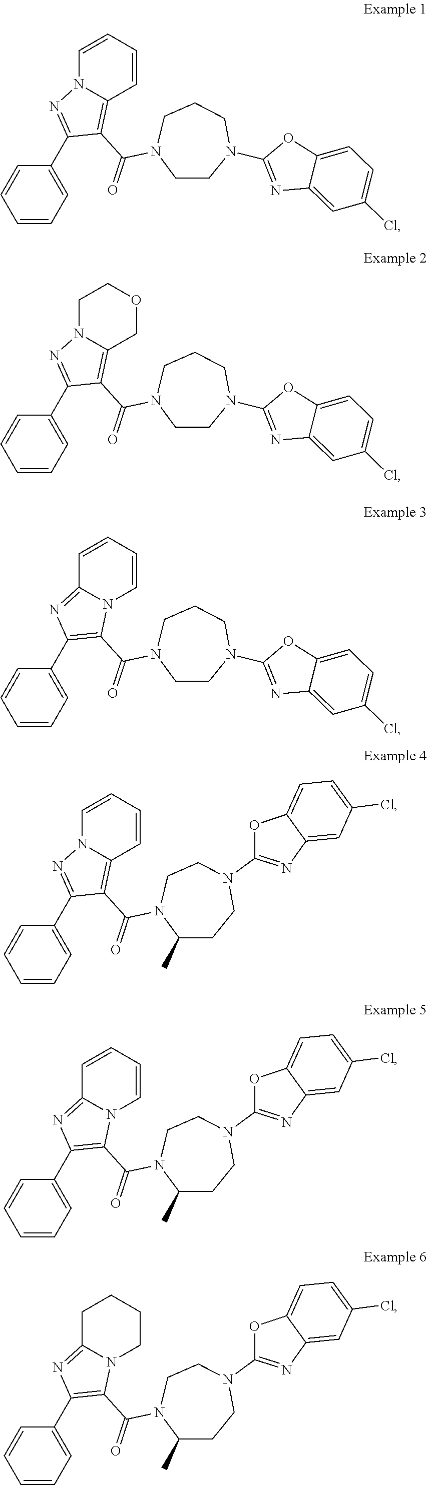 [SMILES: O=C(c1c(-c2ccccc2)nn2ccccc12)N1CCCN(c2nc3cc(Cl)ccc3o2)CC1 ; O=C(c1c(-c2ccccc2)nn2c1COCC2)N1CCCN(c2nc3cc(Cl)ccc3o2)CC1 ; O=C(c1c(-c2ccccc2)nc2ccccn12)N1CCCN(c2nc3cc(Cl)ccc3o2)CC1 ; C[C@@H]1CCN(c2nc3cc(Cl)ccc3o2)CCN1C(=O)c1c(-c2ccccc2)nn2ccccc12 ; C[C@@H]1CCN(c2nc3cc(Cl)ccc3o2)CCN1C(=O)c1c(-c2ccccc2)nc2ccccn12 ; C[C@@H]1CCN(c2nc3cc(Cl)ccc3o2)CCN1C(=O)c1c(-c2ccccc2)nc2n1CCCC2]

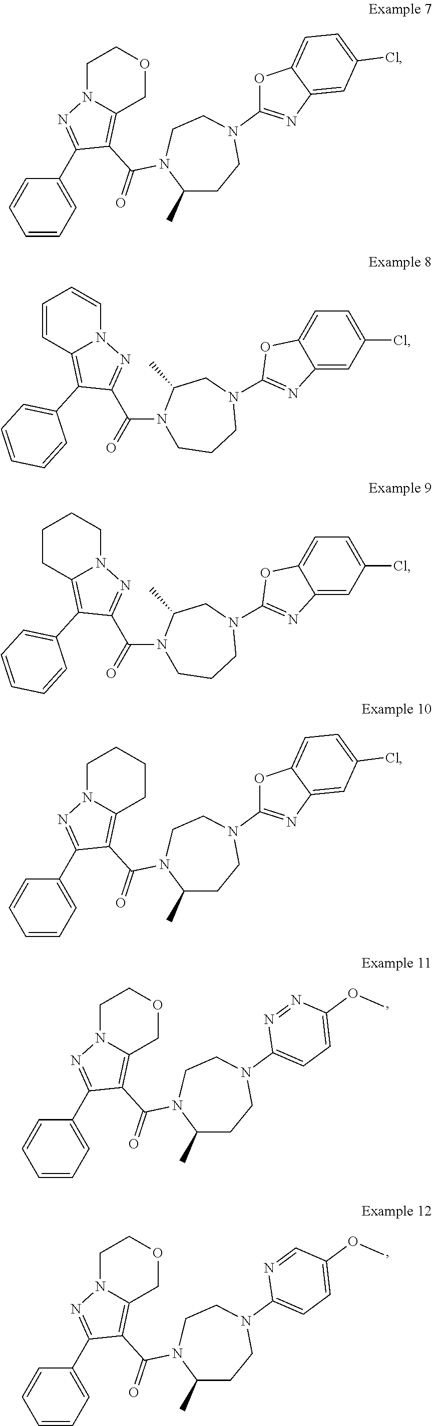 [SMILES: C[C@@H]1CCN(c2nc3cc(Cl)ccc3o2)CCN1C(=O)c1c(-c2ccccc2)nn2c1COCC2 ; C[C@@H]1CN(c2nc3cc(Cl)ccc3o2)CCCN1C(=O)c1nn2ccccc2c1-c1ccccc1 ; C[C@@H]1CN(c2nc3cc(Cl)ccc3o2)CCCN1C(=O)c1nn2c(c1-c1ccccc1)CCCC2 ; C[C@@H]1CCN(c2nc3cc(Cl)ccc3o2)CCN1C(=O)c1c(-c2ccccc2)nn2c1CCCC2 ; COc1ccc(N2CC[C@@H](C)N(C(=O)c3c(-c4ccccc4)nn4c3COCC4)CC2)nn1 ; COc1ccc(N2CC[C@@H](C)N(C(=O)c3c(-c4ccccc4)nn4c3COCC4)CC2)nc1]

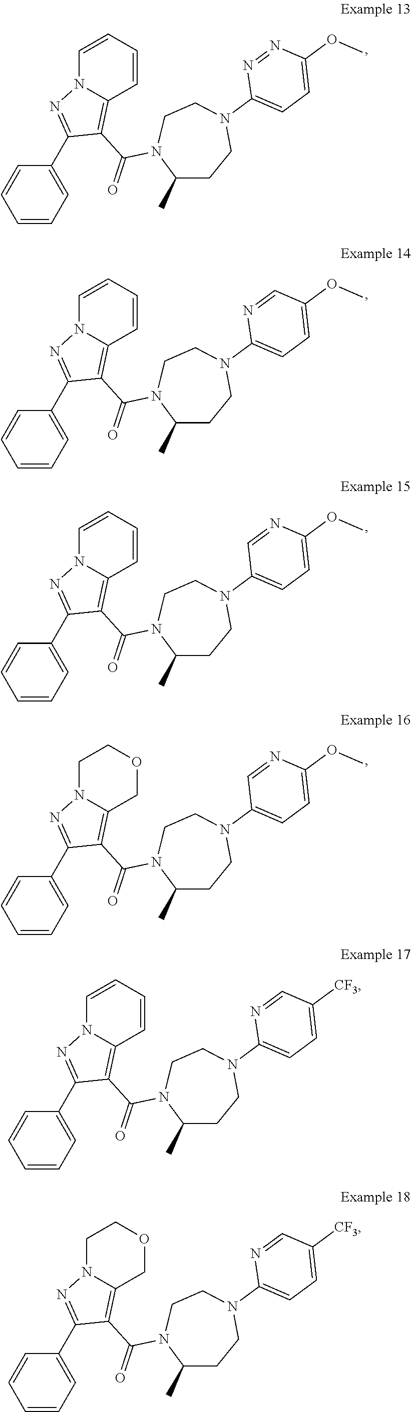 [SMILES: COc1ccc(N2CC[C@@H](C)N(C(=O)c3c(-c4ccccc4)nn4ccccc34)CC2)nn1 ; COc1ccc(N2CC[C@@H](C)N(C(=O)c3c(-c4ccccc4)nn4ccccc34)CC2)nc1 ; COc1ccc(N2CC[C@@H](C)N(C(=O)c3c(-c4ccccc4)nn4ccccc34)CC2)cn1 ; COc1ccc(N2CC[C@@H](C)N(C(=O)c3c(-c4ccccc4)nn4c3COCC4)CC2)cn1 ; C[C@@H]1CCN(c2ccc(C(F)(F)F)cn2)CCN1C(=O)c1c(-c2ccccc2)nn2ccccc12 ; C[C@@H]1CCN(c2ccc(C(F)(F)F)cn2)CCN1C(=O)c1c(-c2ccccc2)nn2c1COCC2]

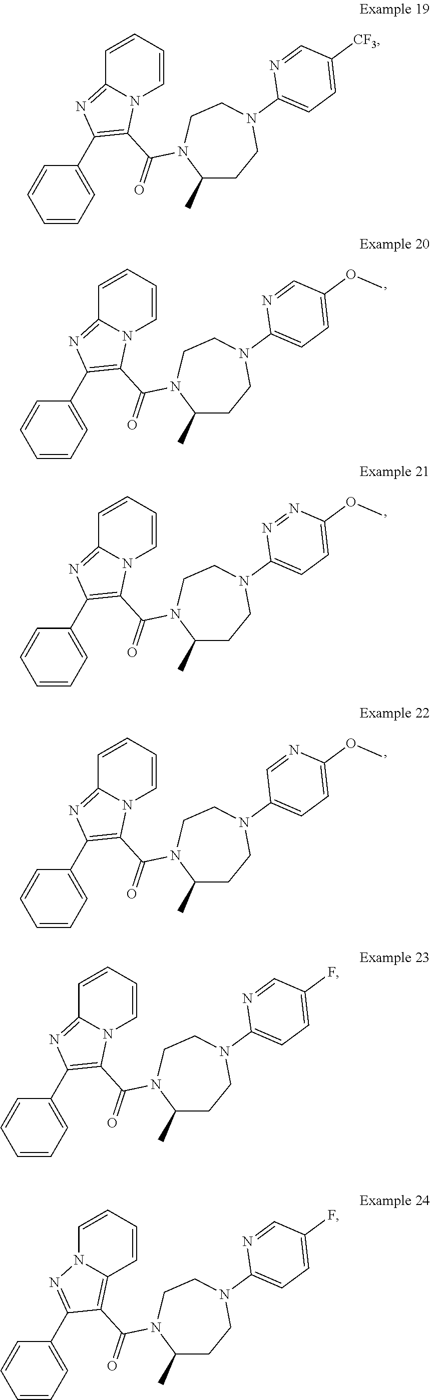 [SMILES: C[C@@H]1CCN(c2ccc(C(F)(F)F)cn2)CCN1C(=O)c1c(-c2ccccc2)nc2ccccn12 ; COc1ccc(N2CC[C@@H](C)N(C(=O)c3c(-c4ccccc4)nc4ccccn34)CC2)nc1 ; COc1ccc(N2CC[C@@H](C)N(C(=O)c3c(-c4ccccc4)nc4ccccn34)CC2)nn1 ; COc1ccc(N2CC[C@@H](C)N(C(=O)c3c(-c4ccccc4)nc4ccccn34)CC2)cn1 ; C[C@@H]1CCN(c2ccc(F)cn2)CCN1C(=O)c1c(-c2ccccc2)nc2ccccn12 ; C[C@@H]1CCN(c2ccc(F)cn2)CCN1C(=O)c1c(-c2ccccc2)nn2ccccc12]

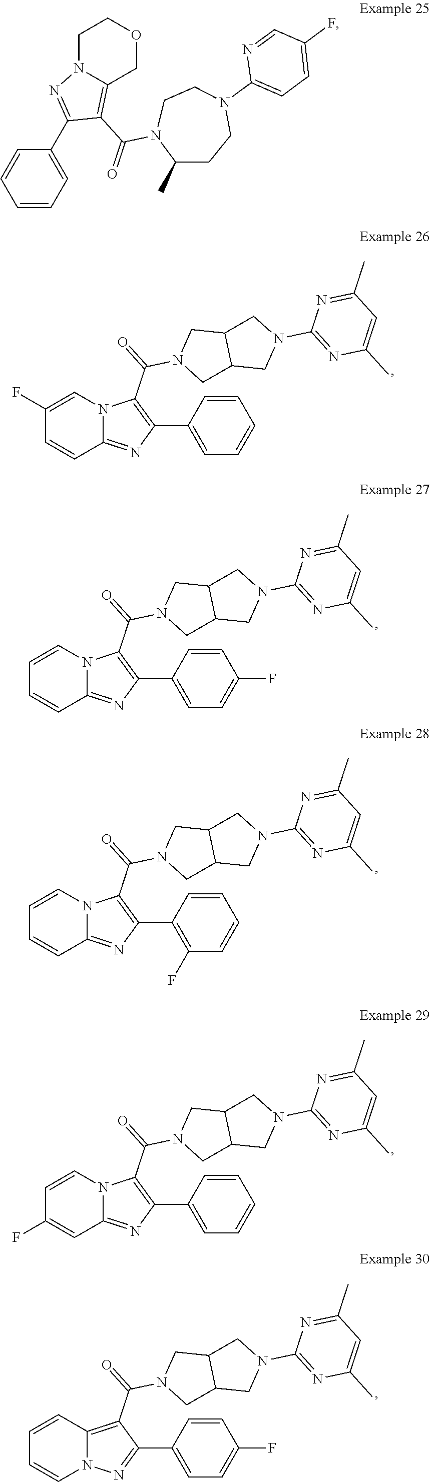 [SMILES: C[C@@H]1CCN(c2ccc(F)cn2)CCN1C(=O)c1c(-c2ccccc2)nn2c1COCC2 ; Cc1cc(C)nc(N2CC3CN(C(=O)c4c(-c5ccccc5)nc5ccc(F)cn45)CC3C2)n1 ; Cc1cc(C)nc(N2CC3CN(C(=O)c4c(-c5ccc(F)cc5)nc5ccccn45)CC3C2)n1 ; Cc1cc(C)nc(N2CC3CN(C(=O)c4c(-c5ccccc5F)nc5ccccn45)CC3C2)n1 ; Cc1cc(C)nc(N2CC3CN(C(=O)c4c(-c5ccccc5)nc5cc(F)ccn45)CC3C2)n1 ; Cc1cc(C)nc(N2CC3CN(C(=O)c4c(-c5ccc(F)cc5)nn5ccccc45)CC3C2)n1]

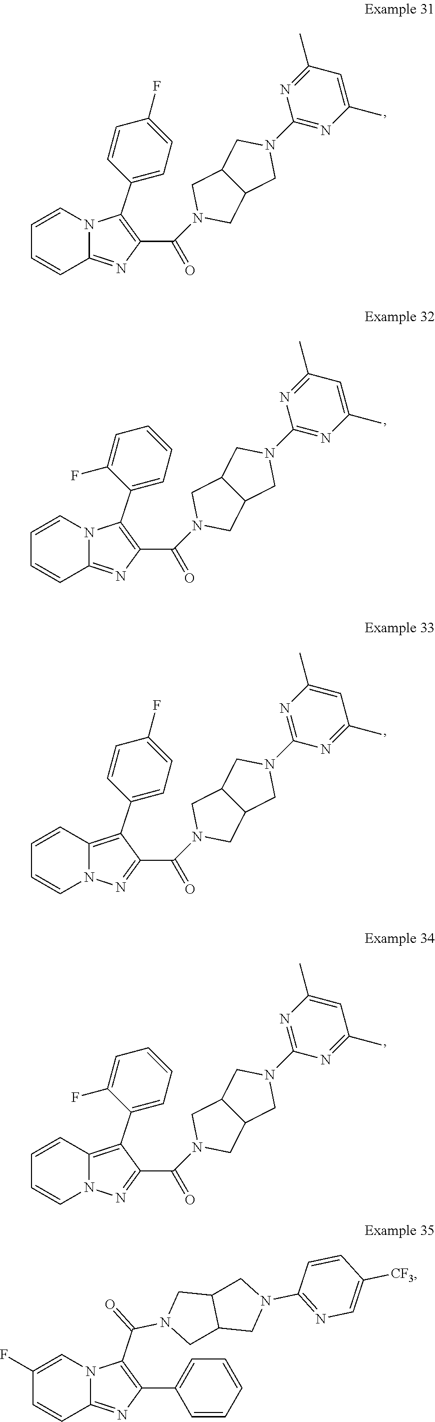 [SMILES: Cc1cc(C)nc(N2CC3CN(C(=O)c4nc5ccccn5c4-c4ccc(F)cc4)CC3C2)n1 ; Cc1cc(C)nc(N2CC3CN(C(=O)c4nc5ccccn5c4-c4ccccc4F)CC3C2)n1 ; Cc1cc(C)nc(N2CC3CN(C(=O)c4nn5ccccc5c4-c4ccc(F)cc4)CC3C2)n1 ; Cc1cc(C)nc(N2CC3CN(C(=O)c4nn5ccccc5c4-c4ccccc4F)CC3C2)n1 ; O=C(c1c(-c2ccccc2)nc2ccc(F)cn12)N1CC2CN(c3ccc(C(F)(F)F)cn3)CC2C1]

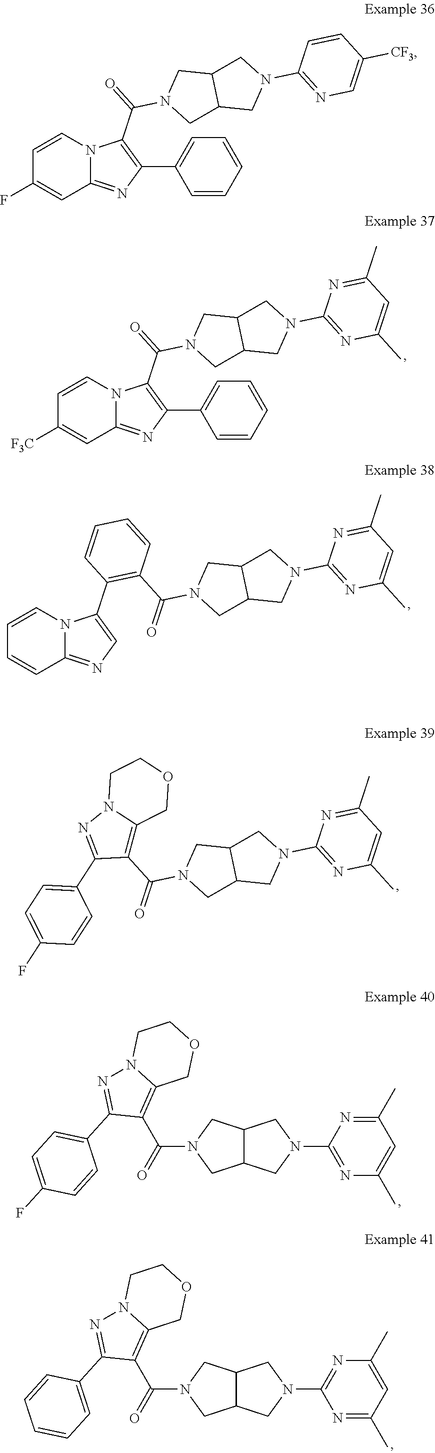 [SMILES: O=C(c1c(-c2ccccc2)nc2cc(F)ccn12)N1CC2CN(c3ccc(C(F)(F)F)cn3)CC2C1 ; Cc1cc(C)nc(N2CC3CN(C(=O)c4c(-c5ccccc5)nc5cc(C(F)(F)F)ccn45)CC3C2)n1 ; Cc1cc(C)nc(N2CC3CN(C(=O)c4ccccc4-c4cnc5ccccn45)CC3C2)n1 ; Cc1cc(C)nc(N2CC3CN(C(=O)c4c(-c5ccc(F)cc5)nn5c4COCC5)CC3C2)n1 ; Cc1cc(C)nc(N2CC3CN(C(=O)c4c(-c5ccc(F)cc5)nn5c4COCC5)CC3C2)n1 ; Cc1cc(C)nc(N2CC3CN(C(=O)c4c(-c5ccccc5)nn5c4COCC5)CC3C2)n1]

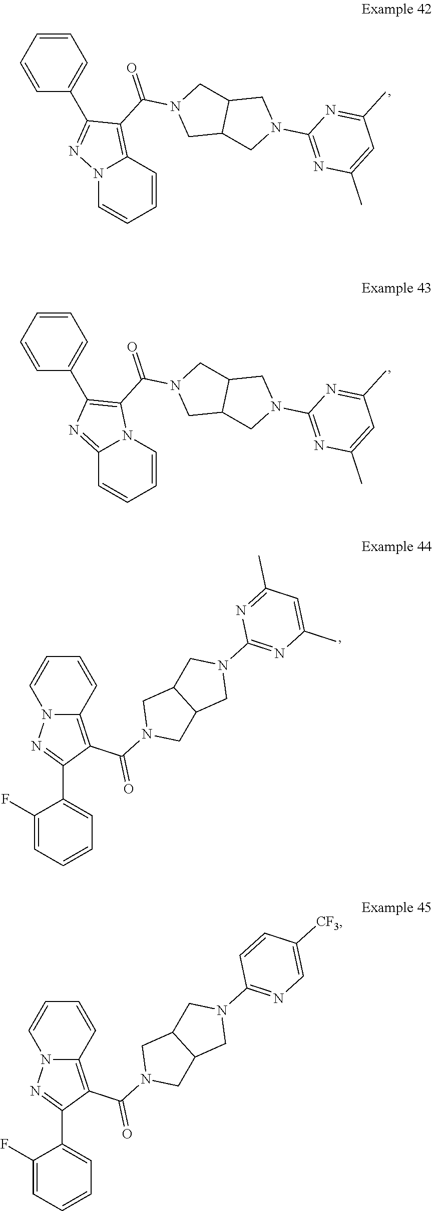 [SMILES: Cc1cc(C)nc(N2CC3CN(C(=O)c4c(-c5ccccc5)nn5ccccc45)CC3C2)n1 ; Cc1cc(C)nc(N2CC3CN(C(=O)c4c(-c5ccccc5)nc5ccccn45)CC3C2)n1 ; Cc1cc(C)nc(N2CC3CN(C(=O)c4c(-c5ccccc5F)nn5ccccc45)CC3C2)n1 ; O=C(c1c(-c2ccccc2F)nn2ccccc12)N1CC2CN(c3ccc(C(F)(F)F)cn3)CC2C1]

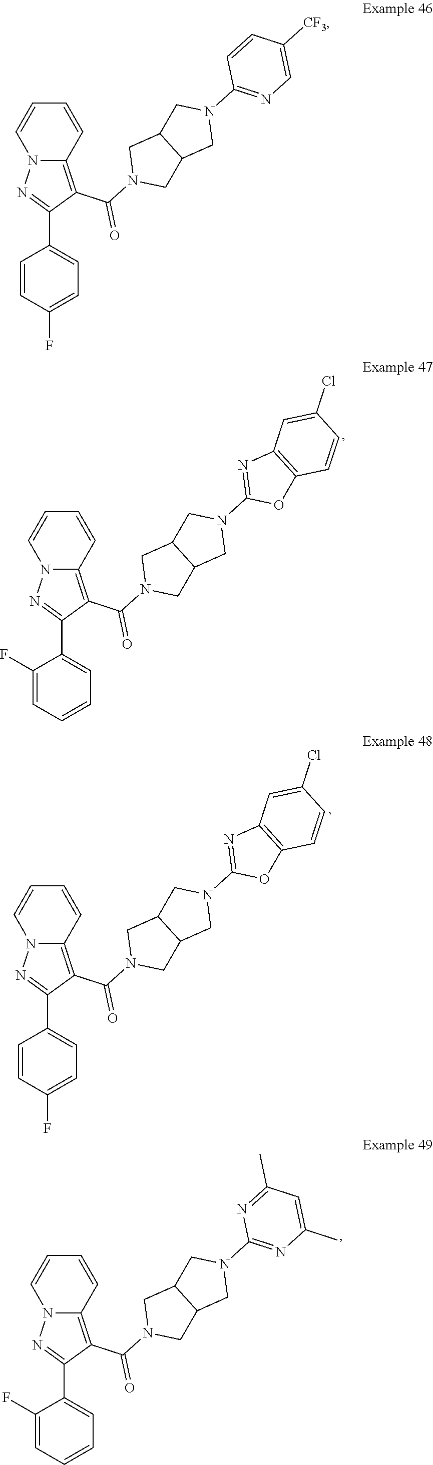 [SMILES: O=C(c1c(-c2ccc(F)cc2)nn2ccccc12)N1CC2CN(c3ccc(C(F)(F)F)cn3)CC2C1 ; O=C(c1c(-c2ccccc2F)nn2ccccc12)N1CC2CN(c3nc4cc(Cl)ccc4o3)CC2C1 ; O=C(c1c(-c2ccc(F)cc2)nn2ccccc12)N1CC2CN(c3nc4cc(Cl)ccc4o3)CC2C1 ; Cc1cc(C)nc(N2CC3CN(C(=O)c4c(-c5ccccc5F)nn5ccccc45)CC3C2)n1]

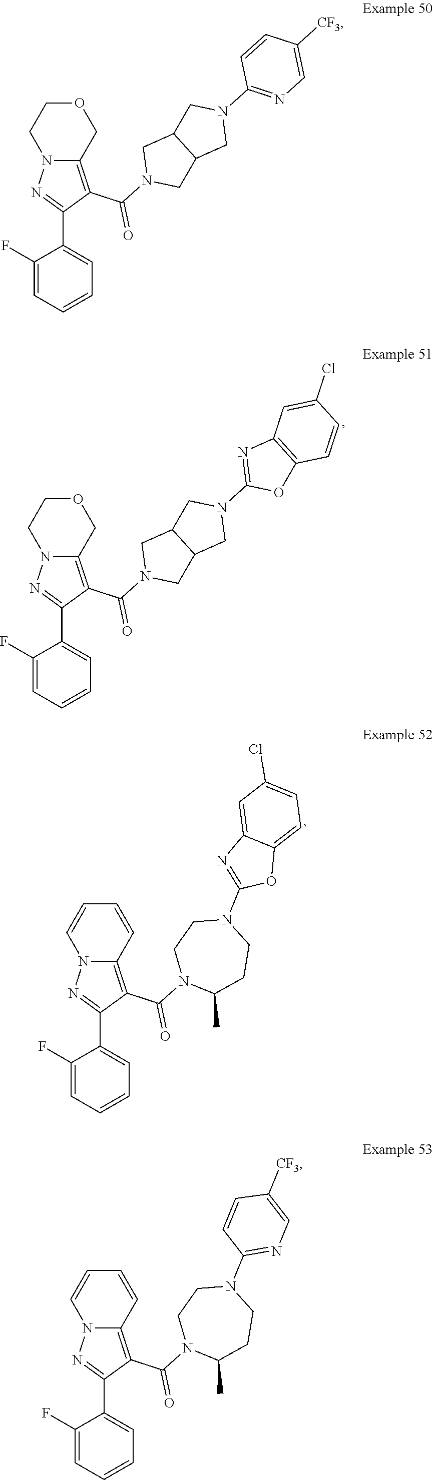 [SMILES: O=C(c1c(-c2ccccc2F)nn2c1COCC2)N1CC2CN(c3ccc(C(F)(F)F)cn3)CC2C1 ; O=C(c1c(-c2ccccc2F)nn2c1COCC2)N1CC2CN(c3nc4cc(Cl)ccc4o3)CC2C1 ; C[C@@H]1CCN(c2nc3cc(Cl)ccc3o2)CCN1C(=O)c1c(-c2ccccc2F)nn2ccccc12 ; C[C@@H]1CCN(c2ccc(C(F)(F)F)cn2)CCN1C(=O)c1c(-c2ccccc2F)nn2ccccc12]

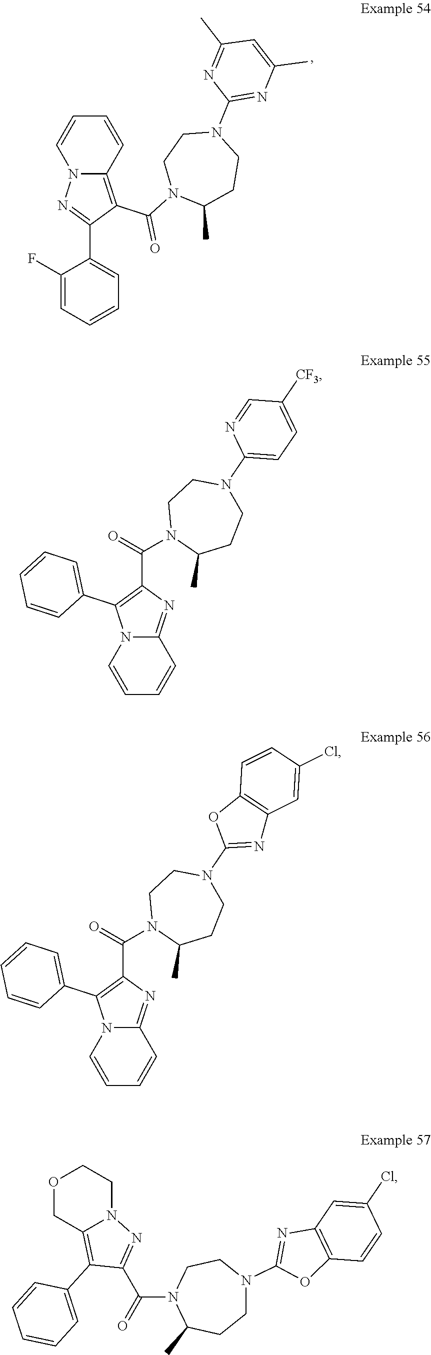 [SMILES: Cc1cc(C)nc(N2CC[C@@H](C)N(C(=O)c3c(-c4ccccc4F)nn4ccccc34)CC2)n1 ; C[C@@H]1CCN(c2ccc(C(F)(F)F)cn2)CCN1C(=O)c1nc2ccccn2c1-c1ccccc1 ; C[C@@H]1CCN(c2nc3cc(Cl)ccc3o2)CCN1C(=O)c1nc2ccccn2c1-c1ccccc1 ; C[C@@H]1CCN(c2nc3cc(Cl)ccc3o2)CCN1C(=O)c1nn2c(c1-c1ccccc1)COCC2]

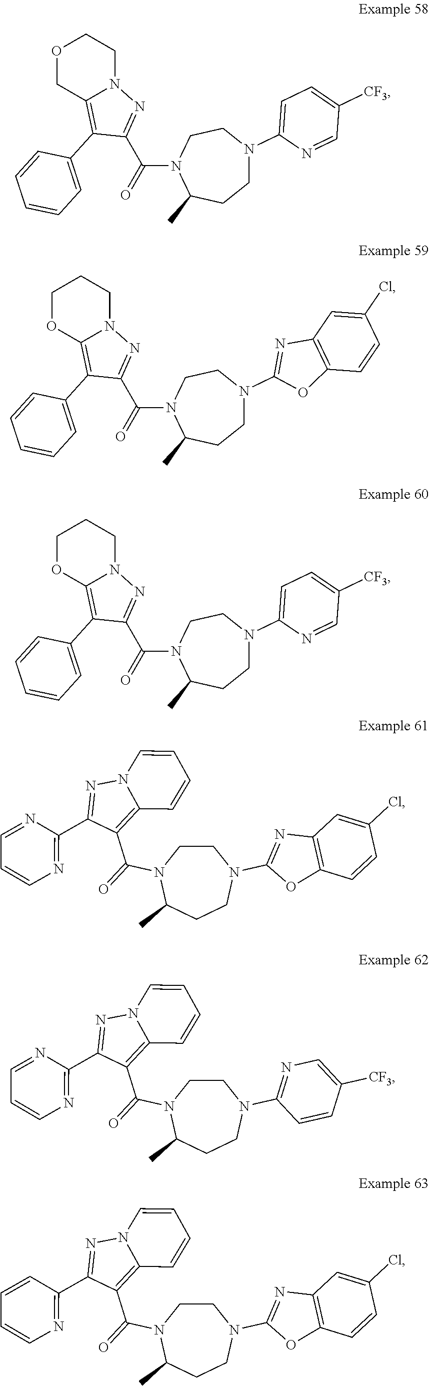

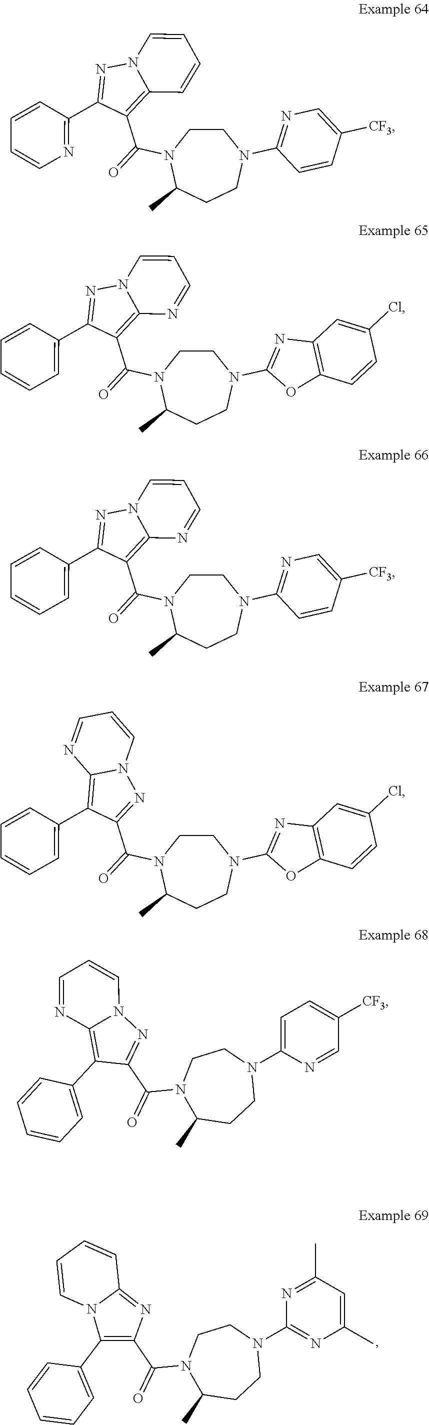 [SMILES: C[C@@H]1CCN(c2ccc(C(F)(F)F)cn2)CCN1C(=O)c1c(-c2ccccn2)nn2ccccc12 ; C[C@@H]1CCN(c2nc3cc(Cl)ccc3o2)CCN1C(=O)c1c(-c2ccccc2)nn2cccnc12 ; C[C@@H]1CCN(c2ccc(C(F)(F)F)cn2)CCN1C(=O)c1c(-c2ccccc2)nn2cccnc12 ; C[C@@H]1CCN(c2nc3cc(Cl)ccc3o2)CCN1C(=O)c1nn2cccnc2c1-c1ccccc1 ; C[C@@H]1CCN(c2ccc(C(F)(F)F)cn2)CCN1C(=O)c1nn2cccnc2c1-c1ccccc1 ; Cc1cc(C)nc(N2CC[C@@H](C)N(C(=O)c3nc4ccccn4c3-c3ccccc3)CC2)n1]

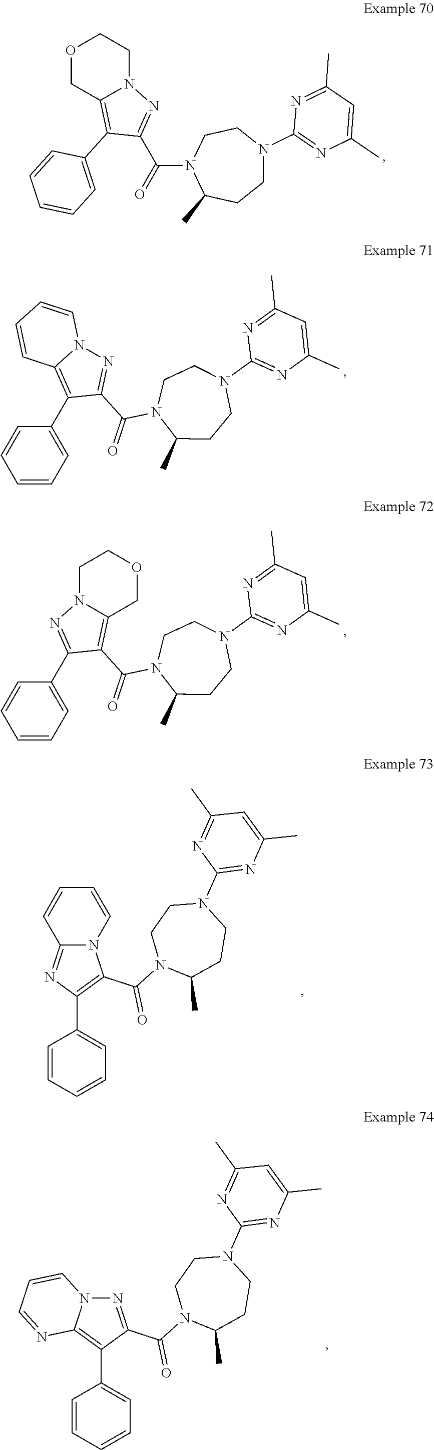 [SMILES: Cc1cc(C)nc(N2CC[C@@H](C)N(C(=O)c3nn4c(c3-c3ccccc3)COCC4)CC2)n1 ; Cc1cc(C)nc(N2CC[C@@H](C)N(C(=O)c3nn4ccccc4c3-c3ccccc3)CC2)n1 ; Cc1cc(C)nc(N2CC[C@@H](C)N(C(=O)c3c(-c4ccccc4)nn4c3COCC4)CC2)n1 ; Cc1cc(C)nc(N2CC[C@@H](C)N(C(=O)c3c(-c4ccccc4)nc4ccccn34)CC2)n1 ; Cc1cc(C)nc(N2CC[C@@H](C)N(C(=O)c3nn4cccnc4c3-c3ccccc3)CC2)n1]

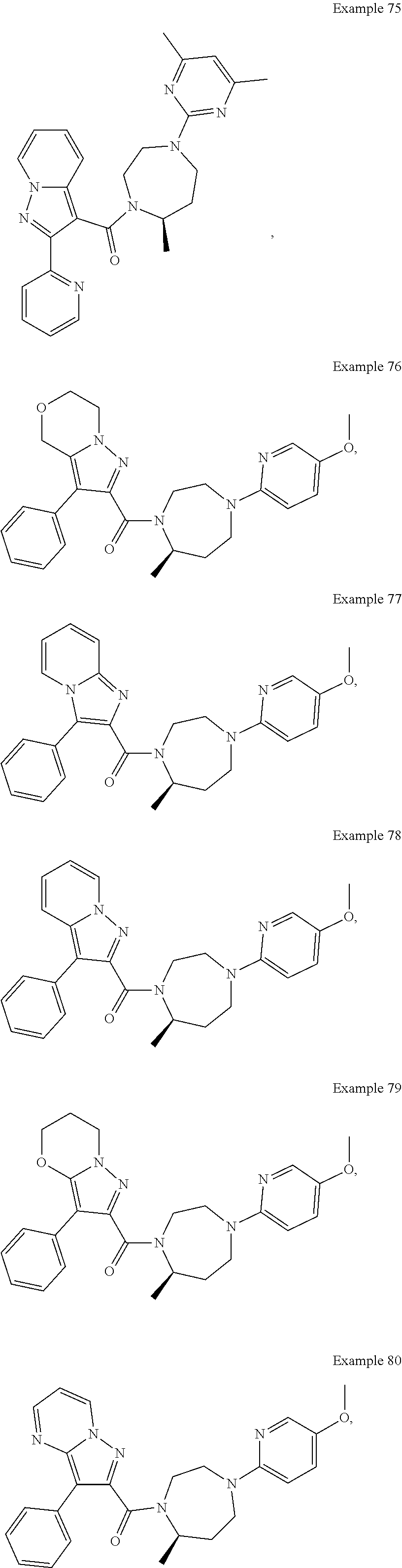 [SMILES: Cc1cc(C)nc(N2CC[C@@H](C)N(C(=O)c3c(-c4ccccn4)nn4ccccc34)CC2)n1 ; COc1ccc(N2CC[C@@H](C)N(C(=O)c3nn4c(c3-c3ccccc3)COCC4)CC2)nc1 ; COc1ccc(N2CC[C@@H](C)N(C(=O)c3nc4ccccn4c3-c3ccccc3)CC2)nc1 ; COc1ccc(N2CC[C@@H](C)N(C(=O)c3nn4ccccc4c3-c3ccccc3)CC2)nc1 ; COc1ccc(N2CC[C@@H](C)N(C(=O)c3nn4c(c3-c3ccccc3)OCCC4)CC2)nc1 ; COc1ccc(N2CC[C@@H](C)N(C(=O)c3nn4cccnc4c3-c3ccccc3)CC2)nc1]

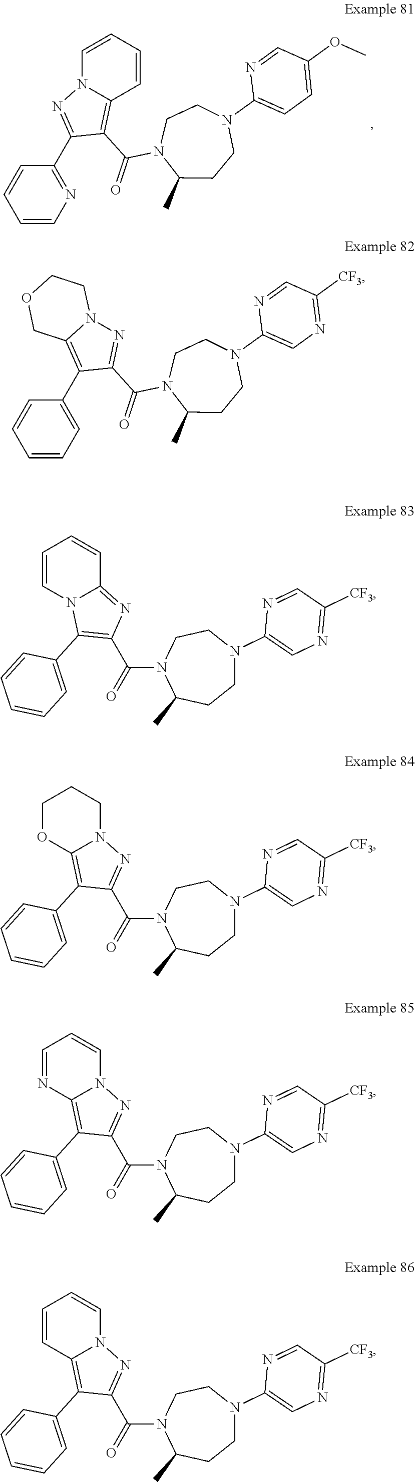 [SMILES: COc1ccc(N2CC[C@@H](C)N(C(=O)c3c(-c4ccccn4)nn4ccccc34)CC2)nc1 ; C[C@@H]1CCN(c2cnc(C(F)(F)F)cn2)CCN1C(=O)c1nn2c(c1-c1ccccc1)COCC2 ; C[C@@H]1CCN(c2cnc(C(F)(F)F)cn2)CCN1C(=O)c1nc2ccccn2c1-c1ccccc1 ; C[C@@H]1CCN(c2cnc(C(F)(F)F)cn2)CCN1C(=O)c1nn2c(c1-c1ccccc1)OCCC2 ; C[C@@H]1CCN(c2cnc(C(F)(F)F)cn2)CCN1C(=O)c1nn2cccnc2c1-c1ccccc1 ; C[C@@H]1CCN(c2cnc(C(F)(F)F)cn2)CCN1C(=O)c1nn2ccccc2c1-c1ccccc1]

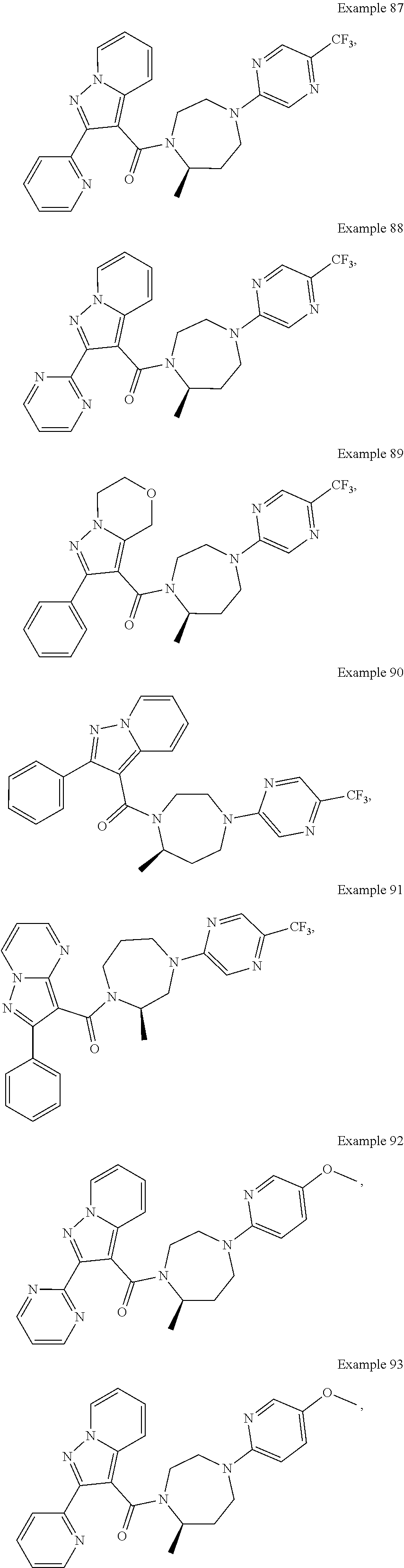

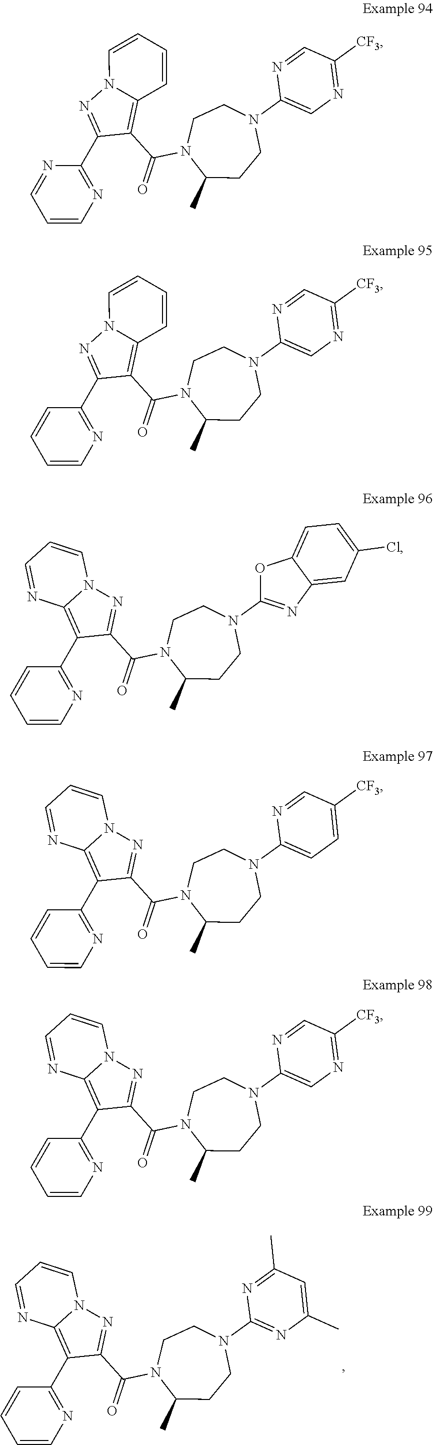 [SMILES: C[C@@H]1CCN(c2cnc(C(F)(F)F)cn2)CCN1C(=O)c1c(-c2ncccn2)nn2ccccc12 ; C[C@@H]1CCN(c2cnc(C(F)(F)F)cn2)CCN1C(=O)c1c(-c2ccccn2)nn2ccccc12 ; C[C@@H]1CCN(c2nc3cc(Cl)ccc3o2)CCN1C(=O)c1nn2cccnc2c1-c1ccccn1 ; C[C@@H]1CCN(c2ccc(C(F)(F)F)cn2)CCN1C(=O)c1nn2cccnc2c1-c1ccccn1 ; C[C@@H]1CCN(c2cnc(C(F)(F)F)cn2)CCN1C(=O)c1nn2cccnc2c1-c1ccccn1 ; Cc1cc(C)nc(N2CC[C@@H](C)N(C(=O)c3nn4cccnc4c3-c3ccccn3)CC2)n1]

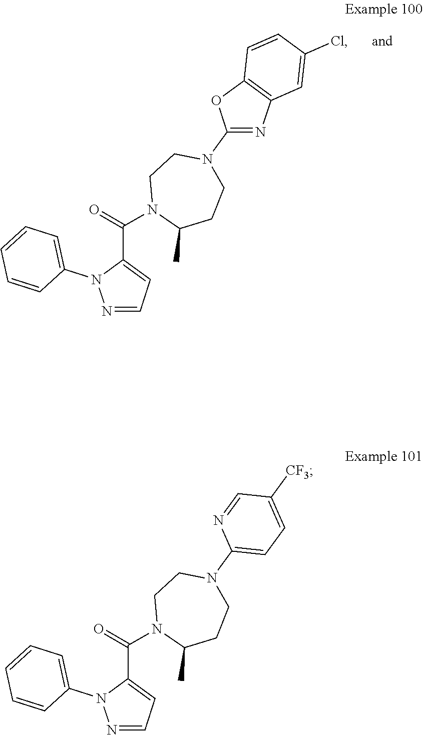 [SMILES: C[C@@H]1CCN(c2nc3cc(Cl)ccc3o2)CCN1C(=O)c1ccnn1-c1ccccc1 ; "and" ; C[C@@H]1CCN(c2ccc(C(F)(F)F)cn2)CCN1C(=O)c1ccnn1-c1ccccc1]

or a pharmaceutically acceptable salt, hydrate, solvate, polymorph, isomer, or combination thereof.
Aspect 6 (especially preferred embodiments). A compound of aspect 5 selected from the group consisting of:

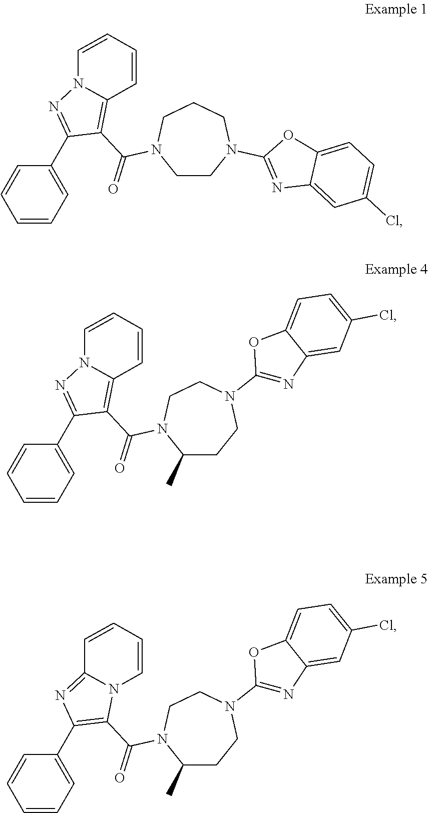 [SMILES: O=C(c1c(-c2ccccc2)nn2ccccc12)N1CCCN(c2nc3cc(Cl)ccc3o2)CC1 ; C[C@@H]1CCN(c2nc3cc(Cl)ccc3o2)CCN1C(=O)c1c(-c2ccccc2)nn2ccccc12 ; C[C@@H]1CCN(c2nc3cc(Cl)ccc3o2)CCN1C(=O)c1c(-c2ccccc2)nc2ccccn12]

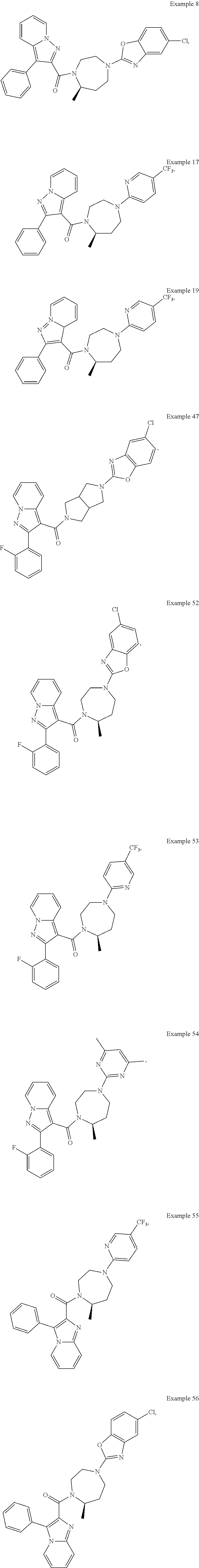

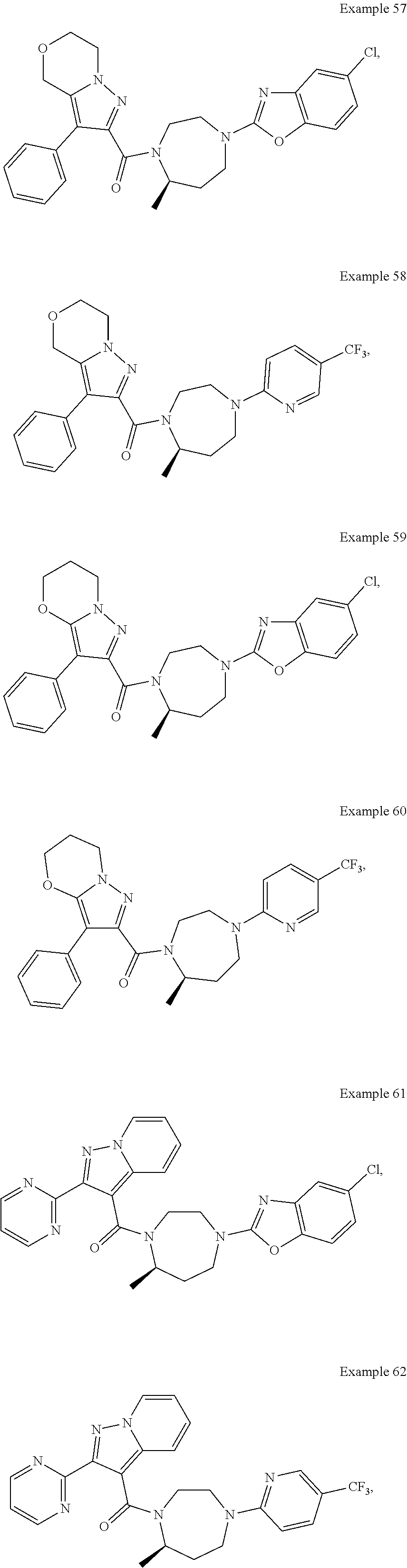 [SMILES: C[C@@H]1CCN(c2nc3cc(Cl)ccc3o2)CCN1C(=O)c1nn2c(c1-c1ccccc1)COCC2 ; C[C@@H]1CCN(c2ccc(C(F)(F)F)cn2)CCN1C(=O)c1nn2c(c1-c1ccccc1)COCC2 ; C[C@@H]1CCN(c2nc3cc(Cl)ccc3o2)CCN1C(=O)c1nn2c(c1-c1ccccc1)OCCC2 ; C[C@@H]1CCN(c2ccc(C(F)(F)F)cn2)CCN1C(=O)c1nn2c(c1-c1ccccc1)OCCC2 ; C[C@@H]1CCN(c2nc3cc(Cl)ccc3o2)CCN1C(=O)c1c(-c2ncccn2)nn2ccccc12 ; C[C@@H]1CCN(c2ccc(C(F)(F)F)cn2)CCN1C(=O)c1c(-c2ncccn2)nn2ccccc12]

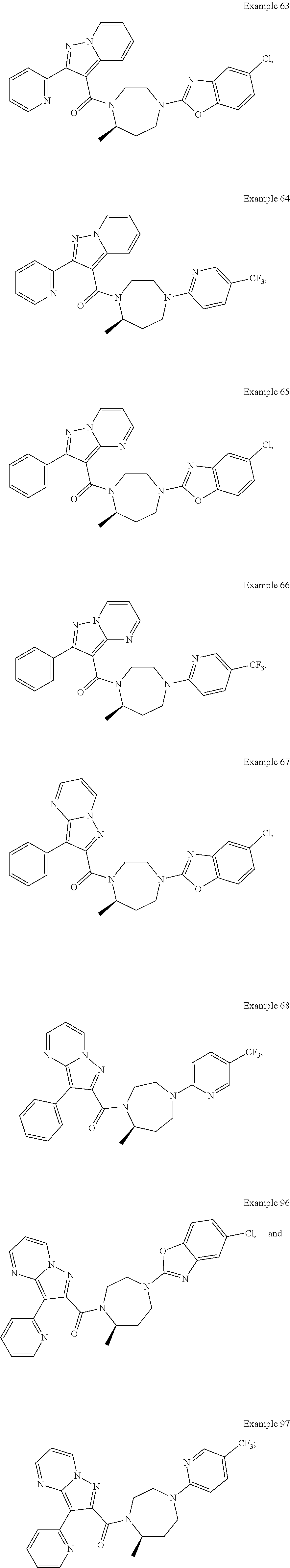

or a pharmaceutically acceptable salt, hydrate, solvate, polymorph, isomer, or combination thereof.
Aspect 7. A pharmaceutical composition comprising a compound according to any preceding claim, or a pharmaceutically acceptable salt, hydrate, solvate, polymorph, isomer, or combination thereof; and an acceptable carrier, adjuvant or vehicle.
Aspect 8. The pharmaceutical composition of aspect 6 comprising a therapeutically effective amount of the compound, pharmaceutically acceptable salt, hydrate, solvate, polymorph, isomer, or combination thereof.
Aspect 9. The pharmaceutical composition according to aspect 7 or 8, wherein said composition further comprises at least one second therapeutic agent.
Aspect 10. A method for antagonizing at least one orexin receptor in a cell, comprising the step of exposing the cell to a compound and/or composition according to any preceding claim, optionally wherein said method is in vitro.
Aspect 11. A method for antagonizing at least one orexin receptor in a subject in need thereof, comprising the step of administering a compound and/or composition according to any preceding claim.
Aspect 12. A method of treating a condition selected from the group consisting of substance addiction, substance dependence, panic, anxiety, depression, posttraumatic stress disorders (PTSD), neurodegeneration, autism, schizophrenia, pain, Alzheimer diseases (AD), and a central nervous system (CNS) disorder in a subject in need thereof, comprising the step of administering a compound and/or composition according to any preceding claim.
Aspect 13. The method of aspect 12 wherein the substance corresponding to the substance addiction or substance dependence is selected from the group consisting of one or more opioids, optionally heroin, morphine, oxycodone, fentanyl, and hydrocodone; one or more stimulants optionally selected from the group consisting of amphetamine, cocaine, crack cocaine, and methamphetamine; one or more sedatives and/or tranquilizers, benzodiazepine, and barbituates.
Aspect 14. The method of any one of aspects 10-13 wherein the compound is an orexin receptor antagonist.
Aspect 15. The method of any one of aspects 10-14 wherein the compound is an antagonist of orexin receptor 1 (OX₁R) or orexin receptor 2 (OX₂R), or an antagonist of both OX₁R or OX₂R; optionally wherein the compound is an antagonist of the K-opioid receptor, and/or exhibits the following characteristics as measured in an in vitro cellular assays: 1) OX₁R Kb<30 nm, OX₂R Kb>1000 nm, KOR Ki<500 nm; 2) OX₁R Kb<30 nm, OX₂R Kb>30 nm, KOR Ki<500 nm; or, 3) OX₁R Kb<30 nm, OX₂R Kb>1000 nm, KOR Ki>1000 nm.
Aspect 16. A method of any one of aspects 10-15 comprising administering to the patient a therapeutically effective amount of a compound and/or composition of any preceding claim.
Aspect 17. A method of any one of aspects 10-16 comprising administering at least one unlabeled form and/or at least one isotopically labeled form of compound of any preceding claim, and/or a composition comprising the same.
Aspect 18. A method for manufacturing a compound of aspect 1 comprising combining at least two intermediates to produce the compound. In preferred embodiments, the intermediates, reaction conditions and the like used to produce the compounds are described in detail in the Examples section, which is not reproduced in detail here but will be understood by those of skill in the art therefrom as if the same was reproduced in describing this preferred aspect (i.e., incorporated herein).
Aspect 19. A method for manufacturing a pharmaceutical composition of aspect 1 comprising combining at least one compound of aspect 1 with at least one pharmaceutically acceptable excipient. Methods for making such combinations (i.e., at least one compound or the like and at least one pharmaceutical composition) are generally known in the art and are therefore not described in detail within this aspect, but are incorporated herein.

The term “therapeutically effective amount” refers to an amount of a compound that, when administered to a subject, is sufficient to prevent, reduce the risk of developing, delay the onset of, slow the progression of or cause regression of the medical condition being treated, or to alleviate to some extent the medical condition or one or more symptoms or complications of that condition, at least in some fraction of the subjects taking that compound. The term “therapeutically effective amount” also refers to an amount of a compound that is sufficient to elicit the biological or medical response of a cell, tissue, organ or human which is sought by a medical doctor or clinician. The terms “treat,” “treating” and “treatment” include alleviating, ameliorating, inhibiting the progress of, reversing or abrogating a medical condition or one or more symptoms or complications associated with the condition, and alleviating, ameliorating or eradicating one or more causes of the condition. Reference to “treatment” of a medical condition includes prevention of the condition. The terms “prevent”, “preventing” and “prevention” include precluding, reducing the risk of developing and delaying the onset of a medical condition or one or more symptoms or complications associated with the condition. The term “medical conditions” (or “conditions” for brevity) includes diseases and disorders. The terms “diseases” and “disorders” are used interchangeably herein.

Throughout this specification the word “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The symbol “=” when used in describing a formula means “is”.

The term “including” is used to mean “including but not limited to “Including” and “including but not limited to” are used interchangeably. The term “agent” is used herein to denote a chemical compound (such as an organic or a mixture of chemical compounds). Agents include, for example, agents that are known with respect to structure, and their orexin antagonist activities of such agents may render them suitable as “therapeutic agents” in the methods and compositions disclosed herein. In addition, those of ordinary skill in the art recognize that it is common to use the following abbreviations:
Me: methyl
Et: ethyl
t-Bu: tert-butyl
Ar: aryl
Ph: phenyl
BINAP: 2,2-bis(diphenylphosphino)-1,1′-binaphthyl
Bn: benzyl
Ac: acetyl
Boc: tert-butyloxy carbonyl
BSA: bovine serum albumin
CbzCl: benzylchloroformate
CDI: carbonyl diimidazole
DCM: dichloromethane
DCE: dichloroethane
DEAD: diethylazodicarboxylate
DIPEA: N,N-diisopropylethylamine
DMF: N,N-dimethylformamide
DMSO: dimethylsulfoxide
CH₂Cl₂: dichloromethane
EDC: N-(3-Dimethylaminopropyl)-N′-ethylcarbodiimide
Et₃N: triethylamine
EtOAc: ethyl acetate
EtOH: ethanol
HCl: hydrogen chloride
HOAt: 1-hydroxy-7-aza-10-benzotriazole
HOBT: hydroxybenzotriazole hydrate
LCMS: Liquid Chromatography Mass Spectrometry
HPLC: High Performance Liquid Chromatography
Hunig's base: N,N-diisopropylethylamine
MeOH: methanol
MgSO₄: magnesium sulfate
MTBE: methyl tert-butyl ether
NaHCO₃: sodium bicarbonate
Na2CO3: sodium carbonate
K2CO3: potassium carbonate
NaOH: sodium hydroxide
NMM: N-methylmorpholine
Pt0₂: Platinum oxide
Pd: Palladium
Pd/C: Palladium over carbon
PyClu: 1-(chloro-1pyrrolidinylmethylene)pyrrolidiniumhexafluorophosphate
RT or rt: room temperature
Rxn: reaction
SOCl₂: thionyl chloride
THF: tetrahydrofuran
TFA: trifluoroacetic acid
X-Phos: 2-(dicyclohexyl-phosphino)-2′,4′,6′-triisopropylbiphenyl
HATU: (1-[Bis(dimethylamino)methylene]-1H-1,2,3-triazolo[4,5-b]pyridinium 3-oxid hexafluorophosphate
NMR: Nuclear Magnetic Resonance
ESI: Electrospray Ionization
MS: Mass spectrometry reaction

All references cited within this disclosure are hereby incorporated by reference in their entirety. Certain embodiments are further described in the following examples. These embodiments are provided as examples only and are not intended to limit the scope of the claims in any way.

### EXAMPLES

### Example 1

For this disclosure, the following intermediates were prepared and used for the synthesis of examples claimed herein:

1. Carboxylic Containing Intermediates prepared—Carboxylic Acids Group

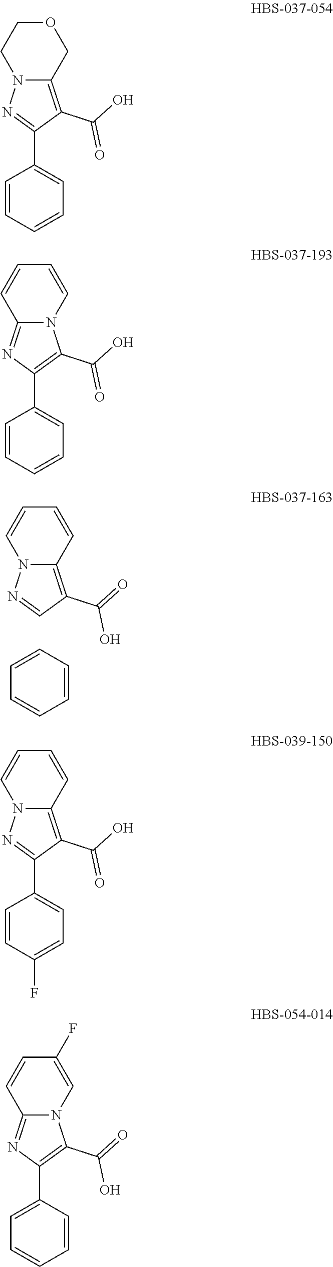

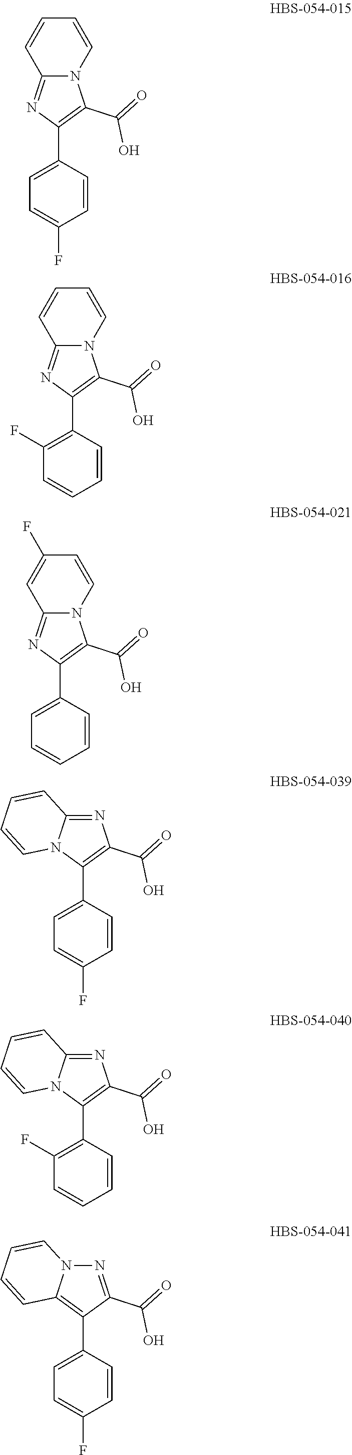

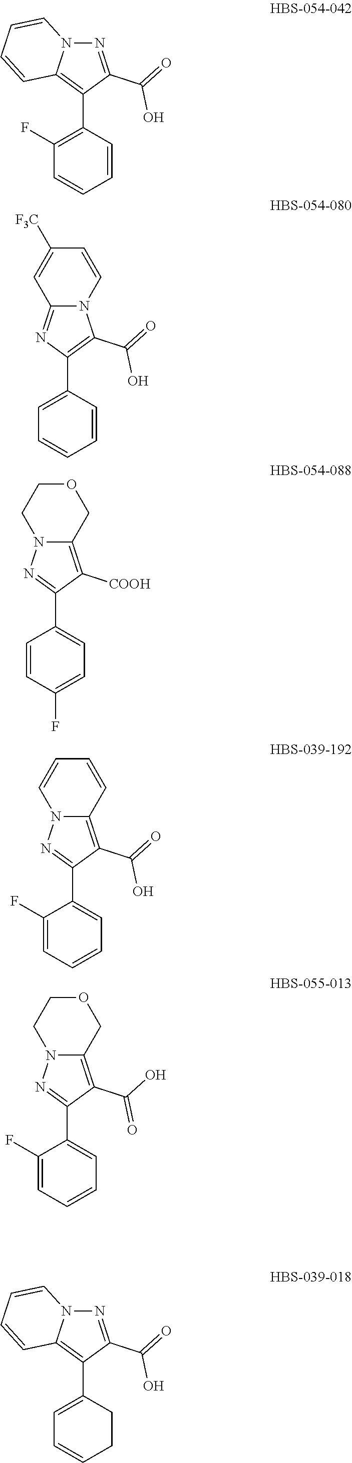

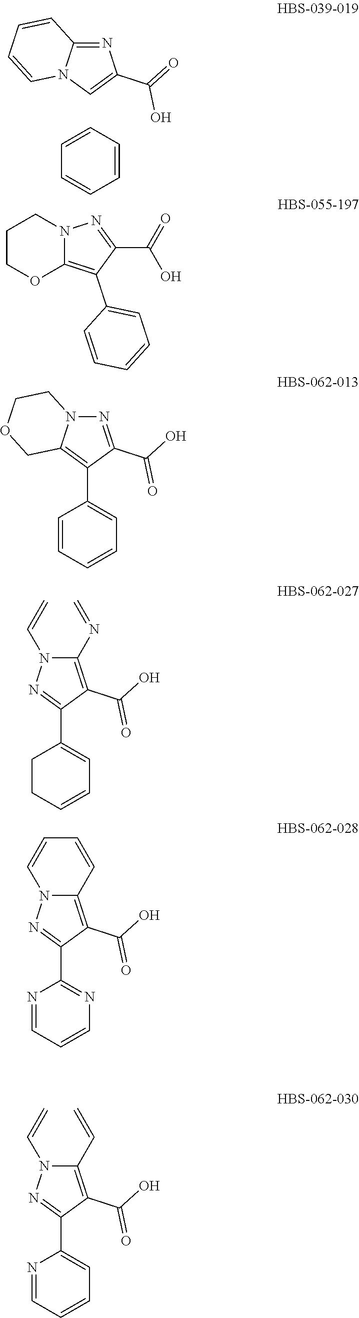

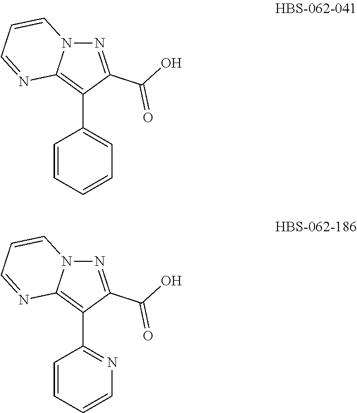 [SMILES: O=C(O)c1nn2cccnc2c1-c1ccccc1 ; O=C(O)c1nn2cccnc2c1-c1ccccn1]

and,
2. Secondary Amine Containing Intermediates prepared—Amine Group

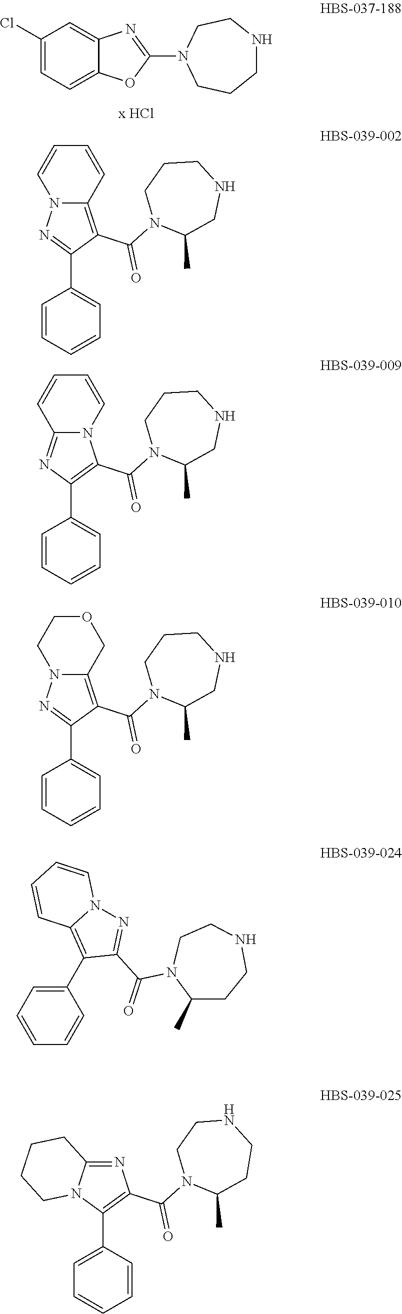

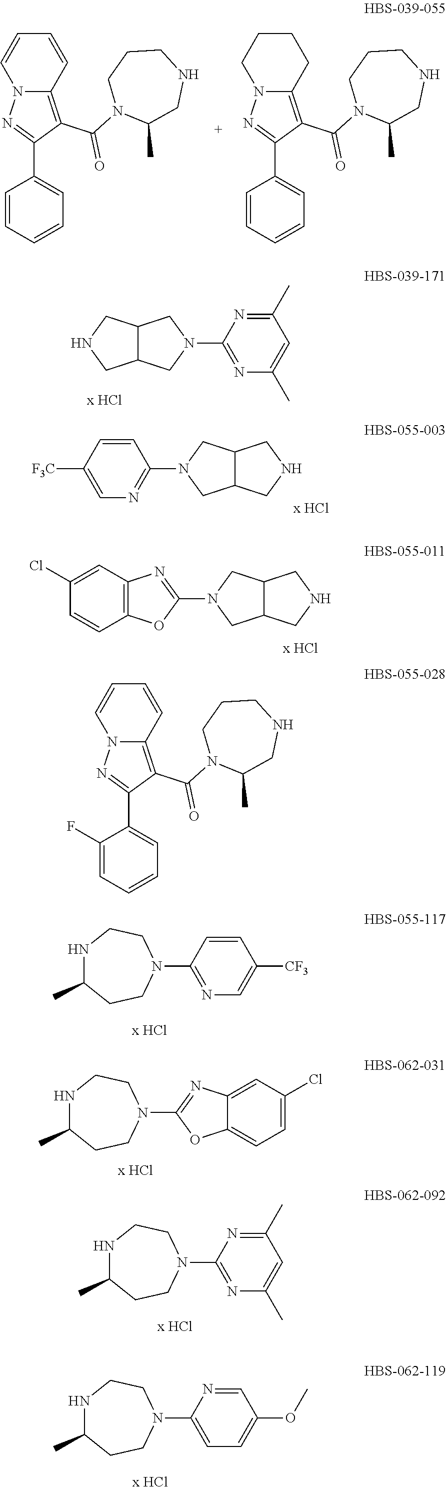

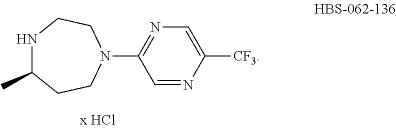 [SMILES: C[C@@H]1CCN(c2cnc(C(F)(F)F)cn2)CCN1]

### I. General Synthetic Methods, Procedures & Experimental

### A. General

All temperatures are stated in ° C. Commercially available starting materials were used as received without further purification. Unless otherwise specified, all reactions were carried out in oven-dried glassware under an atmosphere of nitrogen. Compounds were purified by flash column chromatography on silica gel or by preparative HPLC. Compounds described in this disclosure are characterized by LC-MS data (retention time t_{R} is given in min; molecular weight obtained from the mass spectrum is given in g/mol) using the conditions described below.

### LC-MS Under Acidic Conditions

Method A: Agilent 1100 series with mass spectrometry detection (MS: Agilent single quadrupole). Column: Zorbax SB (3.5 μm, 4.6×150 mm). Conditions: MeCN (0.1% FA) [gradient eluent A]; water (0.1% FA) [gradient eluent B]. Gradient: 95% B+5% B over 5 min (flow: 0.8 ml/min). Detection: UV 280/254 nm+MS.

Method B: Agilent 1100 series with mass spectrometry detection (MS: Agilent single quadrupole). Column: X-Bridge C₁₈ (3.5 μm, 4.6×150 mm). Conditions: MeCN (0.1% FA) [gradient eluent A]; water (0.1% FA) [gradient eluent B]. Gradient: 95% B+5% B over 5 min (flow: 0.8 ml/min). Detection: UV 280/254 nm+MS.

In general, the compounds of this disclosure may be prepared by methods known to those skilled in the art and contemporary technologies in the field. Schemes 1-4 below illustrate synthetic routes to the compounds of the present disclosure. Other equivalent schemes, which will be readily apparent to the ordinary skilled synthetic organic or medicinal chemist, may alternatively be used to synthesize various portions of the molecules as illustrated by the general schemes described herein.

### B. Intermediates Synthesis

### 1. Synthesis of Intermediate HBS-037-054

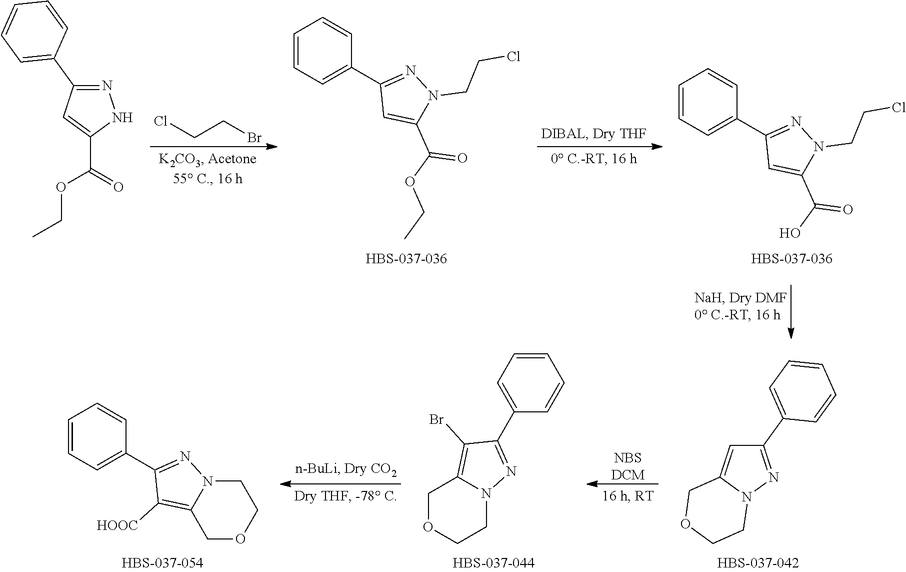

Step 1: Synthesis of Compound HBS-037-036: Ethyl-3-Phenyl-1H-pyrazole-5-carboxylate (0.5 g, 2.31 mmol) was dissolved in acetone (10.0 mL). The K₂CO₃ (0.96 g, 6.9 mmol) was added, followed by 1-bromo-2-chloro-ethane (0.1 mL, 11.6 mmol). The reaction mixture was heated at 55° C. for 16 hours (h). LCMS data shows desired product formation m/z 279.0 and minor amount of side product formation. The reaction mixture was filtered and the solid was washed with ethyl acetate. The filtrate was concentrated under reduced pressure to obtain crude product. The crude product was purified by column chromatography, Mobile Phase: EtOAc:Hexane, gradient. The compound (0.6 g) was obtained as a liquid (Yield 93.2%). MS (ESI) mass calculated for C₁₄H₁₅ClN₂O₂, 278.7; m/found 279.0 [M+H]⁺

Step 2: Synthesis of Compound HBS-037-040: Compound HBS-037-036 (0.55 g, 1.97 mmol) was dissolved in dry THF (6.0 mL). The DIBAL (12.0 mL, 1.0 M solution, 11.8 mmol) was added, under ice cooling bath. The reaction mixture was gradually warmed to room temperature and stirred for 16 h. LCMS data shows desired product formation m/z 237.0. The reaction mixture was quenched with 1.0 N aq. NaOH solution and diluted with ethyl acetate (10.0 mL). The reaction mixture was filtered through celite bed and washed with ethyl acetate (10.0 mL×3). The EtOAc layer was separated and washed with water followed by brine. The organic layer was dried over anhydrous sodium sulfate. Evaporation of solvent gave crude product 0.4 g (Yield 85.6%). MS (ESI) mass calculated for C₁₂H₁₃ClN₂O, 236.7; m/z found 237.0 [M+H]⁺

Step 3: Synthesis of Compound HBS-037-043: Compound HBS-037-040 (0.47 g, 1.97 mmol) was dissolved in dry DMF (12.0 mL). The NaH (0.12 g, 2.96 mmol) was added under ice cooling. The reaction mixture was gradually warmed to room temperature and stirred for 16 h. LCMS data shows desired product formation m/z 201.1. The reaction mixture was diluted with water and the product was extracted with ethyl acetate. The combined ethyl acetate layer was separated and dried over anhydrous sodium sulfate. The evaporation of solvent gave crude product. The crude product was purified by column chromatography, Mobile Phase: EtOAc:Hexane, gradient. The compound (0.26 g) was obtained as a solid (Yield 65.3%). MS (ESI) mass calculated for C₁₂H₁₂N₂O, 200.2; m/z found 201.1 [M+H]⁺

Step 4: Synthesis of Compound HBS-037-043: Compound HBS-037-042 (0.25 g, 1.25 mmol) was dissolved in DCM (5.0 mL). The NBS (0.24 g, 1.37 mmol) was added, and reaction mixture was stirred at room temperature for 16 h. LCMS data shows desired product formation m/z 280.9. Evaporation of solvent gave crude product, which was purified by column chromatography, Mobile Phase: EtOAc:Hexane, gradient. Compound HBS-037-043 was obtained (0.29 g) as a liquid. (Yield 81.7%). MS (ESI) mass calculated for C₁₂H₁₁BrN₂O, 279.1; m/z found 280.9 [M+H]⁺

Step 5: Synthesis of Intermediate HBS-037-054: Compound HBS-037-043 (0.025 g, 0.09 mmol) was dissolved in anhydrous THF (1.0 mL) under N₂ atm. The reaction mixture was cooled at −78.0° C. temperature and n-BuLi (0.12 mL, 1.6 M) was added into the reaction mixture. The reaction mixture was stirred at −78.0° C. temperature for 30.0 min. Dry CO₂ gas was bubbled through the reaction mixture at −65° C. and the reaction mixture was gradually warmed at room temperature. The LCMS data shows desired product formation m/z 245, de-brominated side product (m/z 201) and some unknown product formation. The reaction mixture was quenched by water and extracted with ethyl acetate. The ethyl acetate layer was separated and de-brominated product was recovered. The aq. layer was acidified with 1 M HCl solution and evaporated to dryness to obtain 0.022 g of solid product. MS (ESI) mass calculated for C₁₃H₁₂N₂O₃, 244.3; m/z found 245.0 [M+H]⁺, ¹H NMR (400 MHz, CHLOROFORM-d) δ ppm 4.06-4.15 (m, 2H) 4.16-4.25 (m, 2H) 5.03-5.10 (s, 2H) 7.32-7.40 (m, 3H) 7.60-7.69 (m, 2H).

### 2. Synthesis of Intermediate HBS-037-193:

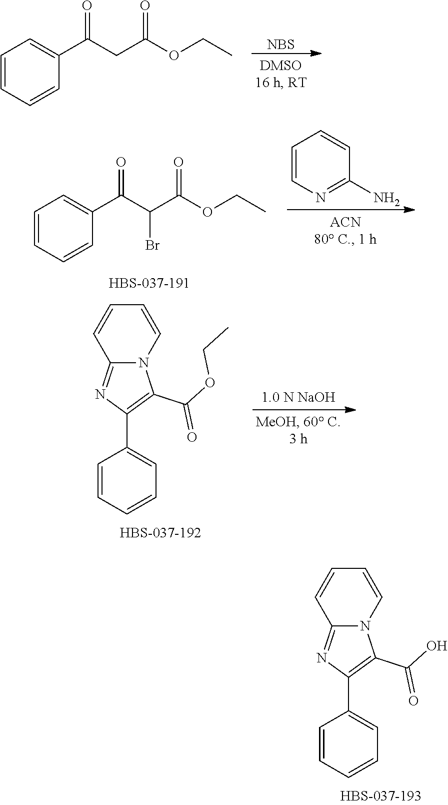

Step 1: Synthesis of Compound HBS-037-191: Ethyl benzoylacetate (0.5 g, 2.6 mmol) was dissolved in DMSO (5.0 mL). The NBS (0.51 g, 2.86 mmol) was added and reaction mixture was stirred at ambient temperature for 24 h. LCMS shows product formation m/z 270.9. The reaction mixture was diluted with water and the product was extracted with ethyl acetate. The ethyl acetate layer was separated and dried over anhydrous Na₂SO₄. Evaporation of solvent gave crude product which was purified by combi-flash system, Mobile phase: EtOAc:Hexane gradient. The 0.42 g of product was obtained (Yield 59.7%). MS (ESI) mass calculated for C₁₁H₁₁BrO₃, 271.1; m/z found 270.9 [M+H]⁺.

Step 2: Synthesis of Compound HBS-037-192: Compound HBS-037-191 (0.42 g, 1.55 mmol) was dissolved in anhydrous acetonitrile (5.0 mL). 2-Amino-pyridine (0.15 g, 1.55 mmol) was added and reaction mixture was stirred at 80° C. for 1 h. LCMS shows product formation m/z 267.1. The reaction mixture was concentrated under reduced pressure to yield crude product. The crude product was purified by combi-flash system, Mobile phase: EtOAc:Hexane gradient. Compound HBS-037-192 (0.24 g) was obtained (Yield 58.0%). MS (ESI) mass calculated for C₁₈H₁₄N₂O₂, 266.3; m/z found 267.1 [M+H]⁺.

Step 3: Synthesis of Intermediate HBS-037-193: Compound HBS-037-192 (0.24 g, 0.9 mmol) was dissolved in MeOH (5.0 mL). The 1.0 N aq. NaOH solution (4.51 mL, 4.51 mmol) was added and reaction mixture was stirred at 60° C. temperature for 3 h. LCMS shows formation of product m/z 239. The reaction mixture was concentrated under reduced pressure and the solid was dissolved in water and acidified with 2.0 M aq. HCl solution (pH=5). The ppts were filtered and washed with water (10.0 mL×3) to obtain 0.2 g of solid product. MS (ESI) mass calculated for C₁₄H₁₀N₂O₂, 238.2; m/z found 239.1 [M+H]⁺, ¹H NMR (400 MHz, CHLOROFORM-d) δ ppm 7.04 (t, J=6.93 Hz, 1H) 7.33-7.48 (m, 4H) 7.70-7.78 (m, 3H) 9.41 (d, J=7.04 Hz, 1H).

### 3. Synthesis of Intermediate HBS-037-163:

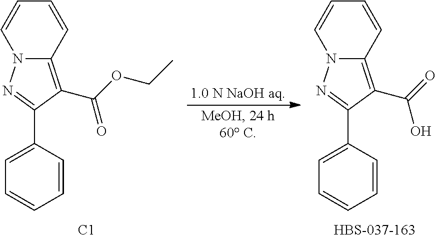

Step 1: Synthesis of Intermediate HBS-037-163: Compound C₁ (synthesized as reported in Journal of Medicinal Chemistry; 2011, 54 (13), 4752-4772) (0.2 g, 0.75 mmol) was dissolved in MeOH (6.0 mL). The 1.0 N aq. NaOH solution (3.8 mL, 3.8 mmol) was added and reaction mixture was stirred at 60° C. temperature for 24 h. LCMS shows product formation m/z 239.1. The reaction mixture was concentrated under reduced pressure and the solid was dissolved in water and acidified with 1.0 M aq. HCl solution (pH=5). The ppts were filtered and washed with water (10.0 mL×3) to obtain 0.18 g of solid product. MS (ESI) mass calculated for C₁₄H₁₀N₂O₂, 238.2; m/z found 239.1 [M+H]⁺, ¹H NMR (400 MHz, CHLOROFORM-d) δ ppm 6.90-7.03 (m, 1H) 7.27-7.47 (m, 4H) 7.75-7.78 (ddd, J=4.86, 3.21, 1.54 Hz, 2H) 8.18-8.28 (dd, J=8.99, 0.70 Hz, 1H) 8.49-8.56 (dd, J=6.90, 0.73 Hz, 1H).

### 4. Synthesis of Intermediate HBS-037-188:

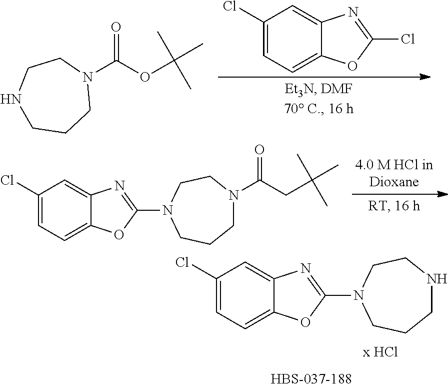

Step 1: Synthesis of Compound HBS-037-187: tert-Butyl-1,4-diazepane-1-carboxylate (0.5 g, 2.5 mmol) and 2,5-dichlorobenzoxazole (0.47 g, 2.5 mmol) were dissolved in anhydrous DMF (10.0 mL). The triethyl amine (1.0 mL 7.5 mmol) was added and reaction mixture was heated at 70° C. temperature for 16 h. LCMS data shows product formation m/z 352.1. The reaction mixture was diluted with water and the product was extracted with ethyl acetate. The combined ethyl acetate layer was washed with water followed by brine. The organic layer was separated and dried over anhydrous sodium sulfate. The evaporation of solvent gave crude product which was purified by column chromatography, Mobile Phase: EtOAc:Hexane, gradient. The 0.88 g of product was isolated (Yield Quant.). MS (ESI) mass calculated for C₁₇H₂₂ClN₃O₃, 351.8; m/z found 352.1 [M+H]⁺

Step 2: Synthesis of Intermediate HBS-037-188: Compound HBS-037-187 (0.88 g, 2.5 mmol) was dissolved in anhydrous dioxane (10.0 mL). The 4.0 M HCl solution in dioxane (6.2 mL, 25.0 mmol) was added and reaction mixture was stirred at ambient temperature for 16 h. LCMS data shows product formation m/z 252. The reaction mixture was concentrated under reduced pressure to obtain the product (0.9 g, Yield Quant.). MS (ESI) mass calculated for C₁₂H₁₄ClN₃O, 251.7; m/z found 252.0 [M+H]⁺

### Synthesis of Intermediate HBS-039-002:

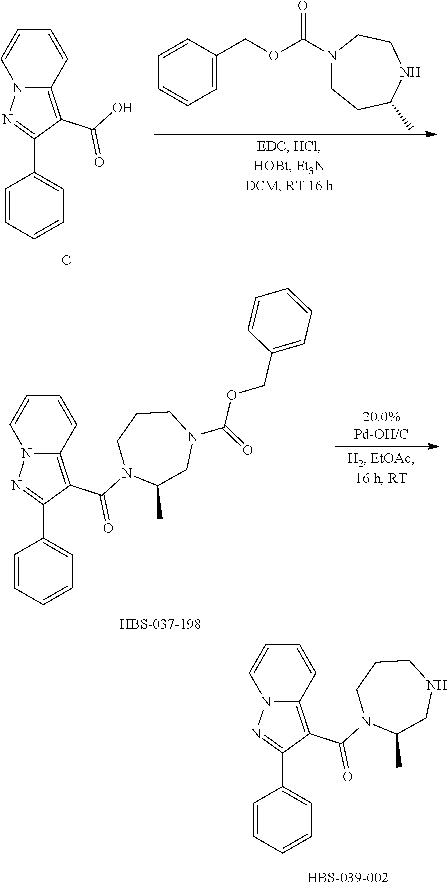

Step 1: Synthesis of Compound HBS-037-198: Intermediate C (0.029 g, 0.12 mmol) was dissolved in anhydrous DCM (2.5 mL). The EDC·HCl (0.046 g, 0.24 mmol) and HOBt (0.032 g, 0.24 mmol) were added followed by Et₃N (0.17 mL, 1.21 mmol). The reaction mixture was stirred at ambient temperature for 10.0 min. The benzyl-(5R)-5-methyl-1,4-diazepane-1-carboxylate (0.03 g, 0.12 mmol) solution in DCM (2.0 mL) was added to the reaction mixture. The reaction mixture was stirred at ambient temperature for 16 h. The LCMS data shows product formation m/469.2. The reaction mixture was diluted with saturated solution of NaHCO₃ and the product was extracted with DCM. The combined DCM layer was separated and dried over anhydrous Na₂SO₄. The evaporation of solvent gave crude product which was purified by ISCO combi-flash system, Mobile phase: EtOAc:Hexane gradient. Product was obtained as a solid (0.055 g) (Yield 97.1%). MS (ESI) mass calculated for C₂₈H₂₈N₄O₃, 468.55; m/z found 469.2 [M+H]⁺

Step 2: Synthesis of Intermediate HBS-039-002: Compound HBS-037-198 (0.055 g, 0.11 mmol) was dissolved in ethyl acetate (3.0 mL). The 20.0% Pd—OH/C (10.0 mg) was added and reaction mixture was stirred at ambient temperature under hydrogen atmosphere for 16 h. The LCMS data shows product formation m/z 335.2. The reaction mixture was filtered over celite bed and washed with ethyl acetate.

The filtrate was evaporated under reduced pressure to obtain 37.1 mg of crude product which was used in next step without purification. MS (ESI) mass calculated for C₂₀H₂₂N₄O, 334.4; m/z found 335.2 [M+H]⁺

### 6. Synthesis of Intermediate HBS-039-011A/B:

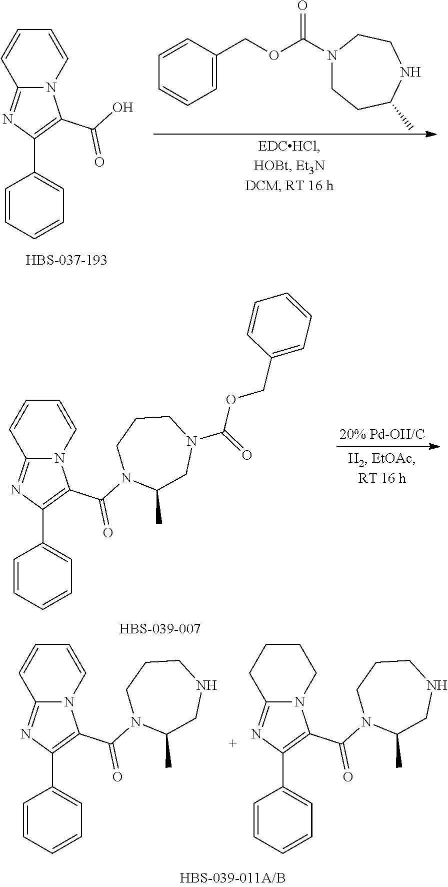

Step 1: Synthesis of Compound HBS-039-007: Intermediate HBS-039-193 (0.029 g, 0.12 mmol) was dissolved in anhydrous DCM (2.0 mL). The EDC·HCl (0.046 g, 0.24 mmol) and HOBt (0.032 g, 0.24 mmol) were added followed by Et₃N (0.17 mL, 1.21 mmol). The reaction mixture was stirred at ambient temperature for 10.0 min. Benzyl-(5R)-5-methyl-1,4-diazepane-1-carboxylate (0.03 g, 0.12 mmol) solution in DCM (2.0 mL) was added to the reaction mixture which was stirred at ambient temperature for 16 h. The LCMS data shows product formation m/z 469.2. The reaction mixture was diluted with saturated solution of NaHCO₃ and the product was extracted with DCM. The combined DCM layer was separated and dried over anhydrous Na₂SO₄. The evaporation of solvent gave crude product which was purified by ISCO combi-flash system, Mobile phase: EtOAc:Hexane gradient. Product HBS-039-007 (0.033 g) was obtained (Yield 58.3%). MS (ESI) mass calculated for C₂₈H₂₈N₄O₃, 468.55; m/z found 469.2 [M+H]⁺.

Step 2: Synthesis of Intermediate HBS-039-011A/B: Compound HBS-039-007 (0.033 g, 0.07 mmol) was dissolved in ethyl acetate (5.0 mL). The 20.0% Pd—OH/C (10.0 mg) was added and reaction mixture was stirred at ambient temperature under hydrogen atmosphere for 16 h. The LCMS data shows product formation m/z 335.2 and over-reduction side product formation m/z 339.2. The reaction mixture was filtered over celite bed and washed with ethyl acetate. The filtrate was evaporated under reduced pressure to obtain 22.0 mg of crude product which was used in the next step without purification. MS (ESI) mass calculated for C₂₀H₂₂N₄O, 334.4; m/z found 335.2 [M+H]⁺.

### 7. Synthesis of Intermediate HBS-039-010:

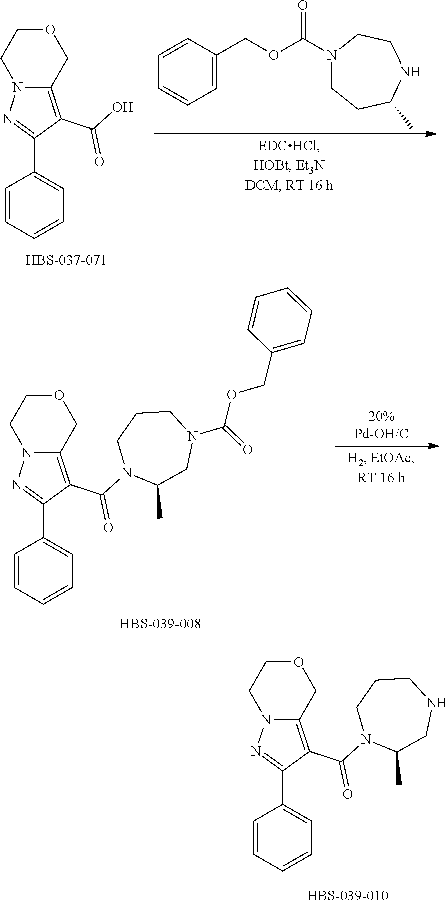

Step 1: Synthesis of Compound HBS-039-008: Intermediate HBS-037-071 (0.044 g, 0.18 mmol) was dissolved in anhydrous DCM (2.0 mL). EDC·HCl (0.046 g, 0.24 mmol) and HOBt (0.032 g, 0.24 mmol) were added followed by Et₃N (0.17 mL, 1.21 mmol). The reaction mixture was stirred at ambient temperature for 10.0 min, then a benzyl-(5R)-5-methyl-1,4-diazepane-1-carboxylate (0.03 g, 0.12 mmol) solution in DCM (2.0 mL) was added to the reaction mixture which was stirred at ambient temperature for 16 h. The LCMS data shows product formation m/z 475.2. The reaction mixture was diluted with saturated solution of NaHCO₃ and the product was extracted with DCM. The combined DCM layer was separated and dried over anhydrous Na₂SO₄. The evaporation of solvent gave crude product which was purified by ISCO combi-flash system, Mobile phase: EtOAc:Hexane gradient. Compound HBS-039-008 (0.023 g) was obtained (Yield 26.7%). MS (ESI) mass calculated for C₂₇H₃₀N₄O₄, 474.55; m/z found 475.2 [M+H]⁺.

Step 2: Synthesis of Intermediate HBS-039-010: Compound HBS-039-008 (0.023 g, 0.048 mmol) was dissolved in ethyl acetate (5.0 mL). 20.0% Pd—OH/C (5.0 mg) was added, and reaction mixture was stirred at ambient temperature under hydrogen atmosphere for 16 h. The LCMS data shows product formation m/z 341.2. The reaction mixture was filtered over celite bed and washed with ethyl acetate.

The filtrate was evaporated under reduced pressure to obtain 14.0 mg of crude product which was used in next step without purification. MS (ESI) mass calculated for C₁₀H₂₄N₄O₂, 340.4; m/z found 341.2 [M+H]⁺.

### 8. Synthesis of Intermediate HBS-039-024:

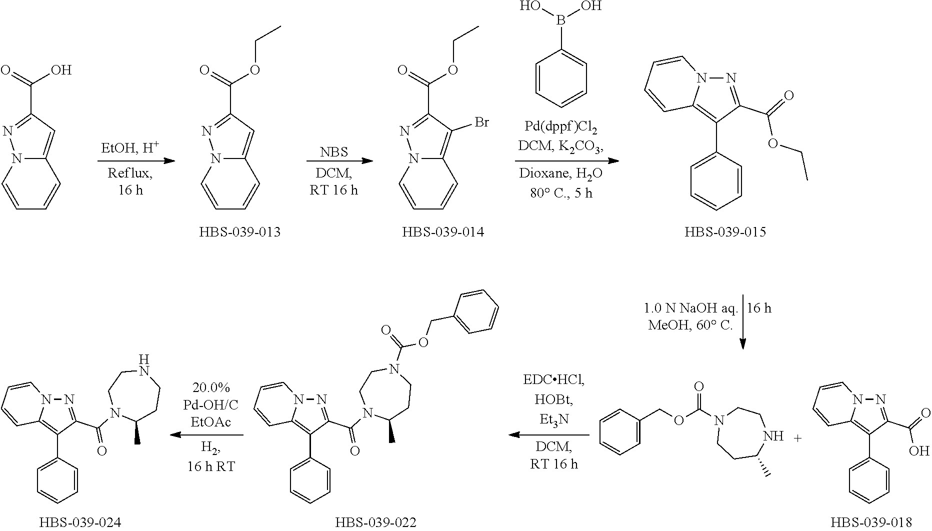

Step 1: Synthesis of Compound HBS-039-013: Pyrazolo[1,5-a]pyridine-2-carboxylic acid (1.0 g, 6.17 mmol) was dissolved in ethanol (20.0 mL). Catalytic amount of conc. sulfuric acid (0.5 mL) was added and reaction mixture was refluxed for 16 h. The LCMS data shows product formation m/z 191.1. The reaction mixture was concentrated under reduced pressure and neutralized with saturated aq. solution of sodium bicarbonate. The product was extracted with ethyl acetate. The ethyl acetate layer was separated and dried over anhydrous Na₂SO₄. Evaporation of solvent gave product (1.2 g) (Yield Quant.) MS (ESI) mass calculated for C₁₀H₁₀N₂O₂, 190.2; m/z found 191.1 [M+H]⁺

Step 2: Synthesis of Compound HBS-039-014: Compound HBS-039-013 (1.17 g, 6.17 mmol) was dissolved in DCM (25.0 mL). NBS (1.1 g, 6.17 mmol) was added and reaction mixture was stirred at ambient temperature for 16 h. The LCMS data shows product formation m/z 271.0. The reaction mixture was concentrated under reduced pressure to obtain the crude product. The crude product was purified by ISCO combi-flash chromatography system, Mobile phase: EtOAc:Hexane gradient. Compound HBS-039-013 (1.5 g) was obtained (Yield 90.4%). MS (ESI) mass calculated for C₁₀H₉BrN₂O₂, 269.1; m/z found 271.0 [M+H]⁺.

Step 3: Synthesis of Compound HBS-039-015: Compound HBS-039-014 (0.2 g, 0.74 mmol) was dissolved in mixture of dioxane/water (8.0:2.0 v/v mL). Phenylboronic acid (0.11 g, 0.89 mmol) and K₂CO₃ (0.3 g, 2.23 mmol) were added followed by Pd(dppf)Cl₂·DCM₂ (0.06 g, 0.07 mmol). The reaction mixture was stirred at 80° C. under N₂ atm. for 5 h. The LCMS data shows product formation m/z 267. The reaction mixture was filtered over celite bed and washed with ethyl acetate. The filtrate was concentrated under reduced pressure to obtain the crude product which was purified by ISCO combi-flash chromatography system, Mobile phase: EtOAc:Hexane gradient. Compound HBS-039-015 was obtained (Yield Quant.). MS (ESI) mass calculated for C₁₆H₁₄N₂O₂, 266.3; m/z found 267.1 [M+H]⁺

Step 4: Synthesis of Compound HBS-039-018: Compound HBS-039-015 (0.2 g, 0.74 mmol) was dissolved in MeOH (6.0 mL). 1.0 N aq. solution of NaOH (3.7 mL, 3.7 mmol) was added and reaction mixture was stirred at 60° C. for 16 h. The LCMS data shows product formation m/z 239.1. The reaction mixture was concentrated under reduced pressure and diluted with water. The aq. layer was acidified with 1.0 M aq. HCl solution (pH=5) and the ppts were filtered and washed with water (5.0 mL×3) to obtain 0.16 g of solid product HBS-039-018 (Yield 92.6%). MS (ESI) mass calculated for C₁₄H₁₀N₂O₂, 238.2; m/z found 239.1 [M+H]⁺

Step 5: Synthesis of Compound HBS-039-022: Compound HBS-039-018 (0.025 g, 0.11 mmol) was dissolved in anhydrous DCM (2.0 mL). EDC·HCl (0.04 g, 0.21 mmol) and HOBt (0.028 g, 0.21 mmol) were added followed by Et₃N (0.15 mL, 1.1 mmol). The reaction mixture was stirred at ambient temperature for 10.0 min. Benzyl-(5R)-5-methyl-1,4-diazepane-1-carboxylate (0.03 g, 0.11 mmol) solution in DCM (2.0 mL) was added to the reaction mixture and was then stirred at ambient temperature for 16 h. The LCMS data shows product formation m/z 469.2. The reaction mixture was diluted with saturated solution of NaHCO₃ and the product was extracted with DCM. The combined DCM layer was separated and dried over anhydrous Na₂SO₄, then evaporated to give crude product which was purified by ISCO combi-flash system, Mobile phase: EtOAc:Hexane gradient. The 0.049 g of product was obtained (Yield Quant.). MS (ESI) mass calculated for C₂₈H₂₈N₄O₃, 468.6; m/z found 469.2 [M+H]⁺

Step 6: Synthesis of Intermediate HBS-039-024: Compound HBS-039-022 (0.05 g, 0.11 mmol) was dissolved in EtOAc (3.0 mL). 20.0% Pd—OH/C (10.0 mg) was added and reaction mixture was stirred at ambient temperature under H₂ atm for 16 h. The LCMS data shows product formation m/z 335.2. The reaction mixture was filtered over celite bed and washed with ethyl acetate. The filtrate was concentrated under reduced pressure to obtain 0.03 g of crude product HBS-039-024 which was used in the next step without purification. MS (ESI) mass calculated for C₂₀H₂₂N₄O, 334.4: m/z found 335.2 [M+H]⁺.

### 9. Synthesis of Intermediate HBS-039-025:

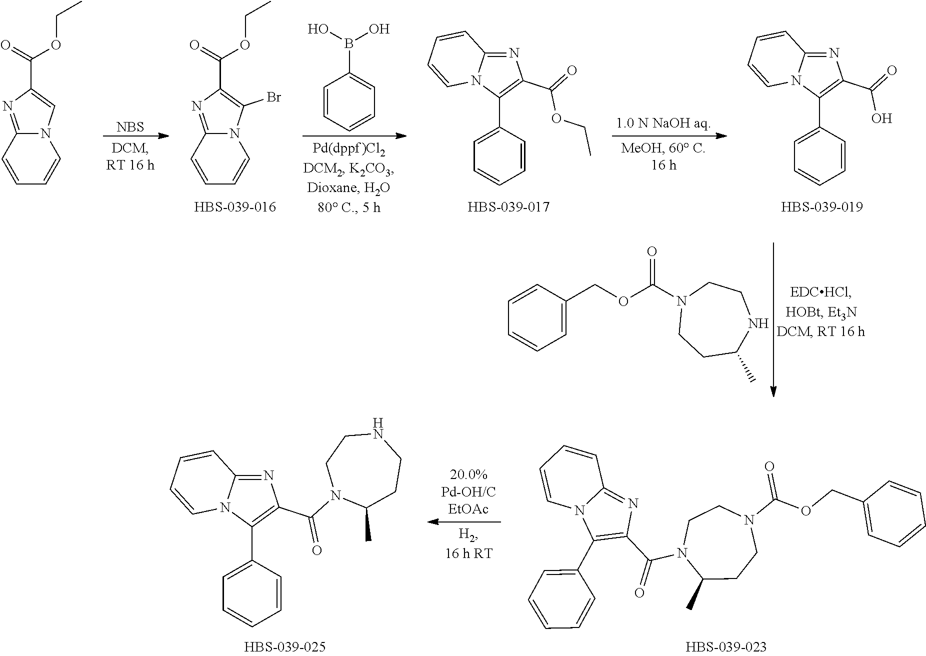

Step 1: Synthesis of Compound HBS-039-016: Imidazo[1,2-a]pyridine-2-carboxylic acid (1.0 g, 5.26 mmol) was dissolved in DCM (20.0 mL). NBS (1.0 g, 5.78 mmol) was added and reaction mixture was stirred at ambient temperature for 16 h. The LCMS data shows product formation m/z 271.0. The reaction mixture was concentrated under reduced pressure to obtain the crude product which was purified by ISCO combi-flash chromatography system, Mobile phase: EtOAc:Hexane gradient. Pure HBS-039-016 (1.4 g) was obtained (Yield 98.9%). MS (ESI) mass calculated for C₁₀H₉BrN₂O₂, 269.1; m/z found 271.0 [M+H]⁺

Step 2: Synthesis of Compound HBS-039-017: Compound HBS-039-016 (0.2 g, 0.74 mmol) was dissolved in mixture of dioxane/water (8.0:2.0 v/v mL). Phenylboronic acid (0.11 g, 0.89 mmol) and K₂CO₃ (0.3 g, 2.23 mmol) were added followed by Pd(dppf)Cl₂·DCM₂ (0.06 g, 0.07 mmol). The reaction mixture was stirred at 80° C. under N₂ atm. for 5 h. The LCMS data shows product formation m/z 267.1. The reaction mixture was filtered over celite bed and washed with ethyl acetate and the filtrate was concentrated under reduced pressure to obtain the crude product which was purified by ISCO combi-flash chromatography system, Mobile phase: EtOAc:Hexane gradient. Compound HBS-039-017 (0.2 g) was obtained (Yield Quant.). MS (ESI) mass calculated for C₁₆H₁₄N₂O₂, 266.3; m/z found 267.1 [M+H]⁺

Step 3: Synthesis of Compound HBS-039-019: Compound HBS-039-017 (0.2 g, 0.74 mmol) was dissolved in MeOH (6.0 mL) and 1.0 N aq. solution of NaOH (3.7 mL, 3.7 mmol) was added and reaction mixture was stirred at 60° C. for 16 h. The LCMS data shows product formation m/z 239.1. The reaction mixture was concentrated under reduced pressure and diluted with water. The aq. layer was acidified with 1.0 M HCl solution (pH=5). The ppts were filtered and washed with water (5.0 mL×3) to obtain 0.11 g of solid product (Yield 62.1%). MS (ESI) mass calculated for C₁₄H₁₀N₂O₂, 238.2; m/z found 239.1 [M+H]⁺

Step 4: Synthesis of Compound HBS-039-023: Compound HBS-039-017 (0.025 g, 0.11 mmol) was dissolved in anhydrous DCM (2.0 mL). EDC·HCl (0.04 g, 0.21 mmol) and HOBt (0.028 g, 0.21 mmol) were added followed by Et₃N (0.15 mL, 1.1 mmol). The reaction mixture was stirred at ambient temperature for 10.0 min. Benzyl-(5R)-5-methyl-1,4-diazepane-1-carboxylate (0.03 g, 0.11 mmol) solution in DCM (2.0 mL) was added to the reaction mixture which was then stirred at ambient temperature for 16 h. The LCMS data shows product formation m/z 469.2. The reaction mixture was diluted with saturated solution of NaHCO₃. The product was extracted with DCM. The combined DCM layer was separated and dried over anhydrous Na₂SO₄. Evaporation of solvent gave crude product which was purified by ISCO combi-flash system, Mobile phase: EtOAc:Hexane gradient. Compound HBS-039-023 (0.049 g) of product was obtained (Yield Quant.). MS (ESI) mass calculated for C₂₈H₂₈N₄O₃, 468.6; m/z found 469.2 [M+H]⁺

Step 5: Synthesis of Intermediate HBS-039-025: Compound HBS-039-023 (0.05 g, 0.11 mmol) was dissolved in EtOAc (3.0 mL). 20.0% Pd—OH/C (10.0 mg) was added and reaction mixture was stirred at ambient temperature under H₂ atm for 16 h. The LCMS data shows product formation m/z 339.2. The reaction mixture was filtered over celite bed and washed with ethyl acetate. The filtrate was concentrated under reduced pressure to obtain 0.03 g of crude product which was used in the next step without purification. MS (ESI) mass calculated for C₂₀H₂₆N₄O, 338.5; m/z found 339.2 [M+H]⁺.

### 10. Synthesis of Intermediate HBS-039-055:

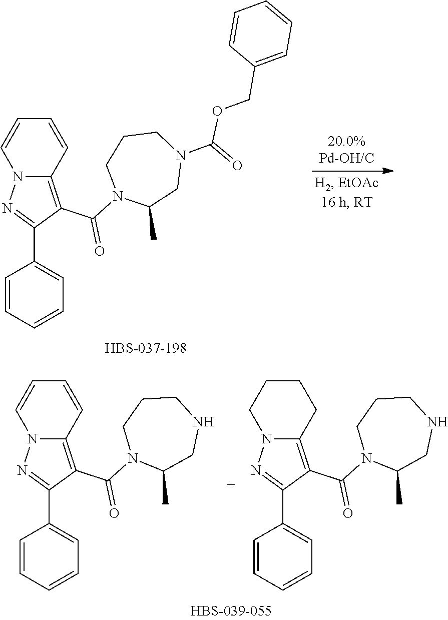 [SMILES: C[C@@H]1CN(C(=O)OCc2ccccc2)CCCN1C(=O)c1c(-c2ccccc2)nn2ccccc12 ; C[C@@H]1CNCCCN1C(=O)c1c(-c2ccccc2)nn2ccccc12 ; C[C@@H]1CNCCCN1C(=O)c1c(-c2ccccc2)nn2c1CCCC2]

Compound HBS-037-198 (0.11 g, 0.24 mmol) was dissolved in ethyl acetate (5.0 mL). 20.0% Pd—OH/C (25.0 mg) was added and reaction mixture was stirred at ambient temperature under hydrogen atmosphere for 16 h. The LCMS data shows product formation m/335.2 and over-reduction side product formation m/z 339.2. The reaction mixture was filtered over celite bed and washed with ethyl acetate. The filtrate was evaporated under reduced pressure to obtain 72.0 mg of crude product which was used as a mixture in next step without purification. MS (ESI) mass calculated for C₂₀H₂₂N₄O, 334.4; m/z found 335.2 [M+H]⁺.

### 11. Synthesis of Intermediate HBS-039-080:

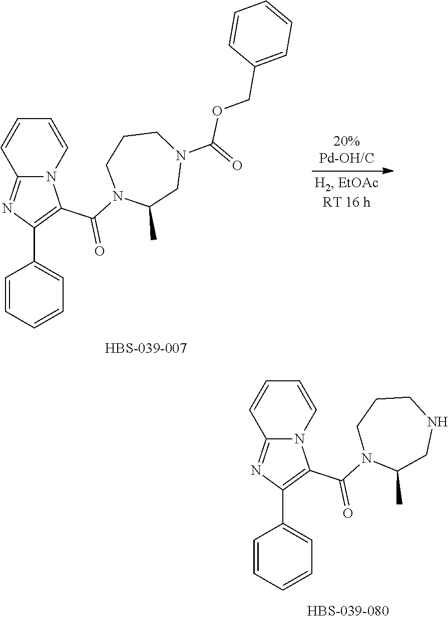 [SMILES: C[C@@H]1CN(C(=O)OCc2ccccc2)CCCN1C(=O)c1c(-c2ccccc2)nc2ccccn12 ; C[C@@H]1CNCCCN1C(=O)c1c(-c2ccccc2)nc2ccccn12]

Step 1: Synthesis of Intermediate HBS-039-080: Compound HBS-039-007 (0.4 g, 0.84 mmol) was dissolved in ethyl acetate (10.0 mL). 20.0% Pd—OH/C (20.0 mg) was added and reaction mixture was stirred at ambient temperature under hydrogen atmosphere for 16 h. The LCMS data shows major product formation m/z 335.2. The reaction mixture was filtered over celite bed and washed with ethyl acetate. The filtrate was evaporated under reduced pressure to obtain 0.25 g of crude product which was used in the next step without purification. MS (ESI) mass calculated for C₂₀H₂₂N₄O, 334.4; m/z found 335.2 [M+H]⁺.

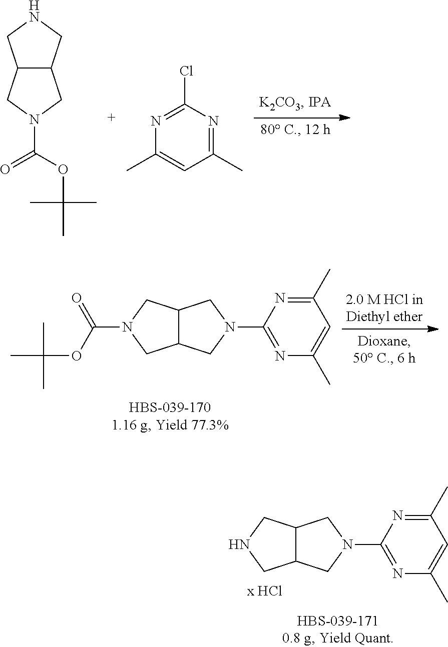

Step 1: Synthesis of HBS-039-170: 2-Boc-hexahydro-pyrrolo[3,4-c]pyrrole (1.0 g, 4.71 mmol) was dissolved in IPA (15.0 mL). The anhydrous K₂CO₃ (0.97 g, 7.07 mmol) was added followed by 2-chloro-4,6-dimethyl pyrimidine (0.067 g, 4.71 mmol). The r×n mixture was stirred at 80° C. temperature for 12 h.

The LCMS data shows product formation m/z 319.3. The r×n mixture was filtered and washed with ethyl acetate. The filtrate was concentrated to obtain the crude product. The crude product was purified by ISCO combi-flash system, Mobile phase: EtOAc:Hexane gradient. The 1.16 g of product was obtained (Yield 77.3%). MS (ESI) mass calcd. for C₁₇H₂₆N₄O₂, 318.41; m/z found 319.3 [M+H]+.

Step 2: Synthesis of HBS-039-171: Compound HBS-039-170 (1.16 g, 3.64 mmol) was dissolved in anhydrous Dioxane (15.0 mL). The 2.0 M HCl solution in Diethyl ether (7.3 mL, 14.6 mmol) was added and r×n mixture was stirred at 55° C. temperature for 6 h. LCMS data shows product formation m/z 219.1. The r×n mixture was cooled at ambient temperature and filtered to obtain the product (0.8 g, Yield Quant.). MS (ESI) mass calcd. for C₁₂H₁₈N₄, 218.3: m/z found 219.1 [M+H]+.

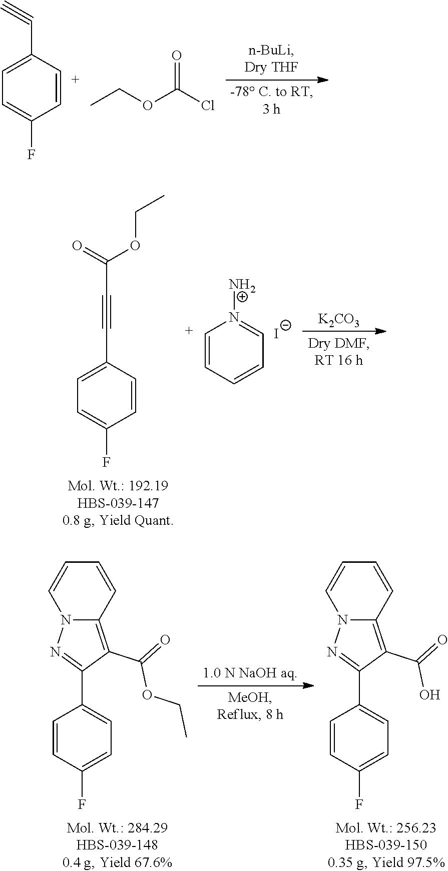

Step 1: Synthesis of HBS-039-147: 1-Ethynyl-4-fluorobenzene (0.5 g, 4.16 mmol) was dissolved in anhydrous THF (5.0 mL). The n-BuLi (5.2 mL, 8.32 mmol) was added at −78° C. temperature and r×n mixture was stirred at −78° C. temperature for 1 h. The ethyl chloroformate (1.59 mL, 16.7 mmol) was added at −78° C. temperature and r×n mixture was gradually warmed to ambient temperature. The LCMS data shows product formation m/z 193.0. The r×n mixture was diluted with aq. NH₄Cl solution and product was extracted with ethyl acetate. The combined ethyl acetate layer was separated and dried over anhydrous sodium sulfate. The evaporation of solvent gave crude product. The 0.8 g of crude product was obtained (Yield Quant.). MS (ESI) mass calcd. for C₁₁H₉FO₂, 192.19; m/z found 193.0 [M+H]+.

Step 2: Synthesis of HBS-039-148: Compound HBS-039-147 (0.4 g, 2.08 mmol) and 1-Aminopyridinium iodide (0.46 g, 2.08 mmol) were dissolved in anhydrous DMF (5.0 mL). The anhydrous K₂CO₃ (0.72 g, 5.2 mmol) was added, and reaction mixture was stirred at ambient temperature for 16 h. The LCMS data shows product formation m/z 285.1. The reaction mixture was diluted with water and ppts were filtered. The drying of ppts gave crude product. The crude product was purified by combi-flash system, Mobile phase: EtOAc:Hexane gradient. The 0.4 g of product was obtained (Yield 67.6%). MS (ESI) mass calcd. for C₁₆H₁₃FN₂O₂, 284.29; m/z found 285.1 [M+H]+.

Step 3: Synthesis of HBS-039-150: Compound HBS-039-148 (0.4 g, 1.41 mmol) was dissolved in MeOH (7.0 mL). The 1.0 N aq. NaOH solution (7.0 mL, 7.0 mmol) was added, and reaction mixture was refluxed for 8 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 0.35 g of solid product (Yield 97.5%). MS (ESI) mass calcd. for C₁₄H₉FN₂O₂, 256.23; m/z found 257.0 [M+H]+.

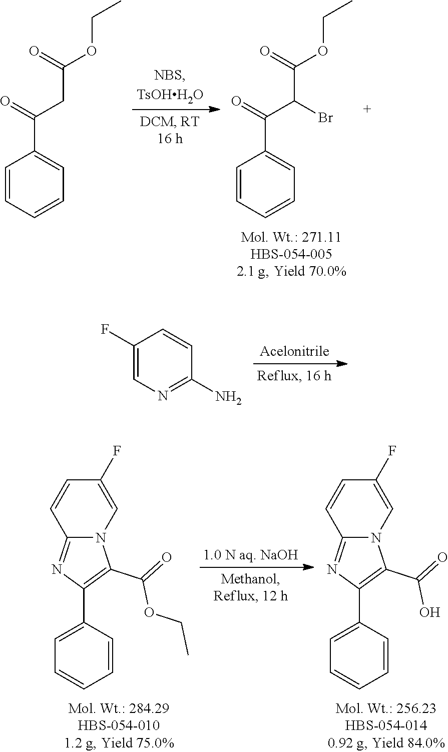

Step 1: Synthesis of HBS-054-005: The ethyl benzoylacetate (2.13 g, 11.1 mmol) was dissolved in DCM (20.0 mL). The NBS (1.8 g, 11.1 mmol) and TsOH·H₂O (0.38 g, 2.0 mmol) were added and reaction mixture was stirred at ambient temperature for 24 h. LCMS shows product formation m/z 272.0. The reaction mixture was diluted with water and the product was extracted with ethyl acetate. The ethyl acetate layer was separated and dried over anhydrous Na₂SO₄. The evaporation of solvent gave crude product. The crude product was purified by combi-flash system, Mobile phase: EtOAc:Hexane gradient. The 2.1 g of product was obtained (Yield 70.0%). MS (ESI) mass calcd. for C₁₁H₁₁BrO₃, 271.1; m/z found 272.0 [M+H]+.

Step 2: Synthesis of HBS-054-010: Compound HBS-054-005 (1.5 g, 5.56 mmol) was dissolved in anhydrous acetonitrile (20.0 mL). The 2-amino-5-fluoropyridine (1.9 g, 16.67 mmol) was added and reaction mixture was stirred at 80° C. for 16 h. LCMS shows product formation m/z 285.0. The reaction mixture was concentrated under reduced pressure to yield crude product. The crude product was purified by combi-flash system, Mobile phase: EtOAc:Hexane gradient. The 1.2 g of product was obtained (Yield 75.0%). MS (ESI) mass calcd. for C₁₆H₁₃FN₂O₂, 284.29; m/z found 285.0 [M+H]+.

Step 3: Synthesis of HBS-054-014: Compound HBS-054-010 (1.2 g, 4.22 mmol) was dissolved in MeOH (12.0 mL). The 1.0 N aq. NaOH solution (8.44 mL, 8.5 mmol) was added, and reaction mixture was stirred at 60° C. temperature for 12 h. LCMS shows product formation m/z 257.0. The reaction mixture was concentrated under reduced pressure. The solid was dissolved in water and acidified with 2.0 M aq.

HCl solution (pH=5). The ppts were filtered and washed with water (5.0 mL×3) to obtain 0.92 g of solid product (Yield 84.0%). MS (ESI) mass calcd. for C₁₄H₉FN₂O₂, 256.23; m/z found 257.0 [M+H]+.

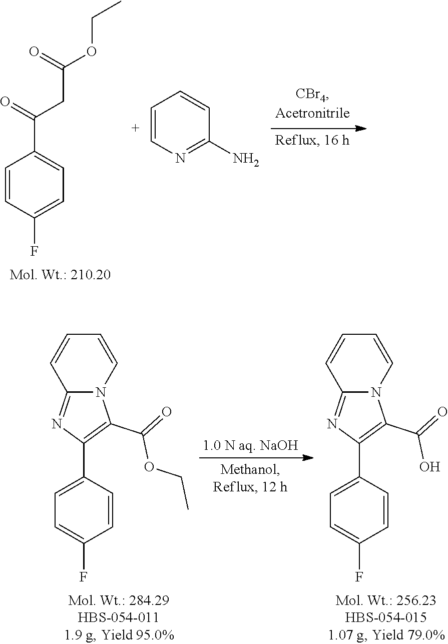 [SMILES: CCOC(=O)CC(=O)c1ccc(F)cc1 ; Nc1ccccn1 ; CCOC(=O)c1c(-c2ccc(F)cc2)nc2ccccn12 ; O=C(O)c1c(-c2ccc(F)cc2)nc2ccccn12]

Step 1: Synthesis of HBS-054-011: Ethyl 3-(4-fluorophenyl)-3-oxopropanoate (1.5 g, 7.14 mmol) was dissolved in anhydrous acetonitrile (20.0 mL). The 2-amino-pyridine (2.0 g, 21.4 mmol) was added followed by CBr₄ (4.7 g, 14.27 mmol) and reaction mixture was stirred at 80° C. for 16 h. LCMS shows product formation m/z 285.0. The reaction mixture was concentrated under reduced pressure to yield crude product. The crude product was purified by combi-flash system, Mobile phase: EtOAc:Hexane gradient. The 1.9 g of product was obtained (Yield 95.0%). MS (ESI) mass calcd. for C₁₆H₁₃FN₂O₂, 284.29; m/z found 285.0 [M+H]+.

Step 2: Synthesis of HBS-054-015: Compound HBS-054-011 (1.5 g, 5.28 mmol) was dissolved in MeOH (15.0 mL). The 1.0 N aq. NaOH solution (10.6 mL, 10.56 mmol) was added, and reaction mixture was stirred at 60° C. temperature for 12 h. LCMS shows product formation m/z 257.0. The reaction mixture was concentrated under reduced pressure. The solid was dissolved in water and acidified with 2.0 M aq.

HCl solution (pH=5). The ppts were filtered and washed with water (5.0 mL×3) to obtain 1.07 g of solid product (Yield 79.0%). MS (ESI) mass calcd. for C₁₄H₉FN₂O₂, 256.23; m/z found 257.0 [M+H]+.

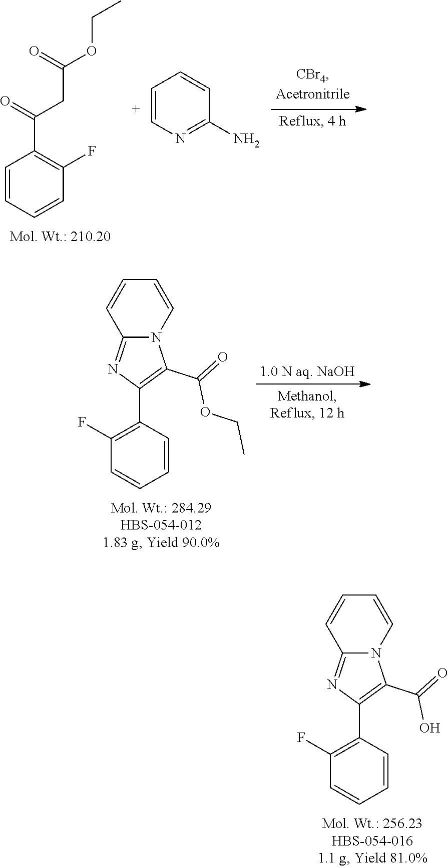 [SMILES: CCOC(=O)CC(=O)c1ccccc1F ; Nc1ccccn1 ; CCOC(=O)c1c(-c2ccccc2F)nc2ccccn12 ; O=C(O)c1c(-c2ccccc2F)nc2ccccn12]

Step 1: Synthesis of HBS-054-012: Ethyl 3-(2-fluorophenyl)-3-oxopropanoate (1.5 g, 7.14 mmol) was dissolved in anhydrous acetonitrile (20.0 mL). The 2-amino-pyridine (2.0 g, 21.4 mmol) was added followed by CBr₄ (4.7 g, 14.27 mmol) and reaction mixture was stirred at 80° C. for 4 h. LCMS shows product formation m/z 285.0. The reaction mixture was concentrated under reduced pressure to yield crude product. The crude product was purified by combi-flash system, Mobile phase: EtOAc:Hexane gradient. The 1.83 g of product was obtained (Yield 90.0%). MS (ESI) mass calcd. for C₁₆H₁₃FN₂O₂, 284.29; m/z found 285.0 [M+H]+.

Step 2: Synthesis of HBS-054-016: Compound HBS-054-012 (1.5 g, 5.28 mmol) was dissolved in MeOH (15.0 mL). The 1.0 N aq. NaOH solution (10.6 mL, 10.56 mmol) was added, and reaction mixture was stirred at 60° C. temperature for 12 h. LCMS shows product formation m/z 257.0. The reaction mixture was concentrated under reduced pressure. The solid was dissolved in water and acidified with 2.0 M aq.

HCl solution (pH=5). The ppts were filtered and washed with water (5.0 mL×3) to obtain 1.1 g of solid product (Yield 81.0%). MS (ESI) mass calcd. for C₁₄H₉FN₂O₂, 256.23; m/z found 257.0 [M+H]+.

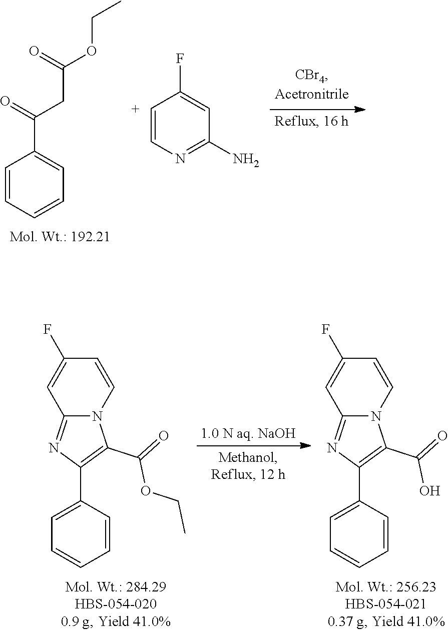 [SMILES: CCOC(=O)CC(=O)c1ccccc1 ; NC1=NC=[C+]C(F)=C1 ; CCOC(=O)c1c(-c2ccccc2)nc2cc(F)ccn12 ; O=C(O)c1c(-c2ccccc2)nc2cc(F)ccn12]

Step 1: Synthesis of HBS-054-020: Ethyl benzoylacetate (1.5 g, 7.81 mmol) was dissolved in anhydrous acetonitrile (20.0 mL). The 2-amino-4-fluoropyridine (2.6 g, 23.4 mmol) was added followed by CBr₄ (5.2 g, 15.6 mmol) and reaction mixture was stirred at 80° C. for 16 h. LCMS shows product formation m/z 285.0. The reaction mixture was concentrated under reduced pressure to yield crude product. The crude product was purified by combi-flash system, Mobile phase: EtOAc:Hexane gradient. The 0.9 g of product was obtained (Yield 41.0%). MS (ESI) mass calcd. for C₁₆H₁₃FN₂O₂, 284.29; m/z found 285.0 [M+H]+.

Step 2: Synthesis of HBS-054-021: Compound HBS-054-020 (0.9 g, 3.17 mmol) was dissolved in MeOH (10.0 mL). The 1.0 N aq. NaOH solution (6.3 mL, 6.34 mmol) was added, and reaction mixture was stirred at 60° C. temperature for 12 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 0.37 g of solid product (Yield 41.0%). MS (ESI) mass calcd. for C₁₄H₉FN₂O₂, 256.23; m/z found 257.0 [M+H]+.

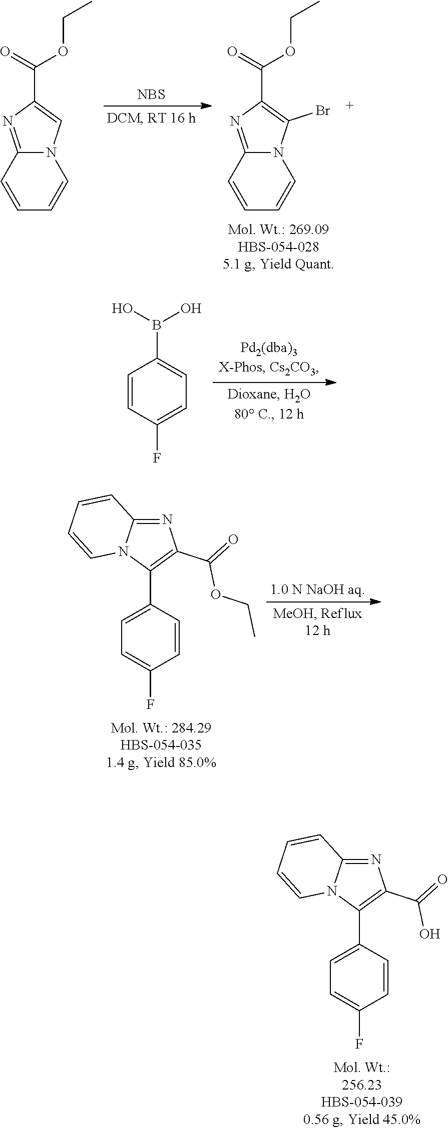 [SMILES: CCOC(=O)c1cn2ccccc2n1 ; "NBS" ; CCOC(=O)c1nc2ccccn2c1Br ; OB(O)c1ccc(F)cc1 ; CCOC(=O)c1nc2ccccn2c1-c1ccc(F)cc1 ; O=C(O)c1nc2ccccn2c1-c1ccc(F)cc1]

Step 1: Synthesis of HBS-054-028: Ethyl imidazo[1,2-a]pyridine-2-carboxylate (3.6 g, 18.94 mmol) was dissolved in DCM (80.0 mL). The NBS (3.4 g, 18.94 mmol) was added and r×n mixture was stirred at ambient temperature for 16 h. The LCMS data shows product formation m/z 270.0. The r×n mixture was concentrated under reduced pressure to obtain the crude product. The crude product was purified by ISCO combi-flash chromatography system, Mobile phase: EtOAc:Hexane gradient. The 5.1 g of product was obtained (Yield Quant.). MS (ESI) mass calcd. for C₁₀H₉BrN₂O₂, 269.1; m/z found 270.0 [M+H]+.

Step 2: Synthesis of HBS-054-035: Compound HBS-054-028 (1.5 g, 5.6 mmol) and 4-fluorophenylboronic acid (1.2 g, 8.4 mmol) were dissolved in mixture of Dioxane/water (24.0:6.0 v/v mL). The anhydrous Cs₂CO₃ (3.8 g, 11.75 mmol) was added followed by Pd₂(dba)₃ (0.26 g, 0.28 mmol) and X-Phos (0.4 g, 0.84 mmol). The r×n mixture was stirred at 80° C. temperature under N₂ atm. for 12 h. The LCMS data shows product formation m/z 285.0. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 1.4 g of product was obtained (Yield 85.0%). MS (ESI) mass calcd. for C₁₆H₁₃FN₂O₂, 284.29; m/z found 285.0 [M+H]+.

Step 3: Synthesis of HBS-054-039: Compound HBS-054-035 (1.4 g, 4.93 mmol) was dissolved in MeOH (15.0 mL). The 1.0 N aq. solution of NaOH (10.0 mL, 9.86 mmol) was added and r×n mixture was refluxed for 12 h. The LCMS data shows product formation m/z 257.0. The r×n mixture was concentrated under reduced pressure and diluted with water. The aq. layer was acidified with 2.0 M aq. HCl solution (pH=5). The ppts were filtered and washed with water (5.0 mL×3) to obtain 0.56 g of solid product (Yield 45.0%). MS (ESI) mass calcd. for C₁₄H₉FN₂O₂, 256.23; m/z found 257.0 [M+H]+.

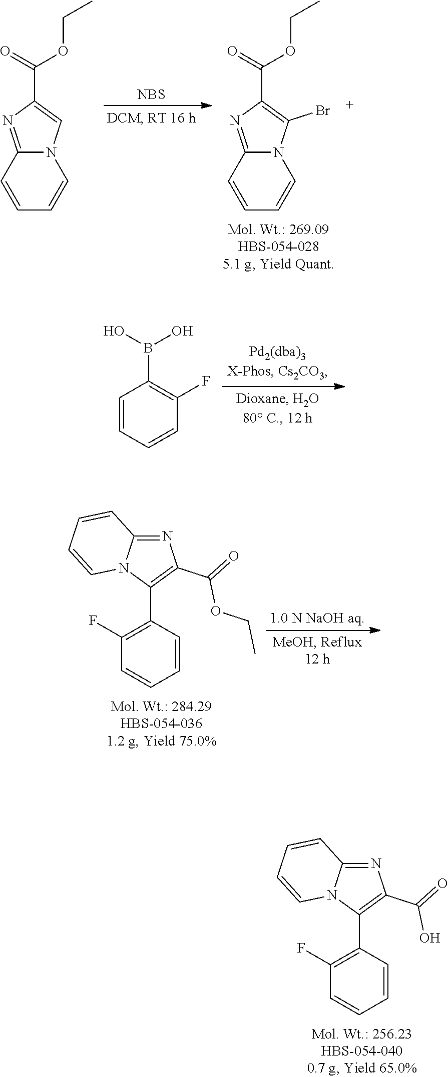 [SMILES: CCOC(=O)c1cn2ccccc2n1 ; "NBS" ; CCOC(=O)c1nc2ccccn2c1Br ; OB(O)c1ccccc1F ; CCOC(=O)c1nc2ccccn2c1-c1ccccc1F ; O=C(O)c1nc2ccccn2c1-c1ccccc1F]

Step 1: Synthesis of HBS-054-028: Ethyl imidazo[1,2-a]pyridine-2-carboxylate (3.6 g, 18.94 mmol) was dissolved in DCM (80.0 mL). The NBS (3.4 g, 18.94 mmol) was added and r×n mixture was stirred at ambient temperature for 16 h. The LCMS data shows product formation m/z 270.0. The r×n mixture was concentrated under reduced pressure to obtain the crude product. The crude product was purified by ISCO combi-flash chromatography system, Mobile phase: EtOAc:Hexane gradient. The 5.1 g of product was obtained (Yield Quant.). MS (ESI) mass calcd. for C₁₀H₉BrN₂O₂, 269.1; m/z found 270.0 [M+H]+.

Step 2: Synthesis of HBS-054-036: Compound HBS-054-028 (1.5 g, 5.6 mmol) and 2-fluorophenylboronic acid (1.2 g, 8.4 mmol) were dissolved in mixture of Dioxane/water (24.0:6.0 v/v mL).

The anhydrous Cs₂CO₃ (3.8 g, 11.75 mmol) was added followed by Pd₂(dba)₃ (0.26 g, 0.28 mmol) and X-Phos (0.4 g, 0.84 mmol). The r×n mixture was stirred at 80° C. temperature under N₂ atm. for 12 h. The LCMS data shows product formation m/z 285.0. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 1.2 g of product was obtained (Yield 75.0%). MS (ESI) mass calcd. for C₁₆H₁₃FN₂O₂, 284.29: m/z found 285.0 [M+H]+.

Step 3: Synthesis of HBS-054-040: Compound HBS-054-036 (1.2 g, 4.22 mmol) was dissolved in MeOH (15.0 mL). The 1.0 N aq. solution of NaOH (8.4 mL, 8.44 mmol) was added and r×n mixture was refluxed for 12 h. The LCMS data shows product formation m/z 257.0. The r×n mixture was concentrated under reduced pressure and diluted with water. The aq. layer was acidified with 2.0 M aq. HCl solution (pH=5). The ppts were filtered and washed with water (5.0 mL×3) to obtain 0.7 g of solid product (Yield 65.0%). MS (ESI) mass calcd. for C₁₄H₉FN₂O₂, 256.23; m/z found 257.0 [M+H]+.

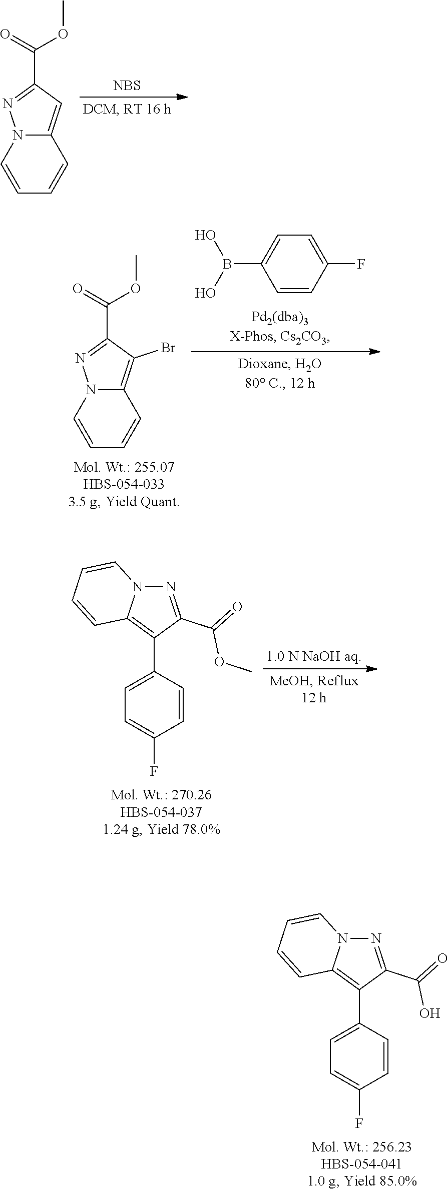

Step 1: Synthesis of HBS-054-033: Pyrazolo[1,5-a]pyridine-2-carboxylic acid methyl ester (2.4 g, 13.6 mmol) was dissolved in DCM (54.0 mL). The NBS (2.5 g, 14.3 mmol) was added and r×n mixture was stirred at ambient temperature for 16 h. The LCMS data shows product formation m/256.0. The r×n mixture was concentrated under reduced pressure to obtain the crude product. The crude product was purified by ISCO combi-flash chromatography system, Mobile phase: EtOAc:Hexane gradient. The 3.5 g of product was obtained (Yield Quant.). MS (ESI) mass calcd. for C₉H₇BrN₂O₂, 255.07; m/z found 256.0 [M+H]+.

Step 2: Synthesis of HBS-054-037: Compound HBS-054-033 (1.5 g, 5.9 mmol) and 4-fluorophenylboronic acid (1.2 g, 8.86 mmol) were dissolved in mixture of Dioxane/water (24.0:6.0 v/v mL). The anhydrous Cs₂CO₃ (4.0 g, 12.39 mmol) was added followed by Pd₂(dba)₃ (0.27 g, 0.29 mmol) and X-Phos (0.4 g, 0.88 mmol). The r×n mixture was stirred at 80° C. temperature under N₂ atm. for 12 h. The LCMS data shows product formation m/z 271.0. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 1.24 g of product was obtained (Yield 78.0%). MS (ESI) mass calcd. for C₁₅H₁₁FN₂O₂, 270.26; m/z found 271.0 [M+H]+.

Step 3: Synthesis of HBS-054-041: Compound HBS-054-037 (1.2 g, 4.59 mmol) was dissolved in MeOH (15.0 mL). The 1.0 N aq. solution of NaOH (9.2 mL, 9.18 mmol) was added and r×n mixture was refluxed for 12 h. The LCMS data shows product formation m/z 257.0. The r×n mixture was concentrated under reduced pressure and diluted with water. The aq. layer was acidified with 2.0 M aq. HCl solution (pH=5). The ppts were filtered and washed with water (5.0 mL×3) to obtain 1.0 g of solid product (Yield 85.0%). MS (ESI) mass calcd. for C₁₄H₉FN₂O₂, 256.23; m/z found 257.0 [M+H]+.

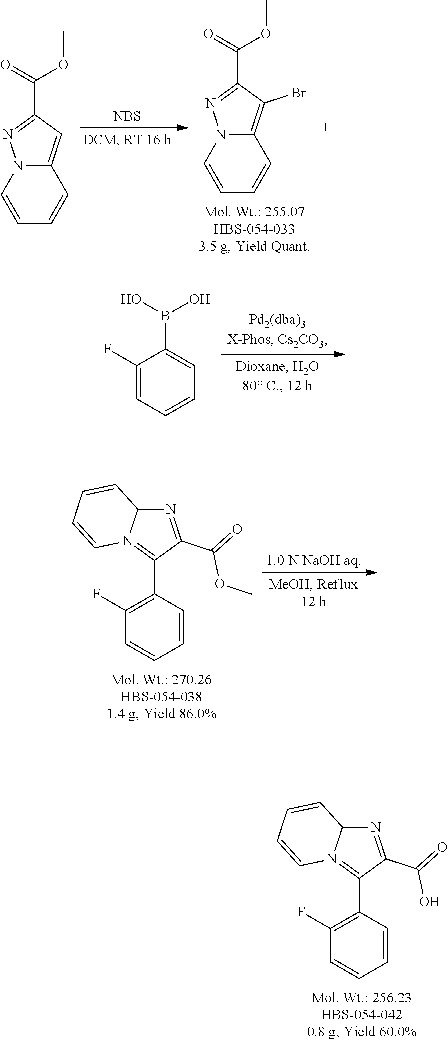

Step 1: Synthesis of HBS-054-033: Pyrazolo[1,5-a]pyridine-2-carboxylic acid methyl ester (2.4 g, 13.6 mmol) was dissolved in DCM (54.0 mL). The NBS (2.5 g, 14.3 mmol) was added and r×n mixture was stirred at ambient temperature for 16 h. The LCMS data shows product formation m/z 256.0. The r×n mixture was concentrated under reduced pressure to obtain the crude product. The crude product was purified by ISCO combi-flash chromatography system, Mobile phase: EtOAc:Hexane gradient. The 3.5 g of product was obtained (Yield Quant.). MS (ESI) mass calcd. for C₉H₇BrN₂O₂, 255.07; m/z found 256.0 [M+H]+.

Step 2: Synthesis of HBS-054-038: Compound HBS-054-033 (1.5 g, 5.9 mmol) and 2-fluorophenylboronic acid (1.2 g, 8.86 mmol) were dissolved in mixture of Dioxane/water (24.0:6.0 v/v mL). The anhydrous Cs₂CO₃ (4.0 g, 12.39 mmol) was added followed by Pd₂(dba)₃ (0.27 g, 0.29 mmol) and X-Phos (0.4 g, 0.88 mmol). The r×n mixture was stirred at 80° C. temperature under N₂ atm. for 12 h. The LCMS data shows product formation m/z 271.0. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 1.4 g of product was obtained (Yield 86.0%). MS (ESI) mass calcd. for C₁₅H₁₁FN₂O₂, 270.26; m/z found 271.0 [M+H]+.

Step 3: Synthesis of HBS-054-042: Compound HBS-054-038 (1.4 g, 5.18 mmol) was dissolved in MeOH (15.0 mL). The 1.0 N aq. solution of NaOH (10.4 mL, 10.36 mmol) was added and r×n mixture was refluxed for 12 h. The LCMS data shows product formation m/z 257.0. The r×n mixture was concentrated under reduced pressure and diluted with water. The aq. layer was acidified with 2.0 M aq. HCl solution (pH=5). The ppts were filtered and washed with water (5.0 mL×3) to obtain 1.0 g of solid product (Yield 85.0%). MS (ESI) mass calcd. for C₁HFN₂O₂, 256.23; m/z found 257.0 [M+H]+.

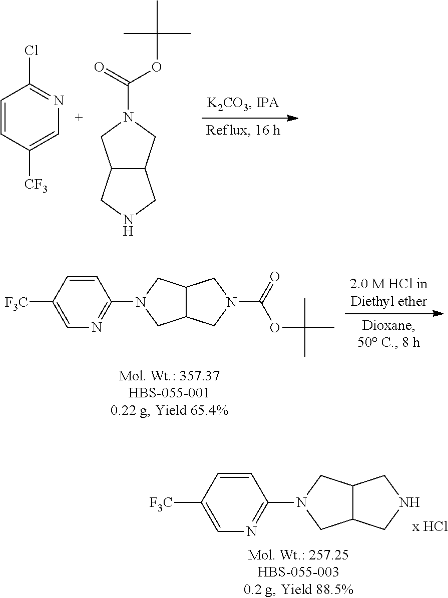

Step 1: Synthesis of HBS-055-001: 2-Boc-hexahydro-pyrrolo[3,4-c]pyrrole (0.2 g, 0.94 mmol) was dissolved in IPA (5.0 mL). The anhydrous K₂CO₃ (0.2 g, 1.41 mmol) was added followed by 2-chloro-5-trifluoromethyl pyridine (0.21 g, 1.13 mmol). The r×n mixture was refluxed for 16 h. The LCMS data shows product formation m/z 319.3. The r×n mixture was cooled and filtered through Buchner funnel. The filtrate was concentrated to obtain the crude product. The crude product was purified by ISCO combi-flash system, Mobile phase: EtOAc:Hexane gradient. The 0.22 g of product was obtained (Yield 65.4%). MS (ESI) mass calcd. for C₁₇H₂₂F₃N₃O₂, 357.37; m/z found 358.3 [M+H]+.

Step 2: Synthesis of HBS-055-003: Compound HBS-055-001 (0.22 g, 0.62 mmol) was dissolved in anhydrous Dioxane (10.0 mL). The 2.0 M HCl solution in Diethyl ether (1.63 mL, 3.27 mmol) was added and r×n mixture was stirred at 55° C. temperature for 8 h. LCMS data shows product formation m/z 258.1.

The r×n mixture was cooled at ambient temperature and filtered to obtain the solid product (0.2 g, Yield 88.5%). MS (ESI) mass calcd. for C₁₂H₁₄F₃N₃, 257.25; m/z found 258.1 [M+H]+.

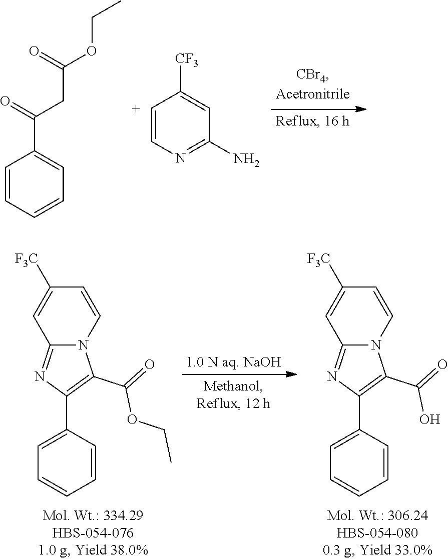

Step 1: Synthesis of HBS-054-076: Ethyl benzoylacetate (1.5 g, 7.81 mmol) was dissolved in anhydrous acetonitrile (20.0 mL). The 2-amino-4-(trifluoromethyl)pyridine (3.8 g, 23.4 mmol) was added followed by CBr₄ (5.2 g, 15.6 mmol) and reaction mixture was stirred at 80° C. for 16 h. LCMS shows product formation m/z 335.0. The reaction mixture was concentrated under reduced pressure to yield crude product. The crude product was purified by combi-flash system, Mobile phase: EtOAc:Hexane gradient. The 1.0 g of product was obtained (Yield 38.0%). MS (ESI) mass calcd. for C₁₇H₁₃F₃N₂O₂, 334.29; m/z found 335.0 [M+H]+.

Step 2: Synthesis of HBS-054-080: Compound HBS-054-076 (1.0 g, 2.99 mmol) was dissolved in MeOH (10.0 mL). The 1.0 N aq. NaOH solution (6.0 mL, 6.0 mmol) was added, and reaction mixture was refluxed for 12 h. LCMS shows product formation m/z 307.0. The reaction mixture was concentrated under reduced pressure. The solid was dissolved in water and acidified with 2.0 M aq. HCl solution (pH=5). The ppts were filtered and washed with water (5.0 mL×3) to obtain 0.3 g of solid product (Yield 33.0%). MS (ESI) mass calcd. for C₁₅H₉F₃N₂O₂, 306.24; m/z found 307.0 [M+H]+.

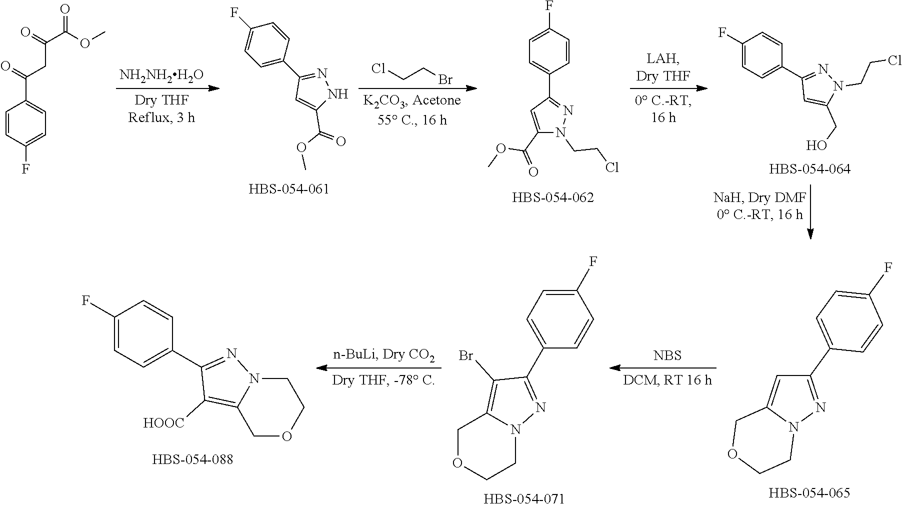

Step 1: Synthesis of HBS-054-061: Methyl 4-(4-fluorophenyl)-2,4-dioxobutanoate (2.2 g, 10.68 mmol) was dissolved in anhydrous THF (40.0 mL). The hydrazine monohydrate (0.56 g, 11.21 mmol) was added. The reaction mixture was heated at reflux for 3 h. LCMS data shows desired product formation m/z 221.0. The reaction mixture was concentrated under reduced pressure to obtain the crude product.

The crude product was purified by column chromatography, Mobile Phase: EtOAc:Hexane, gradient. The 1.2 g of product was isolated (Yield 51.0%). MS (ESI) mass calcd. for C₁₁H₉FN₂O₂, 220.2; m/z found 221.0 [M+H]+.

Step 2: Synthesis of HBS-054-062: Compound HBS-054-061 (0.64 g, 2.91 mmol) was dissolved in Acetone (15.0 mL). The K₂CO₃ (0.8 g, 5.82 mmol) was added, followed by 1-Bromo-2-Chloro-ethane (0.5 g, 3.49 mmol). The reaction mixture was heated at 55° C. for 16 h. LCMS data shows desired product formation m/z 283.0 and minor amount of side product formation. The reaction mixture was filtered and the solid was washed with ethyl acetate. The filtrate was concentrated under reduced pressure to obtain crude product. The crude product was purified by column chromatography, Mobile Phase: EtOAc:Hexane, gradient. The 0.7 g of product was isolated (Yield 85.0%). MS (ESI) mass calcd. for C₁₃H₁₂ClFN₂O₂, 282.7; m/z found 283.0 [M+H]+.

Step 3: Synthesis of HBS-054-064: Compound HBS-054-062 (0.7 g, 2.48 mmol) was dissolved in dry THF (10.0 mL). The 2.0 M LAH solution in THF (1.24 mL, 2.48 mmol) was added, under ice cooling bath. The reaction mixture was gradually warmed to room temperature and stirred for 16 h. LCMS data shows desired product formation m/z 255.0. The reaction mixture was quenched with 1.0 N aq. NaOH solution and diluted with ethyl acetate (10.0 mL). The reaction mixture was filtered through celite bed and washed with ethyl acetate (10.0 mL×3). The organic layer was separated and washed with water followed by brine. The organic layer was dried over anhydrous sodium sulfate. The evaporation of solvent gave crude product 0.6 g (Yield 95.0%). MS (ESI) mass calcd. for C₁₂H₁₂ClFN₂O, 254.69; m/z found 255.0 [M+H]+.

Step 4: Synthesis of HBS-054-065: Compound HBS-054-064 (0.6 g, 2.36 mmol) was dissolved in dry DMF (5.0 mL). The NaH (0.11 g, 4.72 mmol) was added under ice cooling. The reaction mixture was gradually warmed to room temperature and stirred for 16 h. LCMS data shows desired product formation m/z 219.0. The reaction mixture was diluted with water and the product was extracted with ethyl acetate. The combined ethyl acetate layer was separated and dried over anhydrous sodium sulfate. The evaporation of solvent gave crude product. The crude product was purified by column chromatography, Mobile Phase: EtOAc:Hexane, gradient. The 0.46 g of solid product was obtained (Yield 88.0%). MS (ESI) mass calcd. for C₁₂H₁₁FN₂O, 218.23; m/z found 219.0 [M+H]+.

Step 5: Synthesis of HBS-054-071: Compound HBS-054-065 (0.46 g, 2.11 mmol) was dissolved in DCM (7.0 mL). The NBS (0.41 g, 2.32 mmol) was added, and reaction mixture was stirred at room temperature for 16 h. LCMS data shows desired product formation m/z 298.0. The evaporation of solvent gave crude product. The crude product was purified by column chromatography, Mobile Phase: EtOAc:Hexane, gradient. The 0.25 g of product was obtained (Yield 40.0%). MS (ESI) mass calcd. for C₁₂H₁₀BrFN₂O, 297.12: m/z found 298.0 [M+H]+.

Step 6: Synthesis of HBS-054-088: Compound HBS-054-071 (1.15 g, 3.85 mmol) was dissolved in anhydrous THF (20.0 mL) under N₂ atm. The reaction mixture was cooled at −78.0° C. temperature and 1.6 M n-BuLi in Hexane (4.81 mL, 7.7 mmol) was added into the reaction mixture. The reaction mixture was stirred at −78.0° C. temperature for 30.0 min. The dry CO₂ gas bubbled through the reaction mixture at −65° C. and the reaction mixture gradually warmed at room temperature. The LCMS data shows desired product formation m/z 263.0, dibrominated side product and some unknown product formation. The reaction mixture was quenched by water and extracted with ethyl acetate. The ethyl acetate layer was separated and dibrominated product was recovered. The aq. Layer was acidified with 2 M HCl solution to obtain the ppts. The ppts were filtered and dried to obtain 0.6 g of solid product (Yield 60.0%). MS (ESI) mass calcd. for C₁₃H₁₁FN₂O₃, 262.24: m/z found 263.0 [M+H]+.

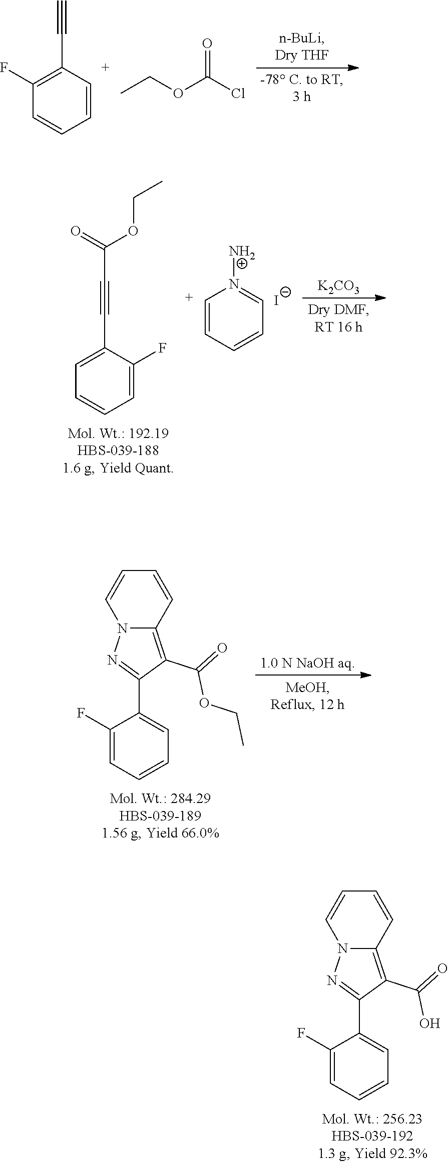

Step 1: Synthesis of HBS-039-188: 1-Ethynyl-2-fluorobenzene (1.0 g, 8.33 mmol) was dissolved in anhydrous THF (10.0 mL). The n-BuLi (10.4 mL, 16.65 mmol) was added at −78° C. temperature and r×n mixture was stirred at −78° C. temperature for 1 h. The ethyl chloroformate (3.8 mL, 40.0 mmol) was added at −78° C. temperature and r×n mixture was gradually warmed to ambient temperature. The LCMS data shows product formation m/z 193.0. The r×n mixture was diluted with aq. NH₄Cl solution and product was extracted with ethyl acetate. The combined ethyl acetate layer was separated and dried over anhydrous sodium sulfate. The evaporation of solvent gave crude product. The 1.6 g of crude product was obtained (Yield Quant.). MS (ESI) mass calcd. for C₁₁H₉FO₂, 192.19; m/z found 193.0 [M+H]+.

Step 2: Synthesis of HBS-039-189: Compound HBS-039-188 (1.6 g, 8.33 mmol) and 1-Aminopyridinium iodide (1.85 g, 8.33 mmol) were dissolved in anhydrous DMF (15.0 mL). The anhydrous K₂CO₃ (2.88 g, 20.81 mmol) was added, and reaction mixture was stirred at ambient temperature for 16 h. The LCMS data shows product formation m/z 285.1. The reaction mixture was diluted with water and the product was extracted with ethyl acetate. The combined ethyl acetate layer was separated and dried over anhydrous sodium sulfate. The evaporation of solvent gave crude product. The crude product was purified by column chromatography, Mobile Phase: EtOAc:Hexane, gradient. The 1.56 g of product was obtained (Yield 66.0%). MS (ESI) mass calcd. for C₁₆H₁₃FN₂O₂, 284.29: m/z found 285.1 [M+H]+.

Step 3: Synthesis of HBS-039-192: Compound HBS-039-189 (1.56 g, 5.5 mmol) was dissolved in MeOH (10.0 mL). The 1.0 N aq. NaOH solution (16.5 mL, 16.5 mmol) was added, and reaction mixture was refluxed for 12 h. LCMS shows product formation m/z 257.0. The reaction mixture was concentrated under reduced pressure. The solid was dissolved in water and acidified with 2.0 M aq. HCl solution (pH=5). The ppts were filtered and washed with water (5.0 mL×3) to obtain 1.3 g of solid product (Yield 92.3%). MS (ESI) mass calcd. for C₁₄HFN₂O₂, 256.23; m/z found 257.0 [M+H]+.

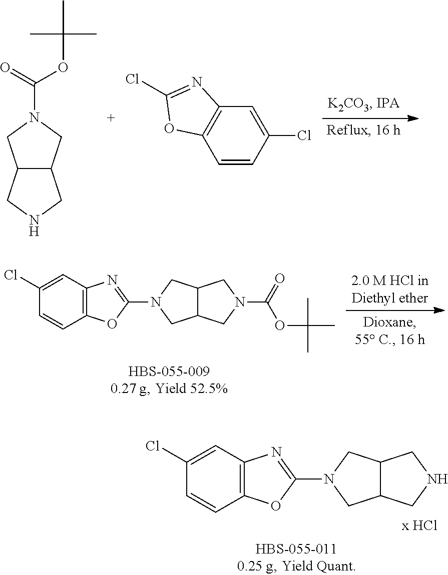 [SMILES: CC(C)(C)OC(=O)N1CC2CNCC2C1 ; Clc1ccc2oc(Cl)nc2c1 ; CC(C)(C)OC(=O)N1CC2CN(c3nc4cc(Cl)ccc4o3)CC2C1 ; Clc1ccc2oc(N3CC4CNCC4C3)nc2c1]

Step 1: Synthesis of HBS-055-009: 2-Boc-hexahydro-pyrrolo[3,4-c]pyrrole (0.3 g, 1.41 mmol) was dissolved in IPA (10.0 mL). The anhydrous K₂O₃ (0.97 g, 7.07 mmol) was added followed by 2.5-Dichlorobenzooxazole (0.38 g, 1.99 mmol). The r×n mixture was stirred at 80 LC temperature for 16 h. The LCMS data shows product formation m/z 364.2. The r×n mixture was filtered at room temperature and washed with ethyl acetate. The filtrate was concentrated to obtain the crude product. The crude product was purified by 1800 combi-flash system, Mobile phase: EtOAc:Hexane gradient. The 0.27 g of product was obtained (Yield 52.5%). MS (ESI) mass calcd. for C₁₈H₂₂ClN₃O₃, 363.84; m/z found 364.2 [M+H]+.

Step 2: Synthesis of HBS-055-011: Compound HBS-055-009 (0.27 g, 0.74 mmol) was dissolved in anhydrous Dioxane (15.0 mL). The 2.0 M H solution in Diethyl ether (1.5 mL, 2.97 mmol) was added and r×n mixture was stirred at 55° C. temperature for 6 h. LCMS data shows product formation m/z 264.1. The r×n mixture was cooled at ambient temperature and filtered to obtain the solid product (0.25 g, Yield Quant.). MS (ESI) mass calcd. for C₁₃H₁₄ClN₃O, 263.7; m/z found 264.1 [M+H]+.

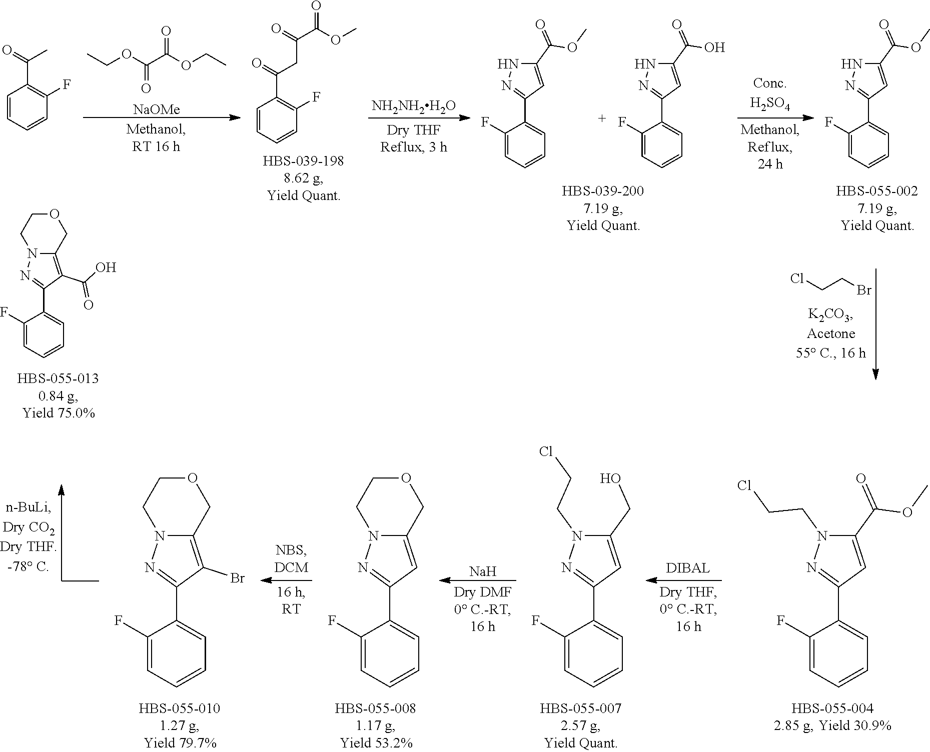

Step 1: Synthesis of HBS-039-198: 2-Fluoroacetophenone (5.0 g, 36.19 mmol) was added dropwise in NaOMe solution (1.25 g Na in 25.0 mL of Methanol). The r×n mixture was stirred at ambient temperature for 30.0 min. The Diethyl oxalate (5.81 g, 39.81 mmol) solution in anhydrous Methanol (25.0 mL) was added and reaction mixture was stirred at ambient temperature for 16 h. LCMS data shows desired product formation m/z 225.0. The reaction mixture was concentrated under reduced pressure to obtain the crude product. The crude product was dissolved in cold water and acidified with 2.0 M aq. HCl solution. The ppts were filtered and dried to obtain 8.62 g of crude product. MS (ESI) mass calcd. for C₁₁H₉FO₄, 224.19; m/z found 225.0 [M+H]+.

Step 2: Synthesis of HBS-039-200: Compound HBS-039-198 (8.62 g, 36.19 mmol) was dissolved in IPA (100.0 mL). The hydrazine monohydrate (2.1 mL, 43.4 mmol) was added, and reaction mixture was heated at reflux for 3 h. LCMS data shows desired product formation m/z 221.0, and hydrolyzed side product m/z 207.0. The reaction mixture was cooled at ambient temperature to obtain ppts. The ppts were filtered to obtain 7.19 g of crude product. (Yield 90.2%). MS (ESI) mass calcd. for C₁₁H₉FN₂O₂, 220.2; m/found 221.0 [M+H]+, and mass calcd. for C₁₀H₇FN₂O₂, 206.17; m/z found 207.0 [M+H]+.

Step 3: Synthesis of HBS-055-002: Compound HBS-039-200 crude (7.19 g, 32.65 mmol) was dissolved in anhydrous Methanol (100.0 mL). The conc. Sulfuric acid (4.0 mL) was added, and reaction mixture was heated at reflux for 24 h. LCMS data shows desired product formation m/z 221.0. The reaction mixture was cooled at ambient temperature and neutralized with aq. saturated solution of sodium bicarbonate to obtain ppts. The ppts were filtered and dried to obtain 7.19 g of solid product. (Yield Quant.). MS (ESI) mass calcd. for C₁₁H₉FN₂O₂, 220.2; m/z found 221.0 [M+H]+.

Step 4: Synthesis of HBS-055-004: Compound HBS-055-002 (7.19 g, 32.65 mmol) was dissolved in Acetone (100.0 mL). The K₂CO₃ (13.53 g, 97.95 mmol) was added, followed by 1-Bromo-2-Chloro-ethane (13.5 mL, 163.3 mmol). The reaction mixture was heated at 65° C. for 24 h. LCMS data shows desired product formation m/z 283.0. The reaction mixture was filtered and the solid was washed with ethyl acetate. The filtrate was concentrated under reduced pressure to obtain crude product. The crude product was purified by column chromatography, Mobile Phase: EtOAc:Hexane, gradient. The 2.85 g of product was isolated (Yield 30.9%). MS (ESI) mass calcd. for C₁₃H₁₂ClFN₂O₂, 282.7; m/z found 283.0 [M+H]+.

Step 5: Synthesis of HBS-055-007: Compound HBS-055-004 (2.85 g, 10.1 mmol) was dissolved in dry THF (25.0 mL). The reaction mixture was cooled at 0° C. temperature in the ice bath. The 1.0 M DIBAL solution in Hexane (25.2 mL, 25.2 mmol) was added. The reaction mixture was gradually warmed to room temperature and stirred for 16 h. LCMS data shows desired product formation m/z 255.1. The reaction mixture was quenched with aq. NH₄Cl solution and diluted with ethyl acetate (100.0 mL). The reaction mixture was filtered through celite bed and washed with ethyl acetate. The organic layer was separated and washed with brine. The organic layer was dried over anhydrous sodium sulfate. The evaporation of solvent gave crude product 2.57 g (Yield Quant.). MS (ESI) mass calcd. for C₁₂H₁₂ClFN₂O, 254.69; m/z found 255.0 [M+H]+.

Step 6: Synthesis of HBS-055-008: Compound HBS-055-007 (2.57 g, 10.1 mmol) was dissolved in dry DMF (20.0 mL). The 60.0% NaH (0.81 g, 20.2 mmol) in mineral oil was added under ice cooling. The reaction mixture was gradually warmed to room temperature and stirred for 16 h. LCMS data shows desired product formation m/z 219.1. The reaction mixture was diluted with water and the product was extracted with ethyl acetate. The combined ethyl acetate layer was separated and dried over anhydrous sodium sulfate. The evaporation of solvent gave crude product. The crude product was purified by column chromatography, Mobile Phase: EtOAc:Hexane, gradient. The 1.17 g of solid product was obtained (Yield 53.2%). MS (ESI) mass calcd. for Cl₂H₁₁FN₂O, 218.23; m/z found 219.1 [M+H]+.

Step 7: Synthesis of HBS-055-010: Compound HBS-055-008 (1.17 g, 5.36 mmol) was dissolved in DCM (15.0 mL). The NBS (1.05 g, 5.9 mmol) was added, and reaction mixture was stirred at ambient temperature for 16 h. LCMS data shows desired product formation m/z 299.0. The evaporation of solvent gave crude product. The crude product was purified by column chromatography, Mobile Phase: EtOAc:Hexane, gradient. The 1.27 g of product was obtained (Yield 79.7%). MS (ESI) mass calcd. for C₁₂H₁₀BrFN₂O, 297.12; m/z found 299.0 [M+H]+.

Step 8: Synthesis of HBS-055-013: Compound HBS-055-010 (1.27 g, 4.27 mmol) was dissolved in anhydrous THF (15.0 mL) under N₂ atm. The reaction mixture was cooled at −78.0° C. temperature. The 1.6 M n-BuLi in Hexane (5.33 mL, 8.54 mmol) was added and reaction mixture was stirred at −78.0° C. temperature for 30.0 min. The dry CO₂ gas bubbled through the reaction mixture at −65° C. temperature and the reaction mixture gradually warmed at room temperature. The LCMS data shows desired product formation m/263.1, dibrominated side product and some unknown product formation. The reaction mixture was quenched by water and extracted with ethyl acetate. The ethyl acetate layer was separated and dibrominated product was recovered. The aq. Layer was acidified with 2 M HCl solution to obtain the ppts. The ppts were filtered and dried to obtain 0.84 g of solid product (Yield 75.0%). MS (ESI) mass calcd. for C₁₃H₁₁FN₂O₃, 262.24; m/z found 263.1 [M+H]+.

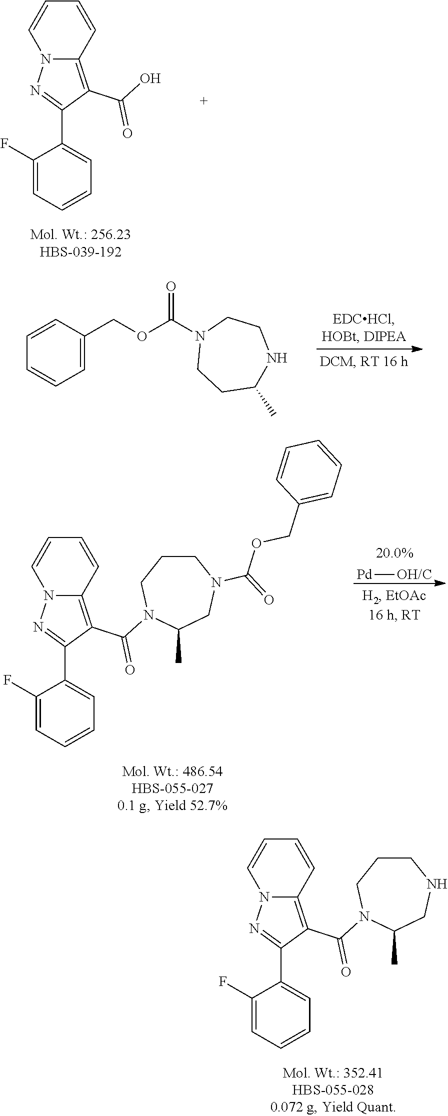

Step 1: Synthesis of HBS-055-027: Intermediate HBS-039-192 (0.1 g, 0.39 mmol) was dissolved in anhydrous DCM (4.0 mL). The EDC·HCl (0.11 g, 0.59 mmol) and HOBt (0.079 g, 0.59 mmol) were added followed by DIPEA (0.34 mL, 1.95 mmol). The r×n mixture was stirred at ambient temperature for 10.0 min. The benzyl-(5R)-5-methyl-1,4-diazepane-1-carboxylate (0.097 g, 0.39 mmol) solution in DCM (1.0 mL) was added to the r×n mixture. The r×n mixture was stirred at ambient temperature for 16 h. The LCMS data shows product formation m/z 487.2. The r×n mixture was diluted with saturated solution of NaHCO₃. The product was extracted with DCM. The combined DCM layer was separated and dried over anhydrous Na₂SO₄. The evaporation of solvent gave crude product. The crude product was purified by ISCO combi-flash system, Mobile phase: EtOAc:Hexane gradient. The 0.1 g of product was obtained (Yield 52.7%). MS (ESI) mass calcd. for C₂₅H₂₇FN₄O₃, 486.54; m/z found 487.2 [M+H]+.

Step 2: Synthesis of HBS-055-028: Compound HBS-055-027 (0.1 g, 0.21 mmol) was dissolved in Ethyl acetate (5.0 mL). The 20.0% Pd—OH/C (10.0 mg) was added and r×n mixture was stirred at ambient temperature under hydrogen atmosphere for 16 h. The LCMS data shows product formation m/z 353.2. The r×n mixture was filtered over celite bed and washed with ethyl acetate. The filtrate was evaporated under reduced pressure to obtain 72.0 mg of crude product. The crude product is used in the next step without purification. MS (ESI) mass calcd. for C₂₀H₂₁FN₄O, 352.41; m/z found 353.2 [M+H]+.

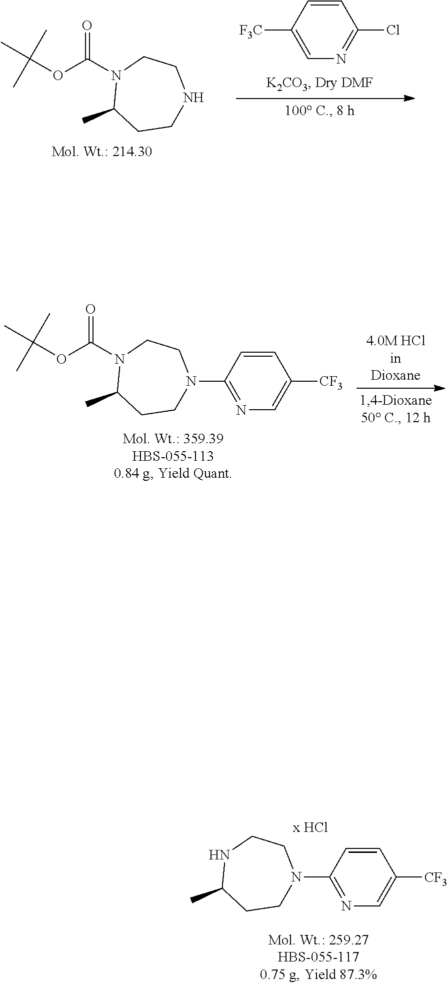 [SMILES: C[C@@H]1CCNCCN1C(=O)OC(C)(C)C ; FC(F)(F)c1ccc(Cl)nc1 ; C[C@@H]1CCN(c2ccc(C(F)(F)F)cn2)CCN1C(=O)OC(C)(C)C ; C[C@@H]1CCN(c2ccc(C(F)(F)F)cn2)CCN1]

Step 1: Synthesis of HBS-055-113: Tert-butyl (7R)-7-methyl-1,4-diazepane-1-carboxylate (0.5 g, 2.33 mmol) and 2-Chloro-5-trifluoromethyl pyridine (0.47 g, 2.57 mmol) were dissolved in anhydrous DMF (10.0 mL). The anhydrous K₂CO₃ (0.64 g, 4.66 mmol) was added, and reaction mixture was stirred at 100° C. temperature for 8 h. The LCMS data shows product formation m/z 360.1. The reaction mixture was diluted with water and the product was extracted with ethyl acetate. The combined ethyl acetate layer was separated and dried over anhydrous sodium sulfate. The evaporation of solvent gave crude product. The crude product was purified by ISCO combi-flash system, Mobile phase: EtOAc:Hexane gradient. The 0.84 g of product was obtained (Yield Quant.). MS (ESI) mass calcd. for C₁₇H₂₄F₃N₃O₂, 359.39; m/z found 360.1 [M+H]+.

Step 2: Synthesis of HBS-055-117: Compound HBS-055-113 (0.84 g, 2.33 mmol) was dissolved in anhydrous Dioxane (10.0 mL). The 4.0 M HCl solution in Dioxane (2.33 mL, 9.32 mmol) was added and reaction mixture was stirred at 50° C. temperature for 12 h. LCMS data shows product formation m/z 259.27. The r×n mixture was cooled at ambient temperature and concentrated to obtain the solid product (0.75 g, Yield 87.3%). MS (ESI) mass calcd. for C₁₂H₁₆F₃N₃, 259.27; m/z found 260.1 [M+H]+.

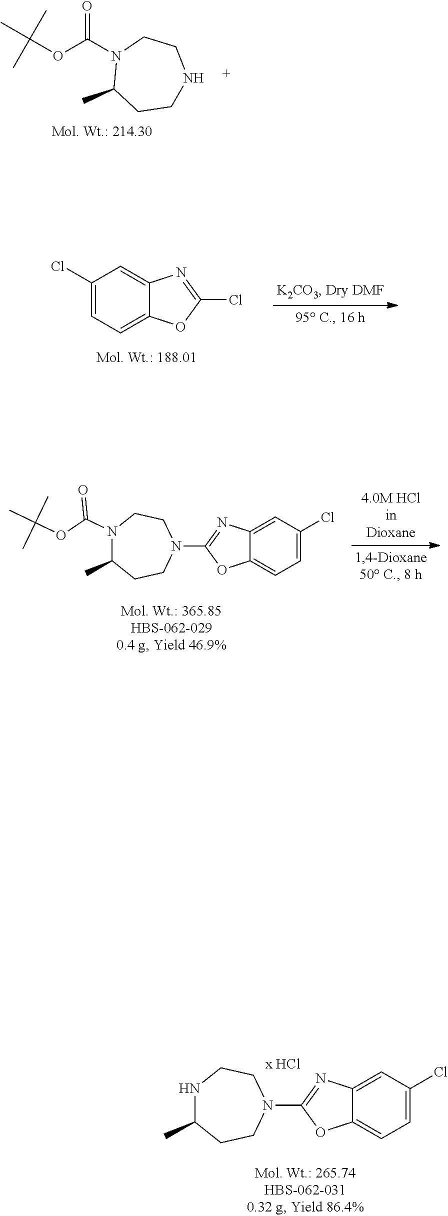 [SMILES: C[C@@H]1CCNCCN1C(=O)OC(C)(C)C ; Clc1ccc2oc(Cl)nc2c1 ; C[C@@H]1CCN(c2nc3cc(Cl)ccc3o2)CCN1C(=O)OC(C)(C)C ; C[C@@H]1CCN(c2nc3cc(Cl)ccc3o2)CCN1]

Step 1: Synthesis of HBS-062-029: Tert-butyl (7R)-7-methyl-1,4-diazepane-1-carboxylate (0.5 g, 2.33 mmol) and 2,5-Dichlorobenzooxazole (0.53 g, 2.79 mmol) were dissolved in anhydrous DMF (8.0 mL). The anhydrous K₂CO₃ (0.64 g, 4.66 mmol) was added, and reaction mixture was stirred at 100° C. temperature for 16 h. The LCMS data shows product formation 4/z 366.1. The reaction mixture was diluted with water and the product was extracted with ethyl acetate. The combined ethyl acetate layer was separated and dried over anhydrous sodium sulfate. The evaporation of solvent gave crude product. The crude product was purified by ISCO combi-flash system, Mobile phase: EtOAc:Hexane gradient. The 0.4 g of product was obtained (Yield 46.9%). MS (ESI) mass calcd. for C₁₆H₂₄ClN₃O₃, 365.85; m/z found 366.1 [M+H]+.

Step 2: Synthesis of HBS-062-031: Compound HBS-062-029 (0.4 g, 1.09 mmol) was dissolved in anhydrous Dioxane (10.0 mL). The 4.0 M HCl solution in Dioxane (0.82 mL, 3.28 mmol) was added and r×n mixture was stirred at 50° C. temperature for 8 h. LCMS data shows product formation m/z 266.1. The r×n mixture was cooled at ambient temperature and concentrated to obtain the solid product (0.32 g, Yield 86.4%). MS ESI) mass calcd. for C₁₃H₁₆ClN₂O, 265.74: m/z found 266.1 [M+H]+.

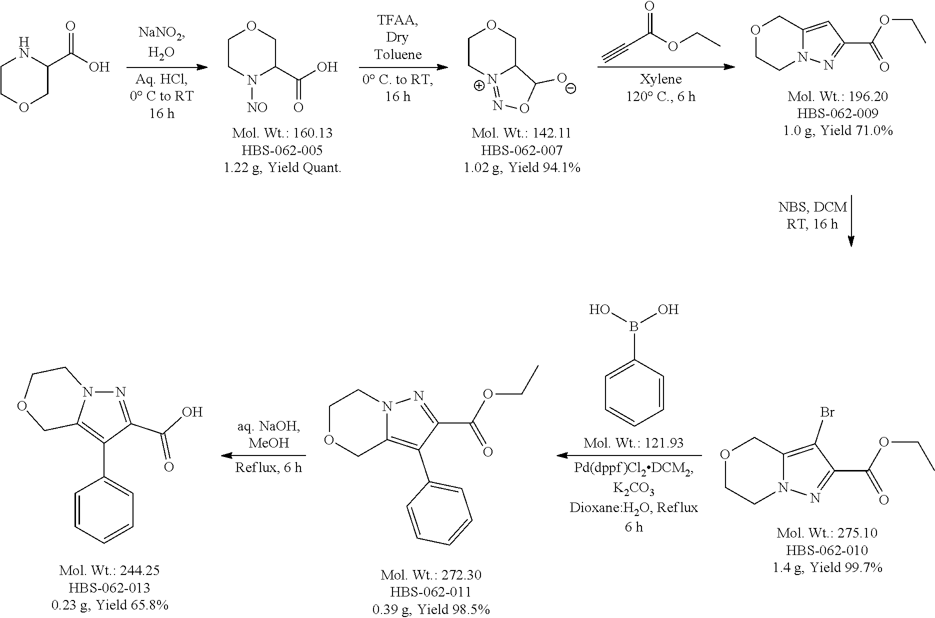

Step 1: Synthesis of HBS-062-005: Morpholine-3-carboxylic acid (1.0 g, 7.63 mmol) was dissolved in water (6.0 mL). Anhydrous NaNO₂ (0.79 g, 11.44 mmol) was added and r×n mixture was cooled at 0° C. temperature in Ice bath. The 12.0 M aq. HCl (1.27 mL, 15.26 mmol) was added, and reaction mixture was gradually warmed at ambient temperature for 16 h. The LCMS data shows product formation m/z 161.1. The reaction mixture was extracted with ethyl acetate. The combined ethyl acetate layer was separated and dried over anhydrous sodium sulfate. The evaporation of solvent gave crude product. The 1.22 g of product was obtained (Yield Quant.). MS (ESI) mass calcd. for C₅H₈N₂O₄, 160.13; m/z found 161.1 [M+H]+.

Step 2: Synthesis of HBS-062-007: Compound HBS-062-005 (1.22 g, 7.63 mmol) was dissolved in anhydrous toluene (10.0 mL). The r×n mixture was cooled at 0° C. temperature in the Ice bath. The anhydrous TFA (1.6 mL, 11.44 mmol) was added, and reaction mixture was gradually warmed at ambient temperature for 16 h. The LCMS data shows product formation m/z 143.1. The reaction mixture was concentrated under reduced pressure to obtain the crude product. The crude product was purified by ISCO combi-flash chromatography system, Mobile phase: EtOAc:Hexane gradient. The 1.02 g of product was obtained (Yield 94.1%). MS (ESI) mass calcd. for C₅H₆N₂O₃, 142.11; m/z found 143.1 [M+H]+.

Step 3: Synthesis of HBS-062-009: Compound HBS-062-007 (1.0 g, 7.18 mmol) was dissolved in Xylene (10.0 mL). The ethyl propiolate (0.95 mL, 9.33 mmol) was added, and reaction mixture was heated at 120° C. temperature for 6 h. The LCMS data shows product formation m/z 197.1. The reaction mixture was concentrated under reduced pressure to obtain the crude product. The crude product was purified by ISCO combi-flash chromatography system, Mobile phase: EtOAc:Hexane gradient. The 1.0 g of product was obtained (Yield 71.0%). MS (ESI) mass calcd. for C₉H₁₂N₂O₃, 196.20; m/z found 197.1 [M+H]+.

Step 4: Synthesis of HBS-062-010: Compound HBS-062-009 (1.0 g, 5.1 mmol) was dissolved in DCM (15.0 mL). The NBS (1.0 g, 5.61 mmol) was added and r×n mixture was stirred at ambient temperature for 16 h. The LCMS data shows product formation m/z 275.0. The reaction mixture was diluted with water and the product was extracted with DCM. The combined DCM layer was separated and dried over anhydrous sodium sulfate. The evaporation of solvent gave crude product. The crude product was purified by ISCO combi-flash system, Mobile phase: EtOAc:Hexane gradient. The 1.4 g of product was obtained (Yield 99.7%). MS (ESI) mass calcd. for C₉H₁₁BrN₂O₃, 275.1; m/z found 275.0 [M+H]+.

Step 5: Synthesis of HBS-052-011: Compound HBS-062-010 (0.4 g, 1.45 mmol) and Phenylboronic acid (0.27 g, 2.18 mmol) were dissolved in mixture of Dioxane/water (9:1 v/v mL). The anhydrous K₂CO₃ (0.6 g, 4.36 mmol) was added followed by Pd(dppf)Cl₂·DCM₂ (0.06 g, 0.073 mmol). The r×n mixture was stirred at 100° C. temperature under N₂ atm. for 6 h. The LCMS data shows product formation m/z 273.1. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.39 g of product was obtained (Yield 95.5%). MS (ESI) mass calcd. for C₁₅H₁₆N₂O₃, 272.3; m/z found 273.1 [M+H]+.

Step 6: Synthesis of HBS-062-013: Compound HBS-062-011 (0.39 g, 1.43 mmol) was dissolved in MeOH (8.0 mL). The 1.0 N aq. solution of NaOH (4.3 mL, 4.3 mmol) was added and r×n mixture was refluxed for 6 h. The LCMS data shows product formation m/z 245.1. The r×n mixture was concentrated under reduced pressure and diluted with water. The aq. layer was acidified with 2.0 M aq. HCl solution (pH=5). The ppts were filtered and washed with water (2.0 mL×3) to obtain 0.23 g of solid product (Yield 65.8%). MS (ESI) mass calcd. for C₁₃H₁₂N₂O₃, 244.25; m/z found 245.1 [M+H]+.

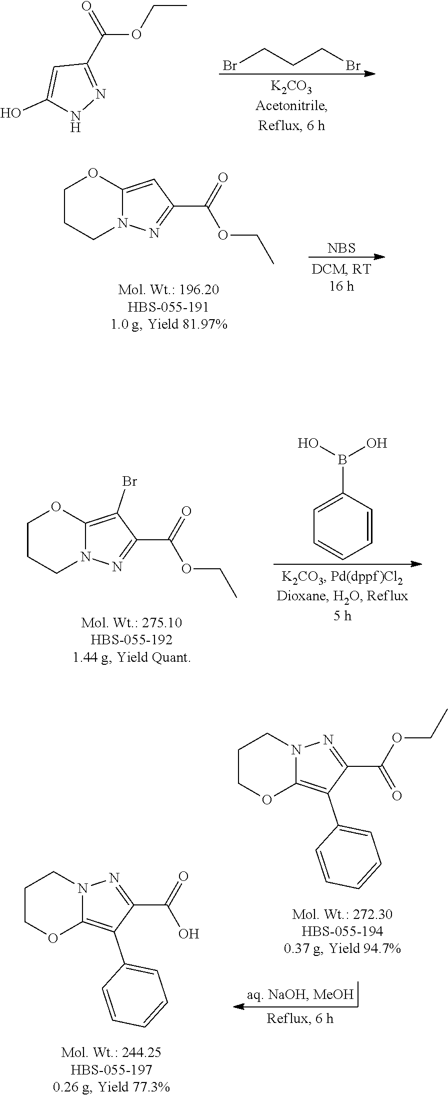

Step 1: Synthesis of HBS-055-191: Ethyl-5-hydroxy-1H-pyrazole-3-carboxylate (1.0 g, 6.40 mmol) was dissolved in anhydrous Acetonitrile (15.0 mL). The anhydrous K₂CO₃ (3.54 g, 25.62 mmol) was added, and reaction mixture was stirred at ambient temperature for 15.0 min. The 1,3-dibromo propane (0.72 mL, 7.05 mmol) was added, and reaction mixture was heated at reflux for 6 h. The LCMS data shows product formation m/z 197.1. The reaction mixture cooled at ambient temperature and filtered. The filtrate was concentrated under reduced pressure to obtain the crude product. The crude product was purified by ISCO combi-flash chromatography system, Mobile phase: EtOAc:Hexane gradient. The 1.03 g of product was obtained (Yield 81.97%). MS (ESI) mass calcd. for C₉H₁₂N₂O₃, 196.20; m/z found 197.1 [M+H]+.

Step 2: Synthesis of HBS-055-192: Compound HBS-055-191 (1.03 g, 5.25 mmol) was dissolved in DCM (15.0 mL). The NBS (0.93 g, 5.25 mmol) was added and r×n mixture was stirred at ambient temperature for 16 h. The LCMS data shows product formation m/z 275.0. The reaction mixture was diluted with water and the product was extracted with DCM. The combined DCM layer was separated and dried over anhydrous sodium sulfate. The evaporation of solvent gave crude product. The crude product was purified by ISCO combi-flash system, Mobile phase: EtOAc:Hexane gradient. The 1.44 g of product was obtained (Yield Quant.). MS (ESI) mass calcd. for C₉H₁₁BrN₂O₃, 275.1; m/z found 275.0 [M+H]+.

Step 3: Synthesis of HBS-055-194: Compound HBS-055-192 (0.4 g, 1.45 mmol) and Phenylboronic acid (0.27 g, 2.18 mmol) were dissolved in mixture of Dioxane/water (9:1 v/v mL). The anhydrous K₂CO₃ (0.6 g, 4.36 mmol) was added followed by Pd(dppf)Cl₂·DCM₂ (0.06 g, 0.073 mmol). The r×n mixture was stirred at 100° C. temperature under N₂ atm. for 6 h. The LCMS data shows product formation m/z 273.1. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.37 g of product was obtained (Yield 94.7%). MS (ESI) mass calcd. for C₁₅H₁₆N₂O₃, 272.3; m/z found 273.1 [M+H]+.

Step 4: Synthesis of HBS-055-197: Compound HBS-055-194 (0.37 g, 1.38 mmol) was dissolved in MeOH (5.0 mL). The 1.0 N aq. solution of NaOH (4.1 mL, 4.1 mmol) was added and r×n mixture was refluxed for 6 h. The LCMS data shows product formation m/z 245.1. The r×n mixture was concentrated under reduced pressure and diluted with water. The aq. layer was acidified with 2.0 M aq. HCl solution (pH=5). The ppts were filtered and washed with water (2.0 mL×3) to obtain 0.26 g of solid product (Yield 77.3%). MS (ESI) mass calcd. for C₁₃H₁₂N₂O₃, 244.25; m/z found 245.1 [M+H]+.

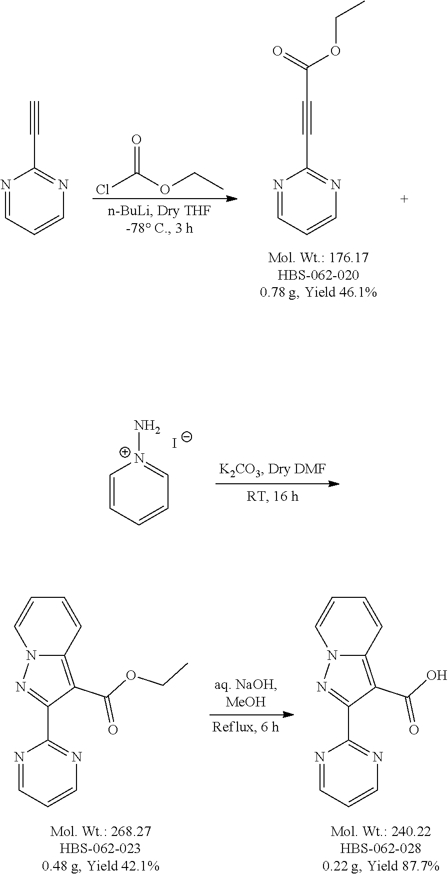

Step 1: Synthesis of HBS-062-020: 1-Ethynyl pyrimidine (1.0 g, 9.61 mmol) was dissolved in anhydrous THF (12.0 mL). The n-BuLi (7.2 mL, 11.53 mmol) was added at −78° C. temperature and r×n mixture was stirred at −78° C. temperature for 30.0 min. The ethyl chloroformate (1.4 mL, 14.41 mmol) was added at −78° C. temperature and r×n mixture was gradually warmed to ambient temperature for 3 h. The LCMS data shows product formation m/z 177.1. The r×n mixture was diluted with aq. NH₄Cl solution and product was extracted with ethyl acetate. The combined ethyl acetate layer was separated and dried over anhydrous sodium sulfate. The evaporation of solvent gave crude product. The crude product was purified by ISCO combi-flash system, Mobile phase: DCM:MeOH gradient. The 0.78 g of product was obtained (Yield 46.1%). MS (ESI) mass calcd. for C₉H₈N₂O₂, 176.17; m/z found 177.1 [M+H]+.

Step 2: Synthesis of HBS-062-023: Compound HBS-062-020 (0.75 g, 4.25 mmol) and 1-Aminopyridinium iodide (1.13 g, 5.11 mmol) were dissolved in anhydrous DMF (10.0 mL). The anhydrous K₂CO₃ (1.47 g, 10.63 mmol) was added, and reaction mixture was stirred at ambient temperature for 16 h. The LCMS data shows product formation m/z 269.1. The r×n mixture was diluted with water and the product was extracted with ethyl acetate. The combined ethyl acetate layer was separated and dried over anhydrous sodium sulfate. The evaporation of solvent gave crude product. The crude product was purified by ISCO combi-flash system, Mobile phase: Ethyl acetate:Hexane gradient. The 0.48 g of product was obtained (Yield 42.1%). MS (ESI) mass calcd. for C₁₄H₁₂N₄O₂, 268.27; m/z found 269.1 [M+H]+.

Step 3: Synthesis of HBS-062-028: Compound HBS-062-023 (0.28 g, 1.04 mmol) was dissolved in MeOH (8.0 mL). The 1.0 N aq. NaOH solution (2.1 mL, 2.09 mmol) was added, and reaction mixture was refluxed for 6 h. LCMS shows product formation m/z 241.1. The reaction mixture was concentrated under reduced pressure. The solid was dissolved in water and acidified with 2.0 M aq. HCl solution (pH=5).

The ppts were filtered and washed with water (2.0 mL×3) to obtain 0.22 g of solid product (Yield 87.5%). MS (ESI) mass calcd. for C₁₂H₈N₄O₂, 240.22; m/z found 241.1 [M+H]+.

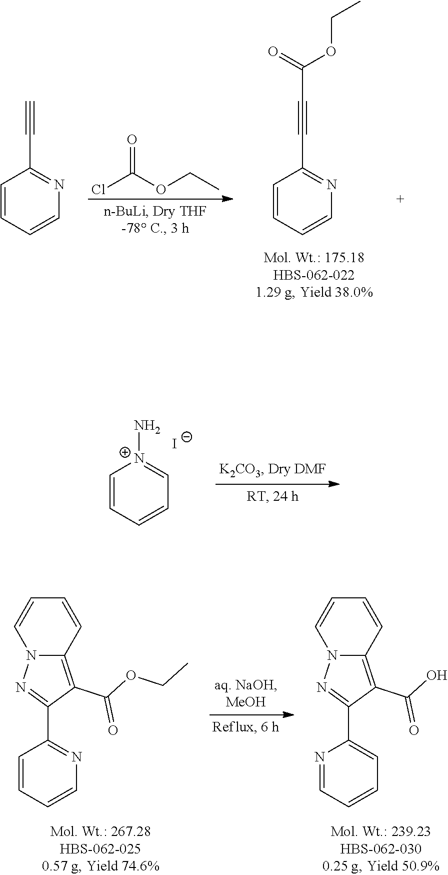

Step 1: Synthesis of HBS-062-022: 1-Ethynyl pyridine (2.0 g, 19.4 mmol) was dissolved in anhydrous THF (15.0 mL). The n-BuLi (14.6 mL, 23.3 mmol) was added at −78° C. temperature and r×n mixture was stirred at −78° C. temperature for 30.0 min. The ethyl chloroformate (2.2 mL, 23.3 mmol) was added at −78° C. temperature and r×n mixture was gradually warmed to ambient temperature for 3 h. The LCMS data shows product formation m/z 176.1. The r×n mixture was diluted with aq. NH₄Cl solution and product was extracted with ethyl acetate. The combined ethyl acetate layer was separated and dried over anhydrous sodium sulfate. The evaporation of solvent gave crude product. The crude product was purified by ISCO combi-flash system, Mobile phase: Ethyl acetate:Hexane gradient. The 1.29 g of product was obtained (Yield 38.0%). MS (ESI) mass calcd. for C₁₀H₉NO₂, 175.18; m/z found 176.1 [M+H]+.

Step 2: Synthesis of HBS-062-025: Compound HBS-062-022 (0.5 g, 2.85 mmol) and 1-Aminopyridinium iodide (0.76 g, 3.43 mmol) were dissolved in anhydrous DMF (8.0 mL). The anhydrous K₂CO₃ (0.79 g, 5.71 mmol) was added, and reaction mixture was stirred at ambient temperature for 16 h. The LCMS data shows product formation m/z 268.1. The r×n mixture was diluted with water and the product was extracted with ethyl acetate. The combined ethyl acetate layer was separated and dried over anhydrous sodium sulfate. The evaporation of solvent gave crude product. The crude product was purified by ISCO combi-flash system, Mobile phase: Ethyl acetate:Hexane and DCM:Methanol gradient. The 0.57 g of product was obtained (Yield 74.6%). MS (ESI) mass calcd. for C₁₅H₁₃N₃O₂, 267.28; m/z found 268.1 [M+H]+.

Step 3: Synthesis of HBS-062-030: Compound HBS-062-025 (0.56 g, 2.1 mmol) was dissolved in MeOH (8.0 mL). The 1.0 N aq. NaOH solution (4.2 mL, 4.19 mmol) was added, and reaction mixture was refluxed for 4 h. LCMS shows product formation m/z 240.1. The reaction mixture was concentrated under reduced pressure. The solid was dissolved in water and acidified with 2.0 M aq. HCl solution (pH=5). The ppts were filtered and washed with water (2.0 mL×3) to obtain 0.26 g of solid product (Yield 50.9%). MS (ESI) mass calcd. for C₁₃H₉N₃O₂, 239.23; m/z found 240.1 [M+H]+.

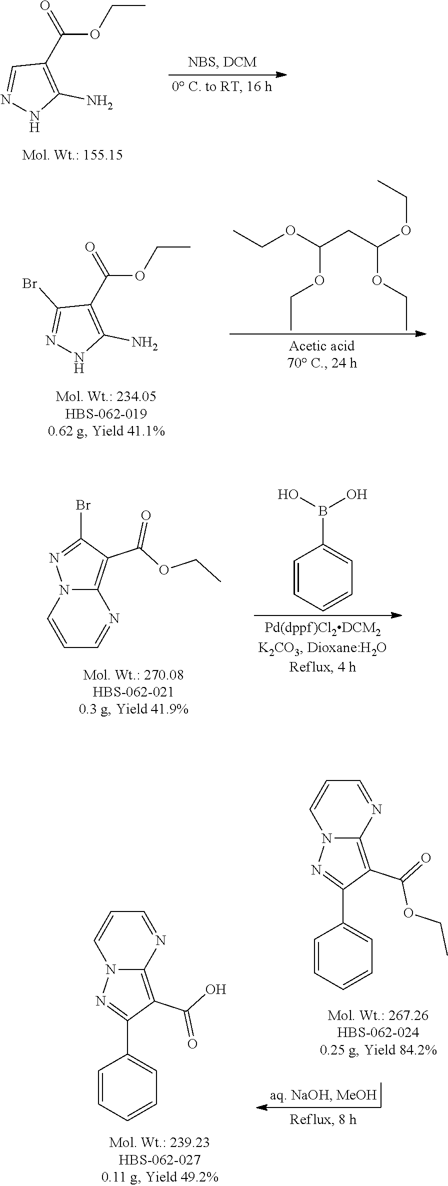

Step 1: Synthesis of HBS-062-019: Ethyl 5-amino-1H-Pyrazole-4-carboxylate (1.0 g, 6.44 mmol) was dissolved in DCM (15.0 mL). The reaction mixture was cooled at 0° C. temperature in Ice bath. The NBS (1.38 g, 7.73 mmol) was added and r×n mixture was stirred at ambient temperature for 16 h. The LCMS data shows product formation m/z 236.0. The reaction mixture was diluted with aq. saturated solution of NaHCO₃. The product was extracted with DCM. The combined DCM layer was separated and dried over anhydrous sodium sulfate. The evaporation of solvent gave crude product. The crude product was purified by ISCO combi-flash system, Mobile phase: DCM:Methanol gradient. The 0.62 g of product was obtained (Yield 41.1%). MS (ESI) mass calcd. for C₆H₈BrN₃O₂, 234.05; m/z found 236.0 [M+H]+.

Step 2: Synthesis of HBS-062-021: Compound HBS-062-019 (0.62 g, 2.65 mmol) and 1,1,3,3-Tetraethoxy propane (0.76 mL, 3.18 mmol) were dissolved in anhydrous Acetic acid (10.0 mL). The reaction mixture was heated at 70° C. temperature for 24 h. The LCMS data shows product formation m/z 272.0. The reaction mixture was concentrated under reduced pressure to obtain the crude product. The crude product was diluted with water and neutralized with aq. saturated solution of NaHCO₃. The ppts were filtered and dried to obtain 0.3 g of product (Yield 41.9%). MS (ESI) mass calcd. for C₉H₆BrN₃O₂, 270.08; m/z found 272.0 [M+H]+.

Step 3: Synthesis of HBS-062-024: Compound HBS-062-021 (0.3 g, 1.11 mmol) and Phenylboronic acid (0.2 g, 1.67 mmol) were dissolved in mixture of Dioxane/water (7:1 v/v mL). The anhydrous K₂CO₃ (0.46 g, 3.33 mmol) was added followed by Pd(dppf)Cl₂·DCM₂ (0.045 g, 0.056 mmol). The r×n mixture was stirred at 100° C. temperature under N₂ atm. for 4 h. The LCMS data shows product formation m/z 268.1. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.25 g of product was obtained (Yield 84.2%). MS (ESI) mass calcd. for C₁₅H₁₃NO₂, 267.28; m/z found 268.1 [M+H]+.

Step 4: Synthesis of HBS-062-027: Compound HBS-062-024 (0.25 g, 0.94 mmol) was dissolved in MeOH (6.0 mL). The 1.0 N aq. solution of NaOH (1.9 mL, 1.87 mmol) was added and r×n mixture was refluxed for 8 h. The LCMS data shows product formation m/z 240.1. The r×n mixture was concentrated under reduced pressure and diluted with water. The aq. layer was acidified with 2.0 M aq. HCl solution (pH=5). The ppts were filtered and washed with water (2.0 mL×3) to obtain 0.11 g of solid product (Yield 49.2%). MS (ESI) mass calcd. for C₁₃H₉N₃O₂, 239.23; m/z found 240.1 [M+H]+.

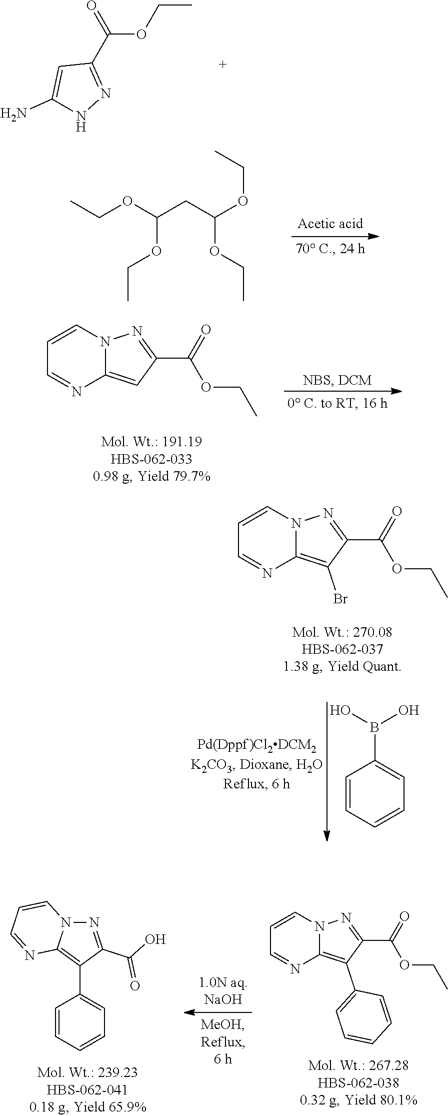

Step 1: Synthesis of HBS-062-033: Ethyl 5-amino-1H-Pyrazole-4-carboxylate (1.0 g, 6.43 mmol) and 1,1,3,3-Tetraethoxy propane (1.85 mL, 7.72 mmol) were dissolved in anhydrous Acetic acid (8.0 mL). The reaction mixture was heated at 70° C. temperature for 24 h. The LCMS data shows product formation m/z 192.1.0. The reaction mixture was concentrated under reduced pressure to obtain the crude product. The crude product was diluted with water and neutralized with aq. saturated solution of NaHCO₃. The product was extracted with DCM. The combined DCM layer was separated and dried over anhydrous sodium sulfate. The evaporation of solvent gave crude product. The crude product was purified by ISCO combi-flash system, Mobile phase: DCM:Methanol gradient. The 0.98 g of product was obtained (Yield 79.7%). MS (ESI) mass calcd. for C₉H₉N₃O₂, 191.19; m/z found 192.1 [M+H]+.

Step 2: Synthesis of HBS-062-037: Compound HBS-062-033 (0.98 g, 5.13 mmol) was dissolved in DCM (15.0 mL). The reaction mixture was cooled at 0° C. temperature in Ice bath. The NBS (1.0 g, 5.64 mmol) was added and r×n mixture was stirred at ambient temperature for 16 h. The LCMS data shows product formation m/z 272.0. The reaction mixture was diluted with aq. saturated solution of NaHCO₃. The product was extracted with DCM. The combined DCM layer was separated and dried over anhydrous sodium sulfate. The evaporation of solvent gave 1.38 g of crude product (Yield Quant.). MS (ESI) mass calcd. for C₉H₈BrN₃O₂, 270.08; m/z found 272.0 [M+H]+.

Step 3: Synthesis of HBS-062-038: Compound HBS-062-037 (0.4 g, 1.48 mmol) and Phenylboronic acid (0.27 g, 2.22 mmol) were dissolved in mixture of Dioxane/water (8:1 v/v mL). The anhydrous K₂CO₃ (0.61 g, 4.44 mmol) was added followed by Pd(dppf)Cl₂·DCM₂ (0.06 g, 0.074 mmol). The r×n mixture was stirred at 100° C. temperature under N₂ atm. for 6 h. The LCMS data shows product formation m/z 268.1. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.32 g of product was obtained (Yield 80.1%). MS (ESI) mass calcd. for C₁₅H₁₃NO₂, 267.28; m/z found 268.1 [M+H]+.

Step 4: Synthesis of HBS-062-041: Compound HBS-062-038 (0.32 g, 1.19 mmol) was dissolved in MeOH (5.0 mL). The 1.0 N aq. solution of NaOH (2.4 mL, 2.37 mmol) was added and r×n mixture was refluxed for 6 h. The LCMS data shows product formation m/z 240.1. The r×n mixture was concentrated under reduced pressure and diluted with water. The aq. layer was acidified with 2.0 M aq. HCl solution (pH=5). The ppts were filtered and washed with water (2.0 mL×3) to obtain 0.18 g of solid product (Yield 65.9%). MS (ESI) mass calcd. for C₁₃H₈N₃O₂, 239.23; m/z found 240.1 [M+H]+.

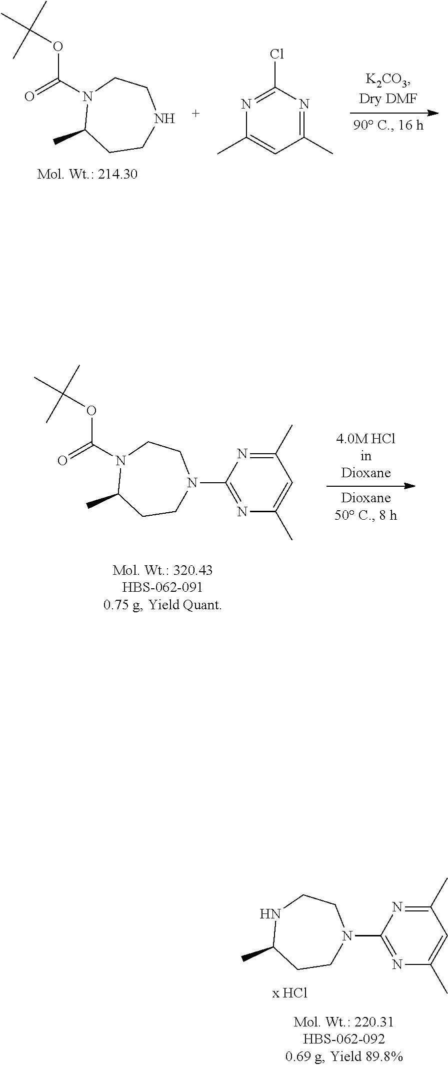

Step 1: Synthesis of HBS-062-091: Tert-butyl (7R)-7-methyl-1,4-diazepane-1-carboxylate (0.5 g, 2.33 mmol) and 2-chloro-4,6-dimethylpyrimidine (0.4 g, 2.8 mmol) were dissolved in anhydrous DMF (8.0 mL). The anhydrous K₂CO₃ (0.64 g, 4.66 mmol) was added, and reaction mixture was stirred at 90° C. temperature for 16 h. The LCMS data shows product formation m/z 321.1. The reaction mixture was diluted with water and the product was extracted with ethyl acetate. The combined ethyl acetate layer was separated and dried over anhydrous sodium sulfate. The evaporation of solvent gave crude product. The crude product was purified by ISCO combi-flash system, Mobile phase: EtOAc:Hexane gradient. The 0.75 g of product was obtained (Yield Quant.). MS (ESI) mass calcd. for C₁₇H₂₆N₄O₂, 320.43; m/z found 321.2 [M+H]+.

Step 2: Synthesis of HBS-062-092: Compound HBS-062-091 (0.75 g, 2.33 mmol) was dissolved in anhydrous Dioxane (6.0 mL). The 4.0 M HCl solution in Dioxane (2.91 mL, 11.65 mmol) was added and r×n mixture was stirred at 50° C. temperature for 8 h. LCMS data shows product formation m/z 221.2. The r×n mixture was cooled at ambient temperature and concentrated to obtain the solid product (0.69 g, Yield 89.8%). MS (ESI) mass calcd. for C₁₂H₂₀N₄, 220.31; m/z found 221.2 [M+H]+.

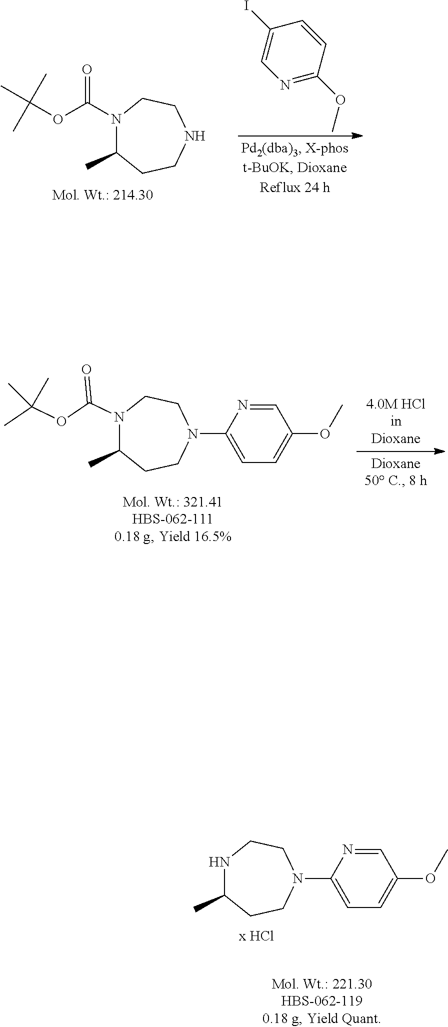

Step 1: Synthesis of HBS-062-111: Tert-butyl (7R)-7-methyl-1,4-diazepane-1-carboxylate (0.73 g, 3.4 mmol) and 2-lodo-5-methoxy pyridine (0.8 g, 3.4 mmol) were dissolved in anhydrous 1,4-Dioxane (2.0 mL). The anhydrous t-BuOK (0.95 g, 8.5 mmol) was added, and reaction mixture was stirred under N₂ atm. The Pd₂(dba)₃ (0.16 g, 0.17 mmol) and X-Phos (0.16 g, 0.34 mmol) were added and r×n mixture was heated at reflux for 24 h. The reagents were added to consume the starting material and to drive the reaction to completion. The LCMS data shows product formation m/z 322.1. The reaction mixture was filtered over celite. The filtrate was concentrated to obtain the crude product. The crude product was purified by ISCO combi-flash system, Mobile phase: DCM:Methanol gradient. The 0.18 g of product was obtained (Yield 16.5%). MS (ESI) mass calcd. for C₁₇H₂₇N₃O₃, 321.41; m/z found 322.2 [M+H]+.

Step 2: Synthesis of HBS-062-119: Compound HBS-062-111 (0.18 g, 0.56 mmol) was dissolved in anhydrous Dioxane (4.0 mL). The 4.0 M HCl solution in Dioxane (0.7 mL, 2.8 mmol) was added and r×n mixture was stirred at 50° C. temperature for 8 h. LCMS data shows product formation m/z 222.2. The r×n mixture was cooled at ambient temperature and concentrated to obtain the product (0.18 g, Yield Quant.). MS (ESI) mass calcd. for C₁₂H₁₉N₃O, 221.30; m/z found 222.2 [M+H]+.

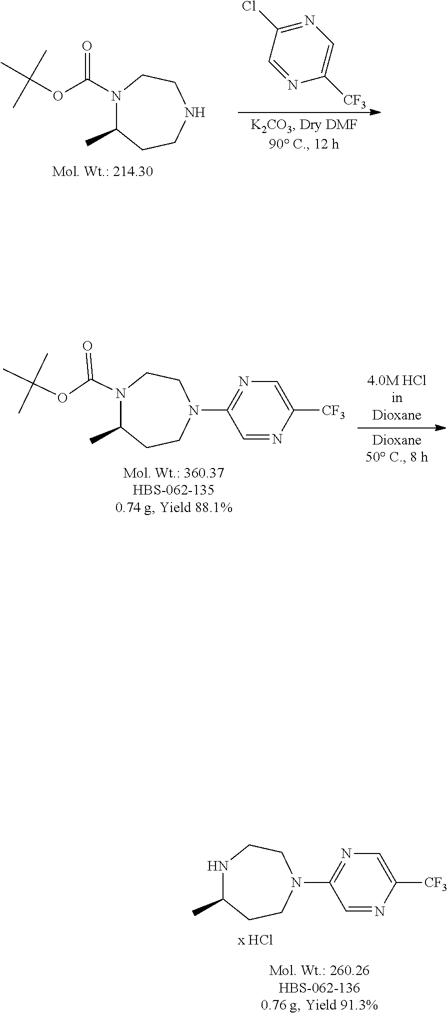 [SMILES: C[C@@H]1CCNCCN1C(=O)OC(C)(C)C ; FC(F)(F)c1cnc(Cl)cn1 ; C[C@@H]1CCN(c2cnc(C(F)(F)F)cn2)CCN1C(=O)OC(C)(C)C ; C[C@@H]1CCN(c2cnc(C(F)(F)F)cn2)CCN1]

Step 1: Synthesis of HBS-062-135: Tert-butyl (7R)-7-methyl-1,4-diazepane-1-carboxylate (0.5 g, 2.33 mmol) and 2-Chloro-5-(trifluoromethyl)pyrazine (0.35 mL, 2.8 mmol) were dissolved in anhydrous DMF (8.0 mL). The anhydrous K₂CO₃ (0.64 g, 4.66 mmol) was added, and reaction mixture was heated at 90° C. temperature for 12 h. The LCMS data shows product formation m/z 305.0. The reaction mixture was diluted with water and the product was extracted with ethyl acetate. The combined ethyl acetate layer was separated and dried over anhydrous sodium sulfate. The evaporation of solvent gave crude product. The crude product was purified by ISCO combi-flash system, Mobile phase: EtOAc:Hexane gradient. The 0.74 g of product was obtained (Yield 88.1%). MS (ESI) mass calcd. for C₁₆H₂₃F₃N₄O₂, 360.37; m/z found 305.0 [M-tButyl]⁺

Step 2: Synthesis of HBS-062-136: Compound HBS-062-135 (0.74 g, 2.05 mml) was dissolved in anhydrous Dioxane (10.0 mL). The 4.0 M HCl solution in Dioxane (2.6 mL, 10.25 mmol) was added and reaction mixture was stirred at 50° C. temperature for 8 h. LCMS data shows product formation m/z 261.0. The r×n mixture was cooled at ambient temperature and concentrated to obtain the solid product (0.76 g, Yield 91.3%). MS (ESI) mass calcd. for C₁₁H₁₅F₃N₄, 260.26; m/z found 261.0 [M+H]+.

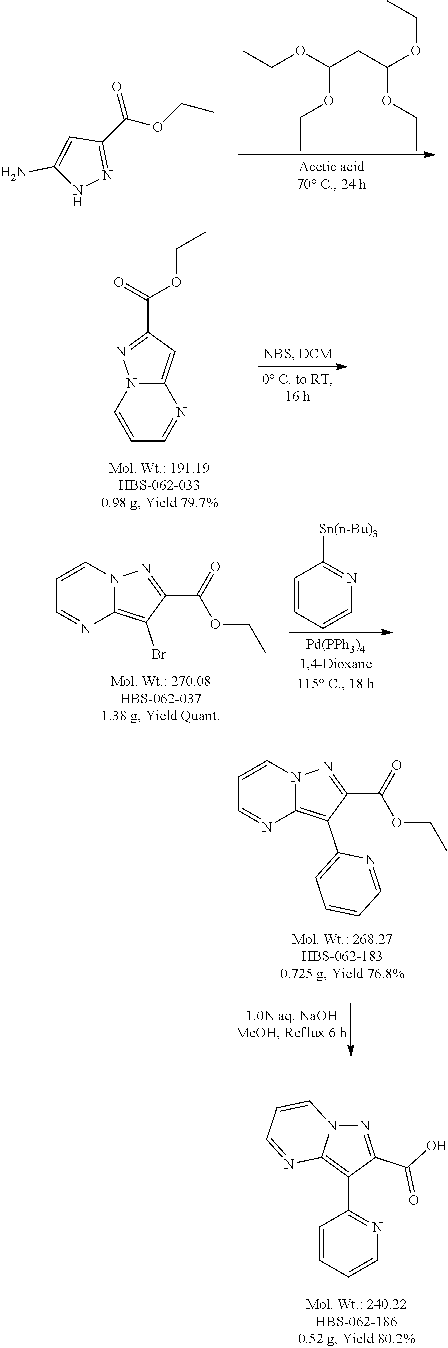

Step 1: Synthesis of HBS-062-033: Ethyl 5-amino-1H-Pyrazole-4-carboxylate (1.0 g, 6.43 mmol) and 1,1,3,3-Tetraethoxy propane (1.85 mL, 7.72 mmol) were dissolved in anhydrous Acetic acid (8.0 mL). The reaction mixture was heated at 70° C. temperature for 24 h. The LCMS data shows product formation m/z 192.1.0. The reaction mixture was concentrated under reduced pressure to obtain the crude product. The crude product was diluted with water and neutralized with aq. saturated solution of NaHCO₃. The product was extracted with DCM. The combined DCM layer was separated and dried over anhydrous sodium sulfate. The evaporation of solvent gave crude product. The crude product was purified by ISCO combi-flash system, Mobile phase: DCM:Methanol gradient. The 0.98 g of product was obtained (Yield 79.7%). MS (ESI) mass calcd. for C₉H₉N₃O₂, 191.19; m/z found 192.1 [M+H]+.

Step 2: Synthesis of HBS-062-037: Compound HBS-062-033 (0.98 g, 5.13 mmol) was dissolved in DCM (15.0 mL). The reaction mixture was cooled at 0° C. temperature in Ice bath. The NBS (1.0 g, 5.64 mmol) was added and r×n mixture was stirred at ambient temperature for 16 h. The LCMS data shows product formation m/z 272.0. The reaction mixture was diluted with aq. saturated solution of NaHCO₃. The product was extracted with DCM. The combined DCM layer was separated and dried over anhydrous sodium sulfate. The evaporation of solvent gave 1.38 g of crude product (Yield Quant.). MS (ESI) mass calcd. for C₉H₈BrN₃O₂, 270.08; m/z found 272.0 [M+H]+.

Step 3: Synthesis of HBS-062-183: Compound HBS-062-037 (0.95 g, 3.52 mmol) and 2-(tributyl stannyl)-pyridine (1.94 g, 5.28 mmol) were dissolved in 1,4-Dioxane (12.0 mL). The Pd(PPh₃)₄ (0.41 g, 0.35 mmol) was added, and reaction mixture was heated at 115° C. temperature under N₂ atm. for 18 h. The 0.05 eq Pd(PPh₃)₄ was added to consume the starting material. The LCMS data shows product formation m/z 269.0. The r×n mixture was filtered over celite bed and washed with ethyl acetate. The filtrate was concentrated under reduced pressure to obtain the crude product. The crude product was purified by ISCO combi-flash chromatography system, Mobile phase: EtOAc:Hexane gradient. The 0.725 g of product was obtained (Yield 76.8%). MS (ESI) mass calcd. for C₁₄H₁₂N₄O₂, 268.27; m/z found 269.0 [M+H]+.

Step 4: Synthesis of HBS-062-186: Compound HBS-062-183 (0.73 g, 2.7 mmol) was dissolved in MeOH (8.0 mL). The 1.0 N aq. solution of NaOH (5.4 mL, 5.4 mmol) was added and r×n mixture was refluxed for 6 h. The LCMS data shows product formation m/z 241.1. The r×n mixture was concentrated under reduced pressure and diluted with water. The aq. layer was acidified with 2.0 M aq. HCl solution (pH=5). The ppts were filtered and washed with water (2.0 mL×3) to obtain 0.52 g of product (Yield 80.2%). MS (ESI) mass calcd. for C₁₂H₈N₄O₂, 240.22; m/z found 241.1 [M+H]+.

### IV. Synthesis of Example Compounds

### A. Example 1

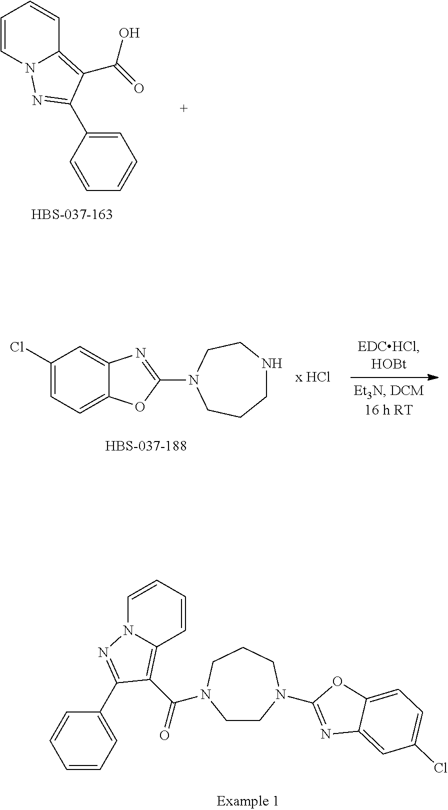 [SMILES: O=C(O)c1c(-c2ccccc2)nn2ccccc12 ; Clc1ccc2oc(N3CCCNCC3)nc2c1 ; O=C(c1c(-c2ccccc2)nn2ccccc12)N1CCCN(c2nc3cc(Cl)ccc3o2)CC1]

Synthesis of Compound Example 1: Intermediate HBS-037-163 (0.025 g, 0.1 mmol) was dissolved in anhydrous DCM (2.5 mL). EDC·HCl (0.04 g, 0.21 mmol) and HOBt (0.03 g, 0.21 mmol) were added followed by Et₃N (0.15 mL, 1.1 mmol). The reaction mixture was stirred at ambient temperature for 10.0 min. Intermediate HBS-037-188 (0.038 g, 0.1 mmol) was added to the reaction mixture. The reaction mixture was stirred at ambient temperature for 16 h. The LCMS data shows product formation m/z 472.2. The reaction mixture was diluted with DCM and washed with saturated solution of NaHCO₃. The DCM layer was separated and dried over anhydrous Na₂SO₄. The evaporation of solvent gave crude product which was purified by ISCO combi-flash system, Mobile phase: EtOAc:Hexane gradient. The 0.05 g of product was obtained (Yield Quant.). MS (ESI) mass calculated for C₂₆H₂₂ClN₅O₂, 471.9; m/z found 472.2 [M+H]⁺

### B. Example 2

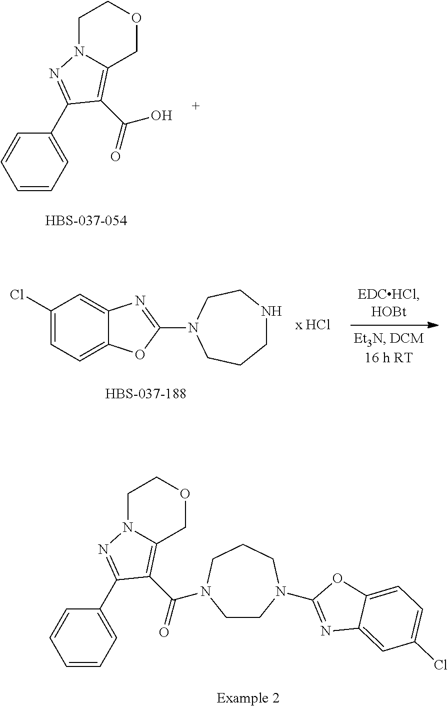 [SMILES: O=C(O)c1c(-c2ccccc2)nn2c1COCC2 ; Clc1ccc2oc(N3CCCNCC3)nc2c1 ; O=C(c1c(-c2ccccc2)nn2c1COCC2)N1CCCN(c2nc3cc(Cl)ccc3o2)CC1]

Synthesis of Compound Example 2: HBS-037-054 (0.026 g, 0.1 mmol) was dissolved in anhydrous DCM (2.5 mL). EDC·HCl (0.04 g, 0.21 mmol) and HOBt (0.03 g, 0.21 mmol) were added followed by Et₃N (0.15 mL, 1.1 mmol). The reaction mixture was stirred at ambient temperature for 10.0 min. HBS-037-188 (0.038 g, 0.1 mmol) was added to the reaction mixture. The reaction mixture was stirred at ambient temperature for 16 h. The LCMS data shows product formation m/z 478.2. The reaction mixture was diluted with saturated solution of NaHCO₃ and the product was extracted with DCM. The combined DCM layer was separated and dried over anhydrous Na₂SO₄. The evaporation of solvent gave crude product which was purified by ISCO combi-flash system, Mobile phase: EtOAc:Hexane gradient. Pure Example 2 was obtained (0.019 g, Yield 37.9%). MS (ESI) mass calculated for C₂₅H₂₄ClN₅O₃, 477.9; m/z found 478.2 [M+H]⁺

### C. Example 3

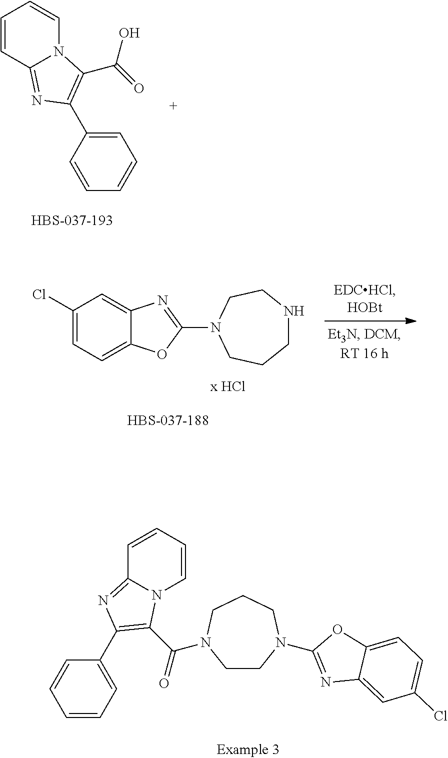 [SMILES: O=C(O)c1c(-c2ccccc2)nc2ccccn12 ; Clc1ccc2oc(N3CCCNCC3)nc2c1 ; O=C(c1c(-c2ccccc2)nc2ccccn12)N1CCCN(c2nc3cc(Cl)ccc3o2)CC1]

Synthesis of Compound Example 3: Intermediate HBS-037-193 (0.025 g, 0.1 mmol) was dissolved in anhydrous DCM (2.5 mL). EDC·HCl (0.04 g, 0.21 mmol) and HOBt (0.03 g, 0.21 mmol) were added followed by Et₃N (0.15 mL, 1.0 mmol). The reaction mixture was stirred at ambient temperature for 10.0 min. HBS-037-188 (0.038 g, 0.1 mmol) was added to the reaction mixture. The reaction mixture was stirred at ambient temperature for 16 h. The LCMS data shows product formation m/z 472.2. The reaction mixture was diluted with saturated solution of NaHCO₃ and the product was extracted with DCM. The combined DCM layer was separated and dried over anhydrous Na₂SO₄. The evaporation of solvent gave crude product which was purified by ISCO combi-flash system, Mobile phase: EtOAc:Hexane gradient. Pure Example 3 was obtained (0.034 g, Yield 68.6%). MS (ESI) mass calculated for C₂₅H₂₂ClN₅O₂, 471.9; m/z found 472.2 [M+H]⁺

### D. Example 4

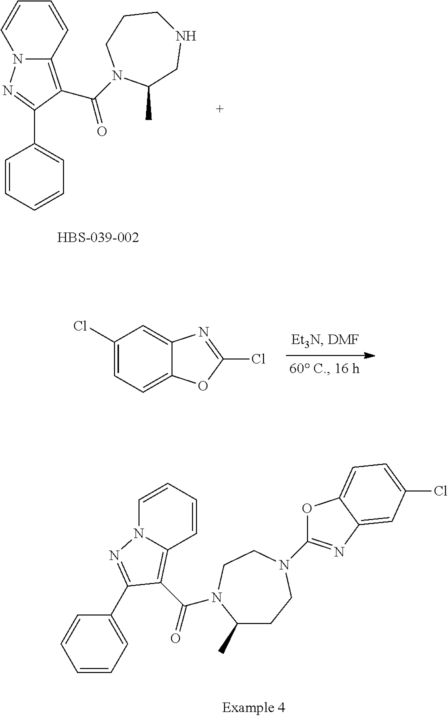 [SMILES: C[C@@H]1CNCCCN1C(=O)c1c(-c2ccccc2)nn2ccccc12 ; Clc1ccc2oc(Cl)nc2c1 ; C[C@@H]1CCN(c2nc3cc(Cl)ccc3o2)CCN1C(=O)c1c(-c2ccccc2)nn2ccccc12]

Synthesis of Compound Example 4: HBS-039-002 (0.037 g, 0.11 mmol) was dissolved in anhydrous DMF (2.5 mL). Triethylamine (0.046 mL, 0.33 mmol) was added followed by 2,5-dichloro-1,3-benzoxazole (0.021 g, 0.11 mmol). The reaction mixture was stirred at 60° C. for 16 h. The LCMS data shows product formation m/z 486. The reaction mixture was diluted with water and the product was extracted with ethyl acetate. The combined ethyl acetate layer was washed with water followed by brine. The organic layer was separated and dried over anhydrous sodium sulfate. Evaporation of solvent gave crude product which was purified by ISCO combi-flash chromatography system, Mobile phase: EtOAc:Hexane gradient. Pure Example 4 was obtained (0.038 g, Yield 69.9%). MS (ESI) mass calculated for C₂₇H₂₄ClN₅O₂, 485.96; m/z found 486.0 [M+H]⁺

### E. Example 5 and Example 6

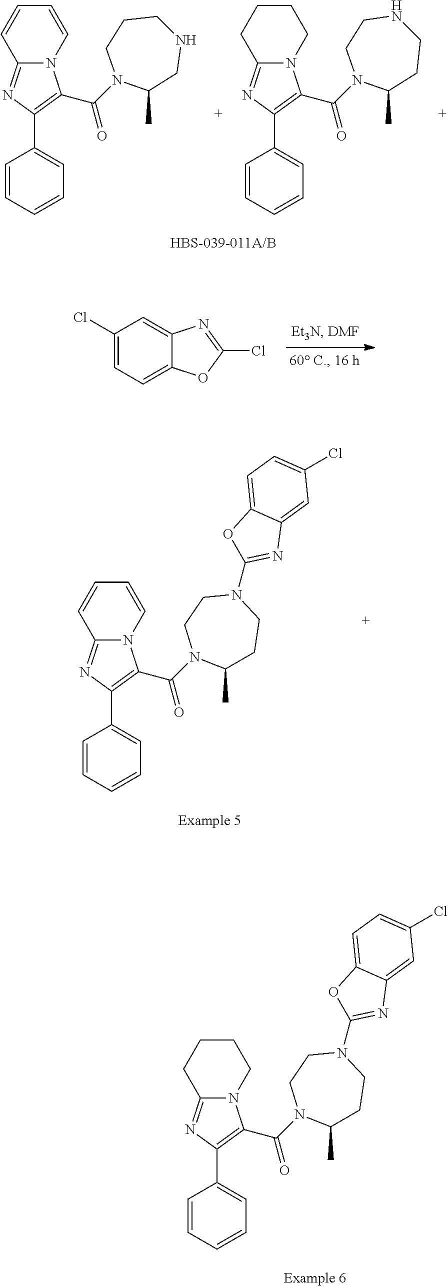 [SMILES: C[C@@H]1CNCCCN1C(=O)c1c(-c2ccccc2)nc2ccccn12 ; C[C@@H]1CCNCCN1C(=O)c1c(-c2ccccc2)nc2n1CCCC2 ; Clc1ccc2oc(Cl)nc2c1 ; C[C@@H]1CCN(c2nc3cc(Cl)ccc3o2)CCN1C(=O)c1c(-c2ccccc2)nc2ccccn12 ; C[C@@H]1CCN(c2nc3cc(Cl)ccc3o2)CCN1C(=O)c1c(-c2ccccc2)nc2n1CCCC2]

Synthesis of Compound Example 5 and Example 6: Intermediate HBS-039-011A/B (0.022 g, 0.07 mmol) was dissolved in anhydrous DMF (2.5 mL). Triethylamine (0.03 mL, 0.2 mmol) was added followed by 2,5-dichloro-1,3-benzoxazole (0.012 g, 0.07 mmol). The reaction mixture was stirred at 60° C. for 16 h. The LCMS data shows mixture of two products formation/z 486 and m/z 490. The reaction mixture was diluted with water and the products were extracted with ethyl acetate. The combined ethyl acetate layer was washed with water followed by brine. The organic layer was separated and dried over anhydrous sodium sulfate and evaporation of solvent gave crude product mixture of Example 5 and Example 6. The mixture was purified by ISCO combi-flash chromatography system, Mobile phase: EtOAc:Hexane gradient and then prep-TLC plate, Mobile Phase: EtOAc:Hexane (60:40, v/v mL). The 0.0053 g of product Example 5 with m/z 486.2 was obtained (Yield 16.7%) and 0.014 g of product Example 6 with m/z 490.2 was obtained (Yield 44.1%). MS (ESI) mass calculated for C₂₇H₂₄ClN₅O₂, 485.96; m/z found 486.2 [M+H]⁺ and MS (ESI) mass calculated for C₂₇H₂₈ClN₅O₂, 490.0; m/z found 490.2 [M+H]⁺

### F. Example 7

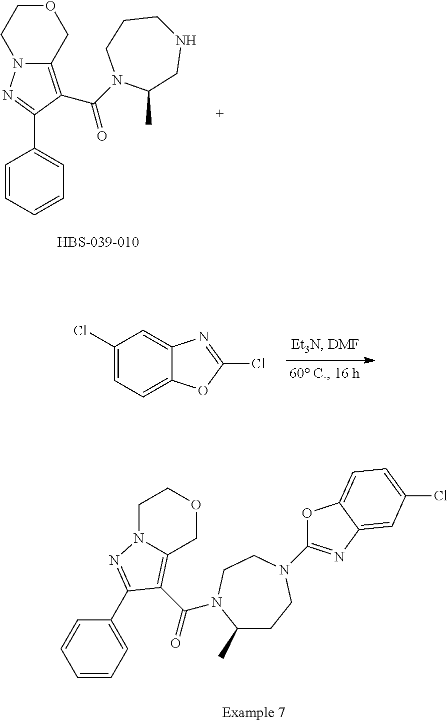 [SMILES: C[C@@H]1CNCCCN1C(=O)c1c(-c2ccccc2)nn2c1COCC2 ; Clc1ccc2oc(Cl)nc2c1 ; C[C@@H]1CCN(c2nc3cc(Cl)ccc3o2)CCN1C(=O)c1c(-c2ccccc2)nn2c1COCC2]

Synthesis of Compound Example 7: HBS-039-010 (0.014 g, 0.04 mmol) was dissolved in anhydrous DMF (2.5 mL). Triethylamine (0.017 mL, 0.12 mmol) was added followed by 2,5-dichloro-1,3-benzoxazole (0.008 g, 0.042 mmol). The reaction mixture was stirred at 60° C. for 16 h. The LCMS data shows product formation m/z 492.2. The reaction mixture was diluted with water and the product was extracted with ethyl acetate. The combined ethyl acetate layer was washed with water followed by brine. The organic layer was separated and dried over anhydrous sodium sulfate. The evaporation of solvent gave crude product which was purified by ISCO combi-flash chromatography system, Mobile phase: EtOAc:Hexane gradient. Pure Example 7 was obtained (12.4 mg, Yield 60.4%). MS (ESI) mass calculated for C₂₆H₂₆ClN₅O₃, 491.97; m/z found 492.2 [M+H]⁺

### G. Example 8

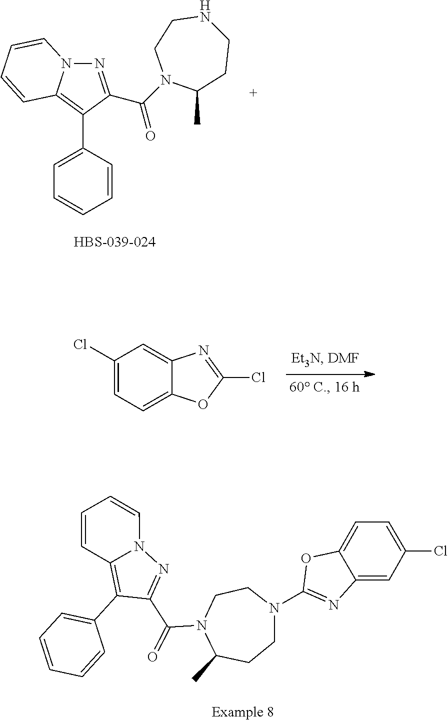 [SMILES: C[C@@H]1CCNCCN1C(=O)c1nn2ccccc2c1-c1ccccc1 ; Clc1ccc2oc(Cl)nc2c1 ; C[C@@H]1CCN(c2nc3cc(Cl)ccc3o2)CCN1C(=O)c1nn2ccccc2c1-c1ccccc1]

Synthesis of Compound Example 8: Intermediate HBS-039-024 (0.032 g, 0.1 mmol) was dissolved in anhydrous DMF (2.5 mL). Triethylamine (0.04 mL, 0.29 mmol) was added followed by 2,5-dichloro-1.3-benzoxazole (0.018 g, 0.1 mmol). The reaction mixture was stirred at 60° C. for 16 h. The LCMS data shows product formation m/z 486.2. The reaction mixture was diluted with water and the product was extracted with ethyl acetate. The combined ethyl acetate layer was washed with water followed by brine. The organic layer was separated and dried over anhydrous sodium sulfate. The evaporation of solvent gave crude product which was purified by prep-TLC plate, Mobile phase: EtOAc:Hexane, (50:50 v/v mL). Pure Example 8 was obtained (0.032 g, Yield 68.8%). MS (ESI) mass calculated for C₂₇H₂₄ClN₅O₂, 485.96: m/z found 486.2 [M+H]⁺

### H. Example 9

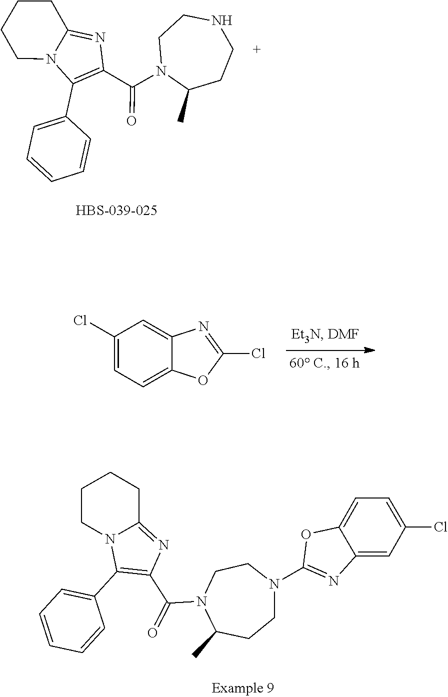 [SMILES: C[C@@H]1CCNCCN1C(=O)c1nc2n(c1-c1ccccc1)CCCC2 ; Clc1ccc2oc(Cl)nc2c1 ; C[C@@H]1CCN(c2nc3cc(Cl)ccc3o2)CCN1C(=O)c1nc2n(c1-c1ccccc1)CCCC2]

Synthesis of Compound Example 9: Intermediate HBS-039-025 (0.017 g, 0.05 mmol) was dissolved in anhydrous DMF (2.5 mL). Triethylamine (0.021 mL, 0.15 mmol) was added followed by 2,5-dichloro-1,3-benzoxazole (0.009 g, 0.05 mmol). The reaction mixture was stirred at 60° C. for 16 h. The LCMS data shows product formation m/z 490.2. The reaction mixture was diluted with water and the product was extracted with ethyl acetate. The combined ethyl acetate layer was washed with water followed by brine. The organic layer was separated and dried over anhydrous sodium sulfate. Evaporation of solvent gave crude product which was purified by prep-TLC plate, Mobile phase: EtOAc:Hexane, (80:20 v/v mL). Example 9 was obtained (0.009 g, Yield 35.7%). MS (ESI) mass calculated for C₂₇H₂₈ClN₅O₂, 490.0; m/z found 490.2 [M+H]⁺

### I. Example 10

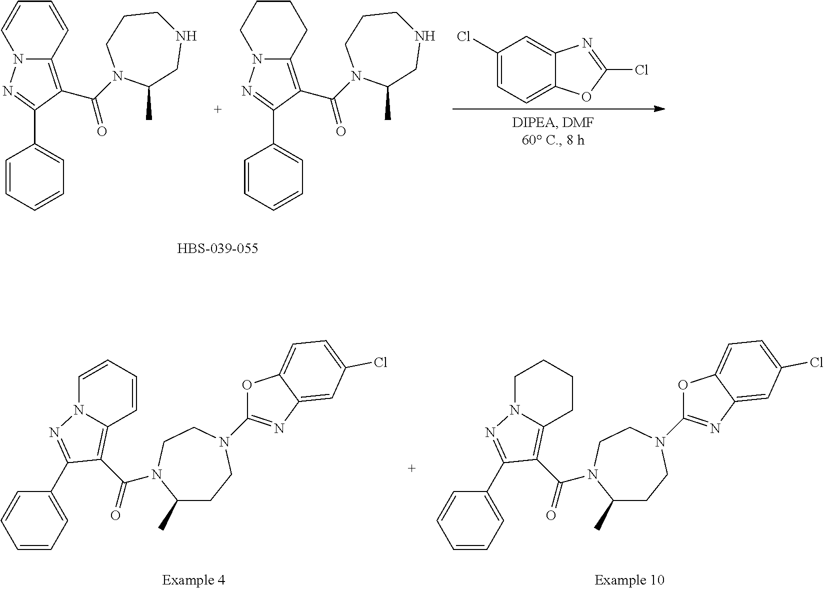 [SMILES: C[C@@H]1CNCCCN1C(=O)c1c(-c2ccccc2)nn2ccccc12 ; C[C@@H]1CNCCCN1C(=O)c1c(-c2ccccc2)nn2c1CCCC2 ; Clc1ccc2oc(Cl)nc2c1 ; C[C@@H]1CCN(c2nc3cc(Cl)ccc3o2)CCN1C(=O)c1c(-c2ccccc2)nn2ccccc12 ; C[C@@H]1CCN(c2nc3cc(Cl)ccc3o2)CCN1C(=O)c1c(-c2ccccc2)nn2c1CCCC2]

Synthesis of Compound Example 10: Intermediate HBS-039-055 (0.07 g, 0.22 mmol) was dissolved in anhydrous DMF (3.0 mL). DIPEA (0.11 mL, 0.65 mmol) was added followed by 2,5-dichloro-1,3-benzoxazole (0.049 g, 0.26 mmol). The reaction mixture was stirred at 60° C. for 8 h. The LCMS data shows two products forming: m/z 486.2 (Major) and m/z 490.2 (Minor). The reaction mixture was diluted with water and the product was extracted with ethyl acetate. The combined ethyl acetate layer was washed with water followed by brine. The organic layer was separated and dried over anhydrous sodium sulfate. Evaporation of solvent gave crude product mixture which was purified by ISCO combi-flash chromatography system, Mobile phase: EtOAc:Hexane gradient. The 0.054 g of Example 4 product was obtained (Yield 51.6%) and 0.015 g of Example 10 product was obtained (Yield 14.1%). MS (ESI) mass calculated for C₂₇H₂₄ClN₅O₂, 485.96; m/z found 486.2 [M+H]⁺ and MS (ESI) mass calculated for C₂₇H₂₈ClN₅O₂, 490.0; m/z found 490.2 [M+H]⁺

### J. Example 11

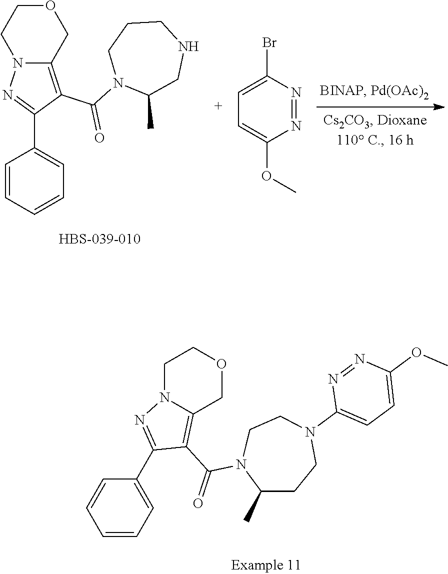

Synthesis of Compound Example 11: HBS-039-010 (0.02 g, 0.06 mmol), 3-bromo-6-methoxy-pyridazine (0.024 g, 0.13 mmol) and Cs₂CO₃ (0.058 g, 0.18 mmol) were suspended in 1,4-dioxane (2.0 mL). Pd(OAc)₂ (0.003 g, 0.012 mmol) and BINAP (0.015 g, 0.024 mmol) were added. The reaction mixture was stirred at 110° C. for 16 h. The LCMS data shows product formation m/z 449.3. The reaction mixture was filtered over celite and washed with ethyl acetate. The filtrate was concentrated to obtain the crude product which was purified by prep-TLC plate, Mobile phase: EtOAc:Hexane, (70:30 v/v mL). Example 11 was obtained (0.004 g, Yield 18.2%). MS (ESI) mass calculated for C₂₄H₂₈N₆O₃, 448.5; m/z found 449.3 [M+H]⁺

### K. Example 12

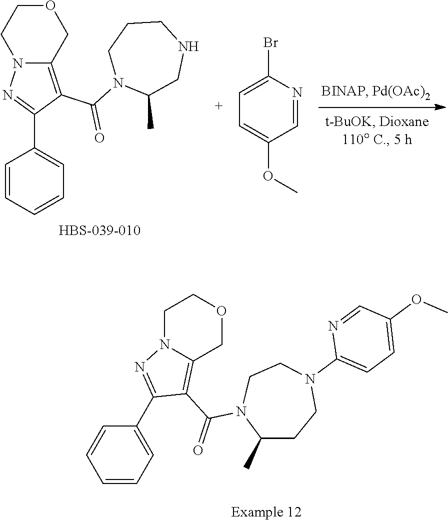

Synthesis of Compound Example 12: HBS-039-010 (0.02 g, 0.06 mmol), 2-bromo-5-methoxy-pyridine (0.022 g, 0.12 mmol) and t-BuOK (0.02 g, 0.18 mmol) were suspended in 1,4-dioxane (2.0 mL). Pd(OAc)₂ (0.003 g, 0.012 mmol) and BINAP (0.015 g, 0.024 mmol) were added. The reaction mixture was stirred at 110° C. for 5 h. The LCMS data shows product formation m/z 448.3. The reaction mixture was filtered over celite and washed with ethyl acetate. The filtrate was concentrated to obtain the crude product. The crude product was purified by prep-TLC plate, Mobile phase: EtOAc:Hexane, (50:50 v/v mL). Example 12 was obtained was obtained (0.005 g Yield 19.8%). MS (ESI) mass calculated for C₂₅H₂₉N₅O₃, 447.5; m/z found 448.3 [M+H]⁺

### L. Example 13

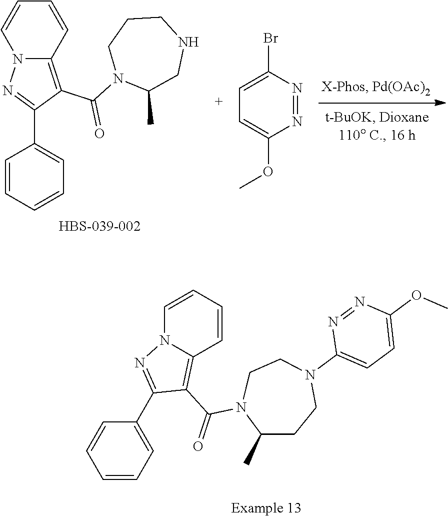

Synthesis of Compound Example 13: HBS-039-002 (0.02 g, 0.06 mmol). 3-bromo-6-methoxy-pyridazine (0.023 g, 0.12 mmol) and t-BuOK (0.017 g, 0.15 mmol) were suspended in 1,4-dioxane (2.0 mL). Pd(OAc)₂ (0.003 g, 0.012 mmol) and X-Phos (0.011 g, 0.024 mmol) were added. The reaction mixture was stirred at 110° C. for 16 h. The reaction progress was monitored by LCMS and reagents were added twice to consume the starting material. LCMS data shows product formation m/z 443.3. The reaction mixture was filtered over celite and washed with ethyl acetate. The filtrate was concentrated to obtain the crude product. The crude product was purified by prep-TLC plate, Mobile phase: EtOAc:Hexane, (35:65 v/v mL). Example 13 was obtained (0.011 g, Yield 42.6%). MS (ESI) mass calculated for C₂₅H₂₆N₆O₂, 442.5; m/z found 443.3 [M+H]⁺

### M. Example 14

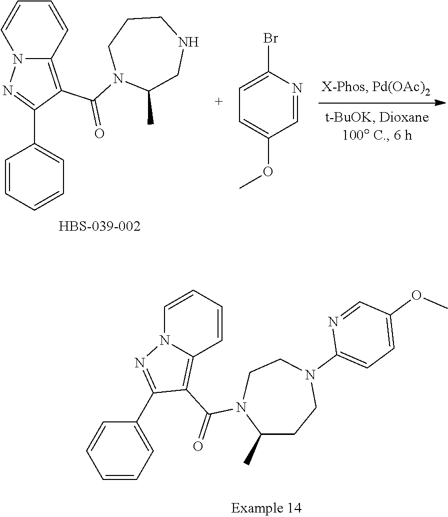

Synthesis of Compound Example 14: HBS-039-002 (0.02 g, 0.06 mmol), 2-bromo-5-methoxy-pyridine (0.022 g, 0.12 mmol) and t-BuOK (0.017 g, 0.15 mmol) were suspended in 1,4-dioxane (2.0 mL). The Pd(OAc)₂ (0.003 g, 0.012 mmol) and X-Phos (0.011 g, 0.024 mmol) were added. The reaction mixture was stirred at 110° C. for 6 h. the LCMS data shows product formation m/z 442.3. The reaction mixture was filtered over celite and washed with ethyl acetate and the filtrate was concentrated to obtain the crude product which was purified by prep-TLC plate, Mobile phase: EtOAc:Hexane, (40:60 v/v mL). Example 14 was obtained (0.009 g, Yield 33.3%). MS (ESI) mass calculated for C₂₆H₂₇N₅O₂, 441.5; m/z found 442.3 [M+H]⁺

### N. Example 15

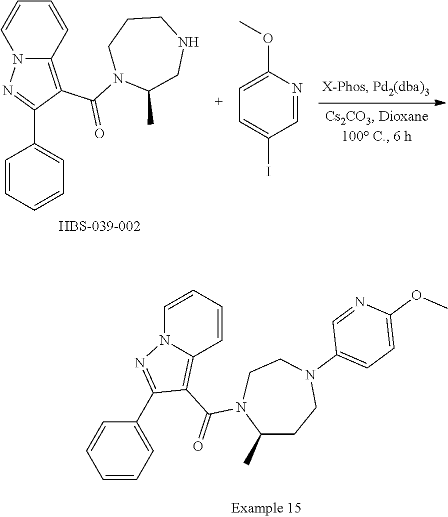

Synthesis of Compound Example 15: HBS-039-002 (0.02 g, 0.06 mmol), 5-iodo-2-methoxy-pyridine (0.028 g, 0.12 mmol) and Cs₂CO3 (0.058 g, 0.18 mmol) were suspended in 1,4-dioxane (2.0 mL). The Pd₂(dba)₃ (0.011 g, 0.012 mmol) and X-Phos (0.011 g, 0.024 mmol) were added. The reaction mixture was stirred at 110° C. for 6 h. The reaction progress was monitored by LCMS and reagents were added twice to consume the starting material. The LCMS data shows product formation m/z 442.3. The reaction mixture was filtered over celite and washed with ethyl acetate and the filtrate was concentrated to obtain the crude product which was purified by ISCO combi-flash system, Mobile phase: EtOAc:Hexane gradient. Example 15 was obtained (0.01 g, Yield 37.8%). MS (ESI) mass calculated for C₂₆H₂₇N₅O₂, 441.5; m/z found 442.3 [M+H]⁺

### O. Example 16

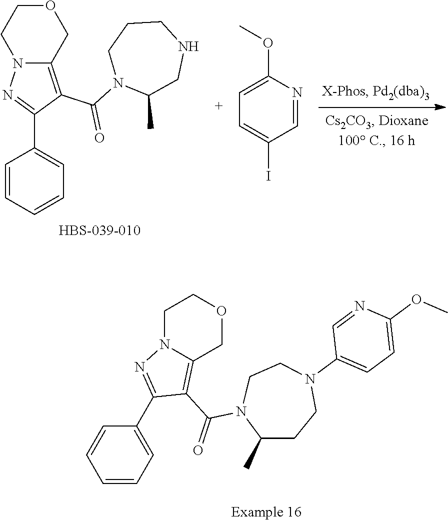

Synthesis of Compound Example 16: HBS-039-010 (0.02 g, 0.06 mmol), 5-iodo-2-methoxy-pyridine (0.04 g, 0.18 mmol) and Cs₂CO₃ (0.057 g, 0.18 mmol) were suspended in 1,4-dioxane (2.0 mL). The Pd₂(dba)₃ (0.011 g, 0.011 mmol) and X-Phos (0.011 g, 0.023 mmol) were added. The reaction mixture was stirred at 100° C. for 16 h. The reaction progress was monitored by LCMS and reagents were added twice to consume the starting material. The LCMS data shows product formation m/z 448.3. The reaction mixture was filtered over celite and washed with ethyl acetate and the filtrate was concentrated to obtain the crude product which was purified by ISCO combi-flash system, Mobile phase: EtOAc:Hexane gradient. Example 16 was obtained (0.005 g, Yield 19.3%). MS (ESI) mass calculated for C₂₅H₂₉N₅O₃, 447.5; m/z found 448.3 [M+H]⁺

### P. Example 17

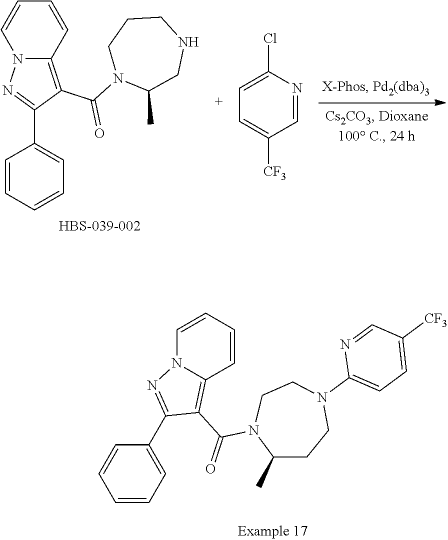 [SMILES: C[C@@H]1CNCCCN1C(=O)c1c(-c2ccccc2)nn2ccccc12 ; FC(F)(F)c1ccc(Cl)nc1 ; C[C@@H]1CCN(c2ccc(C(F)(F)F)cn2)CCN1C(=O)c1c(-c2ccccc2)nn2ccccc12]

Synthesis of Compound Example 17: HBS-039-002 (0.02 g, 0.06 mmol), 2-chloro-5-trifluoromethyl-pyridine (0.022 g, 0.12 mmol) and Cs₂CO₃ (0.058 g, 0.18 mmol) were suspended in 1,4-dioxane (2.0 mL). The Pd₂(dba)₃ (0.006 g, 0.012 mmol) and X-Phos (0.006 g, 0.012 mmol) were added. The reaction mixture was stirred at 100° C. for 24 h. The reaction progress was monitored by LCMS and reagents were added twice to consume the starting material. The LCMS data shows product formation m/z 480.2. The reaction mixture was filtered over celite and washed with ethyl acetate. The filtrate was concentrated to obtain the crude product. The crude product was purified by ISCO combi-flash system, Mobile phase: EtOAc:Hexane gradient Example 17 was obtained (0.01, Yield 36.5%). MS (ESI) mass calculated for C₂H₂₄F₃N₅O, 479.5; m/z found 480.2 [M+H]⁺

### Q. Example 18

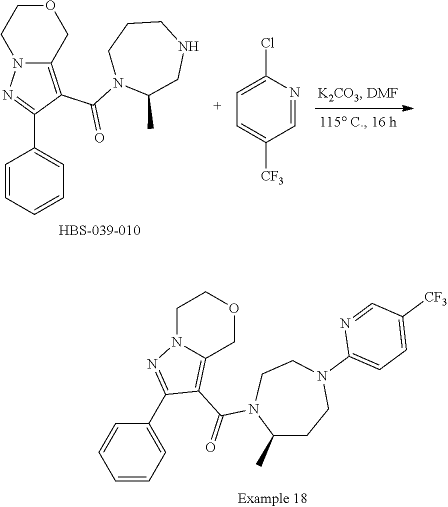 [SMILES: C[C@@H]1CNCCCN1C(=O)c1c(-c2ccccc2)nn2c1COCC2 ; FC(F)(F)c1ccc(Cl)nc1 ; C[C@@H]1CCN(c2ccc(C(F)(F)F)cn2)CCN1C(=O)c1c(-c2ccccc2)nn2c1COCC2]

Synthesis of Compound Example 18: HBS-039-010 (0.017 g, 0.05 mmol) and 2-chloro-5-trifluoromethyl-pyridine (0.018 g, 0.1 mmol) were dissolved in anhydrous DMF (2.0 mL). Anhydrous K₂CO₃ (0.021 g, 0.15 mmol) was added, and the reaction mixture was stirred at 115° C. for 16 h. The LCMS data shows product formation m/z 486.3. The reaction mixture was concentrated under air stream to obtain the crude product which was purified by ISCO combi-flash system, Mobile phase. EtOAc:Hexane gradient Example 18 was obtained (0.014 g, Yield 58.5%). MS (ESI) mass calculated for C₂₅H₂₆F₃N₅O₂, 485.5; m/z found 486.3 [M+H]⁺

### R. Example 19

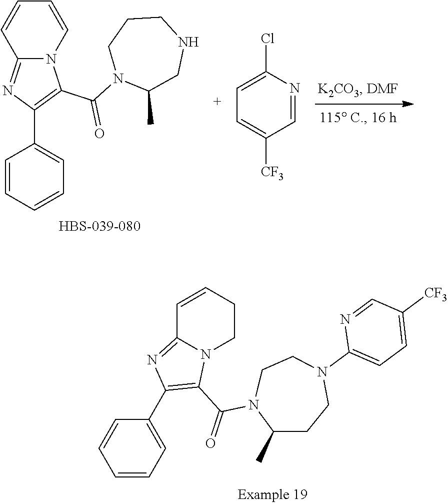 [SMILES: C[C@@H]1CNCCCN1C(=O)c1c(-c2ccccc2)nc2ccccn12 ; FC(F)(F)c1ccc(Cl)nc1 ; C[C@@H]1CCN(c2ccc(C(F)(F)F)cn2)CCN1C(=O)c1c(-c2ccccc2)nc2n1CCC=C2]

Synthesis of Compound Example 19: HBS-039-080 (0.02 g, 0.06 mmol) and 2-chloro-5-trifluoromethyl-pyridine (0.022 g, 0.12 mmol) were dissolved in anhydrous DMF (2.0 mL). Anhydrous K₂CO₃ (0.025 g, 0.18 mmol) was added and the reaction mixture was stirred at 115° C. for 16 h. The LCMS data shows product formation m/z 480.2. The reaction mixture was concentrated under air stream to obtain the crude product which was purified by ISCO combi-flash system, Mobile phase: EtOAc:Hexane gradient Example 19 was obtained (0.01 g, Yield 35.5%). MS (ESI) mass calculated for C₂₅H₂₄F₃N₅O, 479.5; m/z found 480.2 [M+H]⁺

### S. Example 20

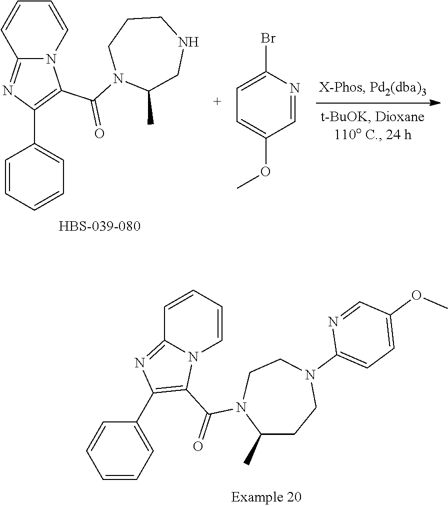

Synthesis of Compound Example 20: HBS-039-080 (0.02 g, 0.06 mmol), 2-bromo-5-methoxy-pyridine (0.034 g, 0.18 mmol) and t-BuOK (0.02 g, 0.18 mmol) were suspended in 1,4-dioxane (2.0 mL). The Pd₂(dba)₃ (0.011 g, 0.012 mmol) and X-Phos (0.006 g, 0.01 mmol) were added and the reaction mixture was stirred at 110° C. for 24 h. The reaction progress was monitored by LCMS and reagents were added twice to consume the starting material. The LCMS data shows product formation m/z 442.3. The reaction mixture was filtered over celite and washed with ethyl acetate. The filtrate was concentrated to obtain the crude product which was purified by ISCO combi-flash system, Mobile phase: EtOAc:Hexane gradient Example 20 was obtained (0.007 g, Yield 26.8%). MS (ESI) mass calculated for C₂₆H₂₇N₅O₂, 441.5; m/z found 442.3 [M+H]⁺.

### T. Example 21

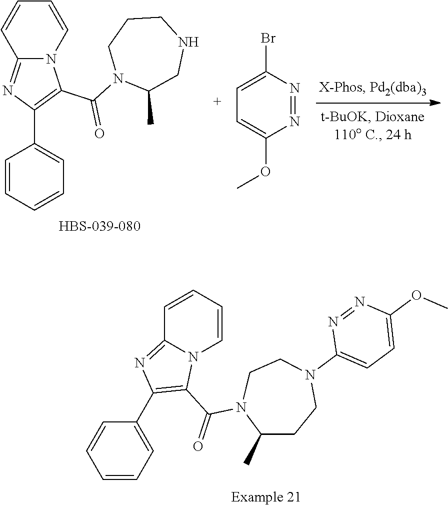

Synthesis of Compound Example 21: HBS-039-080 (0.02 g, 0.06 mmol), 2-bromo-5-methoxy-pyridazine (0.034 g, 0.18 mmol) and t-BuOK (0.02 g, 0.18 mmol) were suspended in 1,4-dioxane (2.0 mL). The Pd₂(dba)₃ (0.011 g, 0.012 mmol) and X-Phos (0.006 g, 0.012 mmol) were added and the reaction mixture was stirred at 110° C. for 24 h. The reaction progress was monitored by LCMS and reagents were added twice to consume the starting material. The LCMS data shows product formation m/z 443.3. The reaction mixture was filtered over celite and washed with ethyl acetate. The filtrate was concentrated to obtain the crude product which was purified by ISCO combi-flash system, Mobile phase: EtOAc:Hexane gradient Example 21 was obtained (0.015 g, Yield 56.2%). MS (ESI) mass calculated for C₂₅H₂₆N₆O₂, 442.5; m/z found 443.3 [M+H]⁺

### U. Example 22

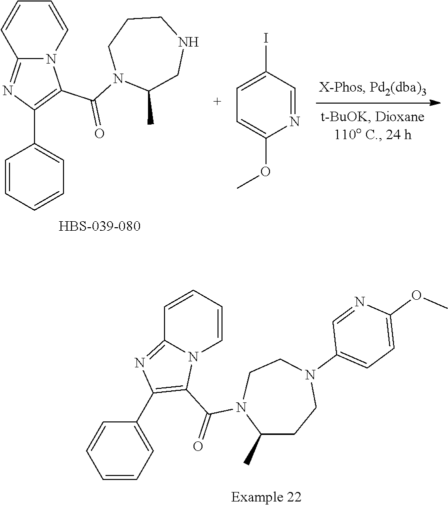

Synthesis of Compound Example 22: HBS-039-080 (0.02 g, 0.06 mmol), 5-lodo-2-methoxy-pyridine (0.04 g, 0.18 mmol) and t-BuOK (0.02 g, 0.18 mmol) were suspended in 1,4-dioxane (2.0 mL). The Pd₂(dba)₃ (0.011 g, 0.012 mmol) and X-Phos (0.006 g, 0.012 mmol) were added and the reaction mixture was stirred at 110° C. for 24 h. The reaction progress was monitored by LCMS and reagents were added twice to consume the starting material. The LCMS data shows product formation m/z 442.3. The reaction mixture was filtered over celite and washed with ethyl acetate. The filtrate was concentrated to obtain the crude product which was purified by ISCO combi-flash system, Mobile phase: EtOAc:Hexane gradient. Example 22 was obtained (0.02 g, Yield 74.6%). MS (ESI) mass calculated for C₂₆H₂₇N₅O₂, 441.5: m/z found 442.3 [M+H]⁺

### V. Example 23

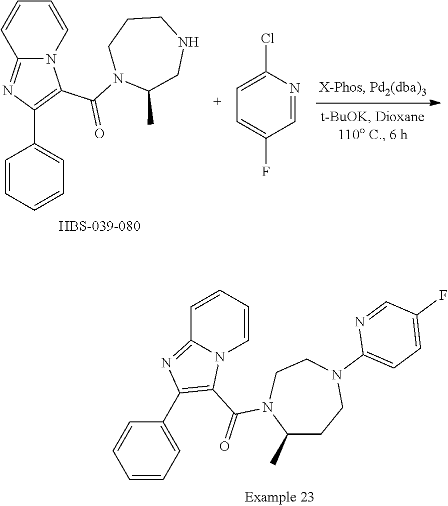 [SMILES: C[C@@H]1CNCCCN1C(=O)c1c(-c2ccccc2)nc2ccccn12 ; Fc1ccc(Cl)nc1 ; C[C@@H]1CCN(c2ccc(F)cn2)CCN1C(=O)c1c(-c2ccccc2)nc2ccccn12]

Synthesis of Compound Example 23: HBS-039-080 (0.025 g, 0.075 mmol), 2-chloro-5-fluoro-pyridine (0.02 g, 0.15 mmol) and t-BuOK (0.025 g, 0.22 mmol) were suspended in 1,4-dioxane (2.5 mL). The Pd₂(dba)₃ (0.014 g, 0.015 mmol) and X-Phos (0.007 g, 0.015 mmol) were added and the reaction mixture was stirred at 110° C. for 6 h. The LCMS data shows product formation m/z 430.3. The reaction mixture was filtered over celite and washed with ethyl acetate. The filtrate was concentrated to obtain the crude product which was purified by ISCO combi-flash system, Mobile phase: EtOAc:Hexane gradient Example 23 was obtained (0.017 g, Yield 51.9%). MS (ESI) mass calculated for C₂₅H₄FN₅O, 429.5; m/z found 430.3 [M+H]⁺

### W. Example 24

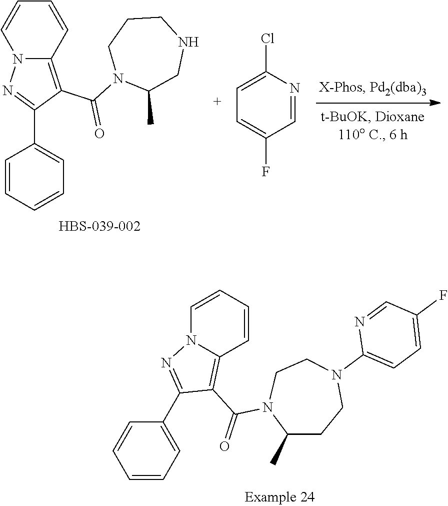 [SMILES: C[C@@H]1CNCCCN1C(=O)c1c(-c2ccccc2)nn2ccccc12 ; Fc1ccc(Cl)nc1 ; C[C@@H]1CCN(c2ccc(F)cn2)CCN1C(=O)c1c(-c2ccccc2)nn2ccccc12]

Synthesis of Compound Example 24: HBS-039-002 (0.025 g, 0.075 mmol), 2-chloro-5-fluoro-pyridine (0.02 g, 0.15 mmol) and t-BuOK (0.025 g, 0.22 mmol) were suspended in 1,4-dioxane (3.0 mL). The Pd₂(dba)₃ (0.014 g, 0.015 mmol) and X-Phos (0.007 g, 0.015 mmol) were added. The reaction mixture was stirred at 110° C. for 6 h. The LCMS data shows product formation m/z 430.3. The reaction mixture was filtered over celite and washed with ethyl acetate. The filtrate was concentrated to obtain the crude product which was purified by ISCO combi-flash system, Mobile phase: EtOAc:Hexane gradient Example 24 was obtained (0.019 g, Yield 59.8%). MS (ESI) mass calculated for C₂₅H₂₄FN₅O, 429.5; m/z found 430.3 [M+H]r

### X. Example 25

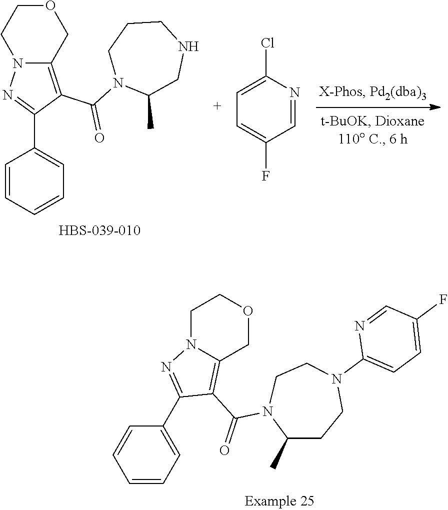 [SMILES: C[C@@H]1CNCCCN1C(=O)c1c(-c2ccccc2)nn2c1COCC2 ; Fc1ccc(Cl)nc1 ; C[C@@H]1CCN(c2ccc(F)cn2)CCN1C(=O)c1c(-c2ccccc2)nn2c1COCC2]

Synthesis of Compound Example 25: HBS-039-010 (0.025 g, 0.073 mmol), 2-bromo-5-fluoro-pyridine (0.026 g, 0.15 mmol) and t-BuOK (0.021 g, 0.18 mmol) were suspended in 1,4-dioxane (2.5 mL). The Pd₂(dba)₃ (0.007 g, 0.007 mmol) and X-Phos (0.004 g, 0.007 mmol) were added and the reaction mixture was stirred at 110° C. for 6 h. The LCMS data shows product formation m/z 436.3. The reaction mixture was filtered over celite and washed with ethyl acetate. The filtrate was concentrated to obtain the crude product which was purified by ISCO combi-flash system, Mobile phase: EtOAc:Hexane gradient Example 25 was obtained (0.026 g, Yield 83.5%). MS (ESI) mass calculated for C₂₄H₂FN₆O₂, 435.5; m/z found 436.3 [M+H]⁺.

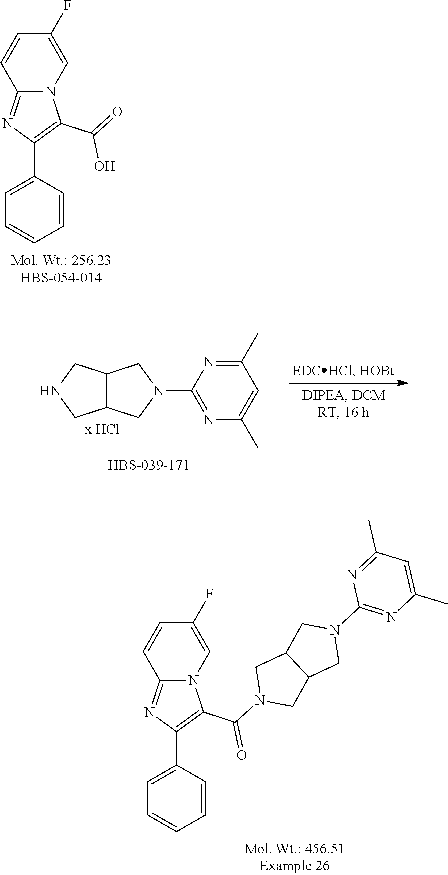 [SMILES: O=C(O)c1c(-c2ccccc2)nc2ccc(F)cn12 ; Cc1cc(C)nc(N2CC3CNCC3C2)n1 ; Cc1cc(C)nc(N2CC3CN(C(=O)c4c(-c5ccccc5)nc5ccc(F)cn45)CC3C2)n1]

Synthesis of Example 26: Intermediate HBS-054-014 (0.025 g, 0.1 mmol) was dissolved in anhydrous DCM (2.0 mL). The EDC·HCl (0.028 g, 0.15 mmol) and HOBt (0.02 g, 0.15 mmol) were added followed by DIPEA (0.07 mL, 0.4 mmol). The r×n mixture was stirred at ambient temperature for 5.0 min.

Intermediate HBS-039-171 (0.031 g, 0.11 mmol) was added to the r×n mixture. The r×n mixture was stirred at ambient temperature for 16 h. The LCMS data shows product formation m/z 457.0. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: EtOAc:Hexane gradient. The 0.045 g of product was obtained (Yield Quant.). MS (ESI) mass calcd. for C₂₈H₂₅FN₈O, 456.5; m/z found 457.0 [M+H]+.

Note: Similar coupling procedure was followed to prepare the compound Example 27 to Example 34 using corresponding Acid intermediates and Amine Intermediate HBO-039-171.

| Acid Intermediate | Amine Intermediate | Final Product |
|---|---|---|
| HBS-054-015 | HBS-039-171 | Example 27 |
| HBS-054-016 | HBS-039-171 | Example 28 |
| HBS-054-021 | HBS-039-171 | Example 29 |
| HBS-039-150 | HBS-039-171 | Example 30 |
| HBS-054-039 | HBS-039-171 | Example 31 |
| HBS-054-040 | HBS-039-171 | Example 32 |
| HBS-054-041 | HBS-039-171 | Example 33 |
| HBS-054-042 | HBS-039-171 | Example 34 |

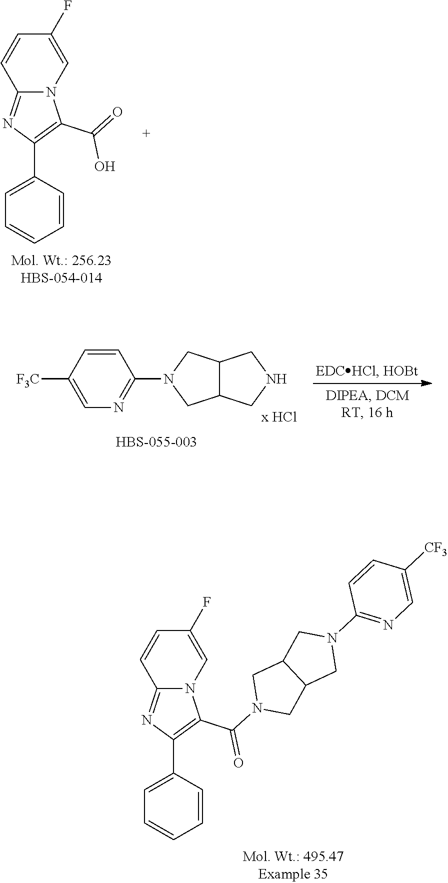 [SMILES: O=C(O)c1c(-c2ccccc2)nc2ccc(F)cn12 ; FC(F)(F)c1ccc(N2CC3CNCC3C2)nc1 ; O=C(c1c(-c2ccccc2)nc2ccc(F)cn12)N1CC2CN(c3ccc(C(F)(F)F)cn3)CC2C1]

Synthesis of Example 35: Intermediate HBS-054-014 (0.015 g, 0.059 mmol) was dissolved in anhydrous DCM (2.0 mL). The EDC·HCl (0.017 g, 0.088 mmol) and HOBt (0.012 g, 0.088 mmol) were added followed by DIPEA (0.04 mL, 0.18 mmol). The r×n mixture was stirred at ambient temperature for 5.0 min. Intermediate HBS-055-003 (0.019 g, 0.064 mmol) was added to the r×n mixture. The r×n mixture was stirred at ambient temperature for 16 h. The LCMS data shows product formation m/z 457.0. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: EtOAc:Hexane gradient. The 0.023 g of product was obtained (Yield 79.0%). MS (ESI) mass calcd. for C₂₆H₂₁F₄N₅O, 495.47; m/z found 496.0 [M+H]+.

Note: Similar coupling procedure was followed to prepare compound Example 36 using corresponding Acid intermediates and Amine Intermediate.

| Acid Intermediate | Amine Intermediate | Final Product |
|---|---|---|
| HBS-054-021 | HBS-055-003 | Example 36 |

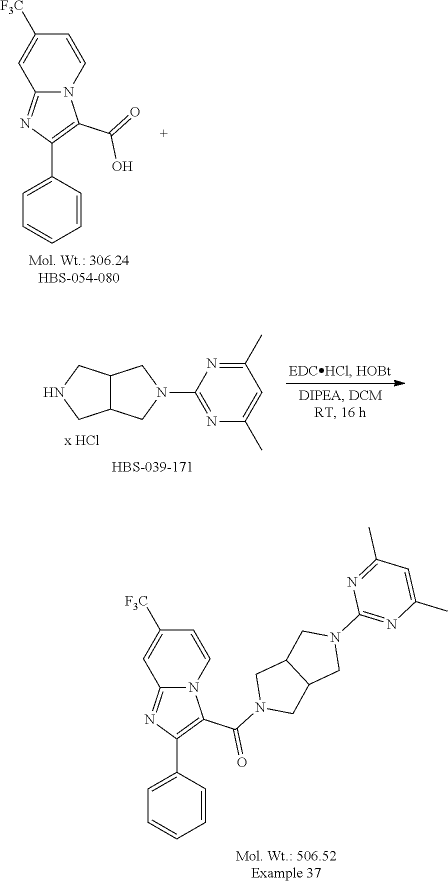 [SMILES: O=C(O)c1c(-c2ccccc2)nc2cc(C(F)(F)F)ccn12 ; Cc1cc(C)nc(N2CC3CNCC3C2)n1 ; Cc1cc(C)nc(N2CC3CN(C(=O)c4c(-c5ccccc5)nc5cc(C(F)(F)F)ccn45)CC3C2)n1]

Synthesis of Example 37: Intermediate HBS-054-080 (0.02 g, 0.07 mmol) was dissolved in anhydrous DCM (2.0 mL). The EDC·HCl (0.019 g, 0.1 mmol) and HOBt (0.013 g, 0.1 mmol) were added followed by DIPEA (0.034 mL, 0.2 mmol). The r×n mixture was stirred at ambient temperature for 5.0 min. Intermediate HBS-039-171 (0.021 g, 0.072 mmol) was added to the r×n mixture. The r×n mixture was stirred at ambient temperature for 16 h. The LCMS data shows product formation m/z 507.0. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: EtOAc:Hexane gradient. The 0.02 g of product was obtained (Yield 61.0%). MS (ESI) mass calcd. for C₂₇H₂₅F₃N₆O, 506.52; m/z found 507.03 [M+H]+.

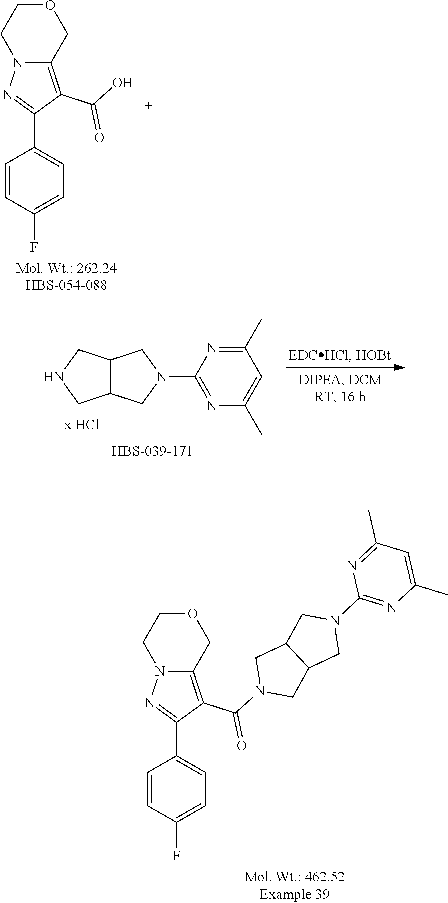 [SMILES: O=C(O)c1c(-c2ccc(F)cc2)nn2c1COCC2 ; Cc1cc(C)nc(N2CC3CNCC3C2)n1 ; Cc1cc(C)nc(N2CC3CN(C(=O)c4c(-c5ccc(F)cc5)nn5c4COCC5)CC3C2)n1]

Synthesis of Example 39: Intermediate HBS-054-088 (0.025 g, 0.095 mmol) was dissolved in anhydrous DCM (2.0 mL). The EDC·HCl (0.027 g, 0.14 mmol) and HOBt (0.019 g, 0.14 mmol) were added followed by DIPEA (0.05 mL, 0.29 mmol). The r×n mixture was stirred at ambient temperature for 5.0 min. Intermediate HBS-039-171 (0.031 g, 0.11 mmol) was added to the r×n mixture. The r×n mixture was stirred at ambient temperature for 16 h. The LCMS data shows product formation m/z 463.0. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: EtOAc:Hexane gradient. The 0.027 g of product was obtained (Yield 61.0%). MS (ESI) mass calcd. for C₂₅H₂₇FN₆O₂, 462.52; m/z found 463.0 [M+H]+.

Note: Similar coupling procedure was followed to prepare compound Example 41, Example 42 and Example 43 using corresponding Acid intermediates and Amine Intermediate.

| Acid Intermediate | Amine Intermediate | Final Product |
|---|---|---|
| 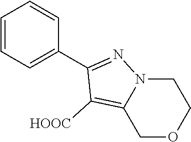 [SMILES: O=C(O)c1c(-c2ccccc2)nn2c1COCC2] | HBS-039-171 | Example 41 |
| 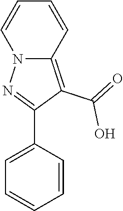 [SMILES: O=C(O)c1c(-c2ccccc2)nn2ccccc12] | HBS-039-171 | Example 42 |
| 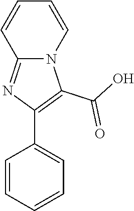 [SMILES: O=C(O)c1c(-c2ccccc2)nc2ccccn12] | HBS-039-171 | Example 43 |

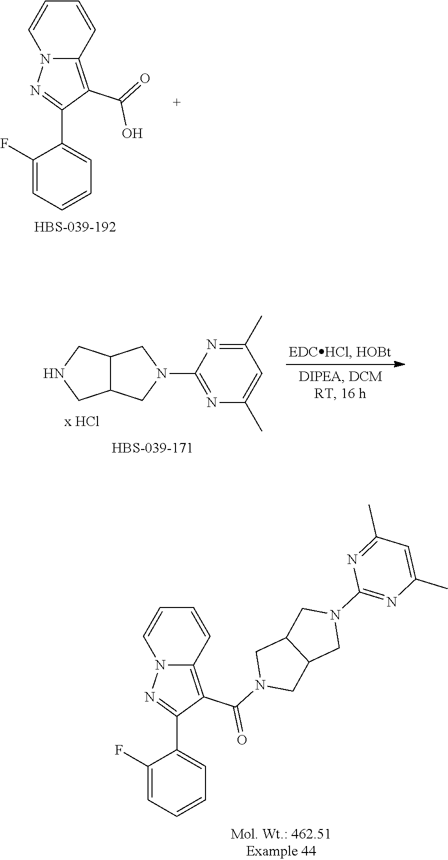 [SMILES: O=C(O)c1c(-c2ccccc2F)nn2ccccc12 ; Cc1cc(C)nc(N2CC3CNCC3C2)n1 ; Cc1cc(C)nc(N2CC3CN(C(=O)c4c(-c5ccccc5F)nn5ccccc45)CC3C2)n1]

Synthesis of Example 44: Intermediate HBS-039-192 (0.025 g, 0.098 mmol) was dissolved in anhydrous DCM (2.0 mL). The EDC·HCl (0.025 g, 0.15 mmol) and HOBt (0.02 g, 0.15 mmol) were added followed by DIPEA (0.17 mL, 0.98 mmol). The r×n mixture was stirred at ambient temperature for 5.0 min. Intermediate HBS-039-171 (0.029 g, 0.098 mmol) was added to the r×n mixture. The r×n mixture was stirred at ambient temperature for 16 h. The LCMS data shows product formation m/z 457.3. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.045 g of product was obtained (Yield Quant.) MS (ESI) mass calcd. for C₂₅H₂₅FN₆O, 456.51; m/z found 457.3 [M+H]+.

Note: Similar coupling procedure was followed to prepare compound Example 45, Example 44 using corresponding Acid intermediates and Amine Intermediate.

| Acid Intermediate | Amine Intermediate | Final Product |
|---|---|---|
| HBS-039-192 | HBS-055-003 | Example 44 |
| HBS-039-150 | HBS-055-003 | Example 45 |

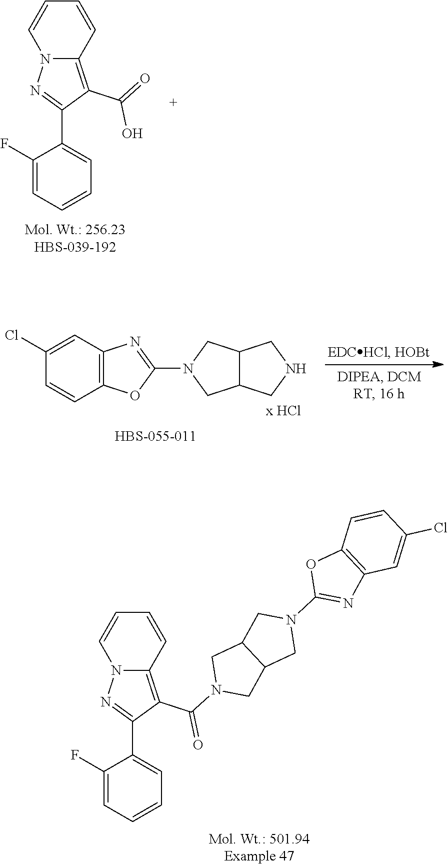 [SMILES: O=C(O)c1c(-c2ccccc2F)nn2ccccc12 ; Clc1ccc2oc(N3CC4CNCC4C3)nc2c1 ; O=C(c1c(-c2ccccc2F)nn2ccccc12)N1CC2CN(c3nc4cc(Cl)ccc4o3)CC2C1]

Synthesis of Example 47: Intermediate HBS-039-192 (0.02 g, 0.078 mmol) was dissolved in anhydrous DCM (2.0 mL). The EDC·HCl (0.022 g, 0.12 mmol) and HOBt (0.016 g, 0.12 mmol) were added followed by DIPEA (0.14 mL, 0.78 mmol). The r×n mixture was stirred at ambient temperature for 5.0 min. Intermediate HBS-055-011 (0.026 g, 0.078 mmol) was added to the r×n mixture. The r×n mixture was stirred at ambient temperature for 16 h. The LCMS data shows product formation m/z 502.2. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: EtOAc:Hexane gradient. The 0.039 g of product was obtained (Yield Quant.). MS (ESI) mass calcd. for C₂₇H₂₁ClFN₅O₂, 501.94; m/z found 502.2 [M+H]+.

Note: Similar coupling procedure was followed to prepare compound HBO-12240 using corresponding Acid and Amine Intermediate.

| Acid Intermediate | Amine Intermediate | Final Product |
|---|---|---|
| HBS-039-150 | HBS-055-011 | Example 48 |

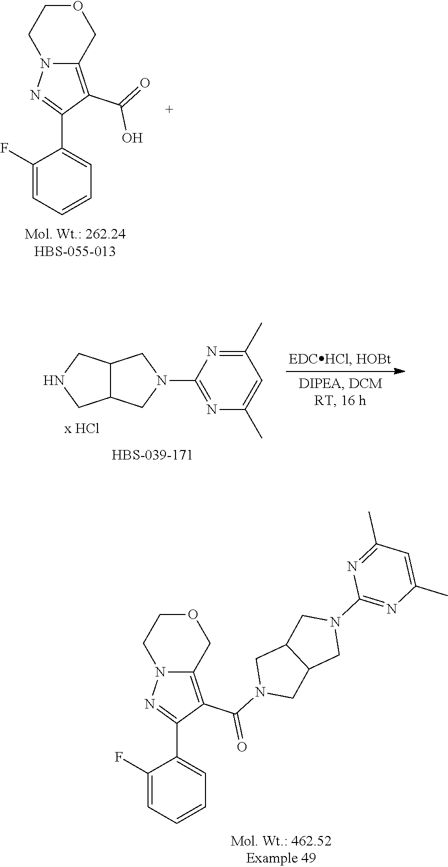 [SMILES: O=C(O)c1c(-c2ccccc2F)nn2c1COCC2 ; Cc1cc(C)nc(N2CC3CNCC3C2)n1 ; Cc1cc(C)nc(N2CC3CN(C(=O)c4c(-c5ccccc5F)nn5c4COCC5)CC3C2)n1]

Synthesis of Example 49: Intermediate HBS-055-013 (0.02 g, 0.076 mmol) was dissolved in anhydrous DCM (2.0 mL). The EDC·HCl (0.022 g, 0.11 mmol) and HOBt (0.015 g, 0.11 mmol) were added followed by DIPEA (0.13 mL, 0.76 mmol). The r×n mixture was stirred at ambient temperature for 5.0 min. Intermediate HBS-039-171 (0.022 g, 0.076 mmol) was added to the r×n mixture. The r×n mixture was stirred at ambient temperature for 16 h. The LCMS data shows product formation m/z 463.3. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.0168 g of product was obtained (Yield 47.6%) MS (ESI) mass calcd. for C₂₅H₂₇FN₆O₂, 462.52; m/z found 463.3 [M+H]+.

Note: Similar coupling procedure was followed to prepare compound Example 50 and Example 51 using corresponding Acid intermediates and Amine Intermediate.

| Acid Intermediate | Amine Intermediate | Final Product |
|---|---|---|
| HBS-055-013 | HBS-055-003 | Example 50 |
| HBS-055-013 | HBS-055-011 | Example 51 |

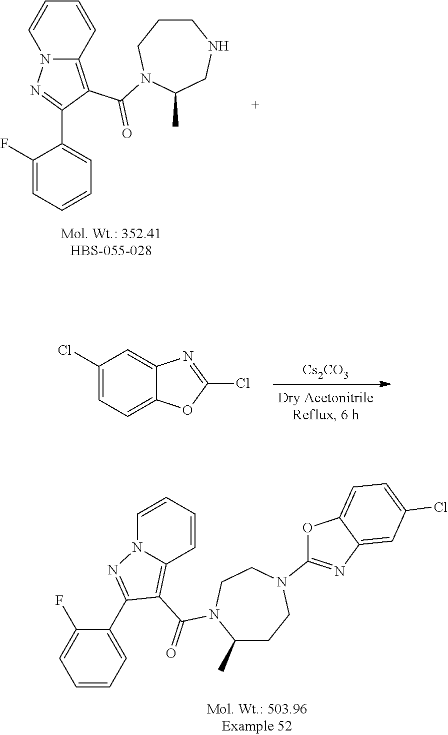

Synthesis of Example 52: Intermediate HBS-055-028 (0.02 g, 0.057 mmol) was dissolved in anhydrous Acetonitrile (2.0 mL). The anhydrous Cs₂CO₃ (0.037 g, 0.11 mmol) was added followed by 2,5-dichloro-1,3-benzooxazole (0.016 g, 0.085 mmol). The reaction mixture was heated at reflux for 6 h. The LCMS data shows product formation m/z 504.2. The reaction mixture was filtered over celite and washed with Ethyl acetate. The filtrate was concentrated under reduced pressure to obtain crude product. The crude product was purified by ISCO combi-flash chromatography system, Mobile phase: EtOAc:Hexane gradient. The 22.2 g of product was obtained (Yield 77.2%). MS (ESI) mass calcd. for C₇H₂₃ClFN₅O₂, 503.96; m/z found 504.2 [M+H]+.

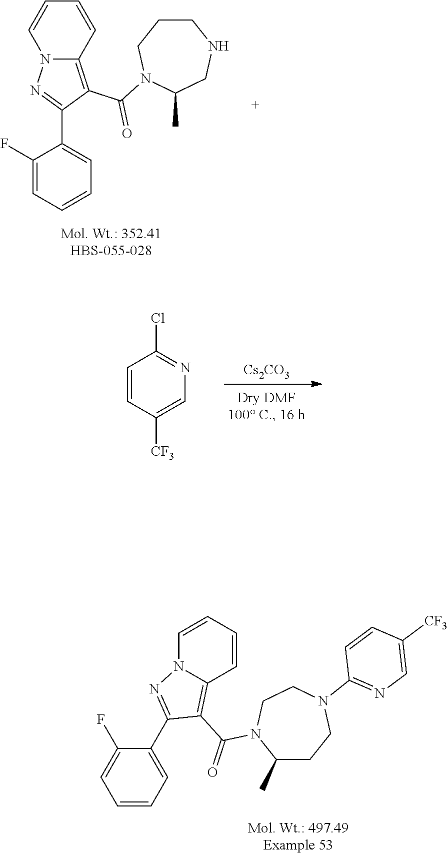

Synthesis of Example 53: Intermediate HBS-055-028 (0.02 g, 0.057 mmol) was dissolved in anhydrous DMF (2.0 mL). The anhydrous Cs₂CO₃ (0.037 g, 0.11 mmol) was added followed by 2-Chloro-5-trifluoromethylpyridine (0.016 g, 0.089 mmol). The reaction mixture was heated at 100° C. for 16 h. The LCMS data shows product formation m/z 498.2. The reaction mixture was diluted with water and the product was extracted with DCM. The combined DCM layer was separated and dried over anhydrous sodium sulfate. The evaporation of solvent gave crude product. The crude product was purified by ISCO combi-flash chromatography system, Mobile phase: EtOAc:Hexane gradient to obtain 17.2 mg of pure product (Yield 60.5%). MS (ESI) mass calcd. for C₂₆H₂₃F₄N₅O₂, 497.49; m/z found 498.2 [M+H]+.

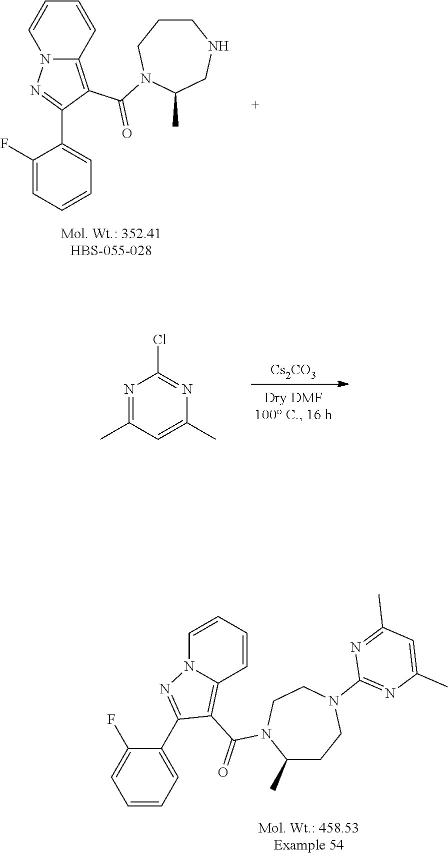

Synthesis of Example 54: Intermediate HBS-055-028 (0.02 g, 0.057 mmol) was dissolved in anhydrous DMF (2.0 mL). The anhydrous Cs₂CO₃ (0.037 g, 0.11 mmol) was added followed by 2-Chloro-4,6-dimethyl pyrimidine (0.018 g, 0.086 mmol). The reaction mixture was heated at 100° C. for 16 h. The LCMS data shows product formation r/z 459.3. The reaction mixture was diluted with water and the product was extracted with DCM. The combined DCM layer was separated and dried over anhydrous sodium sulfate. The evaporation of solvent gave crude product. The crude product was purified by ISCO combi-flash chromatography system, Mobile phase: EtOAc:Hexane gradient to obtain 21.1 mg of pure product (Yield 80.7%). MS (ESI) mass calcd. for C₂₆H₂₇FN₆O, 458.53; m/z found 459.3 [M+H]+.

Note: General EDC·HCl, HOBt·H₂O coupling procedure was followed to prepare Example 54 and Example 55 using corresponding Acid intermediates and Amine Intermediate.

| Acid Intermediate | Amine Intermediate | Final Product |
|---|---|---|
| HBS-039-023 | HBS-055-117 | Example 55 |
| HBS-039-023 | HBS-062-031 | Example 56 |

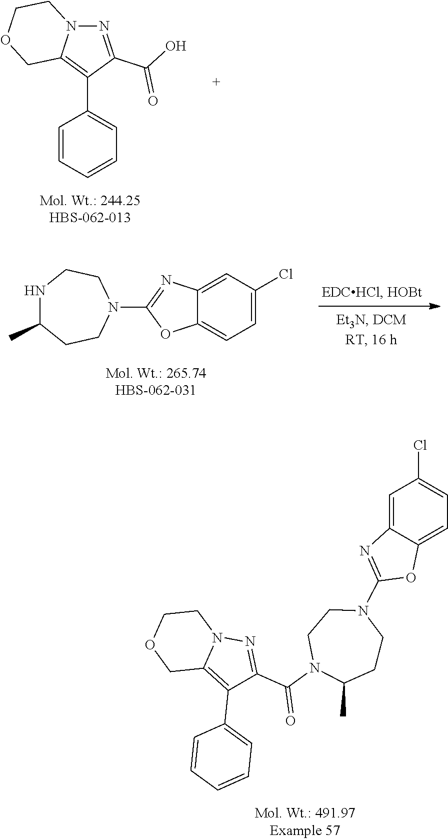

Synthesis of Example 57: Intermediate HBS-062-013 (0.02 g, 0.082 mmol) was dissolved in anhydrous DCM (2.0 mL). The EDC·HCl (0.024 g, 0.13 mmol) and HOBt (0.017 g, 0.13 mmol) were added followed by Et₃N (0.06 mL, 0.42 mmol). The r×n mixture was stirred at ambient temperature for 5.0 min. Intermediate HBS-062-031 (0.028 g, 0.082 mmol) was added to the r×n mixture. The r×n mixture was stirred at ambient temperature for 16 h. The LCMS data shows product formation m/z 492.1. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.023 g of product was obtained (Yield 56.1%) MS (ESI) mass calcd. for C₂H₂₆ClN₅O₃, 491.97; m/z found 492.1 [M+H]+.

Note: Similar coupling procedure was followed to prepare compound Example 58.

| Acid Intermediate | Amine Intermediate | Final Product |
|---|---|---|
| HBS-062-013 | HBS-055-117 | Example 58 |

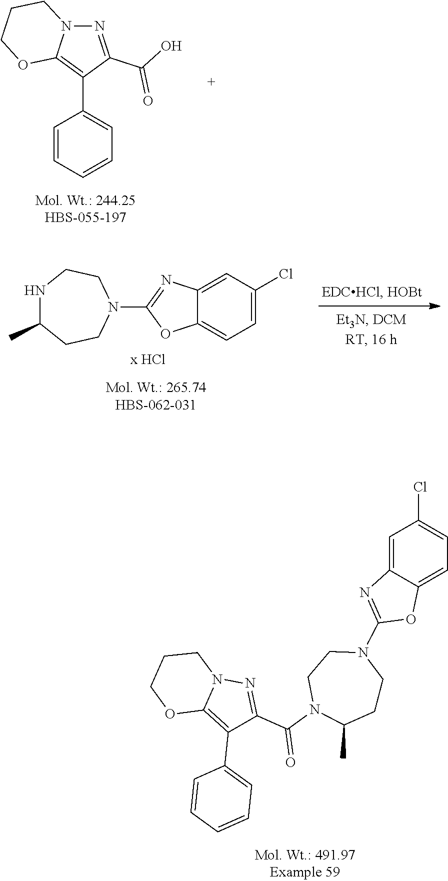

Synthesis of Example 59: Intermediate HBS-055-197 (0.02 g, 0.082 mmol) was dissolved in anhydrous DCM (2.0 mL). The EDC·HCl (0.024 g, 0.12 mmol) and HOBt (0.017 g, 0.13 mmol) were added followed by Et₃N (0.06 mL, 0.41 mmol). The r×n mixture was stirred at ambient temperature for 5.0 min.

Intermediate HBS-062-031 (0.028 g, 0.082 mmol) was added to the r×n mixture. The r×n mixture was stirred at ambient temperature for 16 h. The LCMS data shows product formation m/z 492.1. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.037 g of product was obtained (Yield 91.9%) MS (ESI) mass calcd. for C₂₆H₂₆ClN₅O₃, 491.97; m/z found 492.1 [M+H]+.

Note: Similar coupling procedure was followed to prepare compound Example 60.

| Acid Intermediate | Amine Intermediate | Final Product |
|---|---|---|
| HBS-055-197 | HBS-055-117 | Example 60 |

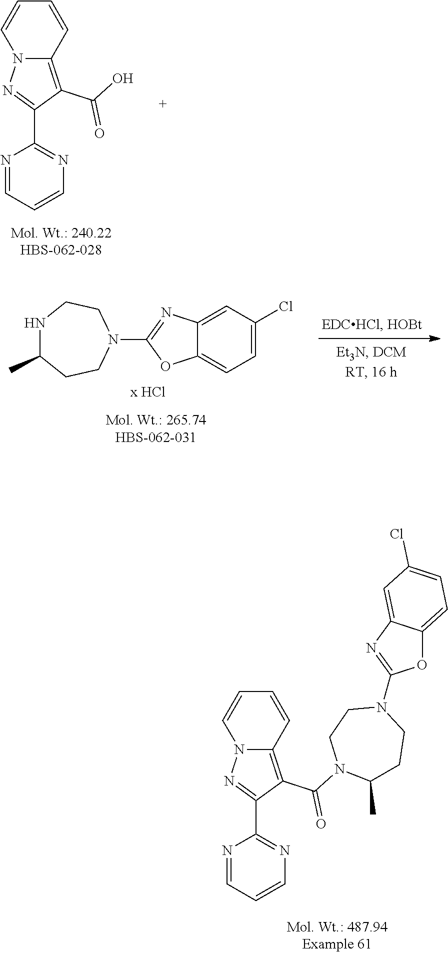

Synthesis of Example 61: Intermediate HBS-062-028 (0.02 g, 0.083 mmol) was dissolved in anhydrous DCM (2.0 mL). The EDC·HCl (0.024 g, 0.13 mmol) and HOBt (0.017 g, 0.13 mmol) were added followed by Et₃N (0.06 mL, 0.42 mmol). The r×n mixture was stirred at ambient temperature for 5.0 min. Intermediate HBS-062-031 (0.028 g, 0.083 mmol) was added to the r×n mixture. The r×n mixture was stirred at ambient temperature for 16 h. The LCMS data shows product formation m/z 488.1. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.0347 g of product was obtained (Yield 85.5%) MS (ESI) mass calcd. for C₂₅H₂₂ClN₇O₂, 487.94; m/z found 488.1 [M+H]+.

Note: Similar coupling procedure was followed to prepare compound Example 62 using following intermediates.

| Acid Intermediate | Amine Intermediate | Final Product |
|---|---|---|
| HBS-062-028 | HBS-055-117 | Example 62 |

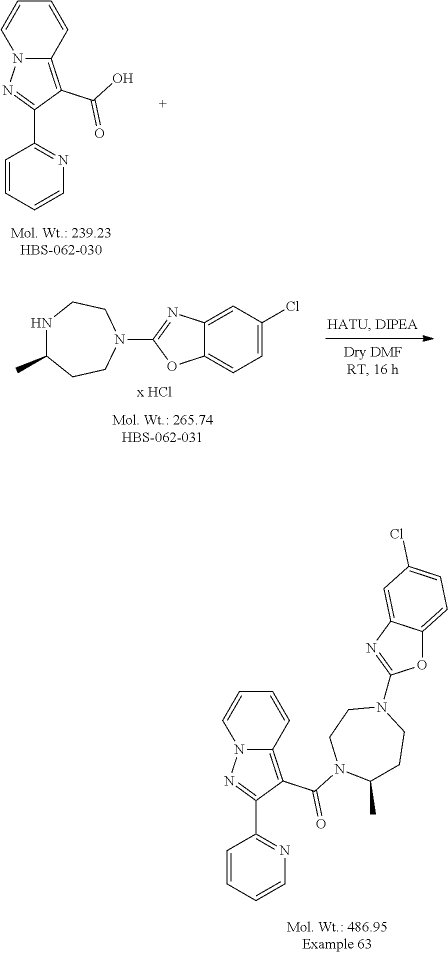

Synthesis of Example 63: Intermediate HBS-062-030 (0.02 g, 0.084 mmol) and HATU (0.038 g, 0.1 mmol) were dissolved in anhydrous DMF (1.5 mL). The DIPEA (0.06 mL, 0.33 mmol) was added and r×n mixture was stirred at ambient temperature for 5.0 min. Intermediate HBS-062-031 (0.028 g, 0.084 mmol) was added to the r×n mixture. The r×n mixture was stirred at ambient temperature for 16 h. The LCMS data shows product formation n/z 487.1. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.041 g of product was obtained (Yield Quant.) MS (ESI) mass calcd. for C₂₆H₂₃ClN₈O₂, 486.95; m/z found 487.1 [M+H]+.

Note: Similar coupling procedure was followed to prepare compound Example 64 using following intermediates.

| Acid Intermediate | Amine Intermediate | Final Product |
|---|---|---|
| HBS-062-030 | HBS-055-117 | Example 64 |

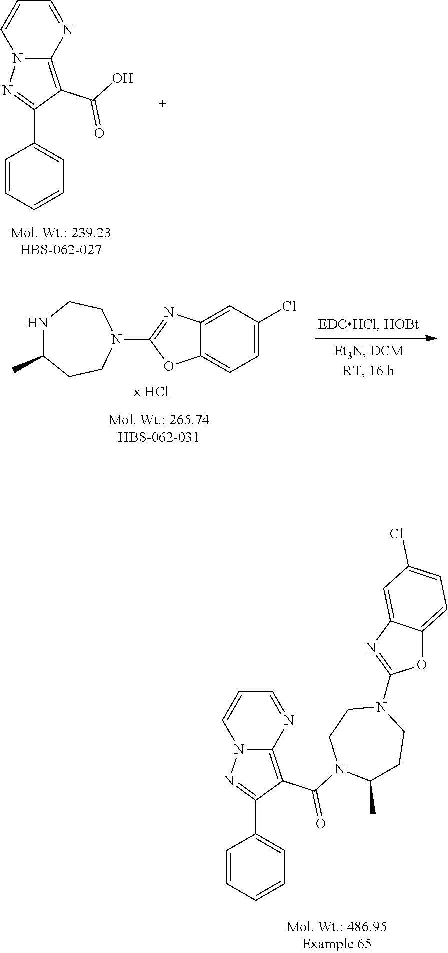

Synthesis of Example 65: Intermediate HBS-062-027 (0.02 g, 0.084 mmol) was dissolved in anhydrous DCM (2.0 mL). The EDC·HCl (0.024 g, 0.13 mmol) and HOBt (0.017 g, 0.13 mmol) were added followed by Et₃N (0.06 mL, 0.42 mmol). The r×n mixture was stirred at ambient temperature for 5.0 min. Intermediate HBS-062-031 (0.028 g, 0.084 mmol) was added to the r×n mixture. The r×n mixture was stirred at ambient temperature for 16 h. The LCMS data shows product formation m/z 487.1. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.0375 g of product was obtained (Yield 92.1%) MS (ESI) mass calcd. for C₂₅H₂₃ClN₆O₂, 486.95; m/z found 487.1 [M+H]+.

Note. Similar coupling procedure was followed to prepare compound Example 66 using following intermediates.

| Acid Intermediate | Amine Intermediate | Final Product |
|---|---|---|
| HBS-062-027 | HBS-055-117 | Example 66 |

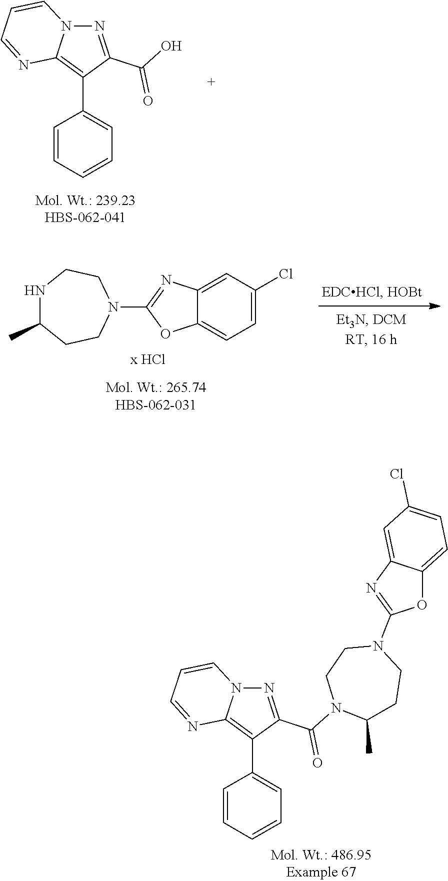

Synthesis of Example 67: Intermediate HBS-062-041 (0.02 g, 0.084 mmol) was dissolved in anhydrous DCM (2.0 mL). The EDC·HCl (0.024 g, 0.13 mmol) and HOBt (0.017 g, 0.13 mmol) were added followed by Et₃N (0.06 mL, 0.42 mmol). The r×n mixture was stirred at ambient temperature for 5.0 min. Intermediate HBS-062-031 (0.028 g, 0.084 mmol) was added to the r×n mixture. The r×n mixture was stirred at ambient temperature for 16 h. The LCMS data shows product formation m/z 487.1. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.0336 g of product was obtained (Yield 82.5%) MS (ESI) mass calcd. for C₂₆H₂₃ClN₆O₂, 486.95; m/z found 487.1 [M+H]+.

Note: Similar coupling procedure was followed to prepare the compound Example 68 using the following intermediates.

| Acid Intermediate | Amine Intermediate | Final Product |
|---|---|---|
| HBS-062-041 | HBS-055-117 | Example 68 |

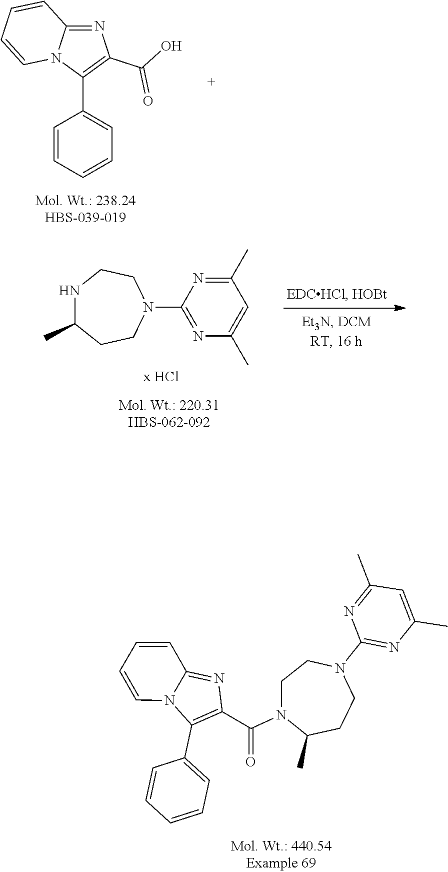

Synthesis of Example 69: HBS-039-019 (0.02 g, 0.084 mmol) was dissolved in anhydrous DCM (2.0 mL). The EDC·HCl (0.024 g, 0.13 mmol) and HOBt (0.017 g, 0.13 mmol) were added followed by Et₃N (0.06 mL, 0.42 mmol). The r×n mixture was stirred at ambient temperature for 5.0 min. Intermediate HBS-062-092 (0.028 g, 0.084 mmol) was added to the r×n mixture. The r×n mixture was stirred at ambient temperature for 16 h. The LCMS data shows product formation m/z 441.2. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.0343 g of product was obtained (Yield 92.7%) MS (ESI) mass calcd. for C₂₈H₂₈N₆O, 440.54; m/z found 441.2 [M+H]+.

Note: Similar coupling procedure was followed to prepare compound Example 70, Example 71, Example 72, Example 73 and Example 74 using the following intermediates.

| Acid Intermediate | Amine Intermediate | Final Product |
|---|---|---|
| HBS-062-013 | HBS-062-092 | Example 70 |
| HBS-039-018 | HBS-062-092 | Example 71 |
| HBS-037-054 | HBS-062-092 | Example 72 |
| HBS-037-093 | HBS-062-092 | Example 73 |
| HBS-062-041 | HBS-062-092 | Example 74 |

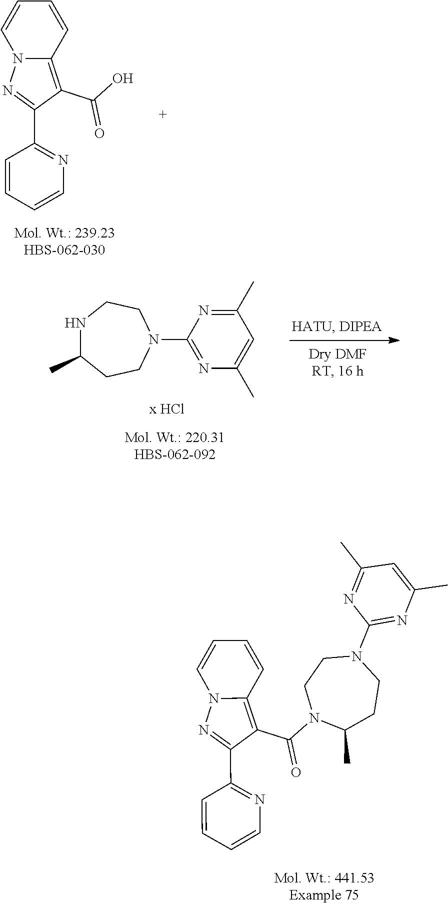

Synthesis of Example 75: Intermediate HBS-062-030 (0.02 g, 0.084 mmol) and HATU (0.038 g, 0.1 mmol) were dissolved in anhydrous DMF (1.5 mL). The DIPEA (0.06 mL, 0.33 mmol) was added and r×m mixture was stirred at ambient temperature for 75.0 m. Intermediate HBS-062-092 (0.028 g, 0.084 mmol) was added to the r×n mixture. The r×n mixture was stirred at ambient temperature for 16 h. The LCMS data shows product formation m/z 442.2. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.037 g of product was obtained (Yield Quant.) MS (ESI) mass calcd. for C₂₅H₂₇N₇O, 441.53; m/z found 442.2 [M+H]+.

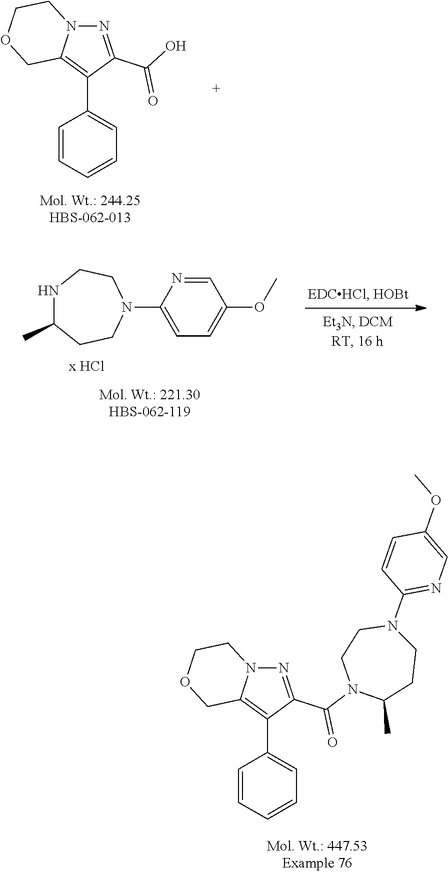

Synthesis of Example 76: Intermediate HBS-062-013 (0.02 g, 0.082 mmol) was dissolved in anhydrous DCM (2.0 mL). The EDC·HCl (0.024 g, 0.13 mmol) and HOBt (0.017 g, 0.13 mmol) were added followed by Et₃N (0.06 mL, 0.41 mmol). The r×n mixture was stirred at ambient temperature for 5.0 min. Intermediate HBS-062-119 (0.027 g, 0.082 mmol) was added to the r×n mixture. The r×n mixture was stirred at ambient temperature for 16 h. The LCMS data shows product formation m/448.2. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.0294 g of product was obtained (Yield 80.2%) MS (ESI) mass calcd. for C₂₅H₂₉N₅O₃, 447.53; m/z found 448.2 [M+H]+.

Note: Similar coupling procedure was followed to prepare compound Example 77, Example 78, Example 79, and Example 80 using the following intermediates.

| Acid Intermediate | Amine Intermediate | Final Product |
|---|---|---|
| HBS-039-019 | HBS-062-119 | Example 77 |
| HBS-039-018 | HBS-062-119 | Example 78 |
| HBS-055-197 | HBS-062-119 | Example 79 |
| HBS-062-041 | HBS-062-119 | Example 80 |

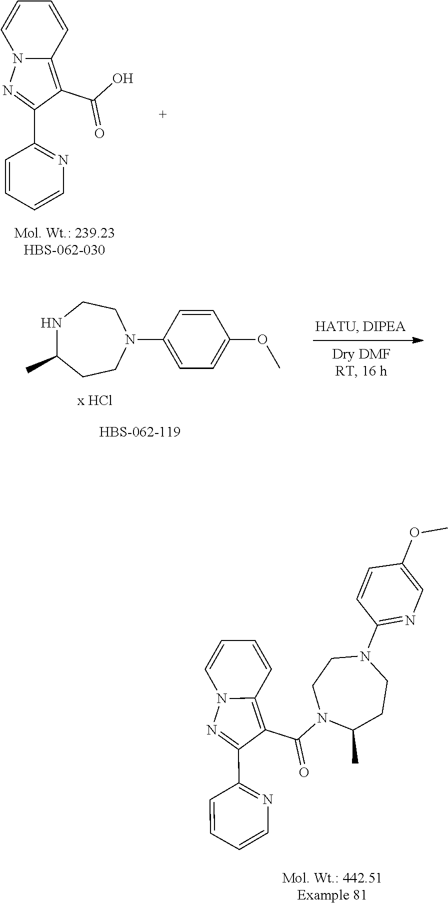

Synthesis of Example 81: Intermediate HBS-062-030 (0.02 g, 0.084 mmol) and HATU (0.038 g, 0.1 mmol) were dissolved in anhydrous DMF (1.5 mL). The DIPEA (0.06 mL, 0.33 mmol) was added and r×n mixture was stirred at ambient temperature for 5.0 min. Intermediate HBS-062-119 (0.028 g, 0.084 mmol) was added to the r×n mixture. The r×n mixture was stirred at ambient temperature for 16 h. The LCMS data shows product formation m/z 442.2. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system. Mobile phase: DCM:MeOH gradient. The 0.0342 g of product was obtained (Yield 92.3%) MS (ESI) mass calcd. for C₂₅H₂₆N₆O₂, 442.51; m/z found 443.4 [M+H]+.

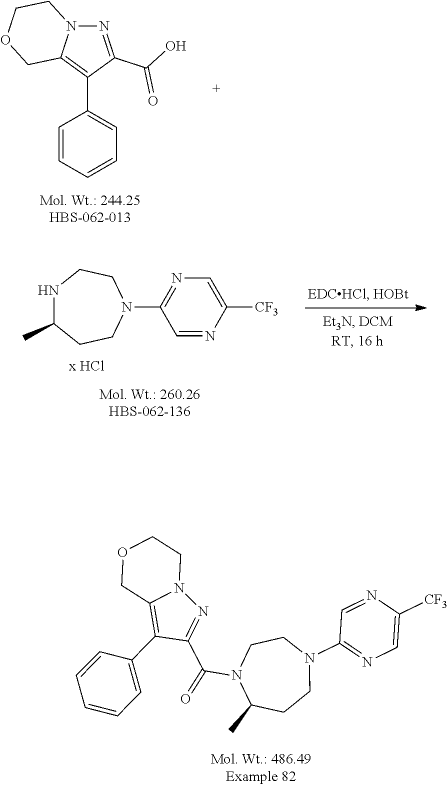 [SMILES: O=C(O)c1nn2c(c1-c1ccccc1)COCC2 ; C[C@@H]1CCN(c2cnc(C(F)(F)F)cn2)CCN1 ; "EDC·HCl, HOBt" ; C[C@@H]1CCN(c2cnc(C(F)(F)F)cn2)CCN1C(=O)c1nn2c(c1-c1ccccc1)COCC2]

Synthesis of Example 82: Intermediate HBS-062-013 (0.02 g, 0.082 mmol) was dissolved in anhydrous DCM (2.0 mL). The EDC·HCl (0.024 g, 0.13 mmol) and HOBt (0.017 g, 0.13 mmol) were added followed by Et₃N (0.06 mL, 0.42 mmol). The r×n mixture was stirred at ambient temperature for 5.0 min. Intermediate HBS-062-136 (0.03 g, 0.082 mmol) was added to the r×n mixture. The r×n mixture was stirred at ambient temperature for 16 h. The LCMS data shows product formation m/z 487.2. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: DCM:MeOH gradient. The 0.0348 g of product was obtained (Yield 87.4%) MS (ESI) mass calcd. for C₂₄H₂₅F₃N₆O₂, 486.49; m/z found 487.2 [M+H]+.

Note: Similar coupling procedure was followed to prepare compound Example 83, Example 84, Example 85, Example 86, Example 87, Example 88, Example 89, Example 90, and Example 91 using the following intermediates.

| Acid Intermediate | Amine Intermediate | Final Product |
|---|---|---|
| HBS-039-019 | HBS-062-136 | Example 83 |
| HBS-055-197 | HBS-062-136 | Example 84 |
| HBS-062-041 | HBS-062-136 | Example 85 |
| HBS-039-018 | HBS-062-136 | Example 86 |
| HBS-062-030 | HBS-062-136 | Example 87 |
| HBS-062-028 | HBS-062-136 | Example 88 |
| HBS-037-054 | HBS-062-136 | Example 89 |
| HBS-037-163 | HBS-062-136 | Example 90 |
| HBS-062-027 | HBS-062-136 | Example 91 |

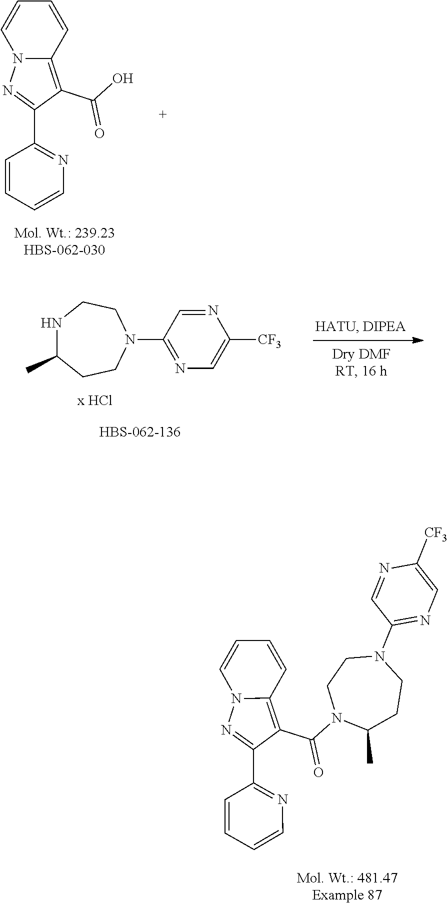 [SMILES: O=C(O)c1c(-c2ccccn2)nn2ccccc12 ; C[C@@H]1CCN(c2cnc(C(F)(F)F)cn2)CCN1 ; "HATU, DIPEA" ; C[C@@H]1CCN(c2cnc(C(F)(F)F)cn2)CCN1C(=O)c1c(-c2ccccn2)nn2ccccc12]

Synthesis of Example 87: Intermediate HBS-062-030 (0.02 g, 0.084 mmol) and HATU (0.038 g, 0.1 mmol) were dissolved in anhydrous DMF (1.5 mL). The DIPEA (0.07 mL, 0.4 mmol) was added and r×n mixture was stirred at ambient temperature for 5.0 min. Intermediate HBS-062-136 (0.034 g, 0.084 mmol) was added to the r×n mixture. The r×n mixture was stirred at ambient temperature for 16 h. The LCMS data shows product formation m/z 482.2. The r×n mixture was diluted with DCM and washed with saturated solution of NaHCO₃. The DCM layer was separated and dried over anhydrous Na₂SO₄. The evaporation of solvent gave crude product. The crude product was purified by ISCO combi-flash system, Mobile phase: Ethyl acetate:Hexane gradient. The 0.04 g of product was obtained (Yield Quant.) MS (ESI) mass calcd. for C₂₄H₁₂F₃N₇O, 481.47; m/z found 482.2 [M+H]+.

Note: General coupling procedure (EDC·HCl or HATU) was followed to prepare compound Example 92 Example 93, Example 94, Example 95, Example 96, Example 97, Example 98, Example 99, Example 100, and Example 101 using the following intermediates.

| Acid Intermediate | Amine Intermediate | Final Product |
|---|---|---|
| HBS-062-028 | HBS-062-119 | Example 92 |
| HBS-062-030 | HBS-062-119 | Example 93 |
| HBS-062-028 | HBS-062-136 | Example 94 |
| HBS-062-030 | HBS-062-136 | Example 95 |
| HBS-062-186 | HBS-062-031 | Example 96 |
| HBS-062-186 | HBS-055-117 | Example 97 |
| HBS-062-186 | HBS-062-136 | Example 98 |
| HBS-062-186 | HBS-062-092 | Example 99 |
| 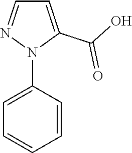 [SMILES: O=C(O)c1ccnn1-c1ccccc1] | HBS-062-031 | Example 100 |
| 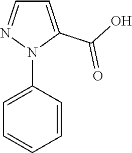 [SMILES: O=C(O)c1ccnn1-c1ccccc1] | HBS-055-117 | Example 101 |

### Example 2

### A. Biological Assays

Antagonistic activities on both orexin receptors (i.e., to identify Orexin Receptor Antagonists) have been measured for each example compound using the in vitro assays (intracellular calcium measurements) as described herein. Other assays may also be used to identify Orexin Receptor Antagonists as would be understood by those of ordinary skill in the art. Chinese hamster ovary (CHO) cells expressing the human orexin-1 receptor and the human orexin-2 receptor, respectively, are grown in culture medium (Ham F-12 with L-Glutamine) containing 300 μg/mL G418, 100 U/mL penicillin, 100 μg/mL streptomycin and 10% heat inactivated fetal calf serum (FCS). The cells are seeded at 20′000 cells/well into 384-well black clear bottom sterile plates (Greiner). The seeded plates are incubated overnight at 37° C. in 5% CO₂. Human orexin-A as an agonist is prepared as 1 mM stock solution in MeOH: water (1:1), diluted in HBSS containing 0.1% bovine serum albumin (BSA), NaHC0₃: 0.375 g/L and 20 mM HEPES for use in the assay at a final concentration of 3 nM.

Antagonists were prepared as 10 mM stock solution in DMSO, then diluted in 384-well plates using DMSO followed by a transfer of the dilutions into in HBSS containing 0.1% bovine serum albumin (BSA), NaHC0₃: 0.375 g/L and 20 mM HEPES. On the day of the assay, 50 μL of staining buffer (HBSS containing 1% FCS, 20 mM HEPES, NaHC0₃: 0.375 g/L, 5 mM probenecid (Sigma) and 3 μM of the fluorescent calcium indicator fluro-4 AM (1 mM stock solution in DMSO, containing 10% pluronic) is added to each well. The 384-well cell-plates are incubated for 50 min at 37° C. in 5% C₀₂ followed by equilibration at RT for 30 min before measurement. Within the Fluorescent Imaging Plate Reader (FLIPR Tetra, Molecular Devices), potential Orexin Receptor Antagonists were added to the plate in a volume of 10 μl/well, incubated for 120 min and finally 10 μL/well of agonist is added. Fluorescence was measured for each well at 1 second intervals, and the height of each fluorescence peak compared to the height of the fluorescence peak induced by 3 nM orexin-A with vehicle in place of antagonist. The IC₅₀ value (the concentration of compound needed to inhibit 50% of the agonistic response) was determined and normalized using the obtained IC₅₀ value of an on-plate reference compound. Optimized conditions were achieved by adjustment of pipetting speed and cell splitting regime. The calculated IC₅₀ values fluctuate depending on the daily cellular assay performance, as is known to those skilled in the art. In the case where IC₅₀ values have been determined several times for the same compound, the geometric mean has been given. Antagonistic activities of example compounds are shown in Table 1.

TABLE 1
Example of Compound data
(% Inhibition at Orexin receptor type 1 and type 2, @ 1 uM, and IC₅₀/K_{b} values)

| | | OXIR % | OXIR | | OX2R | |
|---|---|---|---|---|---|---|
| | | Inh. @ | IC50 /Kb | OX2R % | IC50 /Kb | MW. |
| Example | Structure | 1 uM | (nM) | Inh. @ 1uM | (nM) | (g/mol) |
| 1 | 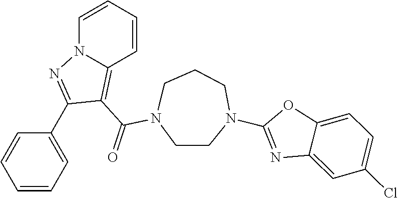 [SMILES: O=C(c1c(-c2ccccc2)nn2ccccc12)N1CCCN(c2nc3cc(Cl)ccc3o2)CC1] | 98.7 | 16/3 | 94.9 | 49/7 | 471.94 |
| 2 | 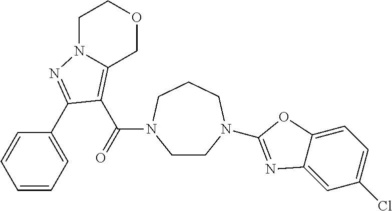 [SMILES: O=C(c1c(-c2ccccc2)nn2c1COCC2)N1CCCN(c2nc3cc(Cl)ccc3o2)CC1] | 64.2 | | 43.4 | | 477.94 |
| 3 | 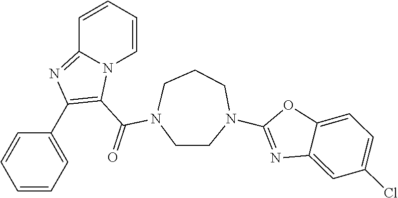 [SMILES: O=C(c1c(-c2ccccc2)nc2ccccn12)N1CCCN(c2nc3cc(Cl)ccc3o2)CC1] | 94.0 | 20/4 | 71.8 | 86/12 | 471.94 |
| 4 | 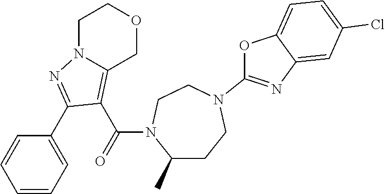 [SMILES: C[C@@H]1CCN(c2nc3cc(Cl)ccc3o2)CCN1C(=O)c1c(-c2ccccc2)nn2c1COCC2] | 98.1 | 14/3 | 96.8 | 29/4 | 485.96 |
| 5 | 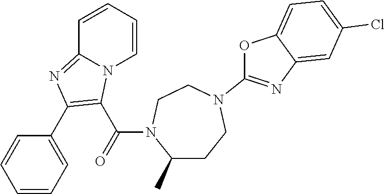 [SMILES: C[C@@H]1CCN(c2nc3cc(Cl)ccc3o2)CCN1C(=O)c1c(-c2ccccc2)nc2ccccn12] | 98.5 | 17/4 | 94.5 | 36/5 | 485.96 |
| 6 | 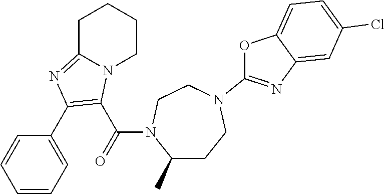 [SMILES: C[C@@H]1CCN(c2nc3cc(Cl)ccc3o2)CCN1C(=O)c1c(-c2ccccc2)nc2n1CCCC2] | 62.5 | | 33.4 | | 490.00 |
| 7 | 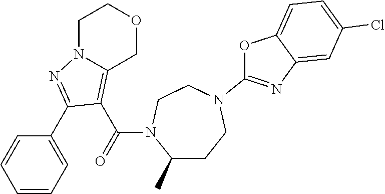 [SMILES: C[C@@H]1CCN(c2nc3cc(Cl)ccc3o2)CCN1C(=O)c1c(-c2ccccc2)nn2c1COCC2] | 91. | 37/8 | 54.5 | | 491.97 |
| 8 | 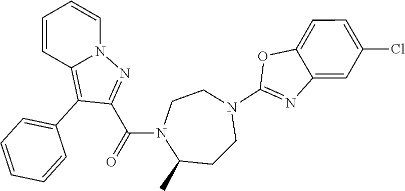 [SMILES: C[C@@H]1CCN(c2nc3cc(Cl)ccc3o2)CCN1C(=O)c1nn2ccccc2c1-c1ccccc1] | 98.0 | 42/9 | 82.7 | 140/19 | 485.96 |
| 9 | 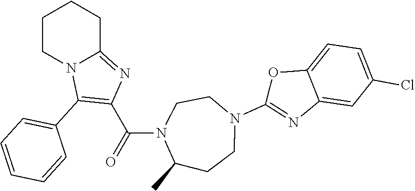 [SMILES: C[C@@H]1CCN(c2nc3cc(Cl)ccc3o2)CCN1C(=O)c1nc2n(c1-c1ccccc1)CCCC2] | 49.6 | | 12.2 | | 490.00 |
| 10 | 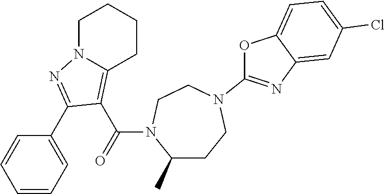 [SMILES: C[C@@H]1CCN(c2nc3cc(Cl)ccc3o2)CCN1C(=O)c1c(-c2ccccc2)nn2c1CCCC2] | 85.9 | 48/10 | 55.1 | 580/80 | 490.0 |
| 11 | 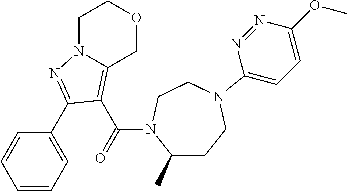 [SMILES: COc1ccc(N2CC[C@@H](C)N(C(=O)c3c(-c4ccccc4)nn4c3COCC4)CC2)nn1] | 12.2 | | 21.5 | | 448.52 |
| 12 | 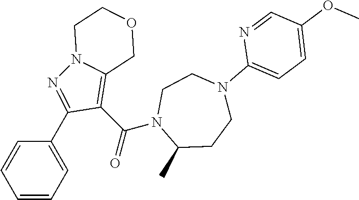 [SMILES: COc1ccc(N2CC[C@@H](C)N(C(=O)c3c(-c4ccccc4)nn4c3COCC4)CC2)nc1] | 14.7 | | 25.0 | | 447.53 |
| 13 | 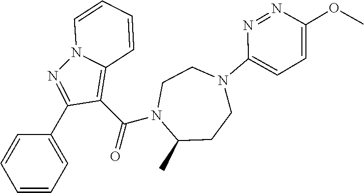 [SMILES: COc1ccc(N2CC[C@@H](C)N(C(=O)c3c(-c4ccccc4)nn4ccccc34)CC2)nn1] | 45.1 | | 38.6 | | 442.51 |
| 14 | 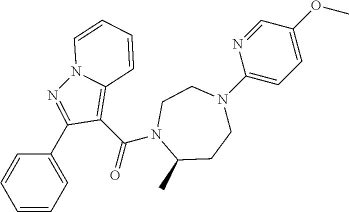 [SMILES: COc1ccc(N2CC[C@@H](C)N(C(=O)c3c(-c4ccccc4)nn4ccccc34)CC2)nc1] | 91.6 | 13/3 | 57.6 | | 441.53 |
| 15 | 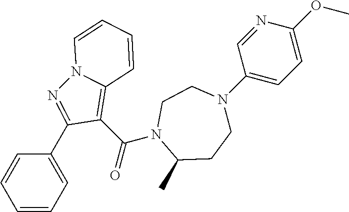 [SMILES: COc1ccc(N2CC[C@@H](C)N(C(=O)c3c(-c4ccccc4)nn4ccccc34)CC2)cn1] | 38.7 | | 37.1 | | 441.53 |
| 16 | 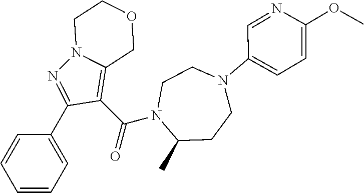 [SMILES: COc1ccc(N2CC[C@@H](C)N(C(=O)c3c(-c4ccccc4)nn4c3COCC4)CC2)cn1] | −21.1 | | 3.3 | | 447.53 |
| 17 | 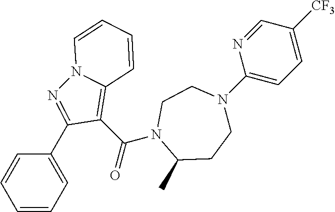 [SMILES: C[C@@H]1CCN(c2ccc(C(F)(F)F)cn2)CCN1C(=O)c1c(-c2ccccc2)nn2ccccc12] | 96.3 | 30/6 | 66.7 | | 479.50 |
| 18 | 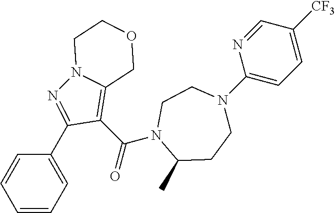 [SMILES: C[C@@H]1CCN(c2ccc(C(F)(F)F)cn2)CCN1C(=O)c1c(-c2ccccc2)nn2c1COCC2] | 82.5 | 39/8 | 35.5 | | 485.50 |
| 19 | 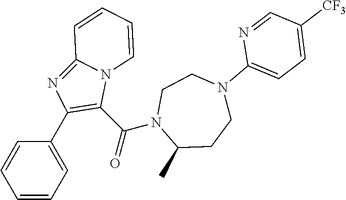 [SMILES: C[C@@H]1CCN(c2ccc(C(F)(F)F)cn2)CCN1C(=O)c1c(-c2ccccc2)nc2ccccn12] | 94.5 | 20/4 | 48.2 | | 479.50 |
| 20 | 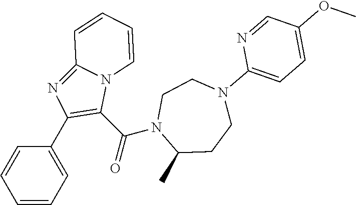 [SMILES: COc1ccc(N2CC[C@@H](C)N(C(=O)c3c(-c4ccccc4)nc4ccccn34)CC2)nc1] | 62.1 | | 32.2 | | 441.53 |
| 21 | 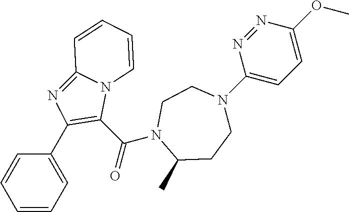 [SMILES: COc1ccc(N2CC[C@@H](C)N(C(=O)c3c(-c4ccccc4)nc4ccccn34)CC2)nn1] | 20.9 | | 23.7 | | 442.51 |
| 22 | 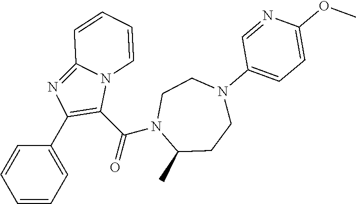 [SMILES: COc1ccc(N2CC[C@@H](C)N(C(=O)c3c(-c4ccccc4)nc4ccccn34)CC2)cn1] | 15.0 | | 28.3 | | 441.53 |
| 23 | 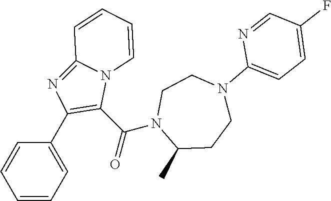 [SMILES: C[C@@H]1CCN(c2ccc(F)cn2)CCN1C(=O)c1c(-c2ccccc2)nc2ccccn12] | 18.6 | | 32.7 | | 429.49 |
| 24 | 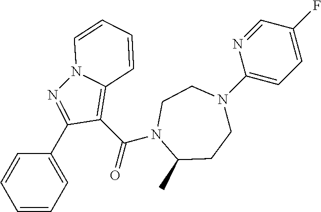 [SMILES: C[C@@H]1CCN(c2ccc(F)cn2)CCN1C(=O)c1c(-c2ccccc2)nn2ccccc12] | 44.2 | | 46.0 | | 429.49 |
| 25 | 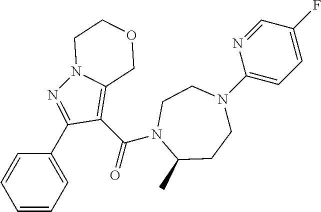 [SMILES: C[C@@H]1CCN(c2ccc(F)cn2)CCN1C(=O)c1c(-c2ccccc2)nn2c1COCC2] | 11.6 | | 7.7 | | 435.49 |
| 26 | 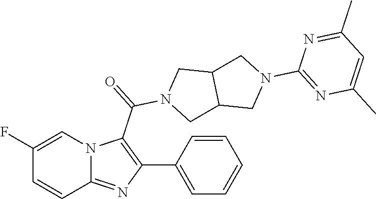 [SMILES: Cc1cc(C)nc(N2CC3CN(C(=O)c4c(-c5ccccc5)nc5ccc(F)cn45)CC3C2)n1] | 1.61 | | 33.83 | | 456.51 |
| 27 | 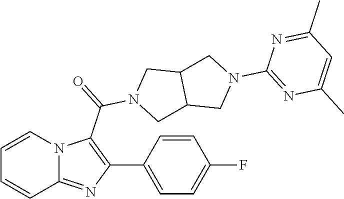 [SMILES: Cc1cc(C)nc(N2CC3CN(C(=O)c4c(-c5ccc(F)cc5)nc5ccccn45)CC3C2)n1] | 6.42 | | 14.24 | | 456.51 |
| 28 | 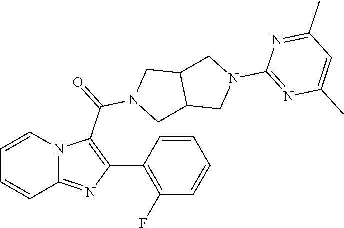 [SMILES: Cc1cc(C)nc(N2CC3CN(C(=O)c4c(-c5ccccc5F)nc5ccccn45)CC3C2)n1] | 18.34 | | 36.65 | | 456.51 |
| 29 | 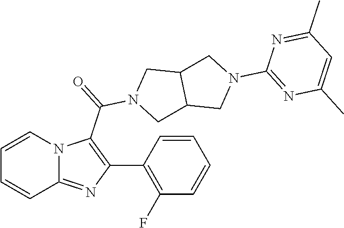 [SMILES: Cc1cc(C)nc(N2CC3CN(C(=O)c4c(-c5ccccc5F)nc5ccccn45)CC3C2)n1] | 19.54 | | 34.14 | | 456.51 |
| 30 | 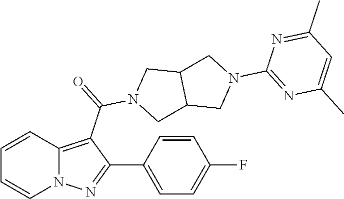 [SMILES: Cc1cc(C)nc(N2CC3CN(C(=O)c4c(-c5ccc(F)cc5)nn5ccccc45)CC3C2)n1] | 28.26 | | 36.79 | | 456.51 |
| 31 | 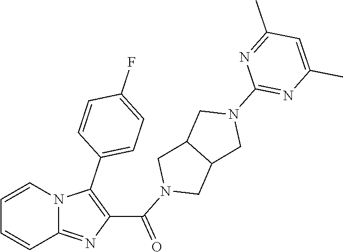 [SMILES: Cc1cc(C)nc(N2CC3CN(C(=O)c4nc5ccccn5c4-c4ccc(F)cc4)CC3C2)n1] | 8.04 | | 3.74 | | 456.51 |
| 32 | 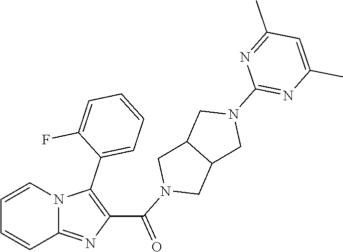 [SMILES: Cc1cc(C)nc(N2CC3CN(C(=O)c4nc5ccccn5c4-c4ccccc4F)CC3C2)n1] | 4.01 | | 12.12 | | 456.51 |
| 33 | 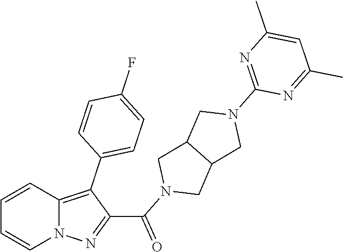 [SMILES: Cc1cc(C)nc(N2CC3CN(C(=O)c4nn5ccccc5c4-c4ccc(F)cc4)CC3C2)n1] | 5 | | 17.64 | | 456.51 |
| 34 | 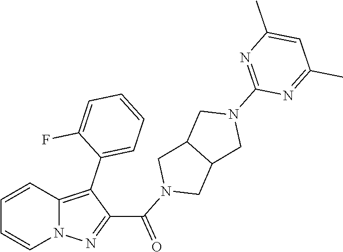 [SMILES: Cc1cc(C)nc(N2CC3CN(C(=O)c4nn5ccccc5c4-c4ccccc4F)CC3C2)n1] | 2.36 | | 31.45 | | 456.51 |
| 35 | 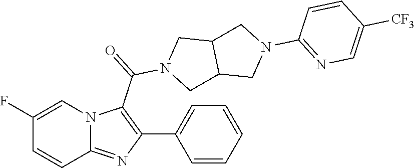 [SMILES: O=C(c1c(-c2ccccc2)nc2ccc(F)cn12)N1CC2CN(c3ccc(C(F)(F)F)cn3)CC2C1] | 59.11 | 1400/295 | 22.47 | | 495.47 |
| 36 | 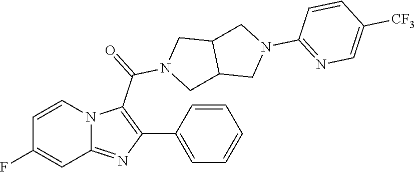 [SMILES: O=C(c1c(-c2ccccc2)nc2cc(F)ccn12)N1CC2CN(c3ccc(C(F)(F)F)cn3)CC2C1] | 35.03 | | 23.58 | | 495,47 |
| 37 | 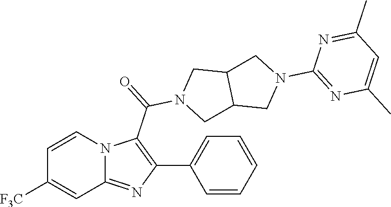 [SMILES: Cc1cc(C)nc(N2CC3CN(C(=O)c4c(-c5ccccc5)nc5cc(C(F)(F)F)ccn45)CC3C2)n1] | 5.33 | | 46.85 | | 506.52 |
| 38 | 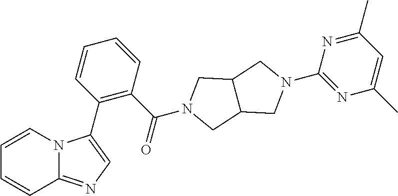 [SMILES: Cc1cc(C)nc(N2CC3CN(C(=O)c4ccccc4-c4cnc5ccccn45)CC3C2)n1] | 6.86 | | 3.32 | | 438.52 |
| 39 | 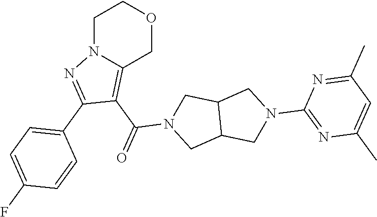 [SMILES: Cc1cc(C)nc(N2CC3CN(C(=O)c4c(-c5ccc(F)cc5)nn5c4COCC5)CC3C2)n1] | 14.89 | | 29.13 | | 462.52 |
| 40 | 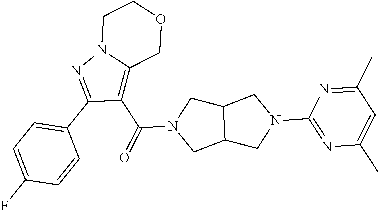 [SMILES: Cc1cc(C)nc(N2CC3CN(C(=O)c4c(-c5ccc(F)cc5)nn5c4COCC5)CC3C2)n1] | 11.58 | | 7.18 | | 462.52 |
| 41 | 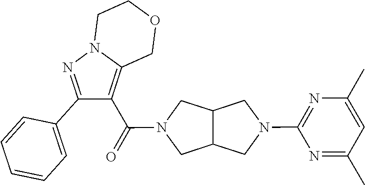 [SMILES: Cc1cc(C)nc(N2CC3CN(C(=O)c4c(-c5ccccc5)nn5c4COCC5)CC3C2)n1] | 6 | | 11.52 | | 444.53 |
| 42 | 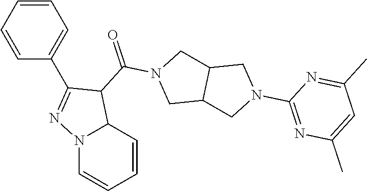 [SMILES: Cc1cc(C)nc(N2CC3CN(C(=O)C4C(c5ccccc5)=NN5C=CC=CC45)CC3C2)n1] | 40.37 | | 68.02 | 82/11 | 438,52 |
| 43 | 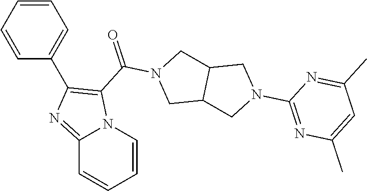 [SMILES: Cc1cc(C)nc(N2CC3CN(C(=O)c4c(-c5ccccc5)nc5ccccn45)CC3C2)n1] | 19.65 | | 38.39 | | 438.52 |
| 44 | 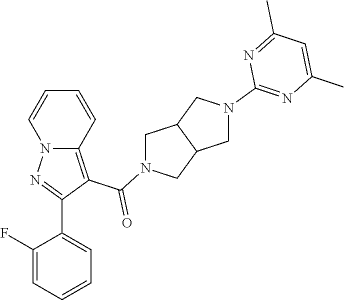 [SMILES: Cc1cc(C)nc(N2CC3CN(C(=O)c4c(-c5ccccc5F)nn5ccccc45)CC3C2)n1] | 19.25 | | 56.14 | 573/79 | 456.51 |
| 45 | 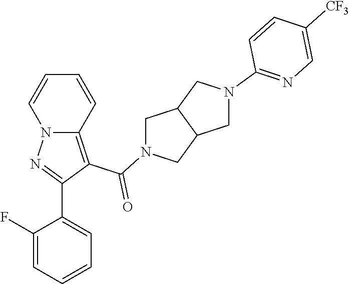 [SMILES: O=C(c1c(-c2ccccc2F)nn2ccccc12)N1CC2CN(c3ccc(C(F)(F)F)cn3)CC2C1] | 78.16 | 194/41 | 27.13 | | 495.47 |
| 46 | 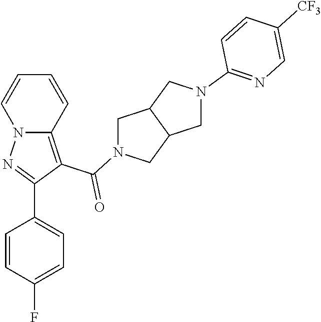 [SMILES: O=C(c1c(-c2ccc(F)cc2)nn2ccccc12)N1CC2CN(c3ccc(C(F)(F)F)cn3)CC2C1] | 53.92 | 311/66 | 14.58 | | 495,47 |
| 47 | 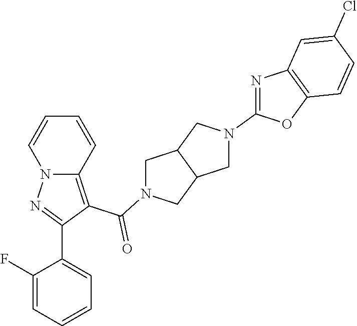 [SMILES: O=C(c1c(-c2ccccc2F)nn2ccccc12)N1CC2CN(c3nc4cc(Cl)ccc4o3)CC2C1] | 97.03 | 43/9 | 88.97 | 52/7 | 501.94 |
| 48 | 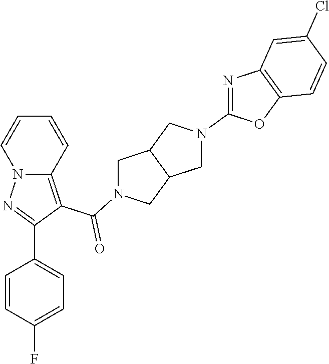 [SMILES: O=C(c1c(-c2ccc(F)cc2)nn2ccccc12)N1CC2CN(c3nc4cc(Cl)ccc4o3)CC2C1] | 87.06 | 86/18 | 63.17 | 177/24 | 501.94 |
| 49 | 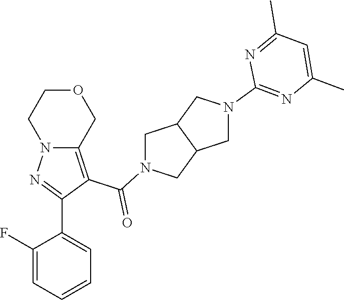 [SMILES: Cc1cc(C)nc(N2CC3CN(C(=O)c4c(-c5ccccc5F)nn5c4COCC5)CC3C2)n1] | −5.54 | | 12.83 | | 462.52 |
| 50 | 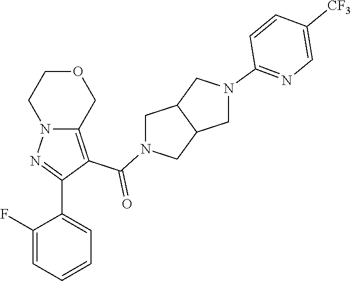 [SMILES: O=C(c1c(-c2ccccc2F)nn2c1COCC2)N1CC2CN(c3ccc(C(F)(F)F)cn3)CC2C1] | 9.55 | | 1.62 | | 501.48 |
| 51 | 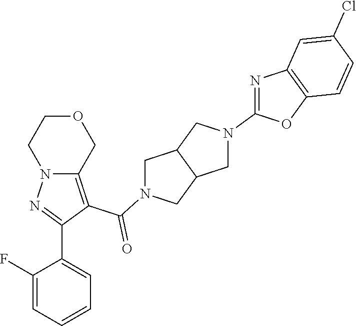 [SMILES: O=C(c1c(-c2ccccc2F)nn2c1COCC2)N1CC2CN(c3nc4cc(Cl)ccc4o3)CC2C1] | 38.53 | | 24.27 | | 507.94 |
| 52 | 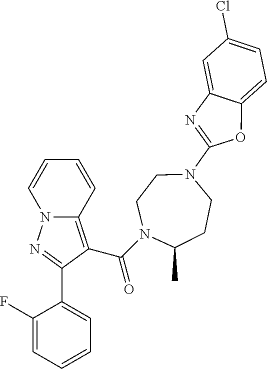 [SMILES: C[C@@H]1CCN(c2nc3cc(Cl)ccc3o2)CCN1C(=O)c1c(-c2ccccc2F)nn2ccccc12] | 100.22 | 12/3 | 98.73 | 16/2 | 503.96 |
| 53 | 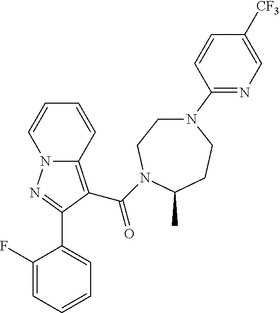 [SMILES: C[C@@H]1CCN(c2ccc(C(F)(F)F)cn2)CCN1C(=O)c1c(-c2ccccc2F)nn2ccccc12] | 101.73 | 9/2 | 98.78 | 21/3 | 497.49 |
| 54 | 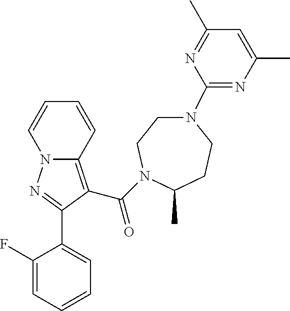 [SMILES: Cc1cc(C)nc(N2CC[C@@H](C)N(C(=O)c3c(-c4ccccc4F)nn4ccccc34)CC2)n1] | 91.92 | 5/1 | 99.68 | 6/0.8 | 458.53 |
| 55 | 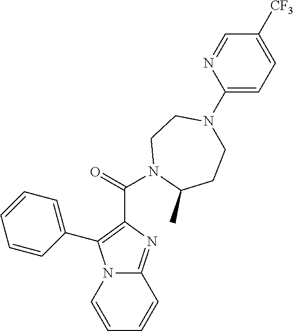 [SMILES: C[C@@H]1CCN(c2ccc(C(F)(F)F)cn2)CCN1C(=O)c1nc2ccccn2c1-c1ccccc1] | 97 | | 37 | | 479.50 |
| 56 | 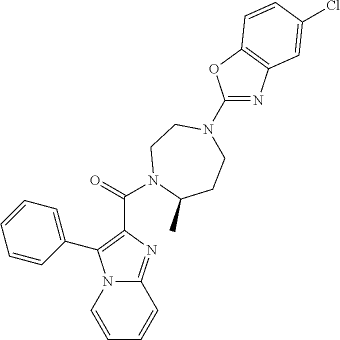 [SMILES: C[C@@H]1CCN(c2nc3cc(Cl)ccc3o2)CCN1C(=O)c1nc2ccccn2c1-c1ccccc1] | 97 | | 27 | | 485.96 |
| 57 | 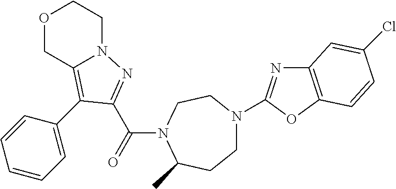 [SMILES: C[C@@H]1CCN(c2nc3cc(Cl)ccc3o2)CCN1C(=O)c1nn2c(c1-c1ccccc1)COCC2] | 98 | | 28 | | 491.97 |
| 58 | 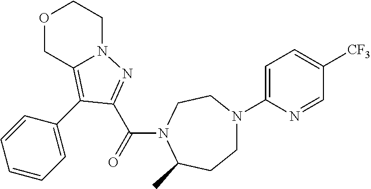 [SMILES: C[C@@H]1CCN(c2ccc(C(F)(F)F)cn2)CCN1C(=O)c1nn2c(c1-c1ccccc1)COCC2] | 94 | | 30 | | 485.50 |
| 59 | 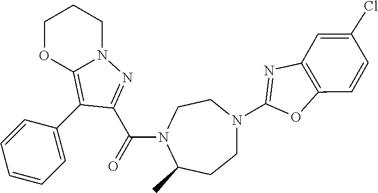 [SMILES: C[C@@H]1CCN(c2nc3cc(Cl)ccc3o2)CCN1C(=O)c1nn2c(c1-c1ccccc1)OCCC2] | 99 | 53/21 | 97 | | 491.97 |
| 60 | 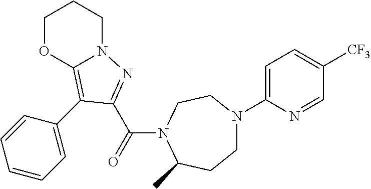 [SMILES: C[C@@H]1CCN(c2ccc(C(F)(F)F)cn2)CCN1C(=O)c1nn2c(c1-c1ccccc1)OCCC2] | 97 | 65/14 | 81 | | 485.5 |
| 61 | 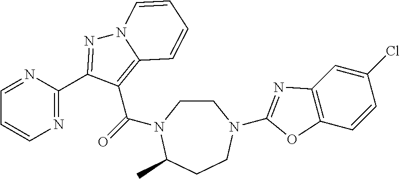 [SMILES: C[C@@H]1CCN(c2nc3cc(Cl)ccc3o2)CCN1C(=O)c1c(-c2ncccn2)nn2ccccc12] | 100 | | 64 | | 487.94 |
| 62 | 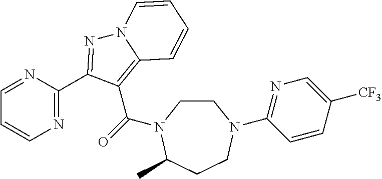 [SMILES: C[C@@H]1CCN(c2ccc(C(F)(F)F)cn2)CCN1C(=O)c1c(-c2ncccn2)nn2ccccc12] | 100 | | 44 | | 481.47 |
| 63 | 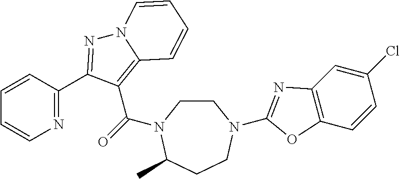 [SMILES: C[C@@H]1CCN(c2nc3cc(Cl)ccc3o2)CCN1C(=O)c1c(-c2ccccn2)nn2ccccc12] | 100 | | 96 | | 486.95 |
| 64 | 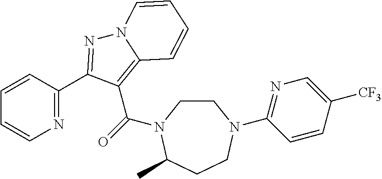 [SMILES: C[C@@H]1CCN(c2ccc(C(F)(F)F)cn2)CCN1C(=O)c1c(-c2ccccn2)nn2ccccc12] | 100 | | 85 | | 480.49 |
| 65 | 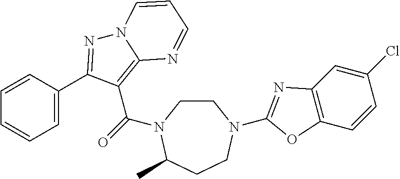 [SMILES: C[C@@H]1CCN(c2nc3cc(Cl)ccc3o2)CCN1C(=O)c1c(-c2ccccc2)nn2cccnc12] | 99 | | 80 | | 486.95 |
| 66 | 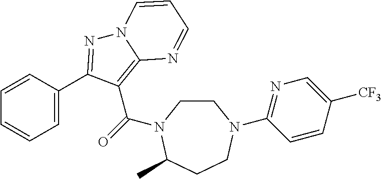 [SMILES: C[C@@H]1CCN(c2ccc(C(F)(F)F)cn2)CCN1C(=O)c1c(-c2ccccc2)nn2cccnc12] | 99 | | 83 | | 480.49 |
| 67 | 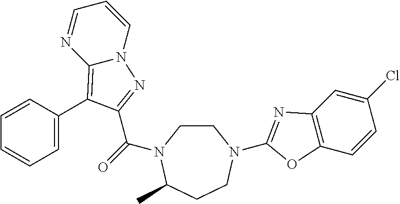 [SMILES: C[C@@H]1CCN(c2nc3cc(Cl)ccc3o2)CCN1C(=O)c1nn2cccnc2c1-c1ccccc1] | 99 | | 80 | | 486.95 |
| 68 | 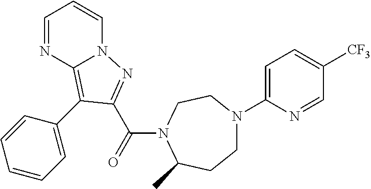 [SMILES: C[C@@H]1CCN(c2ccc(C(F)(F)F)cn2)CCN1C(=O)c1nn2cccnc2c1-c1ccccc1] | 100 | | 68 | | 480.49 |
| 69 | 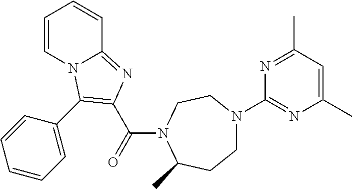 [SMILES: Cc1cc(C)nc(N2CC[C@@H](C)N(C(=O)c3nc4ccccn4c3-c3ccccc3)CC2)n1] | 23 | | 34 | | 440.54 |
| 70 | 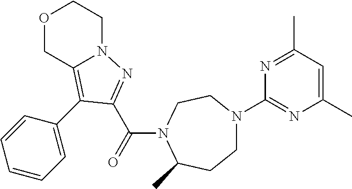 [SMILES: Cc1cc(C)nc(N2CC[C@@H](C)N(C(=O)c3nn4c(c3-c3ccccc3)COCC4)CC2)n1] | 27 | | 44 | | 446.54 |
| 71 | 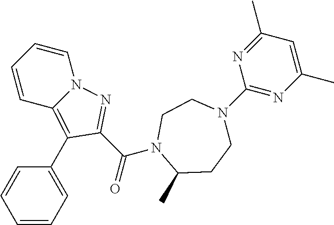 [SMILES: Cc1cc(C)nc(N2CC[C@@H](C)N(C(=O)c3nn4ccccc4c3-c3ccccc3)CC2)n1] | 75 | 218/46 | 96 | 48/8 | 440.54 |
| 72 | 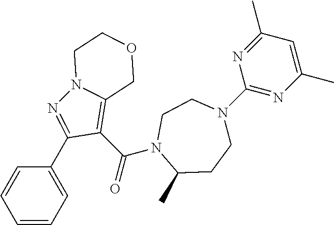 [SMILES: Cc1cc(C)nc(N2CC[C@@H](C)N(C(=O)c3c(-c4ccccc4)nn4c3COCC4)CC2)n1] | 28 | | 65 | | 446.54 |
| 73 | 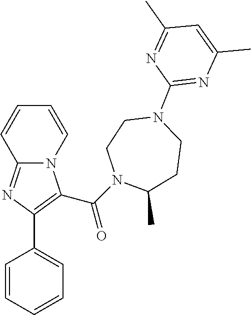 [SMILES: Cc1cc(C)nc(N2CC[C@@H](C)N(C(=O)c3c(-c4ccccc4)nc4ccccn34)CC2)n1] | 65 | | 95 | 140/24 | 440.54 |
| 74 | 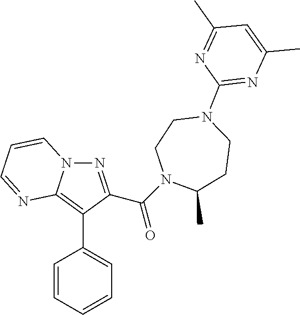 [SMILES: Cc1cc(C)nc(N2CC[C@@H](C)N(C(=O)c3nn4cccnc4c3-c3ccccc3)CC2)n1] | 74 | 149/31 | 95 | 80/14 | 441.53 |
| 75 | 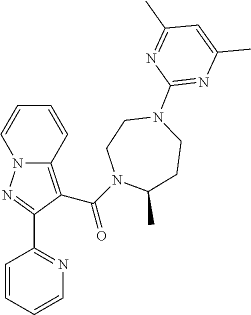 [SMILES: Cc1cc(C)nc(N2CC[C@@H](C)N(C(=O)c3c(-c4ccccn4)nn4ccccc34)CC2)n1] | 79 | 89/19 | 100 | 13/2 | 441.53 |
| 76 | 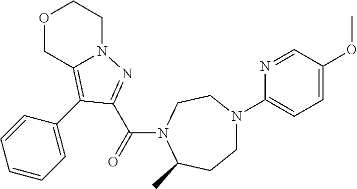 [SMILES: COc1ccc(N2CC[C@@H](C)N(C(=O)c3nn4c(c3-c3ccccc3)COCC4)CC2)nc1] | 23 | | 11 | | 447.53 |
| 77 | 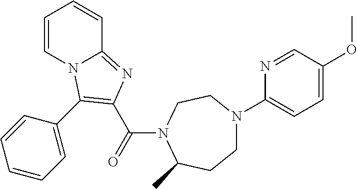 [SMILES: COc1ccc(N2CC[C@@H](C)N(C(=O)c3nc4ccccn4c3-c3ccccc3)CC2)nc1] | 38 | | 16 | | 441.53 |
| 78 | 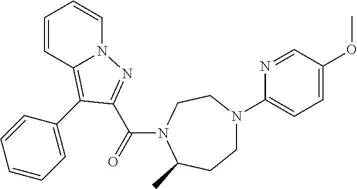 [SMILES: COc1ccc(N2CC[C@@H](C)N(C(=O)c3nn4ccccc4c3-c3ccccc3)CC2)nc1] | 90 | 87/18 | 52 | | 441.53 |
| 79 | 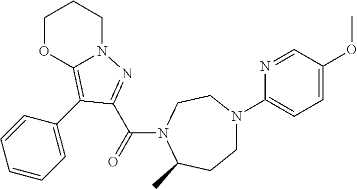 [SMILES: COc1ccc(N2CC[C@@H](C)N(C(=O)c3nn4c(c3-c3ccccc3)OCCC4)CC2)nc1] | 45 | | 21 | | 447.53 |
| 80 | 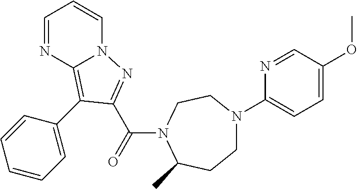 [SMILES: COc1ccc(N2CC[C@@H](C)N(C(=O)c3nn4cccnc4c3-c3ccccc3)CC2)nc1] | 78 | 500/110 | 39 | | 442.51 |
| 81 | 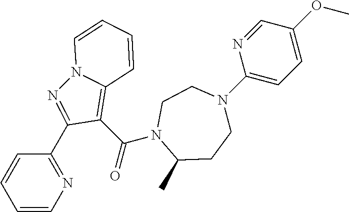 [SMILES: COc1ccc(N2CC[C@@H](C)N(C(=O)c3c(-c4ccccn4)nn4ccccc34)CC2)nc1] | 76 | 147/31 | 47 | | 442.51 |
| 82 | 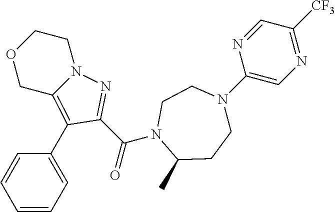 [SMILES: C[C@@H]1CCN(c2cnc(C(F)(F)F)cn2)CCN1C(=O)c1nn2c(c1-c1ccccc1)COCC2] | 39 | | 2 | | 486.49 |
| 83 | 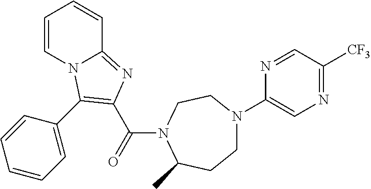 [SMILES: C[C@@H]1CCN(c2cnc(C(F)(F)F)cn2)CCN1C(=O)c1nc2ccccn2c1-c1ccccc1] | 69 | | 27 | | 480.49 |
| 84 | 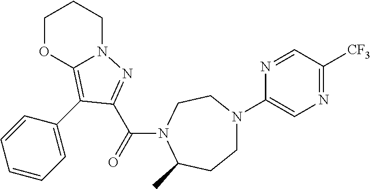 [SMILES: C[C@@H]1CCN(c2cnc(C(F)(F)F)cn2)CCN1C(=O)c1nn2c(c1-c1ccccc1)OCCC2] | 71 | 323/68 | 27 | | 486.49 |
| 85 | 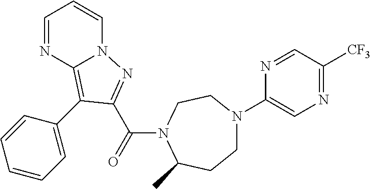 [SMILES: C[C@@H]1CCN(c2cnc(C(F)(F)F)cn2)CCN1C(=O)c1nn2cccnc2c1-c1ccccc1] | 93 | 77/16 | 52 | | 481.47 |
| 86 | 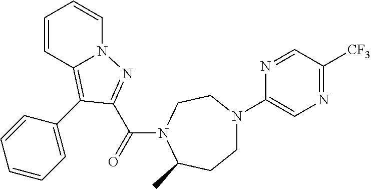 [SMILES: C[C@@H]1CCN(c2cnc(C(F)(F)F)cn2)CCN1C(=O)c1nn2ccccc2c1-c1ccccc1] | 89 | 96/20 | 63 | | 480.49 |
| 87 | 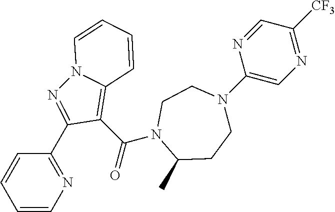 [SMILES: C[C@@H]1CCN(c2cnc(C(F)(F)F)cn2)CCN1C(=O)c1c(-c2ccccn2)nn2ccccc12] | 83 | 134/28 | 57 | | 481.47 |
| 88 | 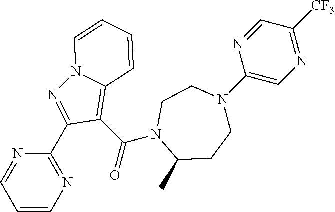 [SMILES: C[C@@H]1CCN(c2cnc(C(F)(F)F)cn2)CCN1C(=O)c1c(-c2ncccn2)nn2ccccc12] | 51 | | 29 | | 482.46 |
| 89 | 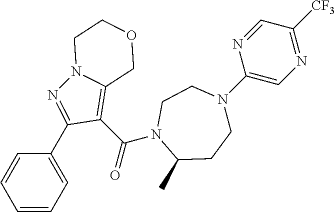 [SMILES: C[C@@H]1CCN(c2cnc(C(F)(F)F)cn2)CCN1C(=O)c1c(-c2ccccc2)nn2c1COCC2] | 25 | | 10 | | 486.49 |
| 90 | 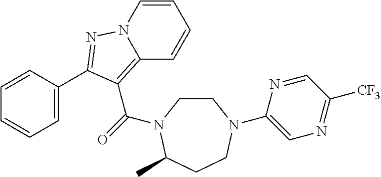 [SMILES: C[C@@H]1CCN(c2cnc(C(F)(F)F)cn2)CCN1C(=O)c1c(-c2ccccc2)nn2ccccc12] | 95 | 48/10 | 80 | 258/45 | 480.49 |
| 91 | 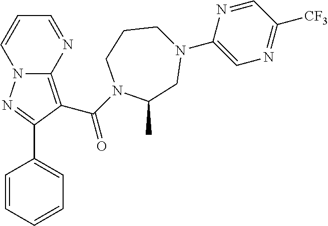 [SMILES: C[C@@H]1CN(c2cnc(C(F)(F)F)cn2)CCCN1C(=O)c1c(-c2ccccc2)nn2cccnc12] | 87 | 52/11 | 74 | | 481.47 |
| 92 | 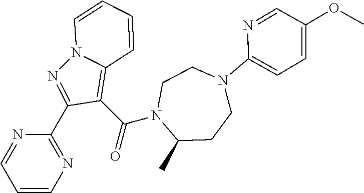 [SMILES: COc1ccc(N2CC[C@@H](C)N(C(=O)c3c(-c4ncccn4)nn4ccccc34)CC2)nc1] | 25 | | 13 | | 443.50 |
| 93 | 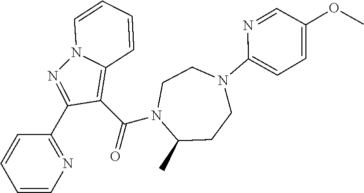 [SMILES: COc1ccc(N2CC[C@@H](C)N(C(=O)c3c(-c4ccccn4)nn4ccccc34)CC2)nc1] | 36 | | 33 | | 442.51 |
| 94 | 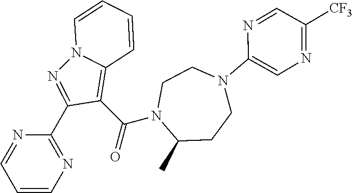 [SMILES: C[C@@H]1CCN(c2cnc(C(F)(F)F)cn2)CCN1C(=O)c1c(-c2ncccn2)nn2ccccc12] | 43 | | 21 | | 482.46 |
| 95 | 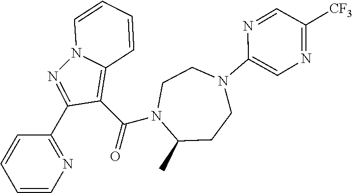 [SMILES: C[C@@H]1CCN(c2cnc(C(F)(F)F)cn2)CCN1C(=O)c1c(-c2ccccn2)nn2ccccc12] | 86 | 59/12 | 61 | | 481.47 |
| 96 | 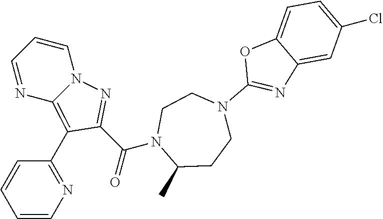 [SMILES: C[C@@H]1CCN(c2nc3cc(Cl)ccc3o2)CCN1C(=O)c1nn2cccnc2c1-c1ccccn1] | 100 | 61/13 | 71 | | 487.94 |
| 97 | 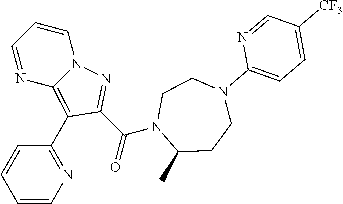 [SMILES: C[C@@H]1CCN(c2ccc(C(F)(F)F)cn2)CCN1C(=O)c1nn2cccnc2c1-c1ccccn1] | 98 | 74/16 | 51 | | 481,47 |
| 98 | 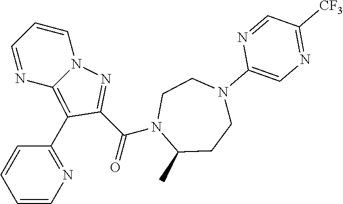 [SMILES: C[C@@H]1CCN(c2cnc(C(F)(F)F)cn2)CCN1C(=O)c1nn2cccnc2c1-c1ccccn1] | 57 | | 26 | | 482.46 |
| 99 | 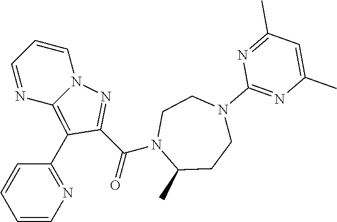 [SMILES: Cc1cc(C)nc(N2CC[C@@H](C)N(C(=O)c3nn4cccnc4c3-c3ccccn3)CC2)n1] | 45 | | 56 | | 442.52 |
| 100 | 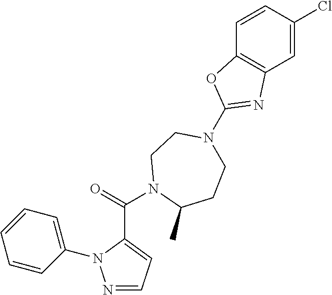 [SMILES: C[C@@H]1CCN(c2nc3cc(Cl)ccc3o2)CCN1C(=O)c1ccnn1-c1ccccc1] | 54 | | 57 | | 435.91 |
| 101 | 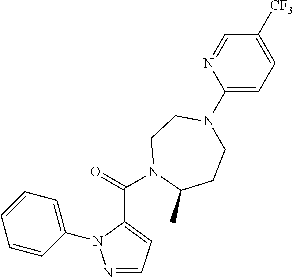 [SMILES: C[C@@H]1CCN(c2ccc(C(F)(F)F)cn2)CCN1C(=O)c1ccnn1-c1ccccc1] | 19 | | 32 | | 429.44 |

While certain embodiments have been described in terms of the preferred embodiments, it is understood that variations and modifications will occur to those skilled in the art. Therefore, it is intended that the appended claims cover all such equivalent variations that come within the scope of the following claims.

What is claimed is:

1. A compound of Formulas (I) or (II), 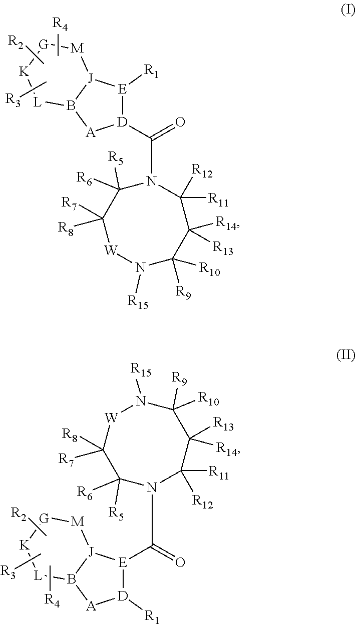
or a pharmaceutically acceptable salt, hydrate, solvate, polymorph, isomer, or combination thereof; wherein:
wherein:
R₁, includes E in which is Carbon (C) but not Nitrogen (N), and E is linked to J or D with a double bond, and R₁ is selected from the group consisting of H, alkyl, alkoxy, cycloalkyl, phenyl, aromatic or aryl, heteroaryl (5-6 membered ring), substituted aromatic or aryl, substituted heteroaryl (5-6 membered ring); or, when R₁ is heteroaryl, R₁ is preferably a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, and R₄ are independently selected from the group consisting of H, halogen (such as F, Cl, Br), alkyl, substituted alkyl, (C₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to three (3) substituents independently selected from one, two, or all R₂, R₃ and R₄;
R₅ and R₆ are independently selected from the group consisting of H, CH, alkyl, substituted alkyl, (C₁₋₃)fluoroalkyl, cycloalkyl, R₆ connected to either R₁₀ or R₁₁ or R₁₃ as alkyl to form a (C₁₋₃)alkyl bridge cyclic structure, and R₅ and R₆ linked together to form a spiro moiety such as but not limited to cyclopropyl, or cyclobutyl;
R₇ is selected from the group consisting of H, CH₃, alkyl, substituted alkyl, (C₁₋₃)fluoroalkyl, cycloalkyl, and R₇ connected to either R₁₀ or R₁₁ or R₁₃ as alkyl to form a (C₁₋₃)alkyl bridge cyclic structure;
R₈ is selected from the group consisting of H, CH₃, alkyl, substituted alkyl, (C₁₋₃)fluoroalkyl, cycloalkyl, and R₈ connected to either R₁₀ or R₁₁ or R₁₃ as alkyl to form a (C₁₋₃)alkyl bridge cyclic structure;
R₉ is selected from the group consisting of H, CH₃, alkyl, substituted alkyl, (C₁₋₃)fluoroalkyl, cycloalkyl, and R₉ connected to either R₁₀ or R₁₁ as alkyl to form a (C₁₋₃)alkyl bridge cyclic structure;
R₁₀ is selected from the group consisting of H, CH₃, alkyl, substituted alkyl, (C₁₋₃)fluoroalkyl, and cycloalkyl;
R₁₁ and R₁₂ are independently selected from the group consisting of H, CH₃, alkyl, substituted alkyl, (C₁₋₃)fluoroalkyl, cycloalkyl, and R₁₁ or R₁₂ are connected to either R₇ or R₈ as alkyl to form a (C₁₋₃)alkyl bridge cyclic structure, wherein in some embodiments R₁₁ and R₁₂ linked together can form a spiro moiety such as but not limited to cyclopropyl or cyclobutyl;
R₁₃ and R₁₄ are independently selected from the group consisting of H, CH₃, alkyl, substituted alkyl (e.g., Fluoroalkyl), cycloalkyl, and R₁₃ or R₁₄ are connected to either R₇ or R₈ as alkyl to form a (C₁₋₃)alkyl bridge cyclic structure;
R₁₅ is aromatic or aryl, such as but not limited to heteroaryl (e.g., preferably a 5 or 6 membered ring), substituted aromatic or aryl, substituted heteroaryl (e.g., preferably a 5 or 6 membered ring), and fused two heteroaryl ring system; wherein said aromatic, aryl or heteroaryl is unsubstituted, mono-, or di-substituted or tri-substituted, and wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl;
W is:
c) selected from the group consisting of CH₂, and R₇, R₈, R₁₃, R₁₄=H, the carbons bearing substituents R₇, R₈, R₁₃, and R₁₄ would be joined directly to provide a 5-5 bridged bicyclic ring system shown below: 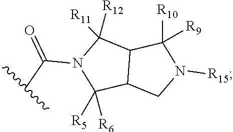
d) absent to provide seven membered ring system shown below: 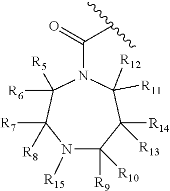
wherein:
fused ring system A-B-J-D-E is five (5) membered heteroaryl, optionally imidazole wherein A and J are Nitrogen while B, E, and D are Carbon, or pyrazole wherein A and B are Nitrogen and D, E, and J are Carbon; said fused or unfused with an additional ring systems;
fused ring system B-J-M-G-K-L is a six (6) membered aromatic or aryl, substituted aromatic or aryl, substituted or unsubstituted heteroaryl, cycloalkyl, heterocycloalkyl;
A is Nitrogen or Carbon:
B is Carbon or Nitrogen:
J is Carbon or Nitrogen;
D is Carbon;
E is Carbon;
M is selected from the group consisting of Carbon, CH, CHR₂, CHR₃, CR₂R₃, CR₂, CR₃, CR₄, O, and N:
G is selected from the group consisting of Carbon, CH, CHR₂, CHR₃, CR₂R₃, CR₂, CR₃, CR₄, and O;
K is selected from the group consisting of Carbon, CH, CHR₂, CHR₃, CR₂R₃, CR₂, CR₃, CR₄, and O;
L selected from the group consisting of Carbon, CH, CHR₂, CHR₃, CR₂R₃, CR₂, CR₃, CR₄, O, and N.

2. A compound of claim 1 of Formula I-a7, Formula I-a8, Formula I-a9, Formula II-a7, Formula II-a8, or Formula II-a9: 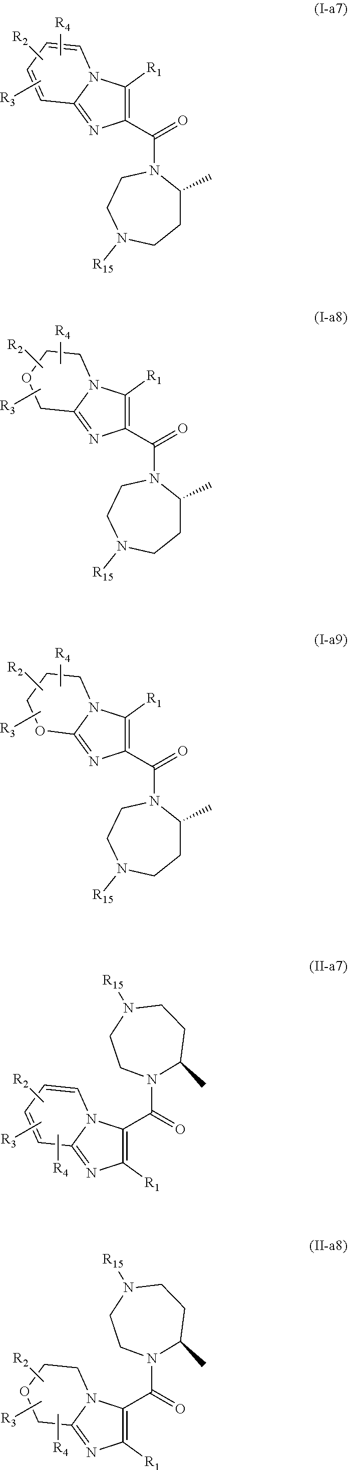 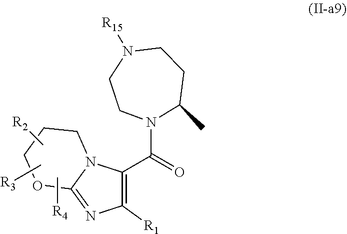
or a pharmaceutically acceptable salt, hydrate, solvate, polymorph, isomer, or combination thereof, wherein:
R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl (5-6 membered ring), substituted aromatic or aryl, substituted heteroaryl (5-6 membered ring); when R₁ is heteroaryl, it is preferred as a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, and (C₃₋₇)cycloalkyl;
R₂, R₃, and R₄ are independently selected from the group consisting of H, halogen (such as F, Cl, Br), alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to 3 substituents independently selected from one or two or all R₂, R₃ & R₄; and,
R₁₅ is selected from the group consisting of aromatic or aryl, heteroaryl, a 5-6 membered heteroaryl, substituted aromatic or aryl, a substituted 5-6 membered heteroaryl or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

3. A compound of claim 1 having Formula I-b7, Formula I-b8, Formula I-b9, Formula I-b10, Formula II-b7, Formula II-b8, Formula II-b9 and Formula I-b10: 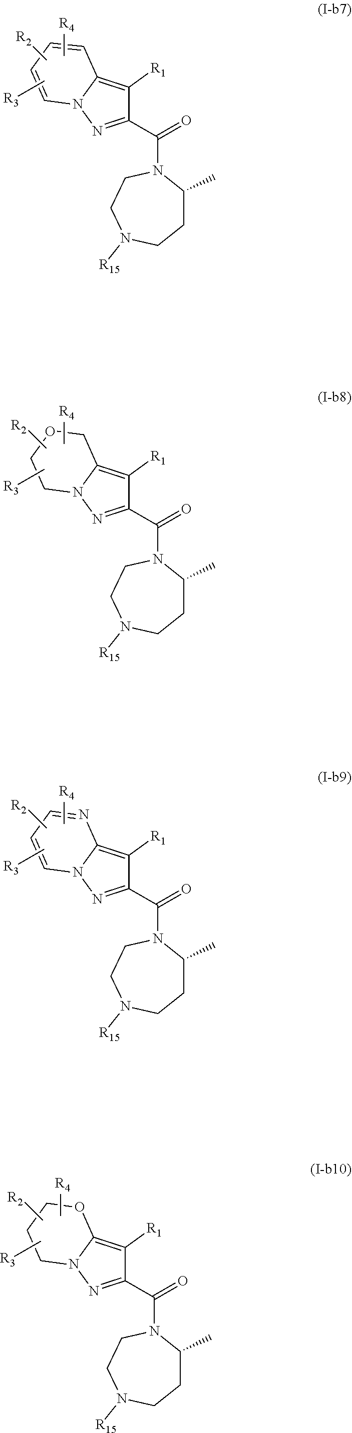 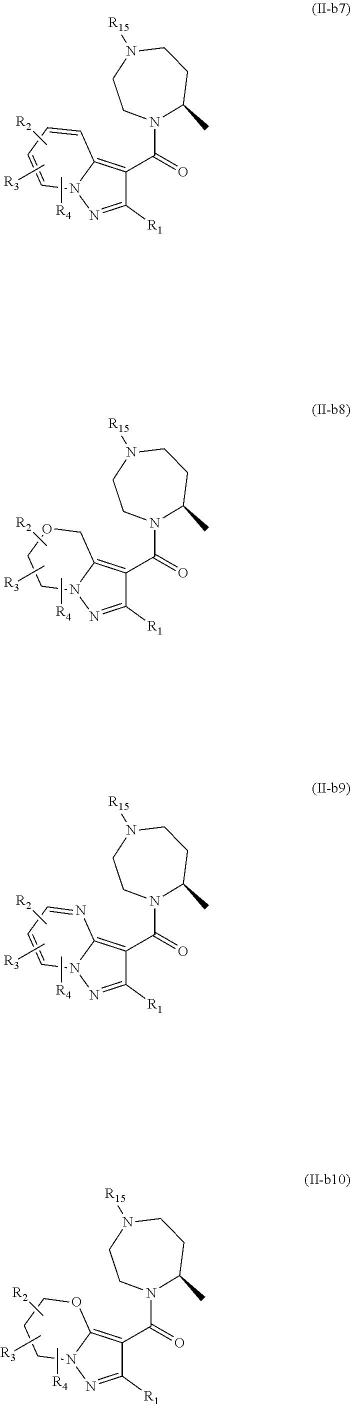
or a pharmaceutically acceptable salt, hydrate, solvate, polymorph, isomer, or combination thereof, wherein:
R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl, a 5-6 membered heteroaryl, substituted aromatic or aryl, substituted heteroaryl, substituted 5-6 membered heteroaryl; when R₁ is heteroaryl, it is optionally a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, and (C₃₋₇)cycloalkyl;
R₂, R₃, R₄ are each independently selected from the group consisting of H, halogen that is optionally F, Cl, or Br, alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R_{Z}, R₃ and R₄ is independently and optionally substituted at each substitutable position with up to 3 substituents independently selected from one or two or all R₂, R₃ and RR; and,
R₁₅ is selected from the group consisting of aromatic or aryl, heteroaryl, 5-6 membered heteroaryl, substituted aromatic or aryl, substituted heteroaryl, substituted 5-6 membered heteroaryl and a fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, (C₃₋₇)heterocycloalkyl.

4. A compound of claim 1 having Formula I-b11, Formula I-b12, Formula I-b13, Formula I-b14, Formula II-b11, Formula II-b12, Formula II-b13 and Formula II-b14: 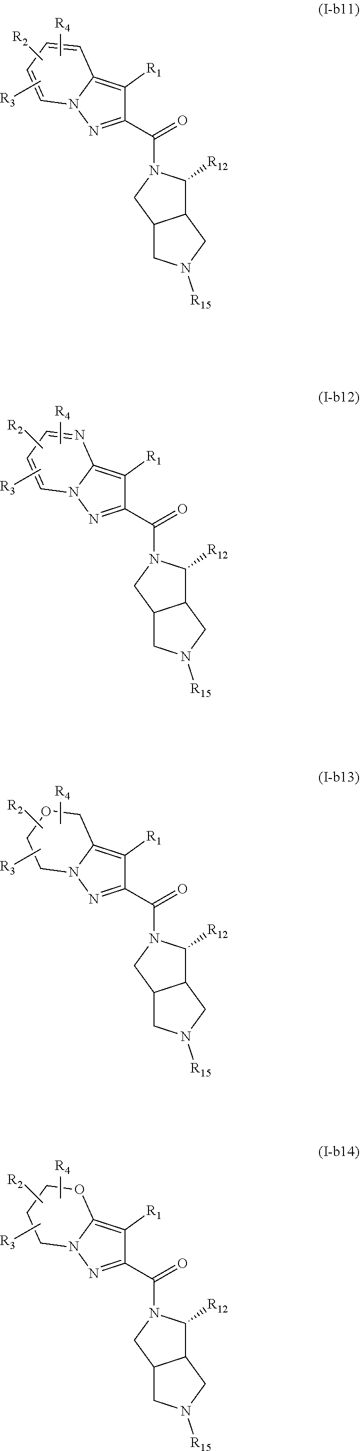 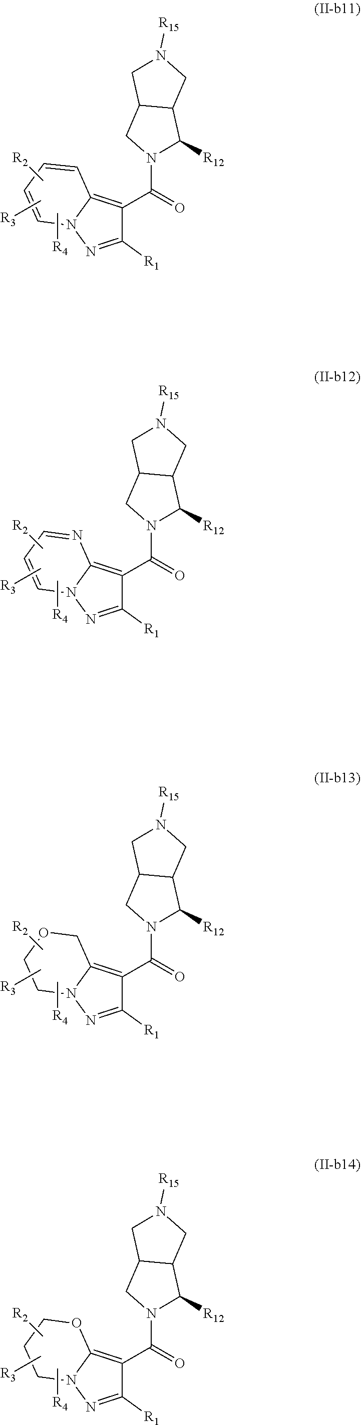
or a pharmaceutically acceptable salt, hydrate, solvate, polymorph, isomer, or combination thereof, wherein:
R₁, in which E is Carbon (C) not Nitrogen (N), and E is linked to J or D with a double bond, is selected from the group consisting of H, alkyl, alkoxy, cycloalkyl, Phenyl, aromatic or aryl, heteroaryl, 5-6 membered heteroaryl, substituted aromatic or aryl, substituted heteroaryl, substituted 5-6 membered heteroaryl; when R₁ is heteroaryl, optionally a 5 or 6-membered heteroaryl selected from the group consisting of pyrazolyl, triazolyl, oxazolyl, thiazolyl, thiophenyl, pyridinyl, pyrimidinyl, pyrazinyl, and pyridazinyl; wherein said aromatic, aryl or heteroaryl is unsubstituted, mono-, or di-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy and (C₃₋₇)cycloalkyl;
R₂, R₃, R₄ are each independently selected from the group consisting of H, halogen, F, Cl, or Br, alkyl group, substituted alkyl, (C₁₋₄)alkyl, (C₁₋₄)alkoxy, (C₁₋₃)fluoroalkyl, (C₁₋₃)fluoroalkoxy and (C₃₋₇)cycloalkyl; wherein each of R₂, R₃ and R₄ is independently and optionally substituted at each substitutable position with up to 3 substituents independently selected from one or two or all R₂, R₃ and R₄;
R₁₂ is selected from the group consisting of H, CH₃, alkyl, substituted alkyl, (C₁₋₃)fluoroalkyl, and cycloalkyl; and,
R₁₆ is selected from the group consisting of aromatic, aryl, heteroaryl, 5-6 membered heteroaryl, substituted aromatic, substituted aryl, substituted heteroaryl, substituted 5-6 membered heteroaryl or fused two heteroaryl ring system; wherein said aromatic, aryl or heteroaryl is unsubstituted, mono-, or di-substituted or tri-substituted, wherein the substituents are independently selected from the group consisting of (C₁₋₄)alkyl, (C₁₋₄)alkoxy, halogen, (C₁₋₃)fluoroalkyl, (C₁₋₃)fluoroalkoxy, (C₃₋₇)cycloalkyl, and (C₃₋₇)heterocycloalkyl.

5. A compound of claim 1 selected from the group consisting of 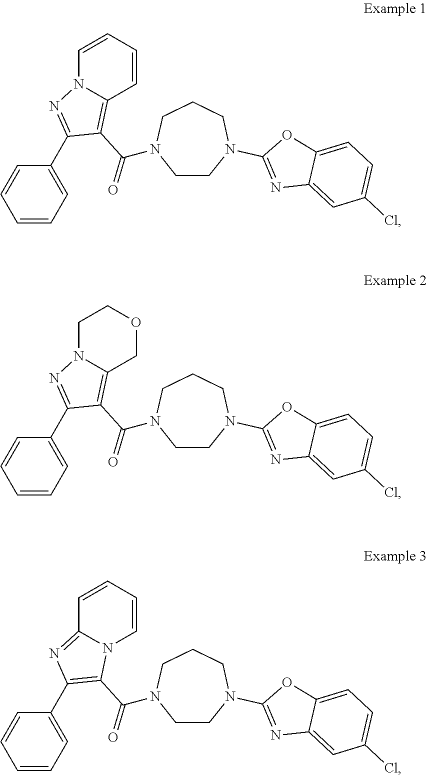 [SMILES: O=C(c1c(-c2ccccc2)nn2ccccc12)N1CCCN(c2nc3cc(Cl)ccc3o2)CC1 ; O=C(c1c(-c2ccccc2)nn2c1COCC2)N1CCCN(c2nc3cc(Cl)ccc3o2)CC1 ; O=C(c1c(-c2ccccc2)nc2ccccn12)N1CCCN(c2nc3cc(Cl)ccc3o2)CC1] 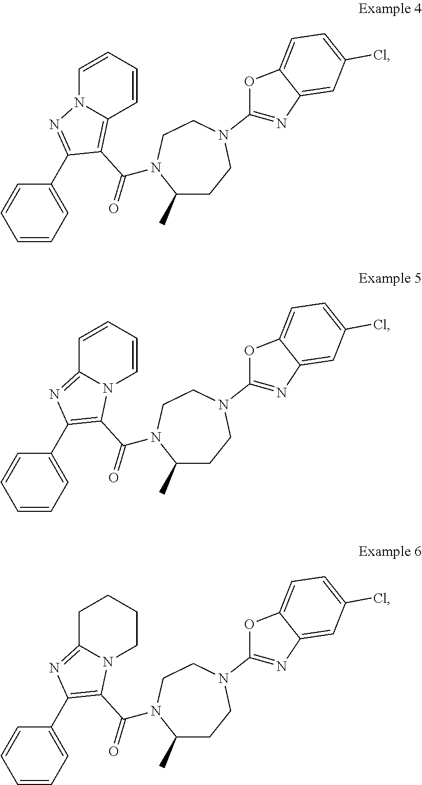 [SMILES: C[C@@H]1CCN(c2nc3cc(Cl)ccc3o2)CCN1C(=O)c1c(-c2ccccc2)nn2ccccc12 ; C[C@@H]1CCN(c2nc3cc(Cl)ccc3o2)CCN1C(=O)c1c(-c2ccccc2)nc2ccccn12 ; C[C@@H]1CCN(c2nc3cc(Cl)ccc3o2)CCN1C(=O)c1c(-c2ccccc2)nc2n1CCCC2] 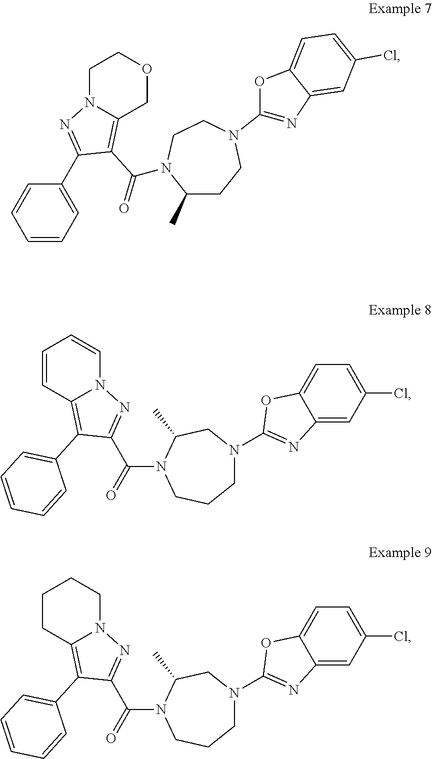 [SMILES: C[C@@H]1CCN(c2nc3cc(Cl)ccc3o2)CCN1C(=O)c1c(-c2ccccc2)nn2c1COCC2 ; C[C@@H]1CN(c2nc3cc(Cl)ccc3o2)CCCN1C(=O)c1nn2ccccc2c1-c1ccccc1 ; C[C@@H]1CN(c2nc3cc(Cl)ccc3o2)CCCN1C(=O)c1nn2c(c1-c1ccccc1)CCCC2] 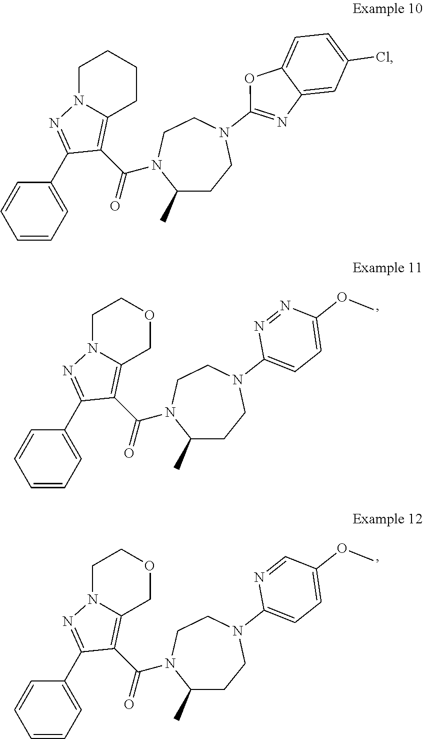 [SMILES: C[C@@H]1CCN(c2nc3cc(Cl)ccc3o2)CCN1C(=O)c1c(-c2ccccc2)nn2c1CCCC2 ; COc1ccc(N2CC[C@@H](C)N(C(=O)c3c(-c4ccccc4)nn4c3COCC4)CC2)nn1 ; COc1ccc(N2CC[C@@H](C)N(C(=O)c3c(-c4ccccc4)nn4c3COCC4)CC2)nc1] 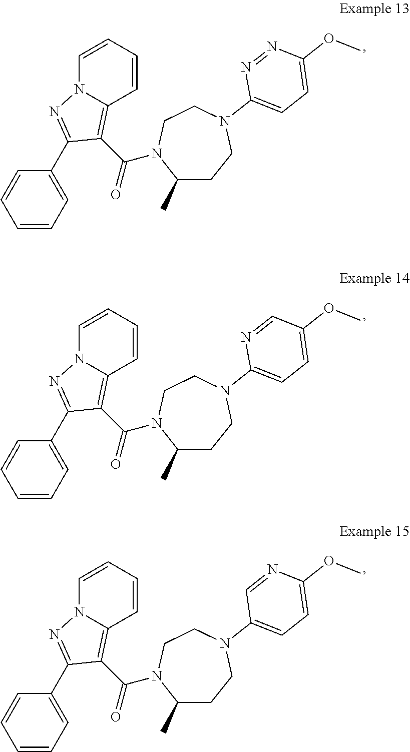 [SMILES: COc1ccc(N2CC[C@@H](C)N(C(=O)c3c(-c4ccccc4)nn4ccccc34)CC2)nn1 ; COc1ccc(N2CC[C@@H](C)N(C(=O)c3c(-c4ccccc4)nn4ccccc34)CC2)nc1 ; COc1ccc(N2CC[C@@H](C)N(C(=O)c3c(-c4ccccc4)nn4ccccc34)CC2)cn1] 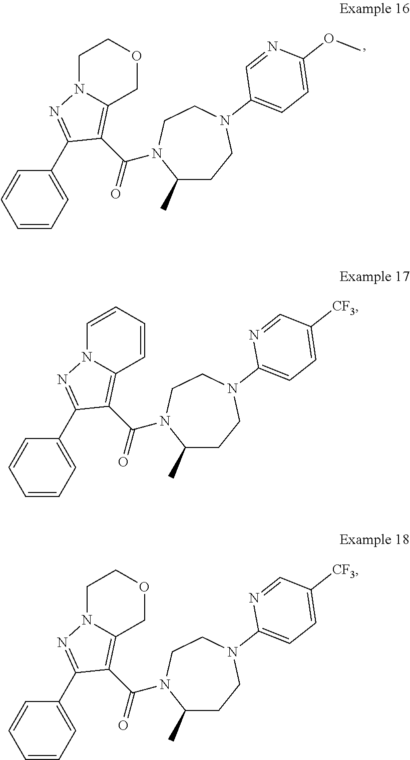 [SMILES: COc1ccc(N2CC[C@@H](C)N(C(=O)c3c(-c4ccccc4)nn4c3COCC4)CC2)cn1 ; C[C@@H]1CCN(c2ccc(C(F)(F)F)cn2)CCN1C(=O)c1c(-c2ccccc2)nn2ccccc12 ; C[C@@H]1CCN(c2ccc(C(F)(F)F)cn2)CCN1C(=O)c1c(-c2ccccc2)nn2c1COCC2] 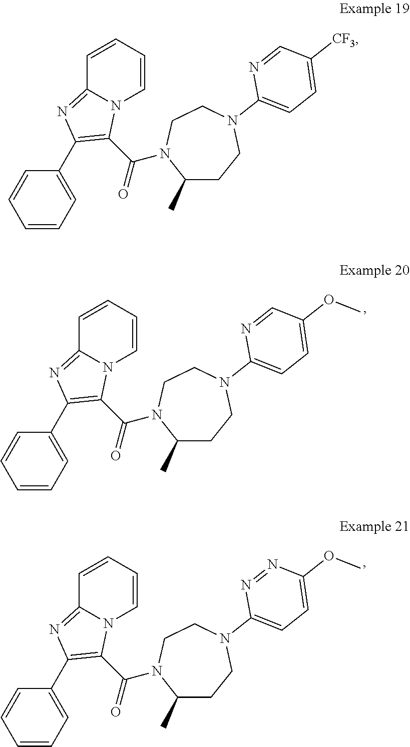 [SMILES: C[C@@H]1CCN(c2ccc(C(F)(F)F)cn2)CCN1C(=O)c1c(-c2ccccc2)nc2ccccn12 ; COc1ccc(N2CC[C@@H](C)N(C(=O)c3c(-c4ccccc4)nc4ccccn34)CC2)nc1 ; COc1ccc(N2CC[C@@H](C)N(C(=O)c3c(-c4ccccc4)nc4ccccn34)CC2)nn1] 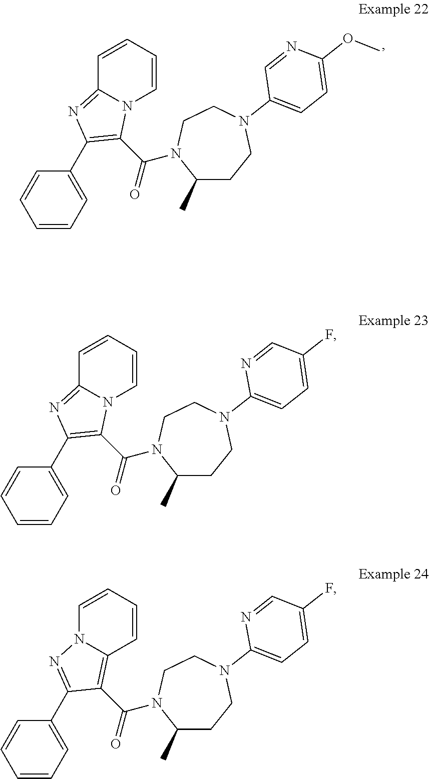 [SMILES: COc1ccc(N2CC[C@@H](C)N(C(=O)c3c(-c4ccccc4)nc4ccccn34)CC2)cn1 ; C[C@@H]1CCN(c2ccc(F)cn2)CCN1C(=O)c1c(-c2ccccc2)nc2ccccn12 ; C[C@@H]1CCN(c2ccc(F)cn2)CCN1C(=O)c1c(-c2ccccc2)nn2ccccc12] 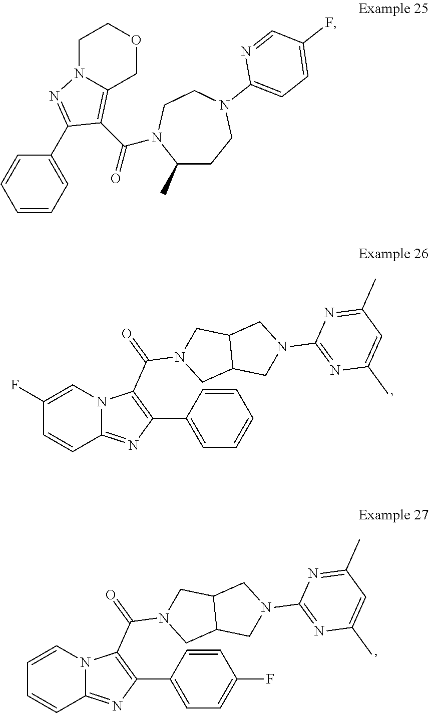 [SMILES: C[C@@H]1CCN(c2ccc(F)cn2)CCN1C(=O)c1c(-c2ccccc2)nn2c1COCC2 ; Cc1cc(C)nc(N2CC3CN(C(=O)c4c(-c5ccccc5)nc5ccc(F)cn45)CC3C2)n1 ; Cc1cc(C)nc(N2CC3CN(C(=O)c4c(-c5ccc(F)cc5)nc5ccccn45)CC3C2)n1] 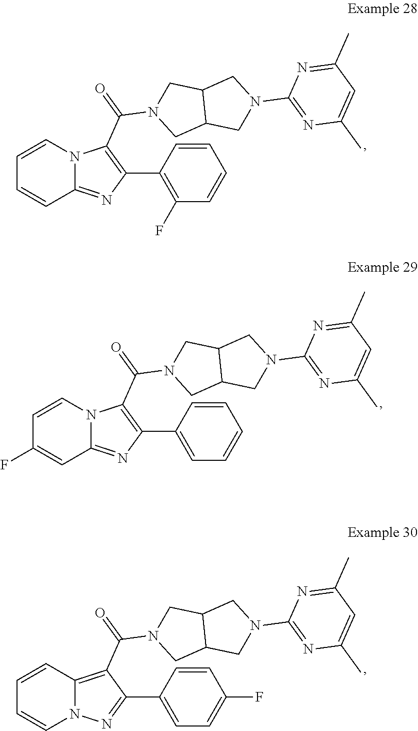 [SMILES: Cc1cc(C)nc(N2CC3CN(C(=O)c4c(-c5ccccc5F)nc5ccccn45)CC3C2)n1 ; Cc1cc(C)nc(N2CC3CN(C(=O)c4c(-c5ccccc5)nc5cc(F)ccn45)CC3C2)n1 ; Cc1cc(C)nc(N2CC3CN(C(=O)c4c(-c5ccc(F)cc5)nn5ccccc45)CC3C2)n1] 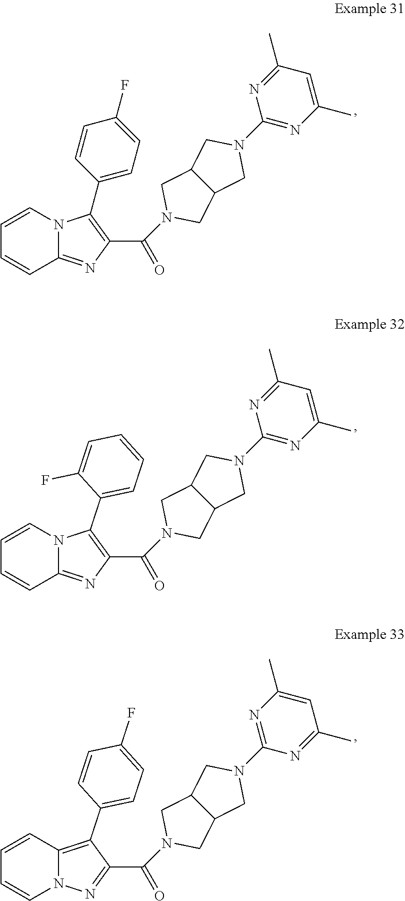 [SMILES: Cc1cc(C)nc(N2CC3CN(C(=O)c4nc5ccccn5c4-c4ccc(F)cc4)CC3C2)n1 ; Cc1cc(C)nc(N2CC3CN(C(=O)c4nc5ccccn5c4-c4ccccc4F)CC3C2)n1 ; Cc1cc(C)nc(N2CC3CN(C(=O)c4nn5ccccc5c4-c4ccc(F)cc4)CC3C2)n1] 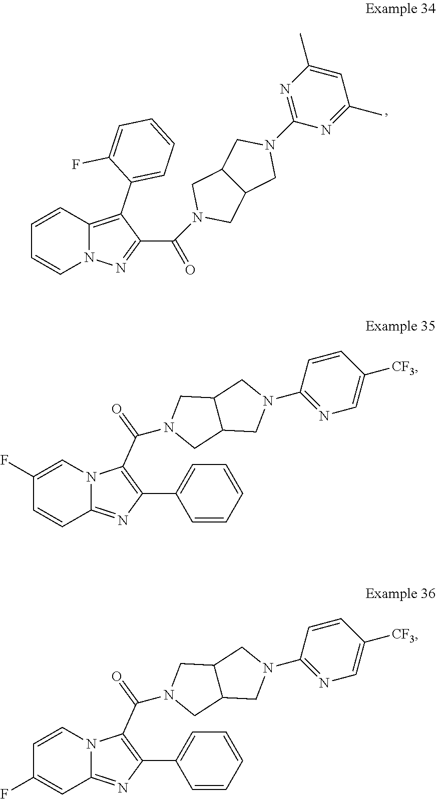 [SMILES: Cc1cc(C)nc(N2CC3CN(C(=O)c4nn5ccccc5c4-c4ccccc4F)CC3C2)n1 ; O=C(c1c(-c2ccccc2)nc2ccc(F)cn12)N1CC2CN(c3ccc(C(F)(F)F)cn3)CC2C1 ; O=C(c1c(-c2ccccc2)nc2cc(F)ccn12)N1CC2CN(c3ccc(C(F)(F)F)cn3)CC2C1] 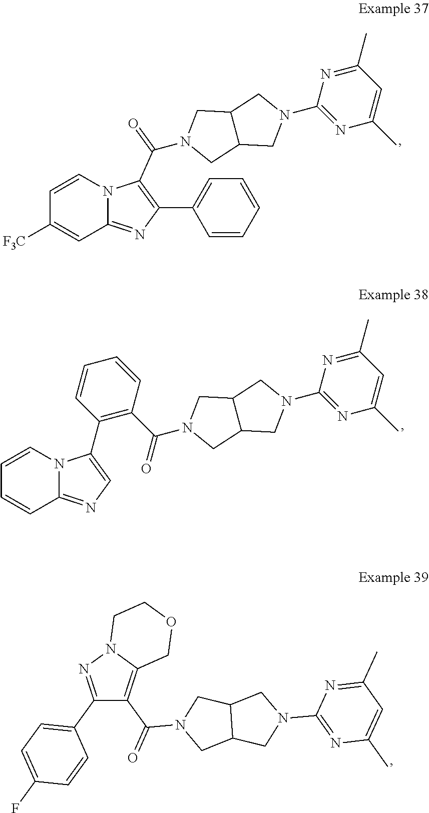 [SMILES: Cc1cc(C)nc(N2CC3CN(C(=O)c4c(-c5ccccc5)nc5cc(C(F)(F)F)ccn45)CC3C2)n1 ; Cc1cc(C)nc(N2CC3CN(C(=O)c4ccccc4-c4cnc5ccccn45)CC3C2)n1 ; Cc1cc(C)nc(N2CC3CN(C(=O)c4c(-c5ccc(F)cc5)nn5c4COCC5)CC3C2)n1] 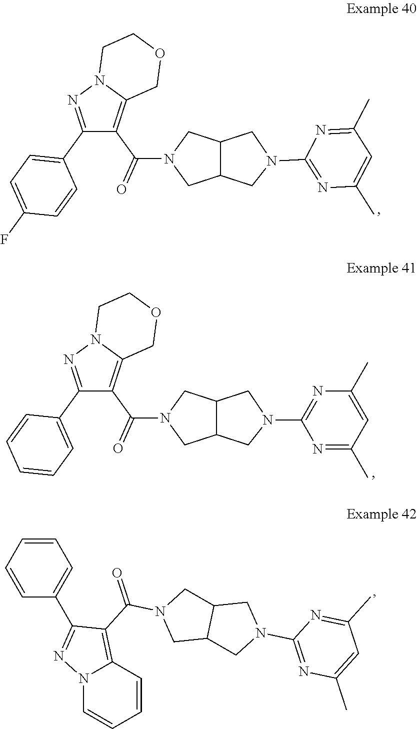 [SMILES: Cc1cc(C)nc(N2CC3CN(C(=O)c4c(-c5ccc(F)cc5)nn5c4COCC5)CC3C2)n1 ; Cc1cc(C)nc(N2CC3CN(C(=O)c4c(-c5ccccc5)nn5c4COCC5)CC3C2)n1 ; Cc1cc(C)nc(N2CC3CN(C(=O)c4c(-c5ccccc5)nn5ccccc45)CC3C2)n1] 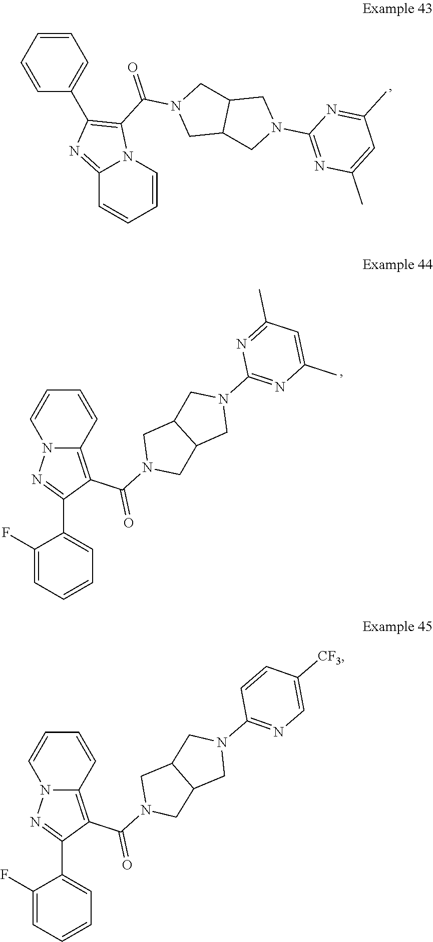 [SMILES: Cc1cc(C)nc(N2CC3CN(C(=O)c4c(-c5ccccc5)nc5ccccn45)CC3C2)n1 ; Cc1cc(C)nc(N2CC3CN(C(=O)c4c(-c5ccccc5F)nn5ccccc45)CC3C2)n1 ; O=C(c1c(-c2ccccc2F)nn2ccccc12)N1CC2CN(c3ccc(C(F)(F)F)cn3)CC2C1] 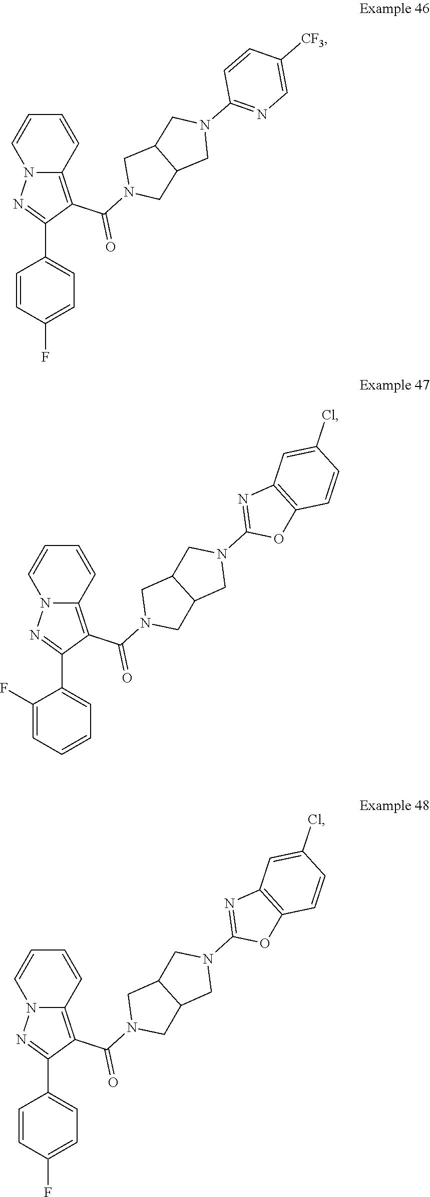 [SMILES: O=C(c1c(-c2ccc(F)cc2)nn2ccccc12)N1CC2CN(c3ccc(C(F)(F)F)cn3)CC2C1 ; O=C(c1c(-c2ccccc2F)nn2ccccc12)N1CC2CN(c3nc4cc(Cl)ccc4o3)CC2C1 ; O=C(c1c(-c2ccc(F)cc2)nn2ccccc12)N1CC2CN(c3nc4cc(Cl)ccc4o3)CC2C1] 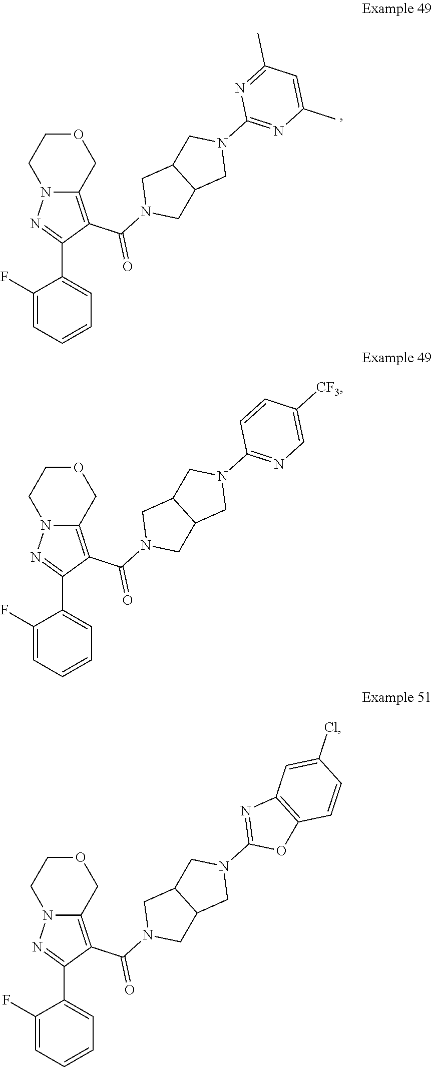 [SMILES: Cc1cc(C)nc(N2CC3CN(C(=O)c4c(-c5ccccc5F)nn5c4COCC5)CC3C2)n1 ; O=C(c1c(-c2ccccc2F)nn2c1COCC2)N1CC2CN(c3ccc(C(F)(F)F)cn3)CC2C1 ; O=C(c1c(-c2ccccc2F)nn2c1COCC2)N1CC2CN(c3nc4cc(Cl)ccc4o3)CC2C1] 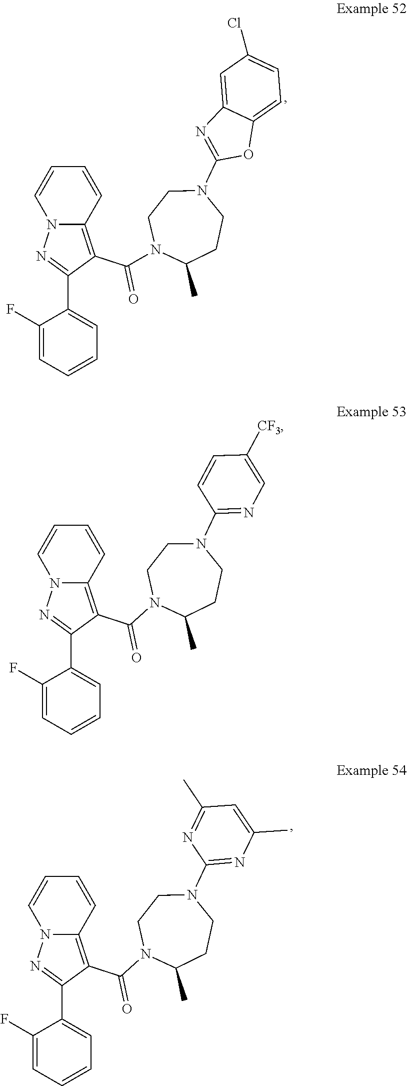 [SMILES: C[C@@H]1CCN(c2nc3cc(Cl)ccc3o2)CCN1C(=O)c1c(-c2ccccc2F)nn2ccccc12 ; C[C@@H]1CCN(c2ccc(C(F)(F)F)cn2)CCN1C(=O)c1c(-c2ccccc2F)nn2ccccc12 ; Cc1cc(C)nc(N2CC[C@@H](C)N(C(=O)c3c(-c4ccccc4F)nn4ccccc34)CC2)n1] 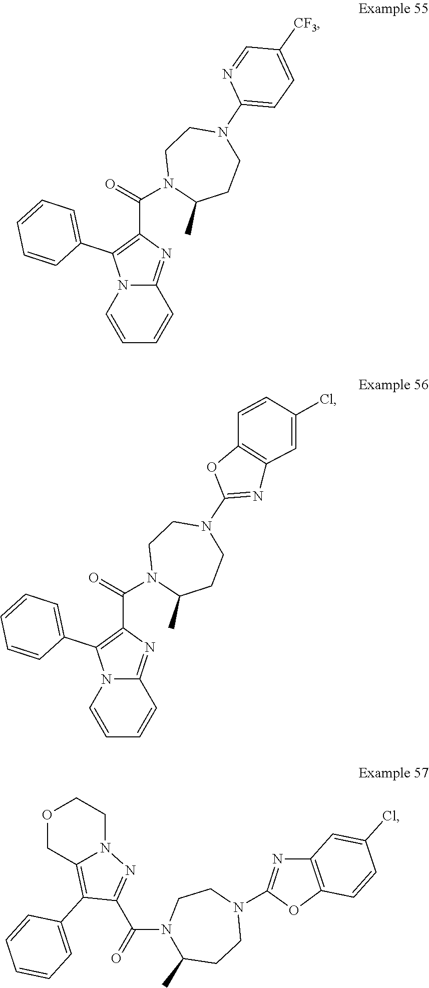 [SMILES: C[C@@H]1CCN(c2ccc(C(F)(F)F)cn2)CCN1C(=O)c1nc2ccccn2c1-c1ccccc1 ; C[C@@H]1CCN(c2nc3cc(Cl)ccc3o2)CCN1C(=O)c1nc2ccccn2c1-c1ccccc1 ; C[C@@H]1CCN(c2nc3cc(Cl)ccc3o2)CCN1C(=O)c1nn2c(c1-c1ccccc1)COCC2] 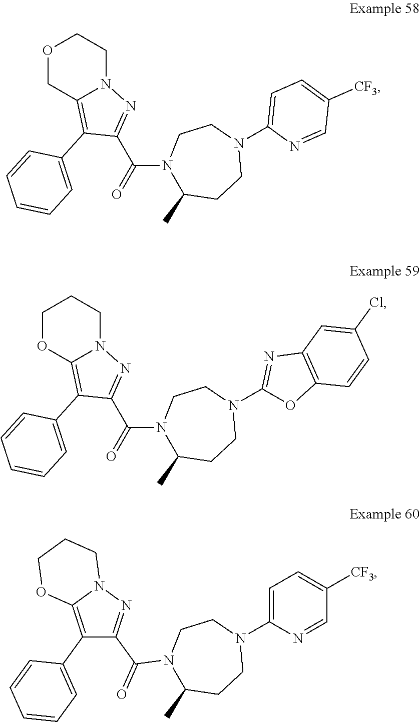 [SMILES: C[C@@H]1CCN(c2ccc(C(F)(F)F)cn2)CCN1C(=O)c1nn2c(c1-c1ccccc1)COCC2 ; C[C@@H]1CCN(c2nc3cc(Cl)ccc3o2)CCN1C(=O)c1nn2c(c1-c1ccccc1)OCCC2 ; C[C@@H]1CCN(c2ccc(C(F)(F)F)cn2)CCN1C(=O)c1nn2c(c1-c1ccccc1)OCCC2] 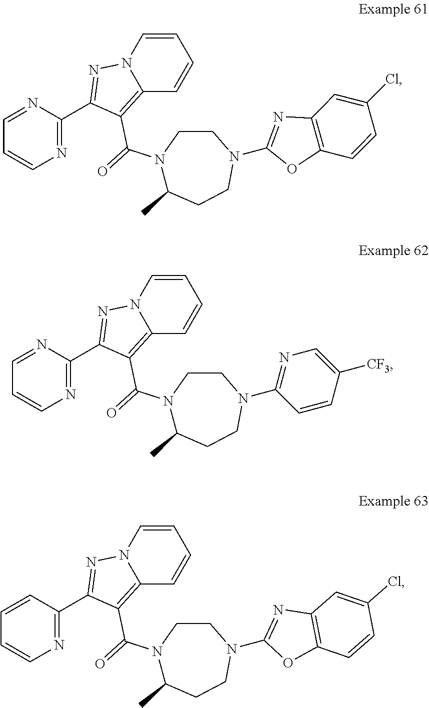 [SMILES: C[C@@H]1CCN(c2nc3cc(Cl)ccc3o2)CCN1C(=O)c1c(-c2ncccn2)nn2ccccc12 ; C[C@@H]1CCN(c2ccc(C(F)(F)F)cn2)CCN1C(=O)c1c(-c2ncccn2)nn2ccccc12 ; C[C@@H]1CCN(c2nc3cc(Cl)ccc3o2)CCN1C(=O)c1c(-c2ccccn2)nn2ccccc12] 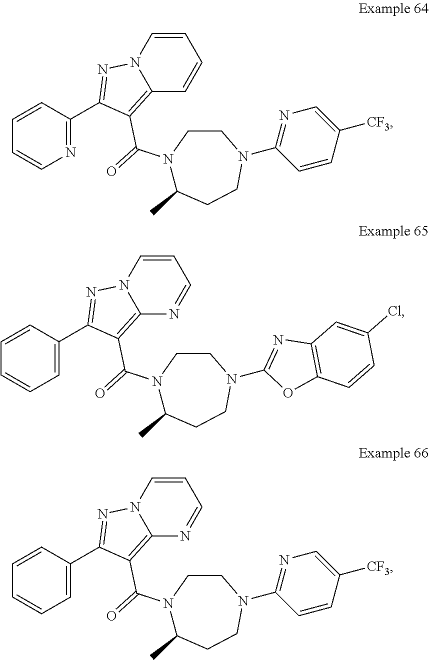 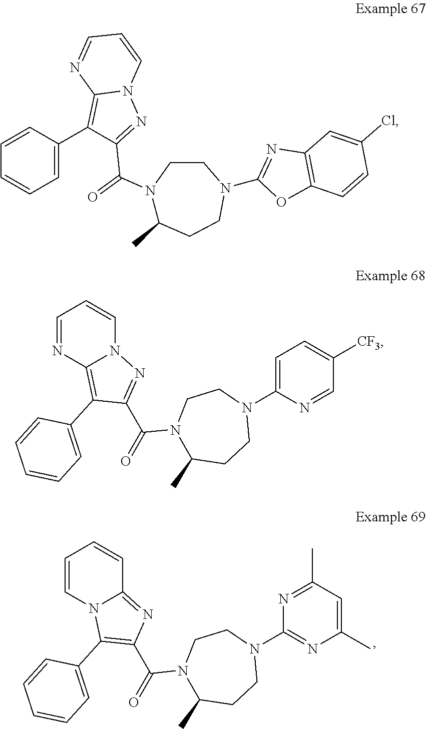 [SMILES: C[C@@H]1CCN(c2nc3cc(Cl)ccc3o2)CCN1C(=O)c1nn2cccnc2c1-c1ccccc1 ; C[C@@H]1CCN(c2ccc(C(F)(F)F)cn2)CCN1C(=O)c1nn2cccnc2c1-c1ccccc1 ; Cc1cc(C)nc(N2CC[C@@H](C)N(C(=O)c3nc4ccccn4c3-c3ccccc3)CC2)n1] 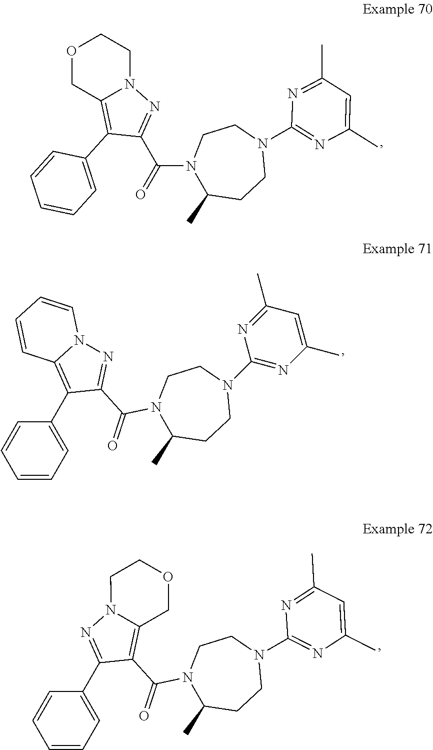 [SMILES: Cc1cc(C)nc(N2CC[C@@H](C)N(C(=O)c3nn4c(c3-c3ccccc3)COCC4)CC2)n1 ; Cc1cc(C)nc(N2CC[C@@H](C)N(C(=O)c3nn4ccccc4c3-c3ccccc3)CC2)n1 ; Cc1cc(C)nc(N2CC[C@@H](C)N(C(=O)c3c(-c4ccccc4)nn4c3COCC4)CC2)n1] 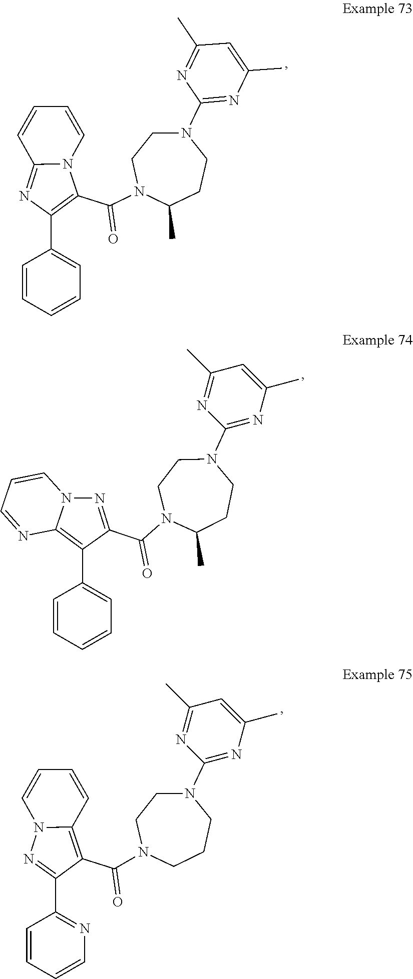 [SMILES: Cc1cc(C)nc(N2CC[C@@H](C)N(C(=O)c3c(-c4ccccc4)nc4ccccn34)CC2)n1 ; Cc1cc(C)nc(N2CC[C@@H](C)N(C(=O)c3nn4cccnc4c3-c3ccccc3)CC2)n1 ; Cc1cc(C)nc(N2CCCN(C(=O)c3c(-c4ccccn4)nn4ccccc34)CC2)n1] 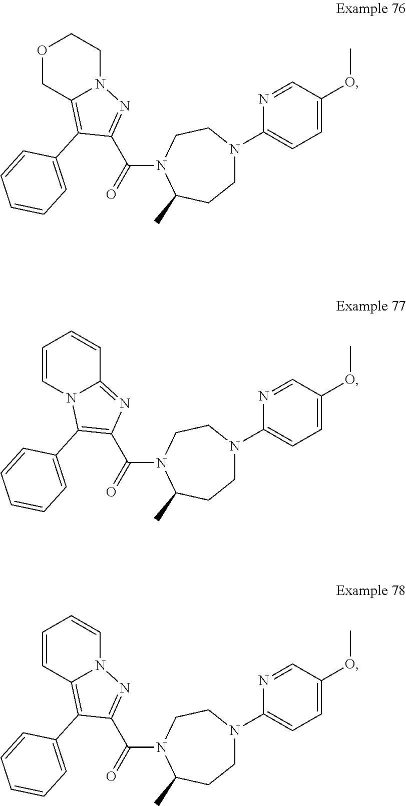 [SMILES: COc1ccc(N2CC[C@@H](C)N(C(=O)c3nn4c(c3-c3ccccc3)COCC4)CC2)nc1 ; COc1ccc(N2CC[C@@H](C)N(C(=O)c3nc4ccccn4c3-c3ccccc3)CC2)nc1 ; COc1ccc(N2CC[C@@H](C)N(C(=O)c3nn4ccccc4c3-c3ccccc3)CC2)nc1] 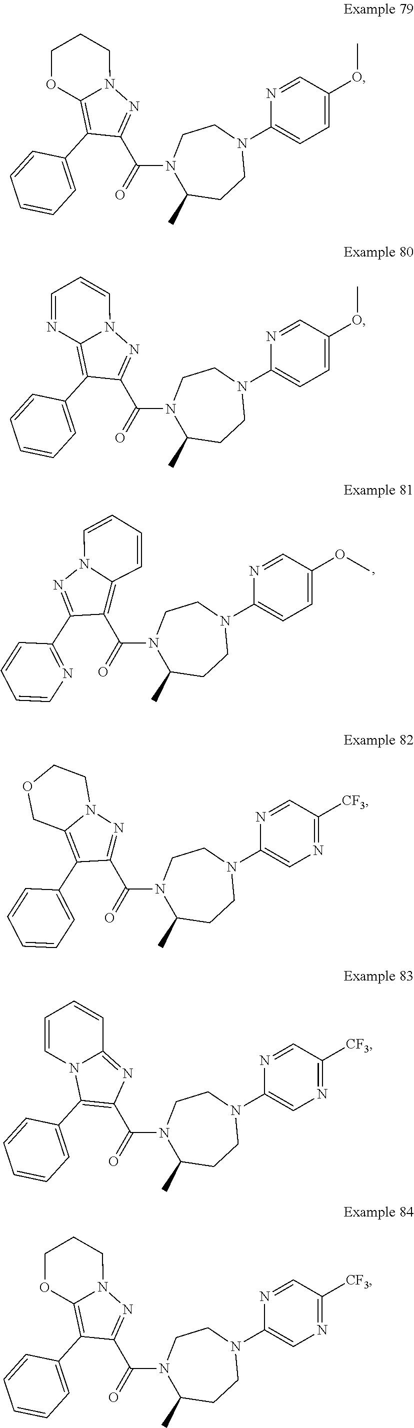 [SMILES: COc1ccc(N2CC[C@@H](C)N(C(=O)c3nn4c(c3-c3ccccc3)OCCC4)CC2)nc1 ; COc1ccc(N2CC[C@@H](C)N(C(=O)c3nn4cccnc4c3-c3ccccc3)CC2)nc1 ; COc1ccc(N2CC[C@@H](C)N(C(=O)c3c(-c4ccccn4)nn4ccccc34)CC2)nc1 ; C[C@@H]1CCN(c2cnc(C(F)(F)F)cn2)CCN1C(=O)c1nn2c(c1-c1ccccc1)COCC2 ; C[C@@H]1CCN(c2cnc(C(F)(F)F)cn2)CCN1C(=O)c1nc2ccccn2c1-c1ccccc1 ; C[C@@H]1CCN(c2cnc(C(F)(F)F)cn2)CCN1C(=O)c1nn2c(c1-c1ccccc1)OCCC2] 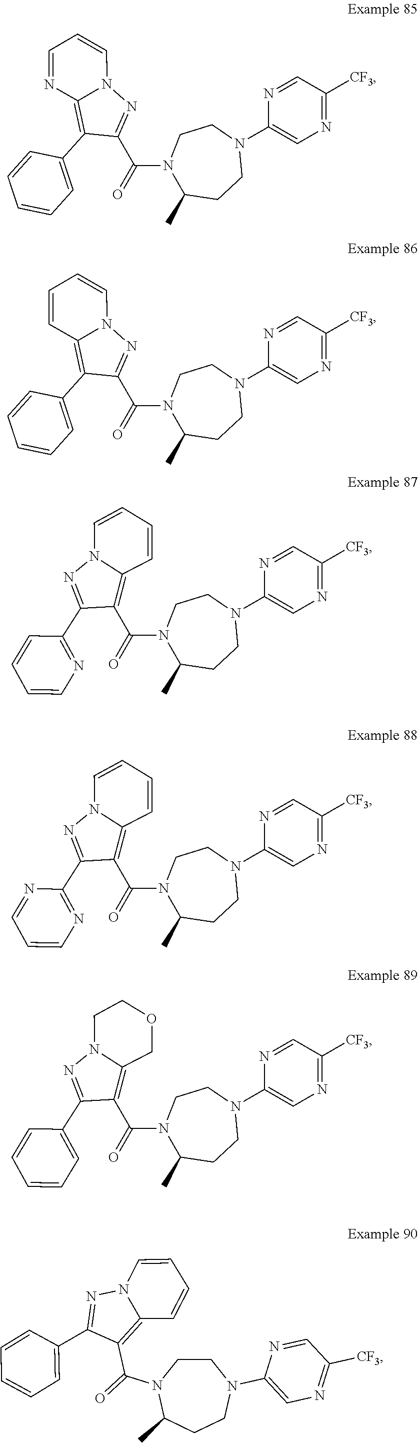 [SMILES: C[C@@H]1CCN(c2cnc(C(F)(F)F)cn2)CCN1C(=O)c1nn2cccnc2c1-c1ccccc1 ; C[C@@H]1CCN(c2cnc(C(F)(F)F)cn2)CCN1C(=O)c1nn2ccccc2c1-c1ccccc1 ; C[C@@H]1CCN(c2cnc(C(F)(F)F)cn2)CCN1C(=O)c1c(-c2ccccn2)nn2ccccc12 ; C[C@@H]1CCN(c2cnc(C(F)(F)F)cn2)CCN1C(=O)c1c(-c2ncccn2)nn2ccccc12 ; C[C@@H]1CCN(c2cnc(C(F)(F)F)cn2)CCN1C(=O)c1c(-c2ccccc2)nn2c1COCC2 ; C[C@@H]1CCN(c2cnc(C(F)(F)F)cn2)CCN1C(=O)c1c(-c2ccccc2)nn2ccccc12] 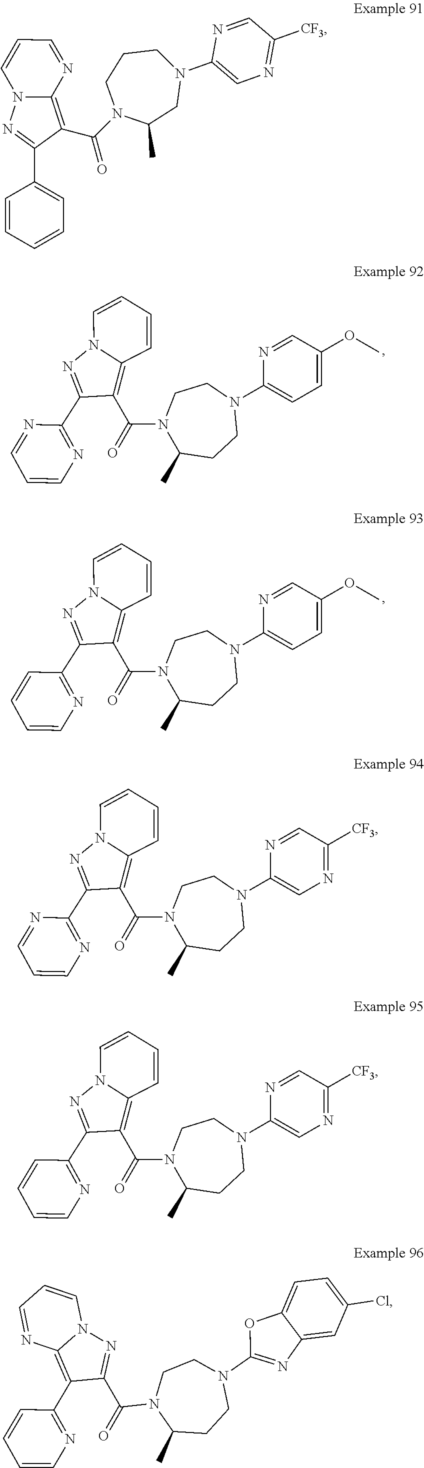 [SMILES: C[C@@H]1CN(c2cnc(C(F)(F)F)cn2)CCCN1C(=O)c1c(-c2ccccc2)nn2cccnc12 ; COc1ccc(N2CC[C@@H](C)N(C(=O)c3c(-c4ncccn4)nn4ccccc34)CC2)nc1 ; COc1ccc(N2CC[C@@H](C)N(C(=O)c3c(-c4ccccn4)nn4ccccc34)CC2)nc1 ; C[C@@H]1CCN(c2cnc(C(F)(F)F)cn2)CCN1C(=O)c1c(-c2ncccn2)nn2ccccc12 ; C[C@@H]1CCN(c2cnc(C(F)(F)F)cn2)CCN1C(=O)c1c(-c2ccccn2)nn2ccccc12 ; C[C@@H]1CCN(c2nc3cc(Cl)ccc3o2)CCN1C(=O)c1nn2cccnc2c1-c1ccccn1] 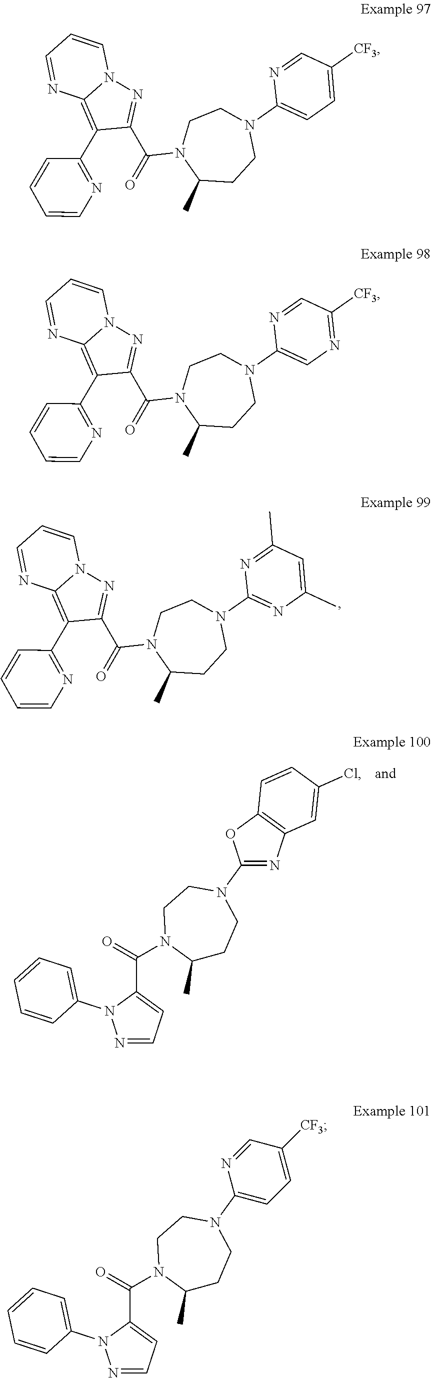 [SMILES: C[C@@H]1CCN(c2ccc(C(F)(F)F)cn2)CCN1C(=O)c1nn2cccnc2c1-c1ccccn1 ; C[C@@H]1CCN(c2cnc(C(F)(F)F)cn2)CCN1C(=O)c1nn2cccnc2c1-c1ccccn1 ; Cc1cc(C)nc(N2CC[C@@H](C)N(C(=O)c3nn4cccnc4c3-c3ccccn3)CC2)n1 ; C[C@@H]1CCN(c2nc3cc(Cl)ccc3o2)CCN1C(=O)c1ccnn1-c1ccccc1 ; "and" ; C[C@@H]1CCN(c2ccc(C(F)(F)F)cn2)CCN1C(=O)c1ccnn1-c1ccccc1]
or a pharmaceutically acceptable salt, hydrate, solvate, polymorph, isomer, or combination thereof.

6. A compound of claim 5 selected from the group consisting of 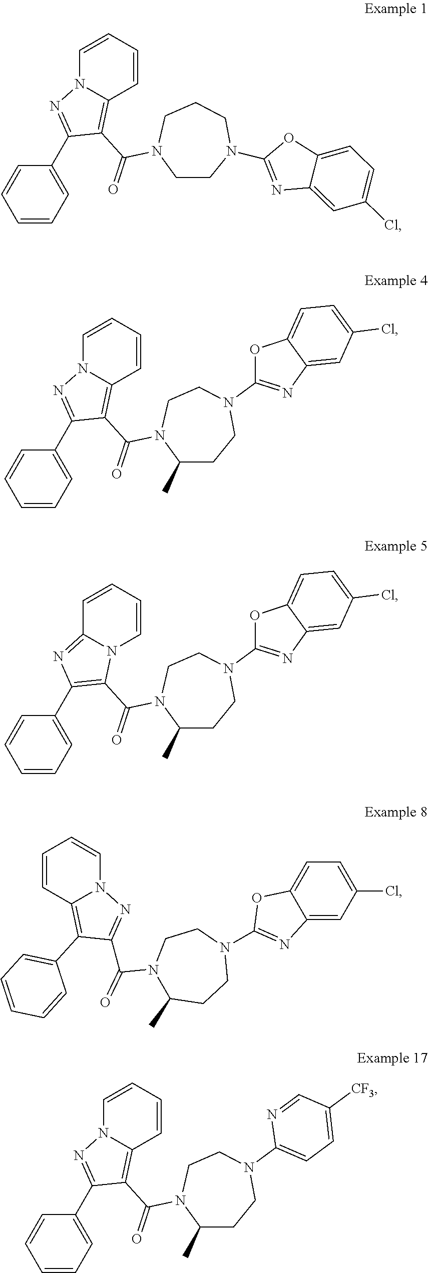 [SMILES: O=C(c1c(-c2ccccc2)nn2ccccc12)N1CCCN(c2nc3cc(Cl)ccc3o2)CC1 ; C[C@@H]1CCN(c2nc3cc(Cl)ccc3o2)CCN1C(=O)c1c(-c2ccccc2)nn2ccccc12 ; C[C@@H]1CCN(c2nc3cc(Cl)ccc3o2)CCN1C(=O)c1c(-c2ccccc2)nc2ccccn12 ; C[C@@H]1CCN(c2nc3cc(Cl)ccc3o2)CCN1C(=O)c1nn2ccccc2c1-c1ccccc1 ; C[C@@H]1CCN(c2ccc(C(F)(F)F)cn2)CCN1C(=O)c1c(-c2ccccc2)nn2ccccc12] 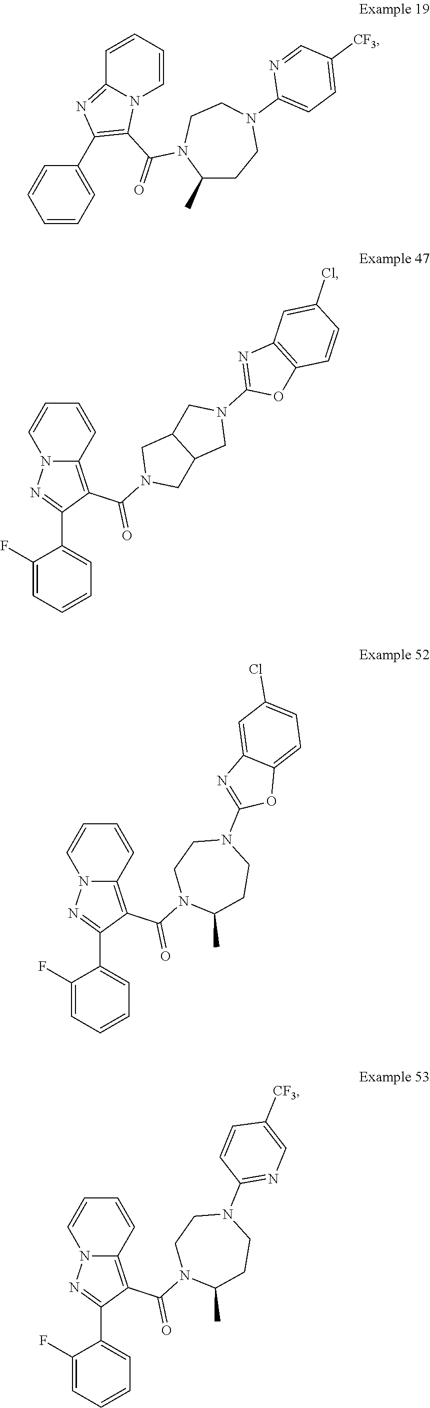 [SMILES: C[C@@H]1CCN(c2ccc(C(F)(F)F)cn2)CCN1C(=O)c1c(-c2ccccc2)nc2ccccn12 ; O=C(c1c(-c2ccccc2F)nn2ccccc12)N1CC2CN(c3nc4cc(Cl)ccc4o3)CC2C1 ; C[C@@H]1CCN(c2nc3cc(Cl)ccc3o2)CCN1C(=O)c1c(-c2ccccc2F)nn2ccccc12 ; C[C@@H]1CCN(c2ccc(C(F)(F)F)cn2)CCN1C(=O)c1c(-c2ccccc2F)nn2ccccc12] 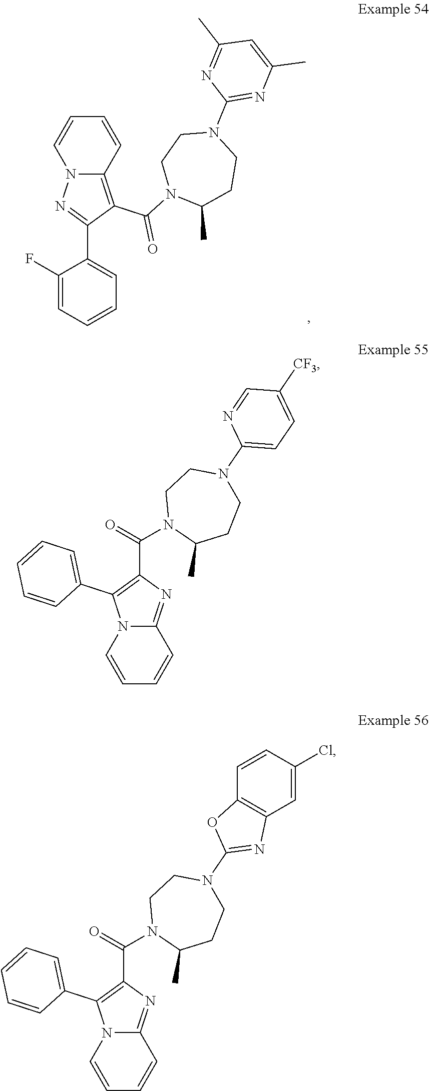 [SMILES: Cc1cc(C)nc(N2CC[C@@H](C)N(C(=O)c3c(-c4ccccc4F)nn4ccccc34)CC2)n1 ; C[C@@H]1CCN(c2ccc(C(F)(F)F)cn2)CCN1C(=O)c1nc2ccccn2c1-c1ccccc1 ; C[C@@H]1CCN(c2nc3cc(Cl)ccc3o2)CCN1C(=O)c1nc2ccccn2c1-c1ccccc1] 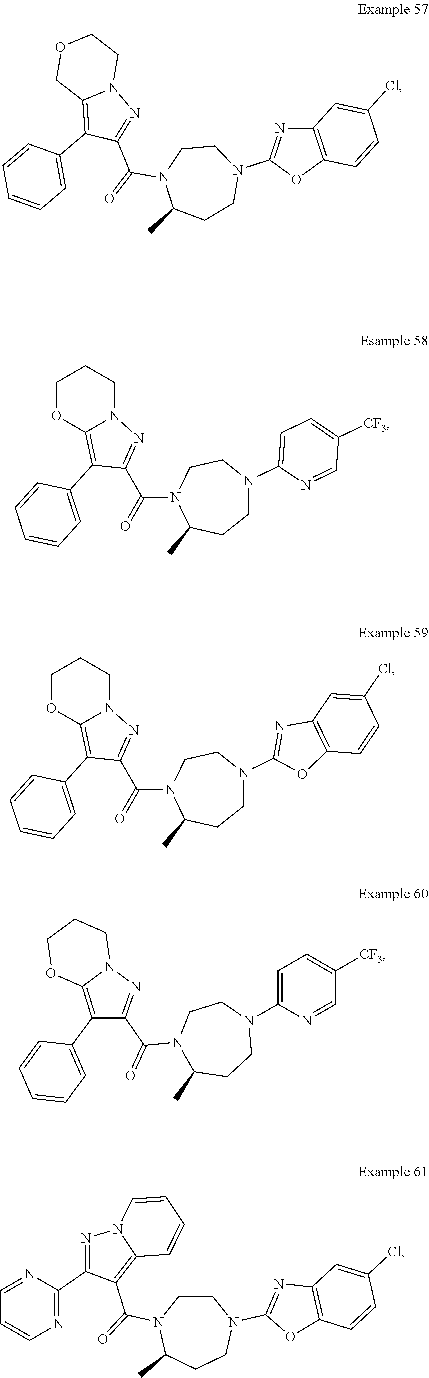 [SMILES: C[C@@H]1CCN(c2nc3cc(Cl)ccc3o2)CCN1C(=O)c1nn2c(c1-c1ccccc1)COCC2 ; C[C@@H]1CCN(c2ccc(C(F)(F)F)cn2)CCN1C(=O)c1nn2c(c1-c1ccccc1)OCCC2 ; C[C@@H]1CCN(c2nc3cc(Cl)ccc3o2)CCN1C(=O)c1nn2c(c1-c1ccccc1)OCCC2 ; C[C@@H]1CCN(c2ccc(C(F)(F)F)cn2)CCN1C(=O)c1nn2c(c1-c1ccccc1)OCCC2 ; C[C@@H]1CCN(c2nc3cc(Cl)ccc3o2)CCN1C(=O)c1c(-c2ncccn2)nn2ccccc12] 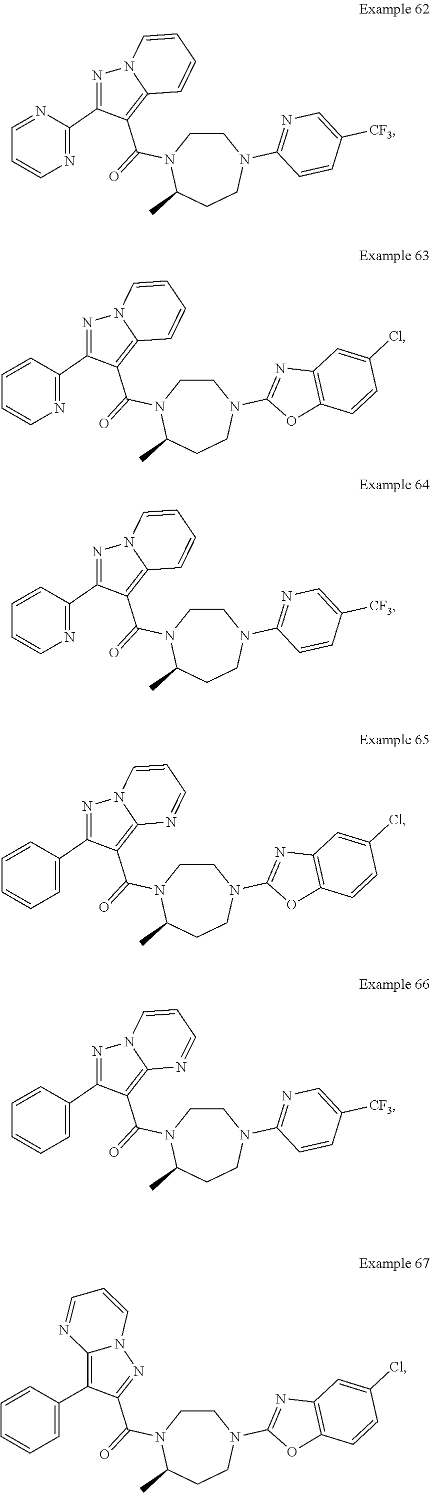 [SMILES: C[C@@H]1CCN(c2ccc(C(F)(F)F)cn2)CCN1C(=O)c1c(-c2ncccn2)nn2ccccc12 ; C[C@@H]1CCN(c2nc3cc(Cl)ccc3o2)CCN1C(=O)c1c(-c2ccccn2)nn2ccccc12 ; C[C@@H]1CCN(c2ccc(C(F)(F)F)cn2)CCN1C(=O)c1c(-c2ccccn2)nn2ccccc12 ; C[C@@H]1CCN(c2nc3cc(Cl)ccc3o2)CCN1C(=O)c1c(-c2ccccc2)nn2cccnc12 ; C[C@@H]1CCN(c2ccc(C(F)(F)F)cn2)CCN1C(=O)c1c(-c2ccccc2)nn2cccnc12 ; C[C@@H]1CCN(c2nc3cc(Cl)ccc3o2)CCN1C(=O)c1nn2cccnc2c1-c1ccccc1] 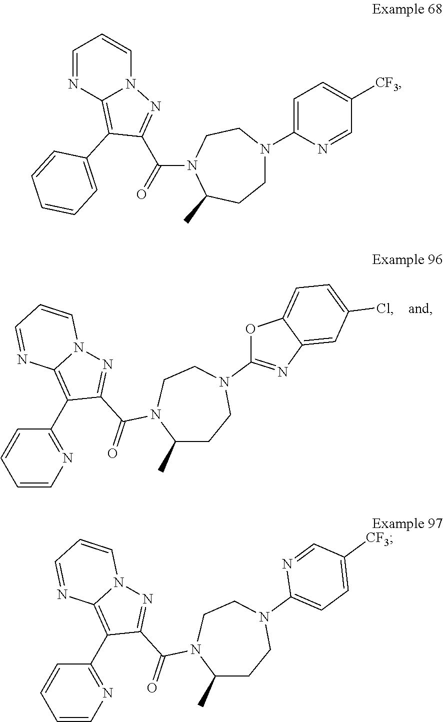 [SMILES: C[C@@H]1CCN(c2ccc(C(F)(F)F)cn2)CCN1C(=O)c1nn2cccnc2c1-c1ccccc1 ; C[C@@H]1CCN(c2nc3cc(Cl)ccc3o2)CCN1C(=O)c1nn2cccnc2c1-c1ccccn1 ; C[C@@H]1CCN(c2ccc(C(F)(F)F)cn2)CCN1C(=O)c1nn2cccnc2c1-c1ccccn1]
or a pharmaceutically acceptable salt, hydrate, solvate, polymorph, isomer, or combination thereof.

7. A pharmaceutical composition comprising a compound according to any preceding claim, or a pharmaceutically acceptable salt, hydrate, solvate, polymorph, isomer, or combination thereof; and an acceptable carrier, adjuvant or vehicle.

8. The pharmaceutical composition of claim 7 comprising a therapeutically effective amount of the compound, pharmaceutically acceptable salt, hydrate, solvate, polymorph, isomer, or combination thereof.

9. The pharmaceutical composition according to claim 7 or 8, wherein said composition further comprises at least one second therapeutic agent.

10. A method for antagonizing at least one orexin receptor in a cell, comprising the step of exposing the cell to a compound and/or composition according to any preceding claim, optionally wherein said method is in vitro.

11. A method for antagonizing at least one orexin receptor in a subject in need thereof, comprising the step of administering a compound and/or composition according to any preceding claim.

12. A method of treating a condition selected from the group consisting of substance addiction, substance dependence, panic, anxiety, depression, posttraumatic stress disorders (PTSD), neurodegeneration, autism, schizophrenia, pain, Alzheimer diseases (AD), and a central nervous system (CNS) disorder in a subject in need thereof, comprising the step of administering a compound and/or composition according to any preceding claim.

13. The method of claim 12 wherein the substance corresponding to the substance addiction or substance dependence is selected from the group consisting of one or more opioids, optionally heroin, morphine, oxycodone, fentanyl, and hydrocodone; one or more stimulants optionally selected from the group consisting of amphetamine, cocaine, crack cocaine, and methamphetamine; one or more sedatives and/or tranquilizers, benzodiazepine, and barbituates.

14. The method of any one of claims 10-13 wherein the compound is an orexin receptor antagonist.

15. The method of any one of claims 10-14 wherein the compound is an antagonist of orexin receptor 1 (OX₁R) or orexin receptor 2 (OX₂R), or an antagonist of both OX₁R or OX₂R; optionally wherein the compound is an antagonist of the K-opioid receptor; and/or exhibits the following characteristics as measured in an in vitro cellular assays: 1) OX₁R Kb<30 nm, OX₂R Kb>1000 nm, KOR Ki<500 nm; 2) OX₁R Kb<30 nm, OX₂R Kb>30 nm, KOR Ki<500 nm; or, 3) OX₁R Kb<30 nm, OX₂R Kb>1000 nm, KOR Ki>1000 nm.

16. A method of any one of claims 10-15 comprising administering to the patient a therapeutically effective amount of a compound and/or composition of any preceding claim.

17. A method of any one of claims 10-16 comprising administering at least one unlabeled form and/or at least one isotopically labeled form of compound of any preceding claim, and/or a composition comprising the same.

18. A method for manufacturing a compound of claim 1 comprising combining at least two intermediates to produce the compound.

19. A method for manufacturing a pharmaceutical composition of any of claims 7-9 comprising combining at least one compound of claim 1 with at least one pharmaceutically acceptable excipient.
